(19) United States
(12) Patent Application Publication
(10) Pub. No.: US 2024/0238264 A1
(43) Pub. Date: Jul. 18, 2024

(54) INHIBITORS OF SARM1 NADASE ACTIVITY AND USES THEREOF

(71) Applicants: Washington University, Saint Louis, MO (US); Disarm Therapeutics, Inc., Indianapolis, IN (US)

(72) Inventors: Jeffrey MILBRANDT, Saint Louis, MO (US); Kow ESSUMAN, Saint Louis, MO (US); Yo SASAKI, Brentwood, MO (US); Aaron DIANTONIO, Olivette, MO (US); Xianrong MAO, Saint Louis, MO (US); Rajesh DEVRAJ, Chesterfield, MO (US); Raul Eduardo KRAUSS, Chestnut Hill, MA (US); Robert Owen HUGHES, Newtown, CT (US)

(21) Appl. No.: 18/406,825

(22) Filed: Jan. 8, 2024

Related U.S. Application Data
(63) Continuation of application No. 17/508,321, filed on Oct. 22, 2021, now Pat. No. 11,903,935.
Application No. 17/508,321 is a continuation of application No. 16/335,754, filed on Mar. 22, 2019, now Pat. No. 11,253,503.
(60) Provisional application No. 62/473,921, filed on Mar. 20, 2017.
(60) Provisional application No. 62/473,916, filed on Mar. 20, 2017.
(60) Provisional application No. 62/473,805, filed on Mar. 20, 2017.
(60) Provisional application No. 62/399,339, filed on Sep. 24, 2016.

(51) Int. Cl. A61K 31/4439; A61K 31/047; A61K 31/145; A61K 31/198; A61K 31/41; A61K 31/437; A61K 31/4375; A61K 31/444; A61K 31/4535; A61K 31/566; A61K 31/7048; A61K 31/7084; A61K 33/243; A61K 33/245; A61K 33/28; A61K 33/30; A61K 47/52; A61K 47/55; A61P 25/28

(52) U.S. Cl. CPC A61K 31/4439; A61K 31/047; A61K 31/145; A61K 31/198; A61K 31/41; A61K 31/437; A61K 31/4375; A61K 31/444; A61K 31/4535; A61K 31/566; A61K 31/7048; A61K 31/7084; A61K 33/243; A61K 33/245; A61K 33/28; A61K 33/30; A61K 47/52; A61K 47/55; A61P 25/28

(57) ABSTRACT

The present disclosure provides compounds useful as inhibitors of SARM1 NADase activity, compositions thereof, and methods of using the same. The present disclosure provides compounds useful for treating a neurodegenerative or neurological disease or disorder, compositions thereof, and methods of using the same.

### CROSS REFERENCE TO RELATED APPLICATIONS

This application is a Continuation Patent Application of U.S. patent application Ser. No. 17/508,321, filed on Oct. 22, 2021, which is a Continuation Patent Application of U.S. application Ser. No. 16/335,754, filed on Mar. 22, 2019, now issued as U.S. Pat. No. 11,253,503, which is a U.S. National Entry claiming priority to International Application PCT/US2017/053098 filed on Sep. 22, 2017, which claims the benefit of U.S. Provisional Application 62/399,339, filed Sep. 24, 2016, U.S. Provisional Application No. 62/473,805, filed Mar. 20, 2017, U.S. Provisional Application No. 62/473,916, filed Mar. 20, 2017 and U.S. Provisional Application No. 62/473,921, filed Mar. 20, 2017, each of which is hereby incorporated by reference in its entirety.

### GOVERNMENT SUPPORT

This invention was made with government support under AG013730, NS065053 and NS087632 awarded by the National Institutes of Health. The government has certain rights in the invention.

### REFERENCE TO A SEQUENCE LISTING

The contents of the electronic sequence listing (sequencelisting.xml; Size: 44.558 bytes; and Date of Creation: Jan. 8, 2024) is herein incorporated by reference in its entirety.

### FIELD

This application relates to various compounds and compositions, and methods, useful for inhibition of SARM1 NADase activity and/or treating a neurodegenerative or neurological disease or disorder.

### BACKGROUND

Axonal degeneration is a hallmark of several neurological disorders including peripheral neuropathy, traumatic brain injury, and neurodegenerative diseases (Gerdts et al., SARM1 activation triggers axon degeneration locally via NAD(+) destruction. Science 348 2016, pp. 453-457, hereby incorporated by reference in its entirety). In Parkinson's disease and Amyotrophic Lateral Sclerosis, for example, axonal degeneration is an early event, preceding symptom onset and widespread neuronal loss (Kurowska et al., 2017; Fischer et al., Axonal degeneration in motor neuron disease Neurodegener. Dis. 4 2007 pp. 431-442; both of which are hereby incorporated by reference in their entireties).

### SUMMARY

In some embodiments, the present disclosure provides enzyme(s) as therapeutic target(s) for many neurological disorders that involve axon degeneration or axonopathy.

In certain embodiments, the present disclosure provides assays for identifying and/or characterizing SARM1 inhibitor. In some embodiments, the present disclosure provides certain vector constructs and polypeptides for use in these assays, including SAM-TIR in which the SARM1 N-terminal auto-inhibitory domain is deleted, as well as tagged versions of the TIR domain. In some embodiments, the present disclosure provides compositions comprising a polypeptide and a solid support which is used for screening SARM1 NADase inhibitors.

In some embodiments, the present disclosure provides methods of using SARM1 NADase inhibitors to treat, prevent or ameliorate axonal degeneration, axonopathies and neurological diseases and disorders that involve axonal degeneration. In some embodiments, the present disclosure provides inhibitors of SARM1 NADase. In some such embodiments, such compounds inhibit axonal degeneration, including axonal degeneration that results from reduction or depletion of NAD. In some embodiments, the present disclosure encompasses the recognition that Nicotinamide Hypoxanthine Dinucleotide (NHD) is useful as an inhibitor of SARM1 NADase activity.

In some embodiments, the present disclosure provides methods of treating a neuropathy or axonopathy associated with axonal degeneration. In some such embodiments, a neuropathy or axonopathy associated with axonal degeneration is selected from hereditary or congenital neuropathies or axonopathies. In some embodiments, a neuropathy or axonopathy associated with axonal degeneration is selected from or associated with Parkinson's disease, Alzheimer's disease, Herpes infection, diabetes, amyotrophic lateral sclerosis, a demyelinating disease, ischemia or stroke, chemical injury, thermal injury, and AIDS. In some embodiments, a neuropathy or axonopathy associated with axonal degeneration is selected from Parkinson's disease or non-Parkinson's diseases, and Alzheimer's disease.

It has now been found that compounds of this disclosure, and pharmaceutically acceptable compositions thereof, are effective as inhibitors of SARM1 NADase activity. In some embodiments, inhibitors of SARM1 NADase activity have the general formula I^{A} or formula I^{B}:

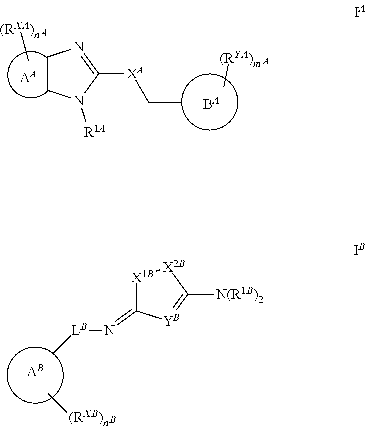

or a pharmaceutically acceptable salt thereof, wherein each variable is as defined and described herein.

In some embodiments, inhibitors of SARM1 NADase activity have the general formula I^{C} or formula I^{D}:

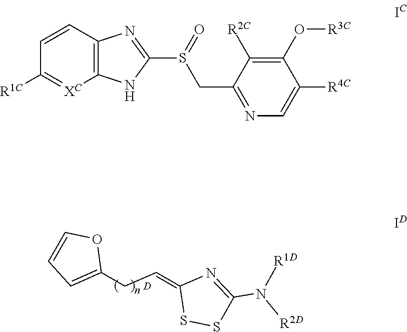

or a pharmaceutically acceptable salt thereof, wherein each variable is as defined and described herein.

In some embodiments, inhibitors of SARM1 NADase activity are selected from

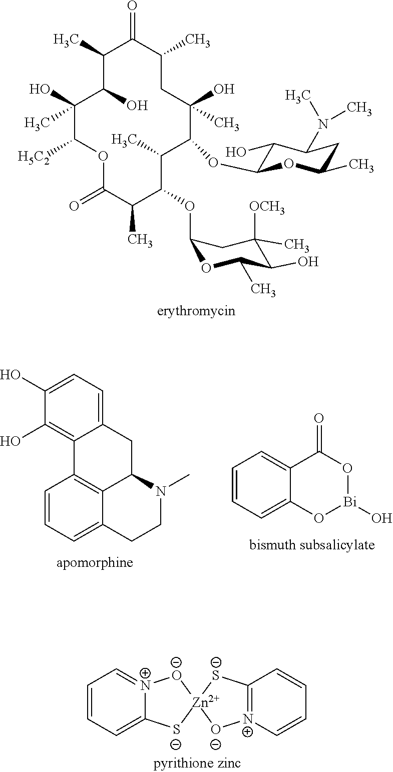

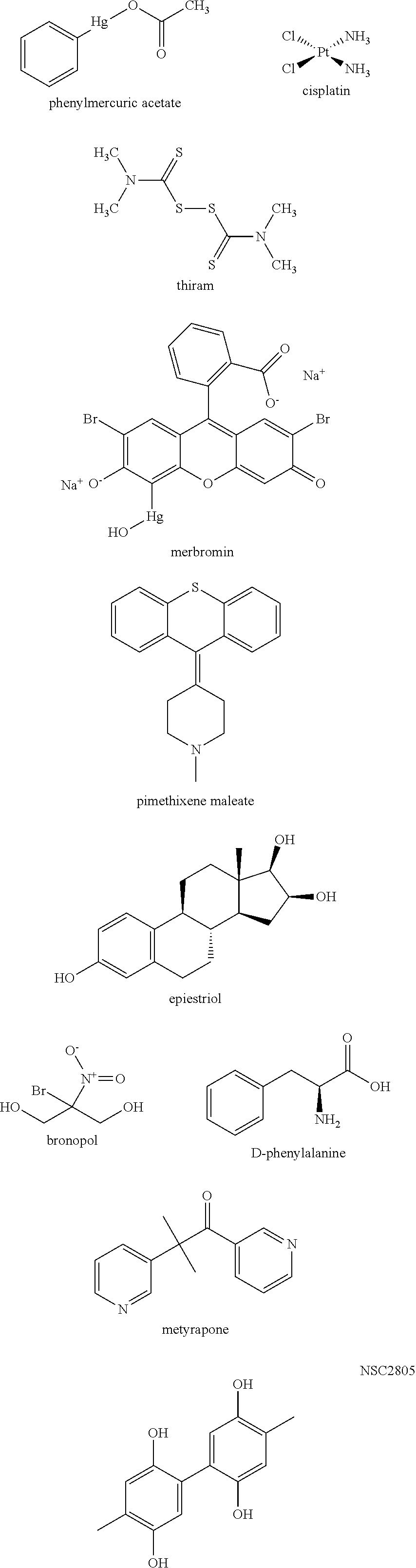

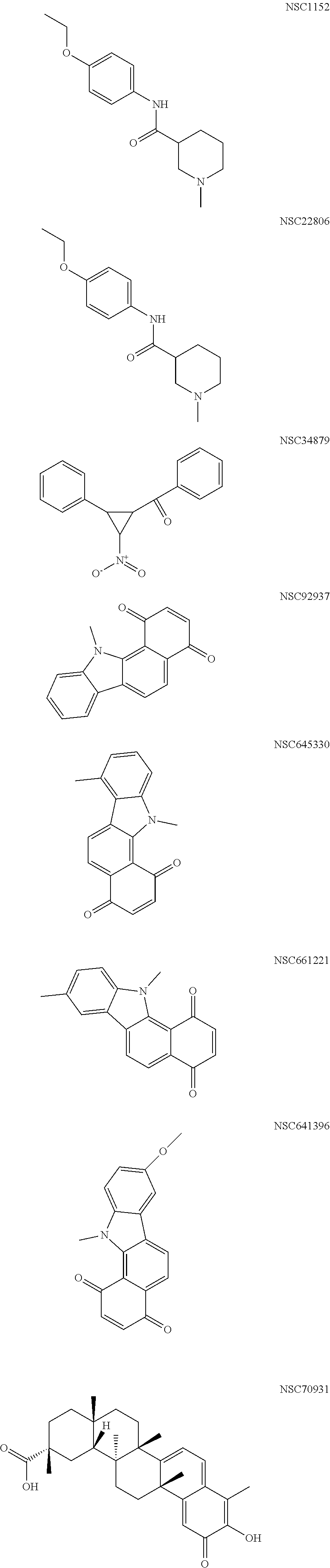

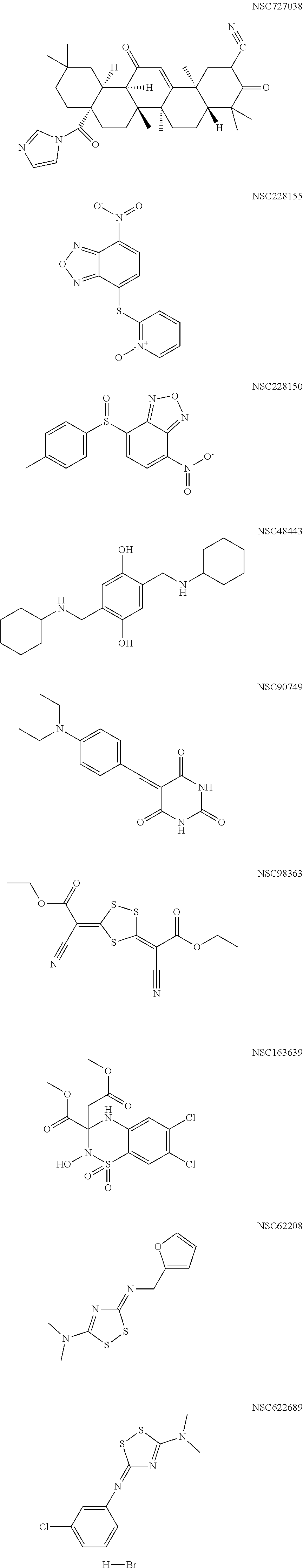

or a pharmaceutically acceptable salt thereof.

Compounds of the present disclosure, and pharmaceutically acceptable compositions thereof, are useful for treating a variety of diseases, disorders or conditions. Such diseases, disorders, or conditions include those described herein.

Compounds provided by this disclosure are also useful for the study of SARM1 NADase activity in biological and pathological phenomena; the study of intracellular signal transduction pathways occurring in lipogenic tissues; and the comparative evaluation of new SARM1 NADase activity inhibitors in vitro or in vivo.

### BRIEF DESCRIPTION OF THE FIGURES

FIG. 1 illustrates the structure of the SARM1 protein.

FIG. 2A-F illustrate that native SARM1-TIR protein complex cleaves NAD+ in an in vitro assay. FIG. 2A illustrates selected pathways of NAD+ synthesis and degradation. FIG. 2B illustrates a procedure for detecting NADase activity and its inhibition. FIG. 2C illustrates NADase activity of StrepTag-hSARM1-TIR. FIG. 2D illustrates that wild type SARM1-TIR complexes do not degrade NaAD. FIG. 2E illustrates an NAD+ reaction time course of human SARM1-TIR G601P, TLR4-TIR, and MyD88-TIR laden beads in in-vitro NADase assay (normalized to control at 0 min). FIG. 2F illustrates representative SYPRO Ruby gel of SARM1-TIR G601P, TLR4-TIR, and MyD88-TIR laden beads used in assay.

FIG. 3 illustrates that a NRK1-HEK293T stable line with NR supplementation maintains higher NAD+ levels upon SARM1-TIR expression.

FIG. 4A-E illustrates cleavage of NAD+ by a cell lysate comprising SARM1 SAM-TIR. FIG. 4A illustrates HPLC traces showing changes over time in levels of ADPR, NAM+ and NAD+. FIG. 4B illustrates HPLC traces showing that NADase activity is not exhibited in control lysates. FIG. 4C illustrates quantitative values of NAD+ and ADPR of HPLC traces of FIG. 4A. FIG. 4D illustrates dose-dependent cleavage of NAD+ by SARM1 SAM-TIR lysate.

FIG. 4E illustrates quantitation of NAD+/ADPR ratio by SAM-TIR lysate and control.

FIG. 5A-B illustrates a screen of candidate SAM-TIR NADase inhibitors from the NCI diversity IV compound library. FIG. 5A illustrates a primary screen of all 1600 compounds from the NCI diversity IV compound library. FIG. 5B illustrates re-testing of 20 positive “hits” from the primary screen.

FIG. 6A-C illustrates structures of 18 compounds that suppress SAM TIR NADase activity. NSC numbers are shown.

FIG. 7A-D illustrates an NAD+ cycling assay as an additional screening assay for SAM-TIR NADase inhibitors. FIG. 7A illustrates that SAM-TIR lysate (STL) but not control (con) lysate decreases NAD+, as determined by a NAD+ Glo assay. FIG. 7B illustrates robustness of the assay. FIG. 7C illustrates that most hits identified in the initial HPLC assay (14/18) showed significant inhibition of SAM-TIR NADase activity in a NAD+-Glo assay.

FIG. 7D illustrates NADase inhibitory activity of two compounds.

FIG. 8 illustrates in vitro NAD+ cleavage by SARM1 TIR protein expressed and purified from bacteria.

FIG. 9 illustrates that NAD+ consumption rate is increased after axotomy in wt axons.

FIG. 10A-C illustrates the effect of candidate inhibitors on axon degeneration. FIG. 10A illustrates testing of effects of compounds on axon degeneration index. FIG. 10B illustrates preventative effects of compound NSC622608 on axonal degeneration. FIG. 10C illustrates dose dependent inhibition of axon degeneration by compound NSC622608.

FIG. 11A-G illustrates that NAD+ cleavage enzymatic activity is intrinsic to SARM1-TIR. FIG. 11A illustrates endogenous NAD+ levels in bacteria after IPTG induction of human SARM1-TIR. FIG. 11B illustrates in vitro NAD+ cleavage reaction by human SARM1-TIR protein expressed and purified from bacteria. FIG. 1C illustrates that bacterially expressed mouse and zebrafish SARM1-TIR proteins cleave NAD+ in the in vitro assay. FIG. 11D illustrates a SYPRO Ruby gel of SARM1-TIR laden beads purified from bacteria used in NADase assay. FIG. 1E illustrates a time course of NAD+ cleavage reaction using bacterially synthesized human SARM1-TIR, purified by TAP, and subjected to 1M and 2M NaCl washes during purification (normalized to control at 0 min). FIG. 11F illustrates a time course of NAD+ cleavage reaction using bacterially synthesized human SARM1-TIR, purified by TAP, and subjected to either 0.5% Triton X-100 or 0.5% Tween-20 washes during purification (normalized to control at 0 min). FIG. 11G illustrates a reaction time course of purified components of the cell-free protein transcription/translation system incubated with NAD+ and non-recombinant plasmid.

FIGS. 12A-M illustrate characterization of the SARM1-TIR NAD+ cleavage reaction. FIGS. 12A-12E depict HPLC chromatograms showing NAD+ cleavage products of human and Drosophila SARM1-TIR. Retention time: Nam t˜2.40 min; cADPR at t˜0.85 min; ADPR at t˜1.10 min. FIGS. 12F-12G illustrate quantification of metabolites generated by human FIG. 12F and drosophila FIG. 12G SARM1-TIR as displayed in FIG. 12A-E (normalized to 0 min NAD+). FIG. 12H illustrates HPLC chromatograms showing that mouse and zebrafish SARM1-TIR NAD+ cleavage reaction generate Nam and ADPR as major products, and cADPR as a minor product. FIG. 12I illustrates that kinetic assays of the SARM1-TIR enzyme revealed saturation kinetics. FIG. 12J illustrates that ADPR does not inhibit SARM1-TIR NADase activity. FIG. 12K illustrates that Nam inhibits SARM1-TIR enzymatic activity. FIG. 12L illustrates Nam dose response inhibition of SARM1-TIR enzymatic activity. FIG. 12M illustrates SARM1 is the axonal NADase.

FIG. 13 illustrates that Nicotinamide Hypoxanthine dinucleotide (NHD) inhibits SARM1 TIR NAD+ cleavage.

FIG. 14 illustrates that Nicotinamide Hypoxanthine dinucleotide (NHD) is a substrate for the SARM1 TIR enzyme.

FIGS. 15A-B illustrate chemical structures of candidate analogs represented in the Table 1, Example 7. FIG. 15A illustrates NAD, NHD, and Etheno-NAD. FIG. 15B illustrates Ex-527, NADPH, Benzamide, Thionicotinamide, 6-aminonicotinamide, 3-acetylpyridine, and Nicotinaldehyde.

FIG. 16 illustrates Amino acid sequence alignment of SARM1-TIR with MilB Cytidine 5′ Monophosphate (CMP) Hydrolase. CMP catalytic glutamic acid is highlighted in red box and aligns to glutamic acid 642 in the SARM1-TIR domain.

FIG. 17 illustrates modeling of the SARM1-TIR domain on the crystal structure of CMP Hydrolase bound to CMP. E642 aligns with a catalytic residue of CMP Hydrolase.

FIG. 18 illustrates NAD+ reaction timecourse of human SARM1-TIR E642A purified from cell-free protein translation system (normalized to control at 0 min).

FIG. 19 illustrates a SYPRO Ruby gel of SARM1-TIR E642A purified from a cell-free protein translation system.

FIG. 20 illustrates Venus expression of indicated constructs in DRG axons, co-stained for Tuj1 to assess total axon area for each field.

FIG. 21 illustrates Venus expression of indicated constructs in DRG cell bodies, co-stained with Hoechst to assess total nuclei in each field.

FIG. 22 illustrates axonal NAD+ levels after axotomy (normalized to control at 0 hr). NC vector, SARM1 WT, and SARM1 E642A constructs were expressed in SARM1−/− DRG neurons, and levels of NAD+ were obtained at indicated timepoints after axotomy.

FIG. 23 illustrates axonal degeneration time course after axotomy, quantified as degeneration index (DI) where a DI of 0.35 (indicated by dotted line) or above represents degenerated axons.

FIG. 24 illustrates bright-field micrographs of axons expressing indicated constructs represented in FIG. 23.

FIG. 25 illustrates axonal degeneration time course after vincristine treatment, quantified as DI. Quantification data were generated from at least three independent biological experiments. Data are presented as mean±SEM; Error bars: SEM.*P<0.05,

**P<0.01, ***P<0.001 one-way ANOVA.

FIG. 26 illustrates bright-field micrographs of axons after vincristine treatment corresponding to selected groups in FIG. 25. Scale bar, 5 μm.

FIG. 27 illustrates selected pathways of NAD+ synthesis and degradation including SARM1 as a NAD+-consuming enzyme.

FIG. 28 illustrates SARM1-TIR NADase inhibition by members of the proton pump inhibitor family.

FIG. 29 shows a dose response curve for rabeprazole inhibition of SARM1-TIR NADase activity.

FIG. 30 shows a schematic of cell-free protein expression system.

FIG. 31 illustrates that human SARM1-TIR purified from a cell-free protein expression system cleaves NAD+ in NADase assay.

FIG. 32 illustrates a SYPRO Ruby gel of SARM1-TIR laden beads purified from a cell-free transcription/translation system.

FIGS. 33A and 33B depict the dose curves of SARM1 NADase activity inhibition by compounds I^{A}-2, I^{A}-3, I^{A}-6 and I^{A}-8, whereby FIG. 33A shows % control of NAD consumption and FIG. 33B shows % control of ADPR production.

FIG. 34 depicts dose curves of SARM1 NADase activity inhibition by compounds I^{B}-1 and I^{B}-2.

FIG. 35 depicts prevention of axonal degeneration by compound I^{B}-2 at intervals of 0 hours, 6 hours, 12 hours and 24 hours.

FIGS. 36A and 36B depict SEM micrographs of injured axons under degenerating conditions with (FIG. 36A) and without (FIG. 36B) exposure to compound I^{B}-2.

FIGS. 37A-37D depict results from an in vitro assay of full-length SARM1. FIG. 37A is a schematic showing SARM1 domains and changes including dimerization of the TIR1 domain after injury. AxD=axonal degeneration. FIG. 37B illustrates HPLC traces showing levels of ADPR, NAM and NAD. FIG. 37C shows relative NADase activity of full-length SARM1 vs that of an active SARM1 mutant (SAM-TIR). FIG. 37D shows relative NADase activity of full-length SARM1 versus that of catalytically inactive mutant (FL-MTS SARM1 (E642A)).

### DETAILED DESCRIPTION OF CERTAIN EMBODIMENTS

The Toll/Interleukin-1 receptor (TIR) domain is an evolutionarily conserved protein domain present in Toll-like receptors (TLR), and their cytosolic adaptor proteins, where as a scaffolding domain, it promotes innate immune signaling to protect hosts against invading pathogens (O'Neill, L. A., et al., Nat. Rev. Immunol., 2013, 13, 453-460). Sterile Alpha and TIR motif-containing 1 (SARM1) belongs to the family of cytosolic adaptor proteins, but is unique among its members because it is the most evolutionary ancient adaptor, paradoxically inhibits TLR signaling, and was recently identified as the central executioner of an injury-induced axon death pathway (O'Neill, L. A. & Bowie, A. G., Nat. Rev. Immunol., 2007, 7, 353-364; Osterloh, J. M., et al., Science, 2012, 337, 481-484; Gerdts, J., et al., J Neurosci. 33, 2013, 13569-13580). Activation of SARM1 by axonal injury or by enforced dimerization of the SARM1-TIR domain promotes the rapid and catastrophic depletion of Nicotinamide Adenine Dinucleotide (NAD+), which is followed soon after by axonal demise (Gerdts, J., et al., Science, 2015, 348, 453-457). Previous attempts to identify the NAD+ depleting enzyme(s) underlying this process were unsuccessful (Gerdts, J., et al., Science, 2015, 348, 453-457). Moreover, neither SARM1 nor TIR domains from other proteins have known enzymatic activity.

Damaged or unhealthy axons are eliminated via an intrinsic self-destruction program that is distinct from traditional cellular death pathways like apoptosis (Gerdts, J., et al., Neuron, 2016, 89, 449-460; Whitmore, A. V. et al., Cell Death Differ., 2003, 10, 260-261). Axon degeneration is a major component of several neurological diseases, such as but not limited to Alzheimer's disease, Parkinson's disease, ALS, Multiple sclerosis, diabetic peripheral neuropathy, chemotherapy-induced peripheral neuropathy, inherited neuropathy, traumatic brain injury, and glaucoma. Among pro-degenerative genes, SARM1 is the central executioner of the degenerative program. Loss of SARM1 blocks axon degeneration for weeks after injury (Osterloh, J. M., et al., Science, 2012, 337, 481-484; Gerdts, J., et al., J Neurosci., 2013, 33, 13569-13580) and also improves functional outcomes in mice after traumatic brain injury (Henninger, N. et al., Brain 139, 2016, 1094-1105). SARM1 is also required for axon degeneration in chemotherapy-induced peripheral neuropathy; loss of SARM1 blocks the development of chemotherapy-induced peripheral neuropathy, both halting axon degeneration and the development of heightened pain sensitivity after treatment with the chemotherapeutic vincristine (Geisler et al, Brain, 2016, 139, 3092-3108). Activation of SARM1 on the other hand, is sufficient to induce axon degeneration in the absence of injury (Gerdts, J., et al., Science, 2015, 348, 453-457). SARM1 also is required for axon degeneration in chemotherapy-induced peripheral neuropathy.

The activation of SARM1 leads to the catastrophic depletion of NAD+(Gerdts, J., et al., Science, 2015, 348, 453-457), thus highlighting the central role of NAD+ homeostasis in axonal integrity as first implied by studies with NMNAT1.

Despite these advances, the enzyme(s) underlying NAD+ breakdown in damaged axons remains unknown.

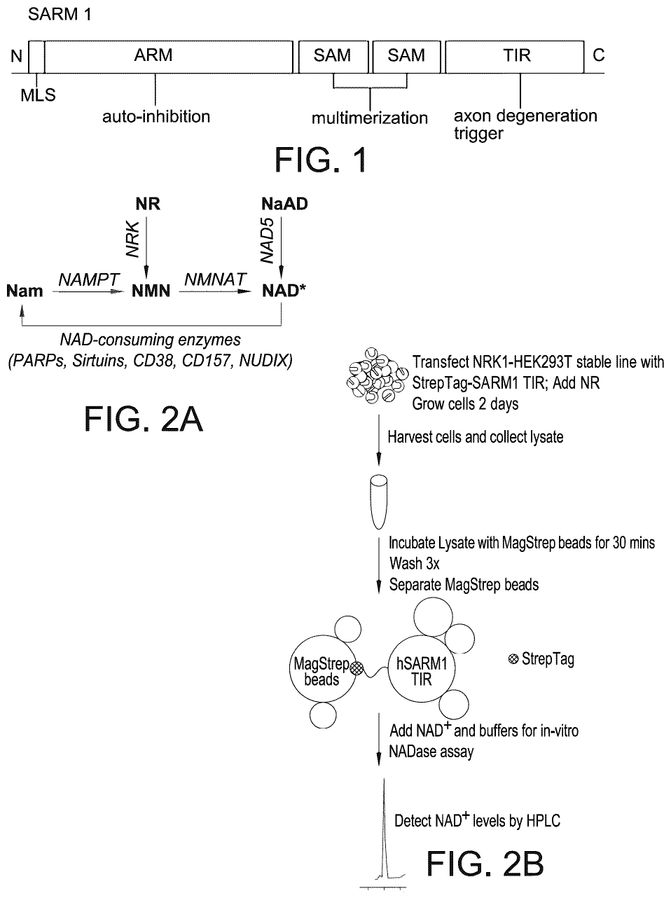

SARM1 contains multiple conserved motifs including SAM domains, ARM/HEAT motifs and a TIR domain (FIG. 1) that mediate oligomerization and protein-protein interactions (O'Neill, L. A. & Bowie, A. G., Nat. Rev. Immunol., 2007, 7, 353-364; Tewari, R., et al., Trends Cell Biol., 2010, 20, 470-481; Qiao, F. & Bowie, J. U., Sci. STKE 2005, re7, 2005). Dimerization of SARM1-TIR domains is sufficient to induce axonal degeneration and to rapidly trigger the degradation of NAD+, demonstrating that the NADase activity is either associated with or induced by dimerized SARM1-TIR domains. TIR domains are common in signaling proteins functioning in innate immunity pathways where they serve as scaffolds for protein complexes (O'Neill, L. A. & Bowie, A. G., Nat. Rev. Immunol., 2007, 7, 353-364).

Several groups have previously measured NAD+ and metabolites such as ADP ribose (ADPR) (for example, Hasan, M. A. et al., Korean J. Physiol. Pharmacol., 2014 18, 497-502; Breen, L. T., et al., Am. J. Physiol. Renal. Physiol., 2006, 290, F486-F495; and Li, P. L., et al., Am. J. Physiol. Heart Circ. Physiol., 2002, 282, H1229-H12236). However none of these groups have specifically done so in conjunction with SARM1 activity. In some embodiments, an ADPR as mentioned herein is a cADPR, e.g., a cyclic ADPR.

Loss of SARM1 blocks axonal degeneration for weeks after injury (Gerdts et al., Sarm1-mediated axon degeneration requires both SAM and TIR interactions J. Neurosci. 33 2013 pp. 13569-13580; Osterloh et al., 2012 both of which are hereby incorporated by reference in their entireties) and improves functional outcomes in mice after both traumatic brain injury (Henninger et al., 2016) and vincristine-induced peripheral neuropathy (Geisler et al., 2016). Axonal injury induces NAD+ loss (Wang et al., 2005), and SARM1 is required for this injury-induced NAD+ depletion both in vitro and in vivo (Gerdts et al., SARM1 activation triggers axon degeneration locally via NAD(+) destruction Science 348 2015 pp. 453-457; Sasaki et al., 2016; both of which are hereby incorporated by reference in their entireties). Moreover, activation of SARM1 signaling, via enforced dimerization of its TIR domain, is sufficient to induce axonal degeneration in the absence of injury due to a catastrophic depletion of axonal NAD+(Gerdts et al., SARM1 activation triggers axon degeneration locally via NAD(+) destruction Science 348 2015 pp. 453-457).

NAD+ is a dinucleotide that is essential for many redox reactions, but it is also consumed by a variety of enzymes (e.g., PARPs, CD38, Sirtuins) where the resulting metabolites influence signaling pathways via their effects on calcium mobilization or protein parylation (Cantó et al., 2015; Verdin, 2015). The identity of the NADase enzyme(s) responding to SARM1 activation and mediating NAD+ loss in injured axons has been unknown, although PARP1 and CD38 were previously eliminated as candidates (Gerdts et al., 2015; Sasaki et al., 2009). Furthermore, SARM1 is not known to have enzymatic activity, nor have TIR domains from any protein ever been associated with enzymatic activity. TIR domains are rather known for their scaffolding properties in Toll-like Receptor signaling, where they activate downstream enzymes to regulate pro-inflammatory and defense genes (O'Neill et al., 2013).

It has now been found, surprisingly, that the TIR domain of SARM1 acts as an enzyme to cleave NAD+, and that SARM1 enzymatic activity promotes axonal NAD+ depletion and axon degeneration after both traumatic and vincristine induced axonal injuries. The findings presented herein identify SARM1 enzymatic activity as novel therapeutic targets against diseases characterized by axonal degeneration including peripheral neuropathy, traumatic brain injury, and neurodegenerative diseases. More broadly, the findings presented herein show that TIR domains can possess intrinsic enzymatic activity.

### 1. General Description of Compounds of the Present Disclosure

In certain embodiments, the present disclosure provides a compound of formula I^{A}

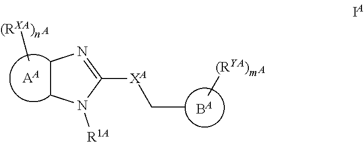

or a pharmaceutically acceptable salt thereof, wherein:
X^{A} is —S—, —SO— or —SO₂—;
R^{1A} is hydrogen, C₁₋₄ aliphatic, alkali metal, alkaline earth metal, ammonium or N⁺(C₁₋₄alkyl)₄;
Ring A^{A} is selected from a benzo fused ring and a 5-6 membered heteroaromatic fused ring having 1-3 heteroatoms independently selected from nitrogen, oxygen, and sulfur;
Ring B^{A} is selected from phenyl, an 8-10 membered bicyclic aromatic carbocyclic ring, a 4-8 membered saturated or partially unsaturated monocyclic heterocyclic ring having 1-2 heteroatoms independently selected from nitrogen, oxygen, or sulfur, a 5-6 membered monocyclic heteroaromatic ring having 1-4 heteroatoms independently selected from nitrogen, oxygen, or sulfur, or an 8-10 membered bicyclic heteroaromatic ring having 1-5 heteroatoms independently selected from nitrogen, oxygen, or sulfur;
R^{XA} and R^{YA} are independently hydrogen, C₁₋₄ aliphatic optionally substituted with 1-4 halogen, —OR^{A}, —SR^{A}, —N(R^{A})₂, —N(R^{A})C(O)R^{A}, —C(O)N(R^{A})₂, —N(R^{A})C(O)N(R^{A})₂, —N(R^{A})C(O)OR^{A}, —OC(O)N(R^{A})₂, —N(R^{A})S(O)₂R^{A}, —S(O)₂N(R^{A})₂, —C(O)R^{A}, —C(O)OR^{A}, —OC(O)R^{A}, —S(O)R^{A}, —S(O)₂R^{A}, phenyl, an 8-10 membered bicyclic aromatic carbocyclic ring, a 4-8 membered saturated or partially unsaturated monocyclic heterocyclic ring having 1-2 heteroatoms independently selected from nitrogen, oxygen, or sulfur, a 5-6 membered monocyclic heteroaromatic ring having 1-4 heteroatoms independently selected from nitrogen, oxygen, or sulfur, or an 8-10 membered bicyclic heteroaromatic ring having 1-5 heteroatoms independently selected from nitrogen, oxygen, or sulfur;
each R^{A} is independently hydrogen or an optionally substituted group selected from C₁₋₆ aliphatic, a 3-8 membered saturated or partially unsaturated monocyclic carbocyclic ring, phenyl, an 8-10 membered bicyclic aromatic carbocyclic ring, a 4-8 membered saturated or partially unsaturated monocyclic heterocyclic ring having 1-2 heteroatoms independently selected from nitrogen, oxygen, or sulfur, a 5-6 membered monocyclic heteroaromatic ring having 1-4 heteroatoms independently selected from nitrogen, oxygen, or sulfur, or an 8-10 membered bicyclic heteroaromatic ring having 1-5 heteroatoms independently selected from nitrogen, oxygen, or sulfur;
m^{A} and n^{A} are independently 0, 1, 2, or 3.

In certain embodiments, the present disclosure provides a compound of formula I^{B}.

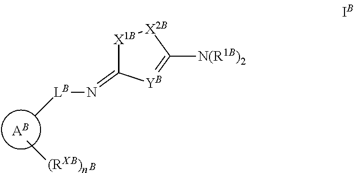

or a pharmaceutically acceptable salt thereof, wherein:
X^{1B} and X^{2B} are independently —O—, —S—, or —NR^{B}—, provided that one of X^{1B} and X^{2B} is —O— or —S— and both of X^{1B} and X^{2B} are not —O—;
Y^{B} is —N— or —CH—;
each R^{1B} is independently hydrogen or optionally substituted C₁₋₄ aliphatic;
Ring A^{B} is selected from phenyl, an 8-10 membered bicyclic aromatic carbocyclic ring, a 4-8 membered saturated or partially unsaturated monocyclic heterocyclic ring having 1-2 heteroatoms independently selected from nitrogen, oxygen, or sulfur, a 5-6 membered monocyclic heteroaromatic ring having 1-4 heteroatoms independently selected from nitrogen, oxygen, or sulfur, or an 8-10 membered bicyclic heteroaromatic ring having 1-5 heteroatoms independently selected from nitrogen, oxygen, or sulfur;
each R^{XB} is independently hydrogen, halogen or an optionally substituted group selected from C₁₋₆ aliphatic, a 3-8 membered saturated or partially unsaturated monocyclic carbocyclic ring, phenyl, an 8-10 membered bicyclic aromatic carbocyclic ring, a 4-8 membered saturated or partially unsaturated monocyclic heterocyclic ring having 1-2 heteroatoms independently selected from nitrogen, oxygen, or sulfur, a 5-6 membered monocyclic heteroaromatic ring having 1-4 heteroatoms independently selected from nitrogen, oxygen, or sulfur, or an 8-10 membered bicyclic heteroaromatic ring having 1-5 heteroatoms independently selected from nitrogen, oxygen, or sulfur;
each R^{B} is independently hydrogen or an optionally substituted group selected from C₁₋₆ aliphatic, a 3-8 membered saturated or partially unsaturated monocyclic carbocyclic ring, phenyl, an 8-10 membered bicyclic aromatic carbocyclic ring, a 4-8 membered saturated or partially unsaturated monocyclic heterocyclic ring having 1-2 heteroatoms independently selected from nitrogen, oxygen, or sulfur, a 5-6 membered monocyclic heteroaromatic ring having 1-4 heteroatoms independently selected from nitrogen, oxygen, or sulfur, or an 8-10 membered bicyclic heteroaromatic ring having 1-5 heteroatoms independently selected from nitrogen, oxygen, or sulfur;
L^{B} is a covalent bond, a C₁₋₆ membered straight or branched bivalent hydrocarbon chain, cyclopropylenyl, cyclobutylenyl, or oxetanylenyl; and
n^{B} is 0, 1, 2, 3 or 4.

### 2. Compounds and Definitions

Compounds of this disclosure include those described generally above, and are further illustrated by the classes, subclasses, and species disclosed herein. As used herein, the following definitions shall apply unless otherwise indicated. For purposes of this disclosure, the chemical elements are identified in accordance with the Periodic Table of the Elements, CAS version, Handbook of Chemistry and Physics, 75^{th} Ed. Additionally, general principles of organic chemistry are described in “Organic Chemistry”, Thomas Sorrell, University Science Books, Sausalito: 1999, and “March's Advanced Organic Chemistry”, 5^{th} Ed., Ed.: Smith, M. B. and March, J., John Wiley & Sons, New York: 2001, the entire contents of which are hereby incorporated by reference.

The term “aliphatic” or “aliphatic group”, as used herein, means a straight-chain (i.e., unbranched) or branched, substituted or unsubstituted hydrocarbon chain that is completely saturated or that contains one or more units of unsaturation, or a monocyclic hydrocarbon or bicyclic hydrocarbon that is completely saturated or that contains one or more units of unsaturation, but which is not aromatic (also referred to herein as “carbocycle,” “cycloaliphatic” or “cycloalkyl”), that has a single point of attachment to the rest of the molecule. Unless otherwise specified, aliphatic groups contain 1-6 aliphatic carbon atoms. In some embodiments, aliphatic groups contain 1-5 aliphatic carbon atoms. In other embodiments, aliphatic groups contain 1-4 aliphatic carbon atoms. In still other embodiments, aliphatic groups contain 1-3 aliphatic carbon atoms, and in yet other embodiments, aliphatic groups contain 1-2 aliphatic carbon atoms. In some embodiments, “cycloaliphatic” (or “carbocycle” or “cycloalkyl”) refers to a monocyclic C₃-C₆ hydrocarbon that is completely saturated or that contains one or more units of unsaturation, but which is not aromatic, that has a single point of attachment to the rest of the molecule. Suitable aliphatic groups include, but are not limited to, linear or branched, substituted or unsubstituted alkyl, alkenyl, alkynyl groups and hybrids thereof such as (cycloalkyl)alkyl, (cycloalkenyl)alkyl or (cycloalkyl)alkenyl.

As used herein, the term “alkyl” refers to a straight-chain (i.e., unbranched) or branched, substituted or unsubstituted hydrocarbon chain that is completely saturated, or a monocyclic hydrocarbon or bicyclic hydrocarbon that is completely saturated (also referred to herein as “cycloalkyl”) and which has a single point of attachment to the rest of the molecule. Unless otherwise specified, alkyl groups contain 1-6 carbon atoms. In some embodiments, alkyl groups contain 1-5 carbon atoms. In other embodiments, alkyl groups contain 1-4 carbon atoms. In still other embodiments, alkyl groups contain 1-3 carbon atoms, and in yet other embodiments, alkyl groups contain 1-2 carbon atoms. In some embodiments, “cycloalkyl” refers to a monocyclic C₃-C₆ hydrocarbon that is completely saturated and has a single point of attachment to the rest of the molecule.

The term “lower alkyl” refers to a C₁₋₄ straight or branched alkyl group. Exemplary lower alkyl groups are methyl, ethyl, propyl, isopropyl, butyl, isobutyl, and tert-butyl.

The term “lower haloalkyl” refers to a C₁₋₄ straight or branched alkyl group that is substituted with one or more halogen atoms.

The term “heteroatom” means one or more of oxygen, sulfur, nitrogen, phosphorus, or silicon (including, any oxidized form of nitrogen, sulfur, phosphorus, or silicon; the quaternized form of any basic nitrogen or; a substitutable nitrogen of a heterocyclic ring, for example N (as in 3,4-dihydro-2H-pyrrolyl), NH (as in pyrrolidinyl) or NR⁺ (as in N-substituted pyrrolidinyl)).

The term “unsaturated,” as used herein, means that a moiety has one or more units of unsaturation.

As used herein, the term “bivalent C₁₋₈ (or C₁₋₆) saturated or unsaturated, straight or branched, hydrocarbon chain”, refers to bivalent alkylene, alkenylene, and alkynylene chains that are straight or branched as defined herein.

The term “alkylene” refers to a bivalent alkyl group. An “alkylene chain” is a polymethylene group, i.e., —(CH₂)ₙ—, wherein n is a positive integer, preferably from 1 to 6, from 1 to 4, from 1 to 3, from 1 to 2, or from 2 to 3. A substituted alkylene chain is a polymethylene group in which one or more methylene hydrogen atoms are replaced with a substituent. Suitable substituents include those described below for a substituted aliphatic group.

The term “alkenylene” refers to a bivalent alkenyl group. A substituted alkenylene chain is a polymethylene group containing at least one double bond in which one or more hydrogen atoms are replaced with a substituent. Suitable substituents include those described below for a substituted aliphatic group.

The term “halogen” means F, Cl, Br, or I.

The term “aryl” used alone or as part of a larger moiety as in “aralkyl,” “aralkoxy,” or “aryloxyalkyl,” refers to monocyclic or bicyclic ring systems having a total of five to fourteen ring members, wherein at least one ring in the system is aromatic and wherein each ring in the system contains 3 to 7 ring members. The term “aryl” may be used interchangeably with the term “aryl ring.” In some embodiments, the term “aryl” refers to a monocyclic or bicyclic ring system having a total of five to ten ring members, wherein at least one ring in the system is aromatic and wherein each ring in the system contains three to seven ring members. In certain embodiments of the present disclosure, “aryl” refers to an aromatic ring system which includes, but not limited to, phenyl, biphenyl, naphthyl, anthracyl and the like, which may bear one or more substituents. Also included within the scope of the term “aryl,” as it is used herein, is a group in which an aromatic ring is fused to one or more non-aromatic rings, such as indanyl, phthalimidyl, naphthimidyl, phenanthridinyl, or tetrahydronaphthyl, and the like.

The terms “heteroaryl” and “heteroar-,” used alone or as part of a larger moiety, e.g., “heteroaralkyl,” or “heteroaralkoxy,” refer to groups having 5 to 10 ring atoms, preferably 5, 6, or 9 ring atoms; having 6, 10, or 14 π electrons shared in a cyclic array; and having, in addition to carbon atoms, from one to five heteroatoms selected from nitrogen, oxygen and sulfur. For instance, heteroaryl may refer to a 5-6 membered monocyclic heteroaromatic ring having 1-4 heteroatoms independently selected from nitrogen, oxygen, or sulfur, or an 8-10 membered bicyclic heteroaromatic ring having 1-5 heteroatoms independently selected from nitrogen, oxygen, or sulfur. Heteroaryl groups include, without limitation, thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, indolizinyl, purinyl, naphthyridinyl, and pteridinyl. The terms “heteroaryl” and “heteroar-”, as used herein, also include groups in which a heteroaromatic ring is fused to one or more aryl, cycloaliphatic, or heterocyclyl rings, where the radical or point of attachment is on the heteroaromatic ring. Nonlimiting examples include indolyl, isoindolyl, benzothienyl, benzofuranyl, dibenzofuranyl, indazolyl, benzimidazolyl, benzthiazolyl, quinolyl, isoquinolyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydroquinolinyl, tetrahydroisoquinolinyl, and pyrido[2,3-b]-1,4-oxazin-3(4H)-one. A heteroaryl group may be mono- or bicyclic. The term “heteroaryl” may be used interchangeably with the terms “heteroaryl ring,” “heteroaryl group,” or “heteroaromatic,” any of which terms include rings that are optionally substituted. The term “heteroaralkyl” refers to an alkyl group substituted by a heteroaryl, wherein the alkyl and heteroaryl portions independently are optionally substituted.

As used herein, the terms “heterocycle,” “heterocyclyl,” “heterocyclic radical,” and “heterocyclic ring” are used interchangeably and refer to a stable 5- to 7-membered monocyclic or 7-10-membered bicyclic heterocyclic moiety that is either saturated or partially unsaturated, and having, in addition to carbon atoms, one or more, preferably one to four, heteroatoms, as defined above. When used in reference to a ring atom of a heterocycle, the term “nitrogen” includes a substituted nitrogen. As an example, in a saturated or partially unsaturated ring having 0-3 heteroatoms selected from oxygen, sulfur or nitrogen, the nitrogen may be N (as in 3,4-dihydro-2H-pyrrolyl), NH (as in pyrrolidinyl), or ⁺NR (as in N-substituted pyrrolidinyl).

A heterocyclic ring can be attached to its pendant group at any heteroatom or carbon atom that results in a stable structure and any of the ring atoms can be optionally substituted. Examples of such saturated or partially unsaturated heterocyclic radicals include, without limitation, tetrahydrofuranyl, tetrahydrothiophenyl pyrrolidinyl, piperidinyl, pyrrolinyl, tetrahydroquinolinyl, tetrahydroisoquinolinyl, decahydroquinolinyl, oxazolidinyl, piperazinyl, dioxanyl, dioxolanyl, diazepinyl, oxazepinyl, thiazepinyl, morpholinyl, and quinuclidinyl. The terms “heterocycle,” “heterocyclyl,” “heterocyclyl ring,” “heterocyclic group,” “heterocyclic moiety,” and “heterocyclic radical,” are used interchangeably herein, and also include groups in which a heterocyclyl ring is fused to one or more aryl, heteroaryl, or cycloaliphatic rings, such as indolinyl, 3H-indolyl, chromanyl, phenanthridinyl, or tetrahydroquinolinyl, where the radical or point of attachment is on the heterocyclyl ring. A heterocyclyl group may be mono- or bicyclic. The term “heterocyclylalkyl” refers to an alkyl group substituted by a heterocyclyl, wherein the alkyl and heterocyclyl portions independently are optionally substituted.

As used herein, the term “measurably inhibit” refers to a measurable change in SARM1 NADase activity between a sample comprising a provided compound or composition, and SARM1 NADase and an equivalent sample comprising SARM1 NADase in the absence of a provided composition or composition. In some embodiments, a compound or composition “measurably inhibits” SARM1 NADase activity by at least 2-fold, 3-fold, 4-fold, or greater as compared to the control. In some embodiments, a compound or composition “measurably inhibits” SARM1 NADase activity by at least 10%, 20%, 25%, 50%, 75% or more as compared to control.

As used herein, the term “partially unsaturated” refers to a ring moiety that includes at least one double or triple bond. The term “partially unsaturated” is intended to encompass rings having multiple sites of unsaturation, but is not intended to include aryl or heteroaryl moieties, as herein defined.

As described herein, compounds of the present disclosure may contain “optionally substituted” moieties. In general, the term “substituted,” whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this disclosure are preferably those that result in the formation of stable or chemically feasible compounds. The term “stable,” as used herein, refers to compounds that are not substantially altered when subjected to conditions to allow for their production, detection, and, in certain embodiments, their recovery, purification, and use for one or more of the purposes disclosed herein.

Suitable monovalent substituents on a substitutable carbon atom of an “optionally substituted” group are independently halogen; —(CH₂)₀₋₄R^{∘}; —(CH₂)₀₋₄OR^{∘}; —O(CH₂)₀₋₄R^{∘}, —O—(CH₂)₀₋₄C(O)OR^{∘}; —(CH₂)₀₋₄CH(OR^{∘})₂; —(CH₂)₀₋₄SR^{∘}; —(CH₂)₀₋₄Ph, which may be substituted with R^{∘}; —(CH₂)₀₋₄O(CH₂)₀₋₁Ph which may be substituted with R^{∘}; —CH═CHPh, which may be substituted with R^{∘}; —(CH₂)₀₋₄O(CH₂)₀₋₁-pyridyl which may be substituted with R^{∘}; —NO₂; —CN; —N₃; —(CH₂)₀₋₄N(R^{∘})₂; —(CH₂)₀₋₄N(R^{∘})C(O)R^{∘}; —N(R^{∘})C(S)R^{∘}; —(CH₂)₀₋₄N(R^{∘})C(O)NR^{∘}₂; —N(R^{∘})C(S)NR^{∘}₂; —(CH₂)₀₋₄N(R^{∘})C(O)OR^{∘}; —N(R^{∘})N(R^{∘})C(O)R^{∘}; —N(R^{∘})N(R^{∘})C(O)NR^{∘}₂; —N(R^{∘})N(R^{∘})C(O)OR^{∘}; —(CH₂)₀₋₄C(O)R^{∘}; —C(S)R^{∘}; —(CH₂)₀₋₄C(O)OR^{∘}; —(CH₂)₀₋₄C(O)SR^{∘}; —(CH₂)₀₋₄C(O)OSiR^{∘}₃; —(CH₂)₀₋₄OC(O)R^{∘}; —OC(O)(CH₂)₀₋₄SR^{∘}—; —(CH₂)₀₋₄SC(O)R^{∘}; —(CH₂)₀₋₄C(O)NR^{∘}₂; —C(S)NR^{∘}₂; —C(S)SR^{∘}; —SC(S)SR^{∘}, —(CH₂)₀₋₄OC(O)NR^{∘}₂; —C(O)N(OR^{∘})R^{∘}; —C(O)C(O)R^{∘}; —C(O)CH₂C(O)R^{∘}; —C(NOR^{∘})R^{∘}; —(CH₂)₀₋₄SSR^{∘}; —(CH₂)₀₋₄S(O)₂R^{∘}; —(CH₂)₀₋₄S(O)₂OR^{∘}; —(CH₂)₀₋₄OS(O)₂R^{∘}; —S(O)₂NR^{∘}₂; —(CH₂)₀₋₄S(O)R^{∘}; —N(R^{∘})S(O)₂NR^{∘}₂; —N(R^{∘})S(O)₂R^{∘}; —N(OR^{∘})R^{∘}; —C(NH)NR^{∘}₂; —P(O)₂R^{∘}; —P(O)R^{∘}₂; —OP(O)R^{∘}₂; —OP(O)(OR^{∘})₂; SiR^{∘}₃; —(C₁₋₄ straight or branched alkylene)O—N(R^{∘})₂; or —(C₁₋₄ straight or branched alkylene)C(O)O—N(R^{∘})₂, wherein each R^{∘} may be substituted as defined below and is independently hydrogen, C₁₋₆ aliphatic, —CH₂Ph, —O(CH₂)₀₋₁Ph, —CH₂-(5-6 membered heteroaryl ring), or a 5-6-membered saturated, partially unsaturated, or aryl ring having 0-4 heteroatoms independently selected from nitrogen, oxygen, or sulfur, or, notwithstanding the definition above, two independent occurrences of R^{∘}, taken together with their intervening atom(s), form a 3-12-membered saturated, partially unsaturated, or aryl mono- or bicyclic ring having 0-4 heteroatoms independently selected from nitrogen, oxygen, or sulfur, which may be substituted as defined below.

Suitable monovalent substituents on R^{∘} (or the ring formed by taking two independent occurrences of R^{∘} together with their intervening atoms), are independently halogen, —(CH₂)₀₋₂R^{●}, -(haloR^{●}), —(CH₂)₀₋₂OH, —(CH₂)₀₋₂OR^{●}, —(CH₂)₀₋₂CH(OR^{●})₂, —O(haloR^{●}), —CN, —N₃, —(CH₂)₀₋₂C(O)R^{●}, —(CH₂)₀₋₂C(O)OH, —(CH₂)₀₋₂C(O)OR^{●}, —(CH₂)₀₋₂SR^{●}, —(CH₂)₀₋₂SH, —(CH₂)₀₋₂NH₂, —(CH₂)₀₋₂NHR^{●}, —(CH₂)₀₋₂NR^{●}₂, —NO₂, —SiR^{●}₃, —OSiR^{●}₃, —C(O)SR^{●}, —(C₁₋₄ straight or branched alkylene)C(O)OR^{●}, or —SSR^{●}; wherein each R^{●} is unsubstituted or where preceded by “halo” is substituted only with one or more halogens, and is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur. Suitable divalent substituents on a saturated carbon atom of R^{∘} include ═O and ═S.

Suitable divalent substituents on a saturated carbon atom of an “optionally substituted” group include the following: ═O, ═S, ═NNR*₂, ═NNHC(O)R*, ═NNHC(O)OR*, ═NNHS(O)₂R*, ═NR*, ═NOR*, —O(C(R*₂))₂₋₃O—, or —S(C(R*₂))₂₋₃S—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur. Suitable divalent substituents that are bound to vicinal substitutable carbons of an “optionally substituted” group include: —O(CR*₂)₂₋₃O—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur.

Suitable substituents on the aliphatic group of R* include halogen, —R^{●}, -(haloR^{●}), —OH, —OR^{●}, —O(haloR^{●}), —CN, —C(O)OH, —C(O)OR^{●}, —NH₂, —NHR^{●}, —NR^{●}₂, or —NO₂, wherein each R^{●} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

Suitable substituents on a substitutable nitrogen of an “optionally substituted” group include —R^{†}, —NR^{†}₂, —C(O)R^{†}, —C(O)OR^{†}, —C(O)C(O)R^{†}, —C(O)CH₂C(O)R^{†}, —S(O)₂R^{†}, —S(O)₂NR^{†}₂, —C(S)NR^{†}₂, —C(NH)NR^{†}₂, or —N(R^{†})S(O)₂R^{†}; wherein each R^{†} is independently hydrogen, C₁₋₆ aliphatic which may be substituted as defined below, unsubstituted —OPh, or an unsubstituted 5-6-membered saturated, partially unsaturated, or aryl ring having 0-4 heteroatoms independently selected from nitrogen, oxygen, or sulfur, or, notwithstanding the definition above, two independent occurrences of R^{†}, taken together with their intervening atom(s) form an unsubstituted 3-12-membered saturated, partially unsaturated, or aryl mono- or bicyclic ring having 0-4 heteroatoms independently selected from nitrogen, oxygen, or sulfur.

Suitable substituents on the aliphatic group of R^{†} are independently halogen, —R^{●}, -(haloR^{●}), —OH, —OR^{●}, —O(haloR^{●}), —CN, —C(O)OH, —C(O)OR^{●}, —NH₂, —NHR^{●}, —NR^{●}₂, or —NO₂, wherein each R^{●} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge et al., describe pharmaceutically acceptable salts in detail in J. Pharmaceutical Sciences, 1977, 66, 1-19, incorporated herein by reference. Pharmaceutically acceptable salts of the compounds of this disclosure include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, pivalate, propionate, stearate, succinate, sulfate, tartrate, thiocyanate, p-toluenesulfonate, undecanoate, valerate salts, and the like.

Salts derived from appropriate bases include alkali metal, alkaline earth metal, ammonium and N⁺(C₁₋₄alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, loweralkyl sulfonate and aryl sulfonate.

Unless otherwise stated, structures depicted herein are also meant to include all isomeric (e.g., enantiomeric, diastereomeric, and geometric (or conformational)) forms of the structure; for example, the R and S configurations for each asymmetric center, Z and E double bond isomers, and Z and E conformational isomers. Therefore, single stereochemical isomers as well as enantiomeric, diastereomeric, and geometric (or conformational) mixtures of the present compounds are within the scope of the present disclosure. Unless otherwise stated, all tautomeric forms of the compounds of the present disclosure are within the scope of the present disclosure. Additionally, unless otherwise stated, structures depicted herein are also meant to include compounds that differ only in the presence of one or more isotopically enriched atoms. For example, compounds having the present structures including the replacement of hydrogen by deuterium or tritium, or the replacement of a carbon by a ¹³C- or ¹⁴C-enriched carbon are within the scope of this disclosure. Such compounds are useful, for example, as analytical tools, as probes in biological assays, or as therapeutic agents in accordance with the present disclosure.

As used herein, the term “full-length,” when used to refer to SARM1, refers to a SARM1 polypeptide that comprises at least: (i) the N-terminal autoinhibitory domain or a functional fragment thereof, (ii) one or more SAM domains or a functional fragment thereof, and (iii) a TIR domain or a functional fragment thereof, of a human SARM1 polypeptide having constitutive NADase activity. In some embodiments, a full-length SARM1 lacks a mitochondrial targeting sequence. In some embodiments, provided are SARM1 polypeptides comprising at least a functional fragment of a SARM1 N-terminal auto-inhibitory domain, at least a functional fragment of one or more SAM domains, and at least a functional fragment of a SARM1 TIR domain, wherein the SARM1 polypeptide lacks a mitochondrial targeting sequence.

### 3. Description of Exemplary Embodiments

In certain embodiments, the present disclosure provides a compound of formula I^{A}

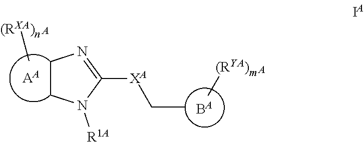

or a pharmaceutically acceptable salt thereof. In some embodiments, a compound of formula I^{A} is an inhibitor of SARM1 NADase activity. It will be appreciated that certain compounds of formula I^{A} are proton pump inhibitors.

As defined generally above, X^{A} is —S—, —SO— or —SO₂—. In some embodiments, X^{A} is —S—. In some embodiments, X^{A} is —SO—. In some embodiments, X^{A} is —SO₂—.

As defined generally above, R^{1A} is hydrogen, C₁₋₄ aliphatic, alkali metal, alkaline earth metal, ammonium or N⁺(C₁₋₄alkyl)₄. It will be appreciated that when R^{1A} is hydrogen or C₁₋₄ aliphatic, R^{1A} is covalently bonded to the nitrogen atom in formula I^{A}. It will further be appreciated that when R^{1A} is an alkali metal, alkaline earth metal, ammonium (i.e., NH₄⁺) or N⁺(C₁₋₄alkyl)₄, R^{1A} is ionically associated with the nitrogen atom in formula I^{A}. In some embodiments, R^{1A} is hydrogen or C₁₋₄ aliphatic. In some embodiments, R^{1A} is selected from an alkali metal, alkaline earth metal, ammonium (i.e., NH₄⁺) or N⁺(C₁₋₄alkyl)₄. In some embodiments, R^{1A} is hydrogen. In some embodiments, R^{1A} is C₁₋₄ aliphatic. In some embodiments, R^{1A} is an alkali metal. In some such embodiments, R^{1A} is sodium (Na⁺). In some embodiments, R^{1A} is an alkaline earth metal. In some embodiments, R^{1A} is ammonium. In some embodiments, R^{1A} is N⁺(C₁₋₄alkyl)₄.

As defined generally above, the Ring A^{A} group of formula I^{A} is a benzo fused ring or a 5-6 membered heteroaromatic fused ring having 1-3 heteroatoms independently selected from nitrogen, oxygen, and sulfur. In some embodiments, Ring A^{A} is a benzo fused ring. In some embodiments, Ring A^{A} is a 5-6 membered heteroaromatic fused ring having 1-3 heteroatoms independently selected from nitrogen, oxygen and sulfur. In some embodiments, Ring A^{A} is a 6 membered heteroaromatic fused ring having 1-2 nitrogens. In some embodiments, Ring A^{A} is a pyrido fused ring, a pyrimidino fused ring, pyridazino or pyrazino fused ring. In some embodiments, Ring A^{A} is a triazino fused ring. In some embodiments, Ring A^{A} is a 5 membered heteroaromatic fused ring containing 1-2 heteroatoms independently selected from oxygen, nitrogen and sulfur. In some embodiments, Ring A^{A} is a pyrrolo fused ring, a thiopheno fused ring, a furano fused ring, a thiazolofused ring, an isothiazolo fused ring, an imidazolo fused ring, a pyrazolo fused ring, an oxazolo fused ring, or an isoxazolo fused ring.

As defined generally above, the Ring B^{A} group of formula I^{A} is selected from phenyl, an 8-10 membered bicyclic aromatic carbocyclic ring, a 4-8 membered saturated or partially unsaturated monocyclic heterocyclic ring having 1-2 heteroatoms independently selected from nitrogen, oxygen, or sulfur, a 5-6 membered monocyclic heteroaromatic ring having 1-4 heteroatoms independently selected from nitrogen, oxygen, or sulfur, or an 8-10 membered bicyclic heteroaromatic ring having 1-5 heteroatoms independently selected from nitrogen, oxygen, or sulfur. In some embodiments, Ring B^{A} is aryl. In some embodiments, Ring B^{A} is phenyl, biphenyl, napthyl or anthracyl. In some embodiments, Ring B^{A} is indanyl, phthalimidyl, naphthimidyl, phenanthridinyl, or tetrahydronaphthyl. In some embodiments, Ring B^{A} is heteroaryl. In some embodiments, Ring B^{A} is thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, indolizinyl, purinyl, naphthyridinyl or pteridinyl.

As defined generally above, R^{XA} and R^{YA} are independently hydrogen, C₁₋₄ aliphatic optionally substituted with 1-4 halogen, —OR^{A}, —SR^{A}, —N(R^{A})₂, —N(R^{A})C(O)R^{A}, —C(O)N(R^{A})₂, —N(R^{A})C(O)N(R^{A})₂, —N(R^{A})C(O)OR^{A}, —OC(O)N(R^{A})₂, —N(R^{A})S(O)₂R^{A}, —S(O)₂N(R^{A})₂, —C(O)R^{A}, —C(O)OR^{A}, —OC(O)R^{A}, —S(O)R^{A}, —S(O)₂R^{A}, phenyl, an 8-10 membered bicyclic aromatic carbocyclic ring, a 4-8 membered saturated or partially unsaturated monocyclic heterocyclic ring having 1-2 heteroatoms independently selected from nitrogen, oxygen, or sulfur, a 5-6 membered monocyclic heteroaromatic ring having 1-4 heteroatoms independently selected from nitrogen, oxygen, or sulfur, or an 8-10 membered bicyclic heteroaromatic ring having 1-5 heteroatoms independently selected from nitrogen, oxygen, or sulfur.

In some embodiments, R^{XA} and R^{YA} are the same. In some embodiments, R^{XA} and R^{YA} are both hydrogen. In some embodiments, R^{XA} and R^{YA} are both C₁₋₄ aliphatic optionally substituted with 1-4 halogen. In some embodiments, R^{XA} and R^{YA} are both —OR^{A}. In some embodiments, R^{XA} and R^{YA} are both aryl. In some embodiments, R^{XA} and R^{YA} are both heteroaryl.

In some embodiments, R^{XA} and R^{YA} are different. In some embodiments, R^{XA} is hydrogen and R^{YA} is C₁₋₄ aliphatic optionally substituted with 1-4 halogen and/or —OR^{A}. In some embodiments, R^{XA} is —OR^{A} and R^{YA} is C₁₋₄ aliphatic optionally substituted with 1-4 halogen. In some embodiments, R^{XA} is aryl and R^{YA} is —OR^{A} and/or C₁₋₄ aliphatic optionally substituted with 1-4 halogen. In some embodiments, R^{XA} is heteroaryl and R^{YA} is —OR^{A} and/or C₁₋₄ aliphatic optionally substituted with 1-4 halogen.

As defined generally above, m^{A} and n^{A} are independently 0, 1, 2, or 3. In some embodiments, m^{A} and n^{A} are the same. In some embodiments, m^{A} and n^{A} are both zero. In some embodiments, m^{A} and n^{A} are both one. In some embodiments, m^{A} and n^{A} are both two. In some embodiments, m^{A} and n^{A} are both three.

In some embodiments, m^{A} and n^{A} are different. In some embodiments, m^{A} is zero and n^{A} is one, two or three. In some embodiments, m^{A} is one and n^{A} is zero, two or three. In some embodiments, m^{A} is two and n^{A} is zero, one or three. In some embodiments, m^{A} is three and n^{A} is zero, one or two. In some embodiments, m^{A} is one, two or three and n^{A} is zero. In some embodiments, m^{A} is zero, two or three and n^{A} is one. In some embodiments, m^{A} is zero, one or three and n^{A} is two. In some embodiments, m^{A} is zero, one or two and n^{A} is three. In some embodiments, m^{A} is one and n^{A} is two or three.

In some embodiments, n^{A} is one and R^{XA} is —OCH₃. In some embodiments, n^{A} is one and R^{XA} is —OCHF₂. In some embodiments, n^{A} is one and R^{XA} is a 5-membered heteroaryl ring. In some such embodiments, n^{A} is one and R^{XA} is pyrrolyl. In some embodiments, n^{A} is one and R^{XA} is —OR^{A}. In some such embodiments, R^{A} is optionally substituted C₁₋₆ aliphatic. In some embodiments, n^{A} is one and R^{XA} is —OR^{A}, wherein R^{A} is C₁₋₆ aliphatic substituted with phenyl.

In some embodiments, m^{A} is two and each R^{YA} is independently selected from —OR^{A} and C₁₋₄ aliphatic optionally substituted with 1-4 halogen. In such embodiments, one R^{YA} is —CH₃ and the other R^{YA} is —OCH₃. In some embodiments, one R^{YA} is —CH₃ and the other R^{YA} is —OCH₂CF₃. In some embodiments, m^{A} is two and each R^{YA} is —OCH₃. In some embodiments, m^{A} is two and each R^{YA} is selected from —OR^{A} and C₁₋₄ aliphatic optionally substituted with 1-4 halogen, wherein R^{A} is C₁₋₆ aliphatic substituted with —(CH₂)₀₋₄OR^{∘}. In some such embodiments, one R^{YA} is —CH₃ and the other R^{YA} is —OCH₂CH₂CH₂OCH₃.

In some embodiments, m^{A} is three and each R^{YA} is independently selected from —OR^{A} and C₁₋₄ aliphatic optionally substituted with 1-4 halogen. In some embodiments, one R^{YA} is —OCH₃ and two R^{YA} are —CH₃. In some embodiments, one R^{YA} is —OCH₂CF₃ and two R^{YA} are —CH₃.

In some embodiments, Ring A^{A} is selected from the Ring A^{A} groups in the compounds depicted in Table 1^{A}, below. In some embodiments, Ring B^{A} is selected from the Ring B^{A} groups in the compounds depicted in Table 1^{A}, below. In some embodiments, R^{XA} is selected from the R^{XA} groups in the compounds depicted in Table 1^{A}, below. In some embodiments, R^{YA} is selected from the R^{y}A groups in the compounds depicted in Table 1^{A}, below. In some embodiments, X^{A} is selected from the X^{A} groups in the compounds depicted in Table 1^{A}, below. In some embodiments, the compounds of formula I^{A} are selected from those depicted in Table 1^{A} below. In some embodiments, the compounds of formula I^{A} are selected from the compounds in Table 1^{A}:

TABLE 1^{A}

| | |
|---|---|
| 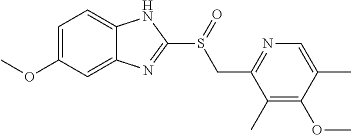 | I^{A}-1 |
| 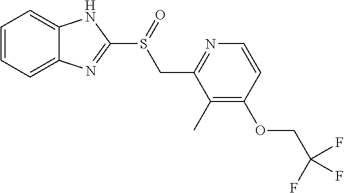 [SMILES: Cc1c(OCC(F)(F)F)ccnc1CS(=O)c1nc2ccccc2[nH]1] | I^{A}-2 |
| 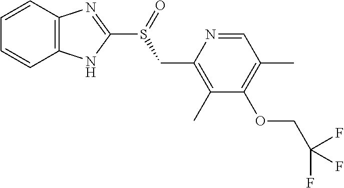 | I^{A}-3 |
| 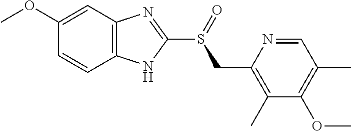 | I^{A}-4 |
| 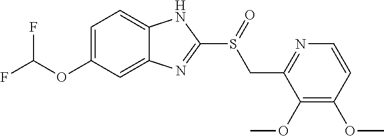 [SMILES: COc1ccnc(CS(=O)c2nc3cc(OC(F)F)ccc3[nH]2)c1OC] | I^{A}-5 |
| 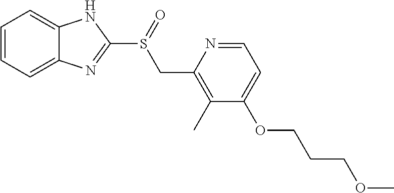 [SMILES: COCCCOc1ccnc(CS(=O)c2nc3ccccc3[nH]2)c1C] | I^{A}-6 |
| 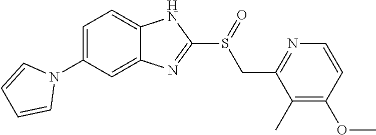 [SMILES: COc1ccnc(CS(=O)c2nc3cc(-n4cccc4)ccc3[nH]2)c1C] | I^{A}-7 |
| 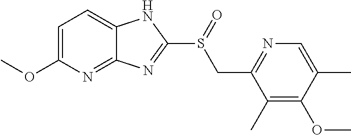 | I^{A}-8 |
| 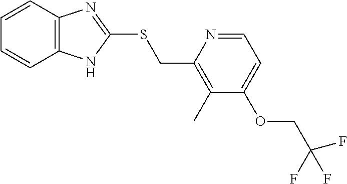 [SMILES: Cc1c(OCC(F)(F)F)ccnc1CSc1nc2ccccc2[nH]1] | I^{A}-9 |
| 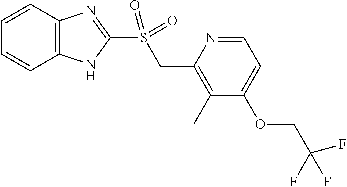 [SMILES: Cc1c(OCC(F)(F)F)ccnc1CS(=O)(=O)c1nc2ccccc2[nH]1] | I^{A}-10 |
| 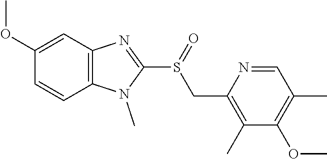 [SMILES: COc1ccc2c(c1)nc(S(=O)Cc1ncc(C)c(OC)c1C)n2C] | I^{A}-11 |
| 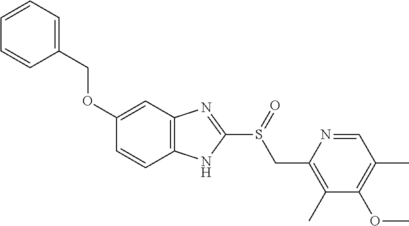 [SMILES: COc1c(C)cnc(CS(=O)c2nc3cc(OCc4ccccc4)ccc3[nH]2)c1C] | I^{A}-12 |
| 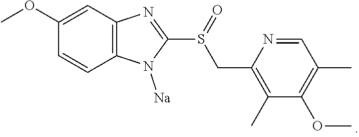 | I^{A}-13 |

In some embodiments, X^{A} is —SO—. In some embodiments, n^{A} is 0 or 1 and m^{A} is 2 or 3. In some embodiments, R^{1A} is hydrogen, C₁₋₄ aliphatic or an alkali metal. In some embodiments, R^{1A} is hydrogen, methyl or sodium. In some embodiments, R^{YA} is hydrogen, C₁₋₄ aliphatic optionally substituted with 1-4 halogen or —OR^{A}; and R^{A} is optionally substituted C₁₋₆ aliphatic. In some embodiments, R^{YA} is hydrogen, —CH₃, —OCH₃, —OCH₂CF₃ or —O(CH₂)₃OCH₃. In some embodiments, R^{XA} is hydrogen, —OR^{A}, or heteroaryl; and R^{A} is optionally substituted C₁₋₆ aliphatic or benzyl. In some embodiments, R^{XA} is hydrogen, —OCH₃, —OCHCF₂, pyrrolyl or —OCH₂-phenyl.

In some embodiments, Ring A^{A} is an arylo fused ring and Ring B^{A} is a heteroaryl ring. In some embodiments, Ring A^{A} is a benzo fused ring and Ring B^{A} is a pyridyl ring. In some embodiments, Ring A^{A} is a heteroaromatic fused ring and Ring B^{A} is a heteroaryl ring.

In some embodiments, Ring A^{A} is selected from the group consisting of a pyrido fused ring, a pyrimidino fused ring, a pyridazino fused ring, pyrazino fused ring, a triazino fused ring, a pyrrolo fused ring, a thiopheno fused ring, a furano fused ring, a thiazolofused ring, an isothiazolo fused ring, an imidazolo fused ring, a pyrazolo fused ring, an oxazolo fused ring and an isoxazolo fused ring.

In some embodiments, Ring B^{A} is selected from the group consisting of phenyl, biphenyl, napthyl, anthracyl, indanyl, phthalimidyl, naphthimidyl, phenanthridinyl, tetrahydronaphthyl, thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, indolizinyl, purinyl, naphthyridinyl and pteridinyl.

In certain embodiments, the present disclosure provides a compound of formula I^{B}:

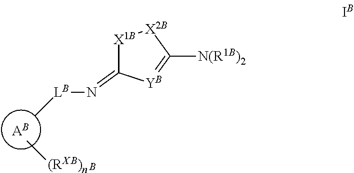

or a pharmaceutically acceptable salt thereof.

As defined generally above, X^{1B} and X^{2B} are independently —O—, —S—, or —NR^{B}_, provided that one of X^{1B} and X^{2B} is —O— or —S— and both of X^{1B} and X^{2B} are not —O—. In some embodiments, X^{1B} and X^{2B} are the same. In some embodiments, X^{1B} and X^{2B} are different. In some embodiments, X^{1B} and X^{2B} are —S—. In some embodiments, X^{1B} is —S— and X^{2B} is —O—. In some embodiments, X^{1B} is —O— and X^{2B} is —S—. In some embodiments, X^{1B} and X^{2B} are selected from the X^{1B} and X^{2B} groups in the compounds depicted in Table 1^{B}, below.

As defined generally above, Y^{B} is —N— or —CH—. In some embodiments, Y^{B} is —N—. In some embodiments, Y^{B} is —CH—. In some embodiments, Y^{B} is selected from the Y^{B} groups in the compounds depicted in Table 1^{B}, below.

As defined generally above, R^{1B} is hydrogen or optionally substituted C₁₋₄ aliphatic. In some embodiments, each R^{1B} is the same. In some embodiments, each R^{1B} is different. In some embodiments, each R^{1B} is hydrogen. In some embodiments, each R^{1B} is optionally substituted C₁₋₄ aliphatic. In some embodiments, one R^{1B} is hydrogen and the other is optionally substituted C₁₋₄ aliphatic. In some embodiments, R^{1B} is selected from the R^{1B} groups in the compounds depicted in Table 1^{B}, below.

As defined generally above, Ring A^{B} is phenyl, an 8-10 membered bicyclic aromatic carbocyclic ring, a 4-8 membered saturated or partially unsaturated monocyclic heterocyclic ring having 1-2 heteroatoms independently selected from nitrogen, oxygen, or sulfur, a 5-6 membered monocyclic heteroaromatic ring having 1-4 heteroatoms independently selected from nitrogen, oxygen, or sulfur, or an 8-10 membered bicyclic heteroaromatic ring having 1-5 heteroatoms independently selected from nitrogen, oxygen, or sulfur. In some embodiments, Ring A^{B} is aryl. In some embodiments, Ring A^{B} is phenyl, biphenyl, napthyl or anthracyl. In some embodiments, Ring A^{B} is indanyl, phthalimidyl, naphthimidyl, phenanthridinyl, or tetrahydronaphthyl. In some embodiments, Ring A^{B} is heteroaryl. In some embodiments, Ring A^{B} is thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, indolizinyl, purinyl, naphthyridinyl or pteridinyl. In some embodiments, Ring A^{B} is selected from the Ring A^{B} groups in the compounds depicted in Table 1^{B}, below.

As defined generally above, each R^{XB} is independently hydrogen, halogen or an optionally substituted group selected from C₁₋₆ aliphatic, a 3-8 membered saturated or partially unsaturated monocyclic carbocyclic ring, phenyl, an 8-10 membered bicyclic aromatic carbocyclic ring, a 4-8 membered saturated or partially unsaturated monocyclic heterocyclic ring having 1-2 heteroatoms independently selected from nitrogen, oxygen, or sulfur, a 5-6 membered monocyclic heteroaromatic ring having 1-4 heteroatoms independently selected from nitrogen, oxygen, or sulfur, or an 8-10 membered bicyclic heteroaromatic ring having 1-5 heteroatoms independently selected from nitrogen, oxygen, or sulfur.

In some embodiments, each R^{XB} is the same. In some embodiments, each R^{XB} is different. In some embodiments, R^{XB} is hydrogen. In some embodiments, R^{XB} is halogen. In some embodiments, R^{XB} is optionally substituted C₁₋₄ aliphatic. In some embodiments, R^{X} is aryl. In some embodiments, R^{XB} is phenyl, biphenyl, napthyl or anthracyl. In some embodiments, R^{XB} is indanyl, phthalimidyl, naphthimidyl, phenanthridinyl, or tetrahydronaphthyl. In some embodiments, R^{XB} is heteroaryl. In some embodiments, R^{XB} is thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, indolizinyl, purinyl, naphthyridinyl or pteridinyl. In some embodiments, R^{XB} is selected from the R^{XB} groups in the compounds depicted in Table 1^{B}, below.

As defined generally above, L^{B} is a covalent bond, a C₁₋₆ membered straight or branched bivalent hydrocarbon chain, cyclopropylenyl, cyclobutylenyl, or oxetanylenyl. In some embodiments, L^{B} is a covalent bond. In some embodiments, L^{B} is a C₁₋₆ membered straight or branched bivalent hydrocarbon chain. In some embodiments, L^{B} is cyclopropylenyl. In some embodiments, L^{B} is cyclobutylenyl. In some embodiments, L^{B} is oxetanylenyl. In some embodiments, L^{B} is —C(CH₃)₂—. In some embodiments, L^{B} is —CH₂—. In some embodiments, L^{B} is —CH(CH₃)—. In some embodiments, L^{B} is —CH(CH₃)— with (S) configuration at the chiral center. In some embodiments, L^{B} is —CH(CH₃)— with an (R) configuration at the chiral center. In some embodiments, L^{B} is selected from the L^{B} groups in the compounds depicted in Table 1^{B}, below.

As defined generally above, n^{B} is 0-4. In some embodiments, n^{B} is 0. In some embodiments, n^{B} is 1. In some embodiments, n^{B} is 2. In some embodiments, n^{B} is 3. In some embodiments, n^{B} is 4.

In some embodiments, the compounds of formula I^{B} are selected from the compounds in Table 1^{B}:

TABLE 1^{B}

| | |
|---|---|
| 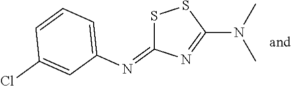 [SMILES: CN(C)c1n/c(=N/c2cccc(Cl)c2)ss1] | I^{B}-1 |
| 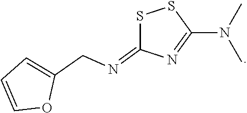 | I^{B}-2 |

In some embodiments, X^{1B} and X^{2B} are —S— an Y^{B} is —N—. In some embodiments, R^{1B} is hydrogen or optionally substituted C₁₋₄ aliphatic. In some embodiments, R^{1B} is hydrogen or methyl. In some embodiments, L^{B} is a covalent bond or a C₁₋₆ membered straight or branched bivalent hydrocarbon chain. In some embodiments, L^{B} is a covalent bond or a methylene group. In some embodiments, R^{XB} is hydrogen, halogen or optionally substituted C₁₋₄ aliphatic. In some embodiments, R^{XB} is hydrogen or —Cl.

In some embodiments, Ring A^{B} is aryl or heteroaryl. In some embodiments, Ring A^{B} is selected from the group consisting of phenyl, biphenyl, napthyl and anthracyl. In some embodiments, Ring A^{B} is selected from the group consisting of indanyl, phthalimidyl, naphthimidyl, phenanthridinyl, tetrahydronaphthyl, thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, indolizinyl, purinyl, naphthyridinyl and pteridinyl.

In some embodiments, the present disclosure provides a compound of formula I^{C}:

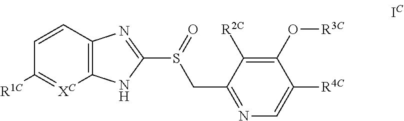

or a pharmaceutically acceptable salt thereof,
wherein:
X^{C} is N or C;
R^{1C} is H, C₁-C₅ alkyl, C₁-C₅ alkoxy, or C₁-C₅ haloalkoxy;
R^{2C} is C₁-C₅ alkyl or C₁-C₅ alkoxy;
R^{3C} is C₁-C₁₀ alkyl, C₁-C₁₀ haloalkyl or an ether; and
R^{4C} is H, C₁-C₅ alkyl or C₁-C₅ alkoxy.

In some embodiments, X^{C} is N. In some embodiments, X^{C} is C.

In some embodiments, R^{1C} is H. In some embodiments, R^{1C} is C₁-C₅ alkyl, C₁-C₅ alkoxy, or C₁-C₅ haloalkoxy.

In some embodiments, R^{1C} is C₁-C₅ alkyl. In some such embodiments, R^{1C} is methyl, ethyl, n-propyl or isopropyl.

In some embodiments, R^{1C} is C₁-C₅ alkoxy. In some such embodiments, R^{1C} is —OCH₃, —OCH₂CH₃, —OCH₂CH₂CH₃, or —OCH(CH₃)₂.

In some embodiments, R^{1C} is C₁-C₅ haloalkoxy. In some embodiments, R^{1C} is C₁-C₅ fluoroalkoxy. In some such embodiments, R^{1C} is fluoromethoxy, difluoromethoxy, trifluoromethoxy, fluoroethoxy, difluoroethoxy, or trifluoromethoxy. In some embodiments, R^{1C} is —OCH₂F, —OCHF₂, —OCF₃, —OCH₂CH₂F, —OCH₂CHF₂, or —OCH₂CF₃.

In some embodiments, R^{2C} is C₁-C₅ alkyl. In some such embodiments, R^{2C} is methyl, ethyl, n-propyl or isopropyl.

In some embodiments, R^{2C} is C₁-C₅ alkoxy. In some such embodiments, R^{2C} is —OCH₃, —OCH₂CH₃, —OCH₂CH₂CH₃, or —OCH(CH₃)₂.

In some embodiments, R^{3C} is C₁-C₁₀ alkyl. In some such embodiments, R^{3C} is methyl, ethyl, n-propyl or isopropyl.

In some embodiments, R^{3C} is C₁-C₁₀ haloalkyl. In some embodiments, R^{3C} is fluoroalkyl. In some such embodiments, R^{3C} is fluoromethyl, difluoromethyl, trifluoromethyl, fluoroethyl, difluoroethyl, or trifluoroethyl. In some embodiments, R^{3C} is —CH₂F, —CHF₂, —CF₃, —CH₂CH₂F, —CH₂CHF₂, or —CH₂CF₃.

In some embodiments, R^{3C} is an ether. In some such embodiments, R^{3C} is methoxypropyl (i.e., —CH₂CH₂CH₂OCH₃).

In some embodiments, R^{4C} is H. In some embodiments, R^{4C} is C₁-C₅ alkyl. In some such embodiments, R^{4C} is methyl, ethyl, n-propyl or isopropyl.

In some embodiments, R^{4C} is C₁-C₅ alkoxy. In some such embodiments, R^{4C} is —OCH₃, —OCH₂CH₃, —OCH₂CH₂CH₃, or —OCH(CH₃)₂.

In some embodiments, the compounds of formula I^{C} are selected from the compounds in Table 1C:

TABLE 1^{C}

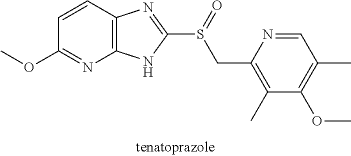

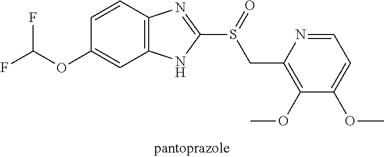 [SMILES: COc1ccnc(CS(=O)c2nc3ccc(OC(F)F)cc3[nH]2)c1OC]

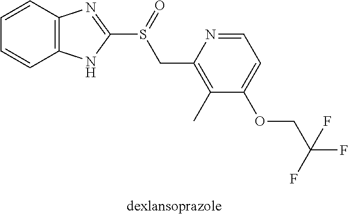 [SMILES: Cc1c(OCC(F)(F)F)ccnc1CS(=O)c1nc2ccccc2[nH]1]

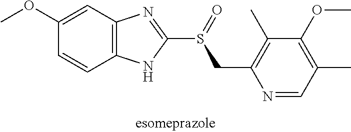

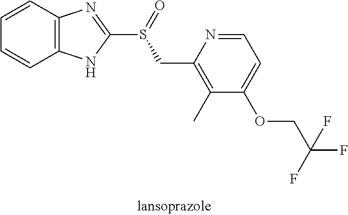 [SMILES: Cc1c(OCC(F)(F)F)ccnc1C[S@@](=O)c1nc2ccccc2[nH]1]

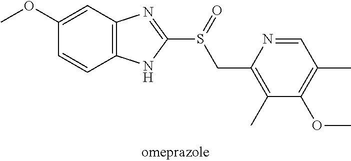

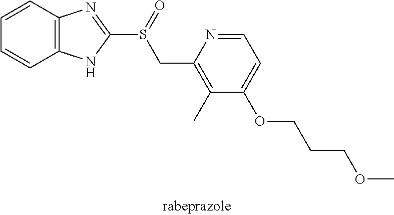 [SMILES: COCCCOc1ccnc(CS(=O)c2nc3ccccc3[nH]2)c1C]

In some embodiments, pantoprazole is in the form of a sodium salt:

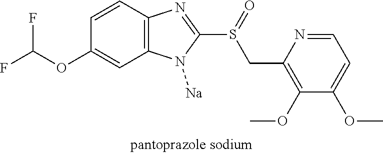

In some embodiments, rabeprazole is in the form of a sodium salt:

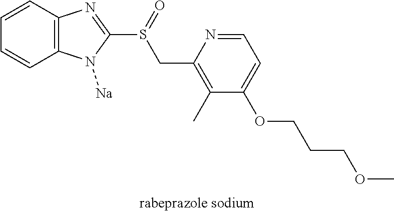

In some embodiments, esomeprazole is in the form of a magnesium hydrate:

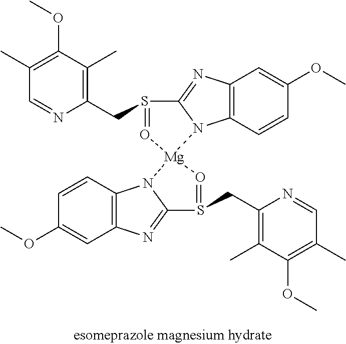

In some embodiments, the present disclosure provides a compound of formula I^{D}:

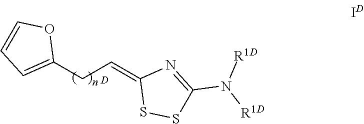

or a pharmaceutically acceptable salt thereof,
wherein:
R^{1D} and R^{2D} are each independently selected from H, C₁-C₅ alkyl, C₁-C₅ alkoxy, C₁-C₅ haloalkyl, or C₁-C₅ haloalkoxy; and
n^{D} is an integer from 1 to 5.

In some embodiments, R^{1D} and R^{2D} are the same. In some embodiments, each of R^{1D} and R^{2D} is hydrogen.

In some embodiments, R^{1D} and R^{2D} are different. In some embodiments, R^{1D} is hydrogen and R^{2D} IS C₁-C₅ alkyl.

In some embodiments, R^{1D} is selected from methyl, ethyl, n-propyl or isopropyl. In some embodiments, R^{1D} is selected from —OCH₃, —OCH₂CH₃, —OCH₂CH₂CH₃, or —OCH(CH₃)₂. In some embodiments, R^{1D} is selected from fluoromethyl, difluoromethyl, trifluoromethyl, fluoroethyl, difluoroethyl, or trifluoroethyl. In some such embodiments, R^{1D} is selected from —CH₂F, —CHF₂, —CF₃, —CH₂CH₂F, —CH₂CHF₂ or —CH₂CF₃. In some embodiments, RD is selected from fluoromethoxy, difluoromethoxy, trifluoromethoxy, fluoroethoxy, difluoroethoxy, or trifluoroethoxy. In some such embodiments, R^{1D} is selected from —OCH₂F, —OCHF₂, —OCF₃, —OCH₂CH₂F, —OCH₂CHF₂, or —OCH₂CF₃.

In some embodiments, R^{2D} is selected from methyl, ethyl, n-propyl or isopropyl. In some embodiments, R^{2D} is selected from —OCH₃, —OCH₂CH₃, —OCH₂CH₂CH₃, or —OCH(CH₃)₂. In some embodiments, R^{2D} is selected from fluoromethyl, difluoromethyl, trifluoromethyl, fluoroethyl, difluoroethyl, or trifluoroethyl. In some such embodiments, R^{2D} is selected from —CH₂F, —CHF₂, —CF₃, —CH₂CH₂F, —CH₂CHF₂ or —CH₂CF₃. In some embodiments, R^{2D} is selected from fluoromethoxy, difluoromethoxy, trifluoromethoxy, fluoroethoxy, difluoroethoxy, or trifluoroethoxy. In some such embodiments, R^{2D} is selected from —OCH₂F, —OCHF₂, —OCF₃, —OCH₂CH₂F, —OCH₂CHF₂, or —OCH₂CF₃.

In some embodiments, n^{D} is 1-2. In some embodiments, n^{D} is 1. In some embodiments, n^{D} is 2. In some embodiments, n^{D} is 3. In some embodiments, n^{D} is 4. In some embodiments, n^{D} is 5.

In some embodiments, a SARM1 NADase inhibitor is selected from the compounds in Table 2:

TABLE 2

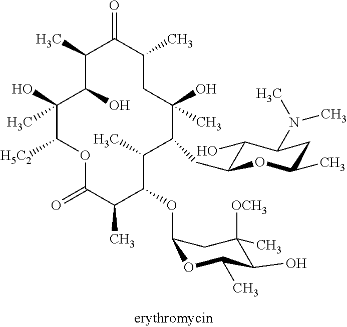

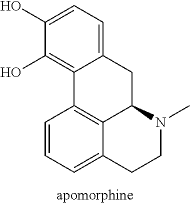 [SMILES: CN1CCc2cccc3c2[C@H]1Cc1ccc(O)c(O)c1-3]

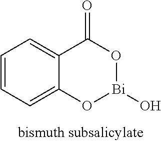

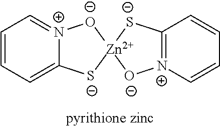

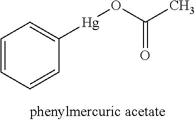

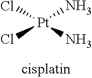

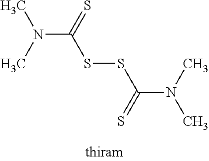

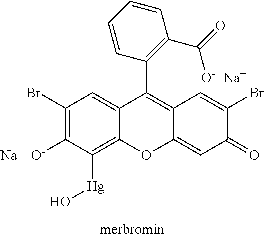

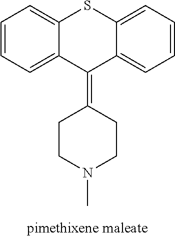 [SMILES: CN1CCC(=C2c3ccccc3Sc3ccccc32)CC1]

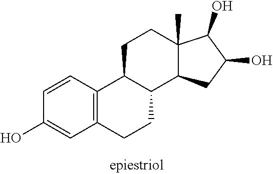 [SMILES: C[C@]12CC[C@@H]3c4ccc(O)cc4CC[C@H]3[C@@H]1C[C@H](O)[C@@H]2O]

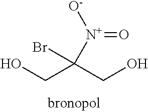

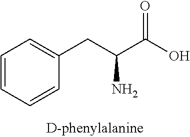 [SMILES: N[C@@H](Cc1ccccc1)C(=O)O]

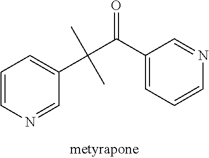 [SMILES: CC(C)(C(=O)c1cccnc1)c1cccnc1]

In some embodiments, a SARM1 NADase inhibitor is selected from the group of compounds in Table 3:

TABLE 3

| | |
|---|---|
| 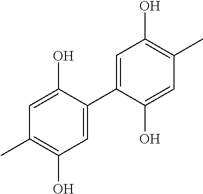 | NSC2805 |
| 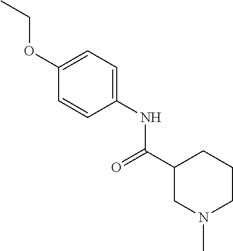 [SMILES: CCOc1ccc(NC(=O)C2CCCN(C)C2)cc1] | NSC1152 |
| 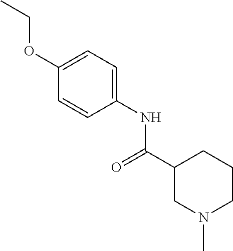 [SMILES: CCOc1ccc(NC(=O)C2CCCN(C)C2)cc1] | NSC22806 |
| 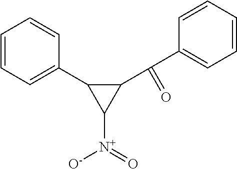 [SMILES: O=C(c1ccccc1)C1C(c2ccccc2)C1[N+](=O)[O-]] | NSC34879 |
| 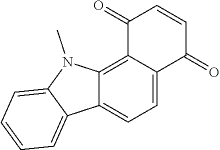 [SMILES: Cn1c2ccccc2c2ccc3c(c21)C(=O)C=CC3=O] | NSC92937 |
| 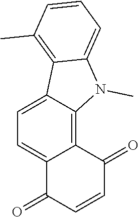 [SMILES: Cc1cccc2c1c1ccc3c(c1n2C)C(=O)C=CC3=O] | NSC645330 |
| 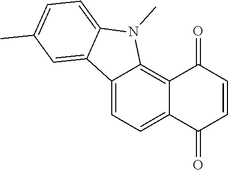 [SMILES: Cc1ccc2c(c1)c1ccc3c(c1n2C)C(=O)C=CC3=O] | NSC661221 |
| 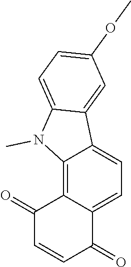 [SMILES: COc1ccc2c(c1)c1ccc3c(c1n2C)C(=O)C=CC3=O] | NSC641396 |
| 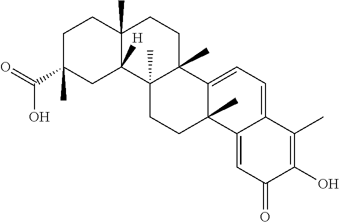 | NSC70931 |
| 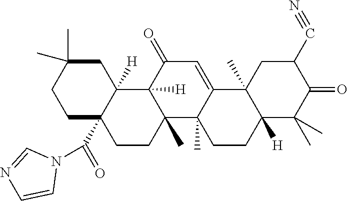 | NSC727038 |
| 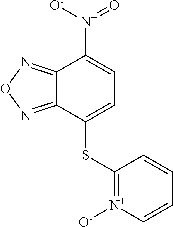 [SMILES: O=[N+]([O-])c1ccc(Sc2cccc[n+]2[O-])c2nonc12] | NSC228155 |
| 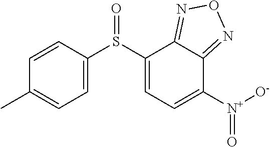 [SMILES: Cc1ccc(S(=O)c2ccc([N+](=O)[O-])c3nonc23)cc1] | NSC228150 |
| 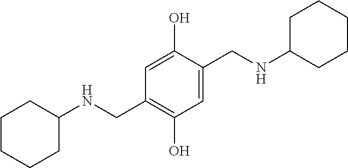 [SMILES: Oc1cc(CNC2CCCCC2)c(O)cc1CNC1CCCCC1] | NSC48443 |
| 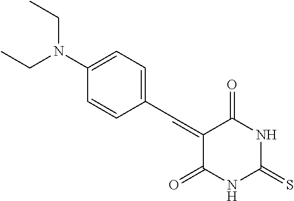 [SMILES: CCN(CC)c1ccc(C=C2C(=O)NC(=S)NC2=O)cc1] | NSC90749 |
| 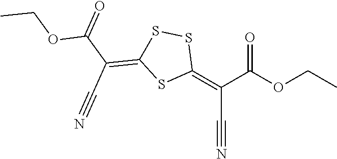 [SMILES: CCOC(=O)/C(C#N)=C1\SS/C(=C(/C#N)C(=O)OCC)S1] | NSC98363 |
| 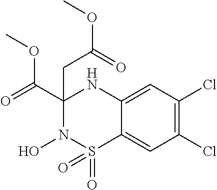 [SMILES: COC(=O)CC1(C(=O)OC)Nc2cc(Cl)c(Cl)cc2S(=O)(=O)N1O] | NSC163639 |
| 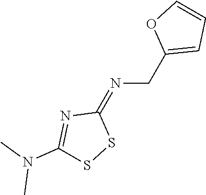 | NSC622608 |
| 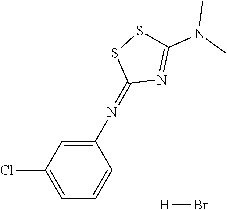 [SMILES: CN(C)c1nc(=Nc2cccc(Cl)c2)ss1 ; Br] | NSC622689 |

In some embodiments, the compounds of any of Formula I^{A}, Formula I^{B}, Formula I^{C} and Formula I^{D} are administered as part of a pharmaceutically acceptable composition. In some embodiments, the compounds of any of Formula I^{A}, Formula I^{B}, Formula I^{C} and Formula I^{D} are administered orally. In some embodiments, the compounds of any of Formula I^{A}, Formula I^{B}, Formula I^{C} and Formula I^{D} are administered in a range of 0.01-100 mg/kg body weight of the patient.

In some embodiments, the neurodegenerative or neurological disease or disorder is associated with axonal degeneration, axonal damage, axonopathy, a demyelinating disease, a central pontine myelinolysis, a nerve injury disease or disorder, a metabolic disease, a mitochondrial disease, metabolic axonal degeneration, axonal damage resulting from a leukoencephalopathy or a leukodystrophy. In some embodiments, the neurodegenerative or neurological disease or disorder is selected from the group consisting of spinal cord injury, stroke, multiple sclerosis, progressive multifocal leukoencephalopathy, congenital hypomyelination, encephalomyelitis, acute disseminated encephalomyelitis, central pontine myelolysis, osmotic hyponatremia, hypoxic demyelination, ischemic demyelination, adrenoleukodystrophy, Alexander's disease, Niemann-Pick disease, Pelizaeus Merzbacher disease, periventricular leukomalacia, globoid cell leukodystrophy (Krabbe's disease), Wallerian degeneration, optic neuritis, transverse myelitis, amyotrophic lateral sclerosis (ALS, Lou Gehrig's disease), Huntington's disease, Alzheimer's disease, Parkinson's disease, Tay-Sacks disease, Gaucher's disease, Hurler Syndrome, traumatic brain injury, post radiation injury, neurologic complications of chemotherapy (chemotherapy induced neuropathy; CIPN), neuropathy, acute ischemic optic neuropathy, vitamin B₁₂ deficiency, isolated vitamin E deficiency syndrome, Bassen-Komzweig syndrome, Glaucoma, Leber's hereditary optic atrophy (neuropathy), Leber congenital amaurosis, neuromyelitis optica, metachromatic leukodystrophy, acute hemorrhagic leukoencephalitis, trigeminal neuralgia, Bell's palsy, cerebral ischemia, multiple system atrophy, traumatic glaucoma, tropical spastic paraparesis human T-lymphotropic virus 1 (HTLV-1) associated myelopathy, west nile virus encephalopathy, La Crosse virus encephalitis, Bunyavirus encephalitis, pediatric viral encephalitis, essential tremor, Charcot-Marie-Tooth disease, motorneuron disease, spinal muscular atrophy (SMA), hereditary sensory and autonomic neuropathy (HSAN), adrenomyeloneuropathy, progressive supra nuclear palsy (PSP), Friedrich's ataxia, hereditary ataxias, noise induced hearing loss, congenital hearing loss, Lewy Body Dementia, frontotemporal dementia, amyloidosis, diabetic neuropathy, HIV neuropathy, enteric neuropathies and axonopathies, Guillain-Barre syndrome, and severe acute motor axonal neuropathy (AMAN).

In certain embodiments, the present disclosure provides any compound selected from those depicted in Table 1^{A}, above, or a pharmaceutically acceptable salt thereof, for the inhibition of SARM1 NADase activity. The compounds shown in Table 1^{A} are known proton pump inhibitors, such as: omeprazole (compound I^{A}-1); lansoprazole (compound I^{A}-2); dexlansoprazole (compound I^{A}-3); esomeprazole (compound I^{A}-4); pantoprazole (compound I^{A}-5); rabeprazole (compound I^{A}-6); ilaprazole (compound I^{A}-7); tenatoprazole (compound I^{A}-8); lansoprazole sulfide (compound I^{A}-9); lansoprazole sulfone (compound I^{A}-10); N-methyl omeprazole (compound I^{A}-11); 5-benzyloxy omeprazole (compound I^{A}-12) and sodium esomeprazole (compound I^{A}-13).

In certain embodiments, the present disclosure provides any compound selected from those depicted in Table 1^{B}, above, or a pharmaceutically acceptable salt thereof, for the inhibition of SARM1 NADase activity.

In certain embodiments, the present disclosure provides any compound selected from those depicted in Table 1^{C}, above, or a pharmaceutically acceptable salt thereof, for the inhibition of SARM1 NADase activity.

In certain embodiments, the present disclosure provides any compound selected from those depicted in Table 2, above, or a pharmaceutically acceptable salt thereof, for the inhibition of SARM1 NADase activity.

In certain embodiments, the present disclosure provides any compound selected from those depicted in Table 3, above, or a pharmaceutically acceptable salt thereof, for the inhibition of SARM1 NADase activity.

In certain embodiments, the present disclosure provides a pharmaceutical composition comprising a compound selected from any of Tables 1^{A}, 1^{B}, I^{C}, 2, or 3, or a pharmaceutically acceptable salt thereof, and a pharmaceutically acceptable carrier.

### 4. General Methods for Providing the Present Compounds

It will be appreciated that certain compounds of formula I^{A} are proton pump inhibitors and are commercially available from various sources.

The compounds of this disclosure and described by formula I^{A} herein may also be synthesized according to known procedures. For instance, U.S. Pat. No. 5,045,552, filed Dec. 28, 1989 and issued on Sep. 3, 1991 (“the '552 patent,” the entirety of which is hereby incorporated herein by reference), describes compounds of formula I^{A} and their synthesis. EP 268956, filed Nov. 13, 1987 and published Jun. 1, 1988 (“EP '256,” the entirety of which is hereby incorporated herein by reference), also describes compounds of formula I^{A} and their synthesis.

### General Preparation of the Compounds of Formula I^{A}

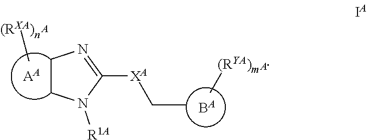

The compounds of formula I^{A} may be prepared according to the steps and intermediates (e.g., Scheme 1^{A}) described below and in the '552 patent and EP '256. In certain embodiments, compounds of the present disclosure of formula I^{A} are generally prepared according to Scheme 1^{A} set forth below:

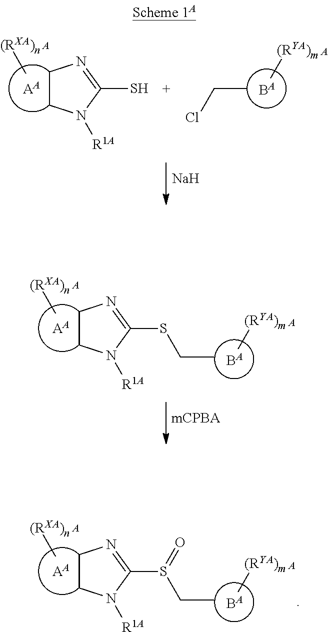

The compounds described by formula I^{B} herein may be prepared or isolated in general by synthetic and/or semi-synthetic methods known to those skilled in the art for analogous compounds and by methods described in detail in the Examples, herein. For instance, the compounds described by formula I^{B} herein may be synthesized according to WO 2006/084854, filed Feb. 8, 2006 and published on Aug. 17, 2006 (“WO '854,” the entirety of which is hereby incorporated herein by reference), describes compounds of formula I^{B} and their synthesis. Also describing synthesis of the compounds of formula I^{B} are Oliver et al., J. Org. Chem., vol. 39, No. 15, 1974, pp. 2225-2228 and Pandeya et al., Pharmaceutical Research, vol. 4, No. 4, 1987, pp. 321-326 (the entireties of both which are hereby incorporated herein by reference).

### General Preparation of the Compounds of Formula I^{B}

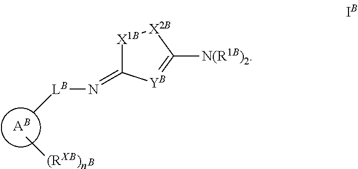

The compounds of formula I^{B} may be prepared according to the steps and intermediates (e.g., Scheme 1^{B}) described below and in WO '854. In certain embodiments, compounds of the present disclosure of formula I^{B} are generally prepared according to Scheme 1^{B} set forth below:

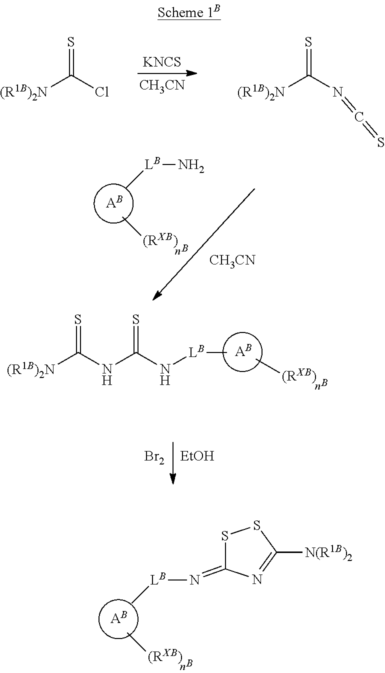

### 5. Uses, Formulation and Administration and Pharmaceutically Acceptable Compositions

According to another embodiment, the present disclosure provides a composition comprising a compound of formula I^{A}, formula I^{B}, formula I^{C}, or formula I^{D}, or any compound selected from Tables 1^{A}, 1^{B}, 1^{C}, 2 and 3, or a pharmaceutically acceptable salt, ester, or salt of ester thereof, and a pharmaceutically acceptable carrier, adjuvant, or vehicle. In some embodiments, the amount of compound in compositions of this disclosure is such that is effective to measurably inhibit SARM1 NADase activity and/or treat a neurodegenerative or neurological disease or disorder, in a biological sample or in a patient. In some embodiments, compositions provided herein contain and/or deliver an amount of a compound of formula I^{A}, formula I^{B}, formula I^{C}, or formula I^{D}, or any compound selected from Tables 1^{A}, 1^{B}, 1^{C}, 2 and 3 that is effective to measurably inhibit SARM1 NADase activity in a biological sample. In some embodiments, compositions provided herein contain and/or deliver an amount of a compound of formula I^{A}, formula I^{B}, formula I^{C}, or formula I^{D}, or any compound selected from Tables 1^{A}, 1^{B}, 1^{C}, 2 and 3 that is effective to measurably inhibit SARM1 NADase activity and/or treat a neurodegenerative or neurological disease or disorder in a patient when administered to the patient in an appropriate dosing regimen. In certain embodiments, a composition of this disclosure is formulated for administration to a patient in need of such composition. In some embodiments, a composition of this disclosure is formulated for oral administration to a patient.

The term “patient,” as used herein, means an animal, preferably a mammal, and most preferably a human.

The term “pharmaceutically acceptable carrier, adjuvant, or vehicle” refers to anon-toxic carrier, adjuvant, or vehicle that does not destroy the pharmacological activity of the compound with which it is formulated. Pharmaceutically acceptable carriers, adjuvants or vehicles that may be used in the compositions of this disclosure include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

A “pharmaceutically acceptable derivative” means any non-toxic salt, ester, salt of an ester or other derivative of a compound of this disclosure that, upon administration to a recipient, is capable of providing, either directly or indirectly, a compound of this disclosure or an inhibitorily active metabolite or residue thereof.

As used herein, the term “inhibitorily active metabolite or residue thereof” means that a metabolite or residue thereof is also an inhibitor of SARM1 NADase activity.

In some embodiments, compositions of the present disclosure may be administered orally, parenterally, by inhalation spray, topically, rectally, nasally, buccally, vaginally or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques. Preferably, the compositions are administered orally, intraperitoneally or intravenously. In some embodiments, sterile injectable forms of the compositions of this disclosure may be aqueous or oleaginous suspension. These suspensions may be formulated according to techniques known in the art using suitable dispersing or wetting agents and suspending agents. In some embodiments, the sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium.

For this purpose, any bland fixed oil may be employed including synthetic mono- or di-glycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as carboxymethyl cellulose or similar dispersing agents that are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation.

In some embodiments, pharmaceutically acceptable compositions of this disclosure may be orally administered in any orally acceptable dosage form including, but not limited to, capsules, tablets, aqueous suspensions or solutions. In some embodiments, in the case of tablets for oral use, carriers commonly used include lactose and corn starch. In some embodiments, lubricating agents, such as magnesium stearate, are also typically added. In some embodiments, for oral administration in a capsule form, useful diluents include lactose and dried cornstarch. In some embodiments, when aqueous suspensions are required for oral use, the active ingredient is combined with emulsifying and suspending agents. In some embodiments, certain sweetening, flavoring or coloring agents may also be added.

In some embodiments, pharmaceutically acceptable compositions of this disclosure may be administered in the form of suppositories for rectal administration. These can be prepared by mixing the agent with a suitable non-irritating excipient that is solid at room temperature but liquid at rectal temperature and therefore will melt in the rectum to release the drug. In some embodiments, such materials include cocoa butter, beeswax and polyethylene glycols.

In some embodiments, pharmaceutically acceptable compositions of this disclosure may also be administered topically, especially when the target of treatment includes areas or organs readily accessible by topical application, including diseases of the eye, the skin, or the lower intestinal tract. Suitable topical formulations are readily prepared for each of these areas or organs.

In some embodiments, topical application for the lower intestinal tract can be effected in a rectal suppository formulation (see above) or in a suitable enema formulation. In some embodiments, topically-transdermal patches may also be used.

For topical applications, provided pharmaceutically acceptable compositions may be formulated in a suitable ointment containing the active component suspended or dissolved in one or more carriers. In some embodiments, carriers for topical administration of compounds of this disclosure include, but are not limited to, mineral oil, liquid petrolatum, white petrolatum, propylene glycol, polyoxyethylene, polyoxypropylene compound, emulsifying wax and water. In some embodiments, provided pharmaceutically acceptable compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol and water.

For ophthalmic use, provided pharmaceutically acceptable compositions may be formulated as micronized suspensions in isotonic, pH adjusted sterile saline, or, preferably, as solutions in isotonic, pH adjusted sterile saline, either with or without a preservative such as benzylalkonium chloride. Alternatively, for ophthalmic uses, the pharmaceutically acceptable compositions may be formulated in an ointment such as petrolatum.

In some embodiments, pharmaceutically acceptable compositions of this disclosure may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents.

Most preferably, pharmaceutically acceptable compositions of this disclosure are formulated for oral administration. Such formulations may be administered with or without food. In some embodiments, pharmaceutically acceptable compositions of this disclosure are administered without food. In other embodiments, pharmaceutically acceptable compositions of this disclosure are administered with food.

In some embodiments, the amount of compounds of the present disclosure that may be combined with the carrier materials to produce a composition in a single dosage form will vary depending upon the host treated, the particular mode of administration. Preferably, provided compositions should be formulated so that a dosage of between 0.01-100 mg/kg body weight/day of the inhibitor can be administered to a patient receiving these compositions.

It should also be understood that a specific dosage and treatment regimen for any particular patient may depend upon a variety of factors, including the activity of the specific compound employed, the age, body weight, general health, sex, diet, time of administration, rate of excretion, drug combination, and the judgment of the treating physician and the severity of the particular disease being treated. In some embodiments, the amount of a compound of the present disclosure in the composition will also depend upon the particular compound in the composition.

In some embodiments, the present disclosure provides methods of identifying a SARM1 NADase inhibitor. Such methods comprise: a) providing a mixture comprising i) a mutant or fragment of SARM1, ii) NAD+, and iii) a candidate inhibitor, wherein the mutant or fragment has constitutive NADase activity; b) incubating the mixture; and c) quantifying NAD+, ADPR (and/or cADPR), nicotinamide or any combination thereof in the mixture after the incubating. In some embodiments, provided methods can further comprise d) determining the molar ratio of NAD+/ADPR (and/or NAD+/cADPR); and e) identifying a candidate inhibitor compound as an NADase inhibitor if the molar ratio of NAD+/ADPR (and/or NAD+/ADPR) is greater than that of a control mixture that does not contain the candidate inhibitor. In some embodiments, one or more of NAD+, ADPR (and/or cADPR), nicotinamide or any combination thereof is quantified by any available analytical method, such as, for example, performing an HPLC analysis, a chemiluminescence assay, a mass spectroscopy analysis, a liquid chromatography-mass spectroscopy analysis, or a combination thereof. In some embodiments, the mixture comprises a cell lysate comprising a mutant or fragment of SARM1. In some embodiments, the cell lysate is a lysate of NRK1-HEK293T cells comprising, consisting of, or consisting essentially of a mutant or fragment of SARM1 that has NADase activity. In some embodiments, the mixture can comprise a purified SAM-TIR polypeptide. In some embodiments, the NRK1-HEK293T cells is treated with nicotinamide riboside (NR), which can be useful for maintaining high NAD+ levels and increasing cell viability in the presence of constitutively active SARM1 molecules. In some embodiments, an inhibitor is identified as an NADase inhibitor if the molar ratio of NAD+ to ADPR (or cADPR) is greater than 4:1. In some embodiments, the candidate inhibitor compound is identified as an NADase inhibitor if the molar ratio of NAD+ to ADPR (or cADPR) is greater than 1:1, 2:1, 3:1, 4:1, 5:1, 6:1, 7:1, 8:1, 9:1 or 10:1.

In some embodiments, the mutant or fragment of SARM1 is a SAM-TIR fragment having constitutive NADase activity.

Fragments of SARM1 having constitutive NADase activity include, for example and without limitation, a SARM1 deleted for the autoinhibitory domain; at least one point mutation of SARM1 that renders the autoinhibitory domain inactive; a fragment of SARM1 consisting of the TIR domain; or a fragment of SARM1 consisting of the SAM and TIR domains. A polypeptide of the present teachings can further include one or more additional amino acid sequences that can act as tags, such as a His tag, a streptavidin tag, or a combination thereof. A polypeptide can include a tag at the amino terminal end, at the carboxy terminal end, or a combination thereof.

In some embodiments, SAM-TIR domains can include human SAM-TIR:

(SEQ ID NO: 1)
VPSWKEAEVQTWLQQIGFSKYCESFREQQVDGDLLLRLTEEELQTDLGMK
SGITRKRFFRELTELKTFANYSTCDRSNLADWLGSLDPRFRQYTYGLVSC
GLDRSLLHRVSEQQLLEDCGIHLGVHRARILTAAREMLHSPLPCTGGKPS
GDTPDVFISYRRNSGSQLASLLKVHLQLHGFSVFIDVEKLEAGKFEDKLI
QSVMGARNFVLVLSPGALDKCMQDHDCKDWVHKEIVTALSCGKNIVPIID
GFEWPEPQVLPEDMQAVLTFNGIKWSHEYQEATIEK IIRFLQGRSSRDS
SAGSDTSLEGAAPMGPT.

The present teachings also provide for the use of isolated TIR domain constructs.

These include constructs including the Human SARM1-TIR domain:

(SEQ ID NO: 2)
TPDVFISYRRNSGSQLASLLKVHLQLHGFSVFIDVEKLEAGKFEDKLIQS
VMGARNFVLVLSPGALDKCMQDHDCKDWVHKEIVTALSCGKNIVPIIDGF
EWPEPQVLPEDMQAVLTFNGIKWSHEYQEATIEKIIRFLQGRSSRDSSAG
SDTSLEGAAPMGPT;

Mouse SARM1-TIR:

(SEQ ID NO: 3)
TPDVFISYRRNSGSQLASLLKVHLQLHGFSVFIDV
EKLEAGKFEDKLIQSVIAARNFVLVLSAGALDKCM
QDHDCKDWVHKEIVTALSCGKNIVPIIDGFEWPEP
QALPEDMQAVLTFNGIKWSHEYQEATIEKIIRFLQ
GRPSQDSSAGSDTSLEGATPMGLP

and Zebrafish SARM1-TIR:

(SEQ ID NO: 4)
PDVFISYRRTTGSQLASLLKVHLQLRGFSVFIDVE
KLEAGRFEEKLITSVQRARNFILVLSANALDKCMG
DVAMKDWVHKEIVTALNGKKNIVPVTDNFVWPDPT
SLPEDMSTILKFNGIKWSHEYQEATIEKILRFLEG
CPSQEKPDGAKTDKKEPQKK.

A skilled artisan will be able to identify mutations or fragments which lack NADase activity.

In some embodiments, an active mutant or fragment of a SARM1 protein is hSARM1-TIR (561-724), mSARM1-TIR (561-724), zfSARM1-TIR (554-713), MyD88-TIR (148-296), or TLR4-TIR (670-839).

In some embodiments, an active mutant or fragment of a SARM1 protein is hSARM1-TIR (561-724), mSARM1-TIR (561-724), zfSARM1-TIR (554-713), MyD88-TIR (148-296), or TLR4-TIR (670-839).

For ease in purification, a SARM1-TIR domain can be engineered with various protein tags. These tags include, such as and without limitation, FLAG, His, Strep-tag, and VENUS tag.

As used herein, a streptavidin tag is a protein domain that has affinity for a bioengineered streptavidin protein. It can have a sequence, such as but without limitation, of Trp-Ser-His-Pro-Gln-Phe-Glu-Lys (SEQ ID NO: 5). Expression vectors and resins are sold under the trade names such as Strep-Tag® and Strep-Tactin® (IBA, Gottingen, Germany).

As used herein, NRK1-HEK293T cells refer to an HEK293 cell line that expresses a Nicotinamide Riboside Kinase 1 (NRK1). NRK1 has sequence

(SEQ ID NO: 6)
MKRFVIGIGGVTNGGKTTLAKSLQKHLPNCSVISQDDFFKPESEIDIDEN
GFLQYDVLEALNMEKMMSAVSCWMENPGSSAGPAALESAQGVPILIIEGF
LLFNYKPLDTIWNRSYFLTVPYEECKRRRSTRVYEPPDPPGYFDGHVWPM
YLKHRQEMSSITWDIVYLDGTRSEEDLFSQVYEDVKQELEKQNGL.

These cells can be stably transformed or transfected with NRK1 or transiently transformed or transfected with NRK1. In some configurations, NRK1 can be transformed or transfected from an expression vector such as but without limitation an FCIV expression vector (Araki, T., et al., Science 305:1010-1013, 2004). In some configurations, NRK1-HEK293T cells can comprise a polyclonal cell line that has been stably transfected with an FCIV expression vector that expresses human Nicotinamide Riboside Kinase 1 (NRK1).

In some embodiments, the mixture can comprise a purified SAM-TIR polypeptide. In some embodiments, the mutant or fragment of SARM1 can consist of or consist essentially of human SARM1 residues 410 to 721 (SEQ ID NO:8). In some embodiments, the mutant or fragment of SARM1 can consist of or consist essentially of human SARM1 residues 560-724. In some embodiments, the mutant or fragment of SARM1 can consist of or consist essentially of human SARM1 residues 560-723. In some embodiments, the mutant or fragment of SARM1 can consist of or consist essentially of human SARM1 residues 560-722. In some embodiments, the mutant or fragment of SARM1 can consist of or consist essentially of human SARM1 residues 560-721. In some embodiments, the mutant or fragment of SARM1 can consist of or consist essentially of a mutant or fragment of SARM1 from any species which has a polypeptide homologous to human SARM1, such as, for example and without limitation, a murine SARM1 polypeptide fragment homologous to human residues 410 to 721. In some embodiments, the SARM1 mutant or SARM1 fragment is a human SARM1 mutant or fragment, a mouse SARM1 mutant or fragment, a zebrafish SARM1 mutant or fragment, a chimpanzee SARM1 mutant or fragment, a Rhesus monkey SARM1 mutant or fragment, a canine SARM1 mutant or fragment, a rat SARM1 mutant or fragment, a chicken SARM1 mutant or fragment, Drosophila SARM1 mutant or fragment, a mosquito SARM1 mutant or fragment, a C. elegans SARM1 mutant or fragment, or a frog SARM1 mutant or fragment. In some embodiments, the mutant or fragment of SARM1 is a SARM1 polypeptide deleted for an N-terminal auto-inhibitory domain. In some embodiments, a SARM1 polypeptide having constitutive NADase activity is from about 150 to about 300 amino acid residues in length. In some embodiments, a SARM1 polypeptide having constitutive NADase activity is from about 160 to about 310 amino acid residues in length. In some embodiments, a SARM1 polypeptide having constitutive NADase activity is from about 160 to about 320 amino acid residues in length.

In some embodiments, a SARM1 polypeptide having constitutive NADase activity has a sequence that has at least 70% sequence identity with a human SARM1 polypeptide having constitutive NADase activity. In some embodiments, a SARM1 polypeptide having constitutive NADase activity has a sequence that has at least 80% sequence identity with a human SARM1 polypeptide having constitutive NADase activity. In some embodiments, a SARM1 polypeptide having constitutive NADase activity has a sequence that has at least 90% sequence identity with a human SARM1 polypeptide having constitutive NADase activity. In some embodiments, a SARM1 polypeptide having constitutive NADase activity has a sequence that has at least 95% sequence identity with a human SARM1 polypeptide having constitutive NADase activity. In some embodiments, a SARM1 polypeptide having constitutive NADase activity and at least 70% sequence identity with a human SARM1 polypeptide having constitutive NADase activity, has conservative amino acid substitutions, insertions, deletions, or a combination thereof. In some embodiments, a SARM1 polypeptide having constitutive NADase activity and at least 80% sequence identity with a human SARM1 polypeptide having constitutive NADase activity, has conservative amino acid substitutions, insertions, deletions, or a combination thereof. In some embodiments, a SARM1 polypeptide having constitutive NADase activity and at least 90% sequence identity with a human SARM1 polypeptide having constitutive NADase activity, has conservative amino acid substitutions, insertions, deletions, or a combination thereof. In some embodiments, a SARM1 polypeptide having constitutive NADase activity and at least 95% sequence identity with a human SARM1 polypeptide having constitutive NADase activity, has conservative amino acid substitutions, insertions, deletions, or a combination thereof. In some embodiments, a SARM1 polypeptide having constitutive NADase activity and a sequence that has at least 70%, at least 80%, at least 90% or at least 95% sequence identity with a human SARM1 polypeptide having constitutive NADase activity, has an artificial sequence, or has a sequence identical to a homologous or orthologous sequence from SARM1 of a non-human species.

In some embodiments, a SARM1 polypeptide having constitutive NADase activity is a full-length SARM1 polypeptide.

In some embodiments, the present teachings include a host cell, e.g., a bacterium such as an E. coli that harbors a nucleic acid that encodes a mutant or fragment of SARM1 of eukaryotic origin has constitutive NADase activity. In some embodiments, the present teachings include a bacterium such as an E. coli that harbors a mutant SARM1 polypeptide of eukaryotic origin that has constitutive NADase activity.

In some embodiments, a method of identifying a SARM1 NADase inhibitor comprises: a) providing a mixture comprising i) a mutant or fragment of SARM1, ii) NAD+ and iii) a candidate inhibitor, wherein the mutant or fragment has constitutive NADase activity; b) incubating the mixture; c) quantifying NAD+ in the mixture after the incubating; and d) identifying the candidate inhibitor compound as an NADase inhibitor if the amount of NAD+ is greater than that of a control mixture that does not contain the candidate inhibitor.

In some embodiments, provided are methods of identifying a SARM1 NADase inhibitor, comprising: a) providing a mixture comprising i) a full-length SARM1, ii) NAD+ and iii) a candidate inhibitor, wherein the full-length SARM1 has constitutive NADase activity; b) incubating the mixture; c) quantifying NAD+ and ADPR (or cADPR) in the mixture after the incubating; d) determining the molar ratio of NAD+:ADPR (or cADPR); and e) identifying the candidate inhibitor compound as an NADase inhibitor if the molar ratio is greater than that of a control mixture that does not contain the candidate inhibitor.

In some embodiments, provided are methods of identifying a SARM1 NADase inhibitor, comprising: a) providing a mixture comprising a solid support to which is bound i) a full-length SARM1 and at least one tag, ii) NAD+, and iii) a candidate inhibitor; b) incubating the mixture; c) quantifying the NAD+ after the incubating; and d) identifying the candidate inhibitor compound as an NADase inhibitor if the concentration of NAD+ is greater than that of a control.

In some embodiments, provided are methods of identifying a SARM1 NADase inhibitor, comprising: a) providing a mixture comprising i) a full-length SARM1, ii) NAD+ and iii) a candidate inhibitor, wherein the full-length SARM1 has constitutive NADase activity; b) incubating the mixture; c) quantifying NAD+ in the mixture after the incubating; and d) identifying the candidate inhibitor compound as an NADase inhibitor if the amount of NAD+ is greater than that of a control mixture that does not contain the candidate inhibitor.

In some embodiments, provided are methods of identifying a SARM1 NADase inhibitor, comprising: a) providing a mixture comprising i) a full-length SARM1 that has constitutive NADase activity, ii) NAD+ and iii) a candidate inhibitor, wherein the full-length SARM1 has constitutive NADase activity; b) incubating the mixture; c) quantifying NAD+ and at least one NADase cleavage product in the mixture after the incubating; and d) identifying the candidate inhibitor compound as an NADase inhibitor if the molar ratio of NAD+ to the at least one NADase cleavage product is greater than that of a control mixture that does not contain the candidate inhibitor.

In some embodiments, the quantifying NAD+ in the mixture comprises, consists of, or consists essentially of performing a chemiluminescence assay. In some embodiments, the quantifying NAD+ in the mixture comprises, consists of, or consists essentially of performing an HPLC analysis. In some embodiments, the mixture can comprise a purified SAM-TIR fragment. In some embodiments, the mixture comprises a cell lysate comprising the mutant or fragment of SARM1. In some embodiments, the cell lysate is a lysate of NRK1-HEK293T cells comprising the mutant or fragment of SARM1. In some embodiments, the NRK1-HEK293T cells comprising the mutant or fragment of SARM1 is treated with NR. In some embodiments, the mutant or fragment of SARM1 is a SAM-TIR fragment. In some embodiments, the mutant or fragment of SARM1 comprises, consists of, or consists essentially of, human SARM1 residues 410 to 721 (SEQ ID NO:8). In some embodiments, the mutant or fragment of SARM1 comprises, consists of, or consists essentially of murine SARM1 residues homologous to those of human SARM1. In some embodiments, the mutant or fragment of SARM1 is a SARM1 polypeptide deleted for an N-terminal auto-inhibitory domain.

In some embodiments, a polypeptide comprises, consists of, or consists essentially of a) a mutant or fragment of SARM1, wherein the mutant or fragment has constitutive NADase activity; and b) at least one tag. In some embodiments, the at least one tag is selected from the group consisting of a streptavidin tag, a His tag, and a combination thereof. In some embodiments, the mutant or fragment of SARM1 is a SAM-TIR fragment. In some embodiments, a mutant or fragment comprises, consists of, or consists essentially of a SAM-TIR fragment, a His tag, and a streptavidin tag. In some embodiments, the streptavidin tag is a tandem streptavidin tag. In some embodiments, a polypeptide comprises, consists of, or consists essentially of an amino terminal tandem streptavidin, a SAM-TIR fragment, and a C-terminal His tag. In some embodiments, the mutant or fragment of SARM1 is a SARM1 polypeptide deleted for an N-terminal auto-inhibitory domain. In some embodiments, the mutant or fragment of SARM1 comprises, consists of, or consists essentially of human SARM1 residues 410 to 721 (SEQ ID NO:8). In some embodiments, the mutant or fragment of SARM1 comprises, consists of, or consists essentially of murine SARM1 residues which are homologous to those of human SARM1 residues 410 to 721 (SEQ ID NO:8). In some embodiments, the mutant or fragment of SARM1 comprises, consists of, or consists essentially of human SARM1 residues 410 to 721 In some embodiments, the polypeptide is immobilized on a solid support. In some embodiments, the solid support is a bead. In some embodiments, vectors include a plasmid or virus comprising a sequence encoding a polypeptide described herein.

In some embodiments, the present disclosure provides methods of identifying a SARM1 NADase inhibitor, which comprises: a) providing a mixture comprising NAD+ and a bead to which is bound a polypeptide consisting of a mutant or fragment of SARM1 having constitutive NADase activity; b) adding a candidate inhibitor to the mixture; c) incubating the mixture; d) quantifying the NAD+ in the mixture; and e) identifying the candidate inhibitor compound as a SARM1 inhibitor if the concentration of NAD+ is greater than that of a control. In some embodiments, provided methods include stopping NADase activity (if any) in the mixture after the incubating. In some embodiments, the polypeptide further includes at least one tag, such as an N-terminal tag. In some embodiments, the N-terminal tag is a streptavidin tag. In some embodiments, the N-terminal tag is a tandem streptavidin tag. In some embodiments, the at least one tag is a C-terminal tag. In some embodiments, the C-terminal tag is a polyhistidine tag. In some embodiments, the bead is a histidine tag purification bead. In some embodiments, the at least one tag is at least two tags. In some embodiments, the at least two tags is an N-terminal tag and a C-terminal tag. In some embodiments, the N-terminal tag is a tandem streptavidin tag and the C-terminal tag is a polyhistidine tag. In some embodiments, the quantifying NAD+ comprises performing an HPLC-based analysis. In some embodiments, the quantifying NAD+ and ADPR (or cADPR) comprises performing an LC/MS-based analysis. In some embodiments, a candidate inhibitor compound is identified as a SARM1 inhibitor if the molar ratio of NAD to ADPR (or cADPR) is greater than 4:1. In some embodiments, a candidate inhibitor compound is identified as a SARM1 inhibitor if the molar ratio of NAD to ADPR (or cADPR) is greater than 1:1, 2:1, 3:1, 4:1, 5:1, 6:1, 7:1, 8:1, 9:1, or 10:1.

In some embodiments, the present disclosure provides a SARM1 polypeptide mutant or fragment. In some embodiments, a SARM1 polypeptide mutant or fragment may be bound to a solid support such as a bead. In some embodiments, the SARM1 polypeptide mutant or fragment bound to a solid support comprises, consists of, or consists essentially of SAM-TIR, a TIR domain, or a SARM1 polypeptide deleted for an autoinhibitory domain. In some embodiments, the SARM1 polypeptide mutant or fragment is selected from the group consisting of a human SARM1 polypeptide mutant or fragment, a mouse SARM1 polypeptide mutant or fragment, and a zebrafish SARM1 polypeptide mutant or fragment. In some embodiments, the SARM1 polypeptide mutant or fragment further comprises, consists of, or consists essentially of a tag. In some embodiments, a SARM1 polypeptide mutant or fragment having NADase activity comprises, consists of, or consists essentially of a SARM1 mutant or fragment bound to a solid support via a protein tag.

In some embodiments, a method of identifying a SARM1 NADase inhibitor comprises: a) providing a mixture comprising at least one cultured neuron comprising an axon; b) adding a candidate SARM1 NADase inhibitor to the mixture; c) adding a labeled NAM to the mixture and transecting the axon; d) incubating the mixture; and e) quantifying the amount of labeled and unlabeled NAD+ in the mixture. In some embodiments, provided methods can further comprise f) calculating the net rate of NAD+ consumption, for example by calculating the % decrease of unlabeled over total NAD+(e.g., light NAD over total (light plus heavy) NAD+) over time. In some embodiments, the calculation is expressed, for example, as %/hr. In some embodiments, an inhibitor of SARM1 is identified when there is a decrease in the post-injury NAD+ consumption rate compared to that of a control mixture that does not contain the candidate inhibitor. In some embodiments, the labeled NAM is deuterium labeled (“heavy”) NAM. In some embodiments, the labeled NAM is d₄-NAM. In some embodiments, the quantifying of labeled and unlabeled NAD+ is performed using analytical methods such as LC-MS/MS. In some embodiments, the at least one cultured neuron is at least one dorsal root ganglion cultured neuron.

In some embodiments, a method of identifying an inhibitor of axonal degeneration comprises, consists of, or consists essentially of: a) providing a mixture comprising at least one cultured neuron comprising an axon; b) adding a candidate inhibitor to the mixture; c) disrupting the neuron; d) calculating the degeneration index using at least one microscope image (Sasaki, Y. et al, Journal of Neuroscience 2009 29(17): 5525-5535); and f) identifying an inhibitor of axon degeneration when there is a significant decrease in the degeneration index compared to a control with no inhibitor. In some embodiments, disrupting the neuron comprises transecting the axon. In some embodiments, disrupting the neuron comprises adding vincristine to the mixture.

In some embodiments, the present disclosure also provides an NRK1-HEK293 cell line comprising HEK293T cells transformed with a Nicotinamide Riboside Kinase 1 (NRK1). In some embodiments, the NRK1-HEK293 cells transformed or transfected with a DNA sequence encoding Nicotinamide Riboside Kinase 1 (NRK1). In some embodiments, the DNA encoding NRK1 can be genomic or cDNA. In some embodiments, an NRK1-HEK293 cell is stably or transiently transformed or transfected with DNA encoding NRK1 from a source exogenous to the host cell. In some embodiments, an NRK1-HEK293 cell is stably or transiently transformed or transfected with DNA encoding NRK1 such that the cell expresses NRK1 at an elevated level compared to control cells. In some embodiments, the DNA encoding NRK1 is under the control of one or more exogenous regulatory sequences such as a promoter, an enhancer or a combination thereof. In some embodiments, a combination of a DNA sequence encoding NRK1 and regulatory sequences is a non-naturally occurring combination. In some embodiments, DNA encoding NRK1, either genomic or cDNA, comprises an expression vector such as an FCIV expression vector. In some embodiments, DNA encoding NRK1 originates from genomic DNA or cDNA, and can be from a vertebrate or invertebrate species such as but not limited to human, mouse, zebrafish or a Drosophila. In some configurations, the NRK1 DNA is a human NRK1 DNA.

### Pharmaceutical Uses

In some embodiments, the present disclosure provides inhibitors of SARM1 NADase activity for treatment of neurodegenerative or neurological diseases or disorders that involve axon degeneration or axonopathy. The present disclosure also provides methods of using inhibitors of SARM1 NADase activity to treat, prevent or ameliorate axonal degeneration, axonopathies and neurodegenerative or neurological diseases or disorders that involve axonal degeneration.

In some embodiments, the present disclosure provides methods of treating neurodegenerative or neurological diseases or disorders related to axonal degeneration, axonal damage, axonopathies, demyelinating diseases, central pontine myelinolysis, nerve injury diseases or disorders, metabolic diseases, mitochondrial diseases, metabolic axonal degeneration, axonal damage resulting from a leukoencephalopathy or a leukodystrophy.

Such neurodegenerative or neurological diseases or disorders may include spinal cord injury, stroke, multiple sclerosis, progressive multifocal leukoencephalopathy, congenital hypomyelination, encephalomyelitis, acute disseminated encephalomyelitis, central pontine myelolysis, osmotic hyponatremia, hypoxic demyelination, ischemic demyelination, adrenoleukodystrophy, Alexander's disease, Niemann-Pick disease, Pelizaeus Merzbacher disease, periventricular leukomalacia, globoid cell leukodystrophy (Krabbe's disease), Wallerian degeneration, optic neuritis, transverse myelitis, amyotrophic lateral sclerosis (ALS, Lou Gehrig's disease), Huntington's disease, Alzheimer's disease, Parkinson's disease, Tay-Sacks disease, Gaucher's disease, Hurler Syndrome, traumatic brain injury, post radiation injury, neurologic complications of chemotherapy (chemotherapy induced neuropathy; CIPN), neuropathy, acute ischemic optic neuropathy, vitamin B₁₂ deficiency, isolated vitamin E deficiency syndrome, Bassen-Komzweig syndrome, Glaucoma, Leber's hereditary optic atrophy, Leber congenital amaurosis, neuromyelitis optica, metachromatic leukodystrophy, acute hemorrhagic leukoencephalitis, trigeminal neuralgia, Bell's palsy, cerebral ischemia, multiple system atrophy, traumatic glaucoma, tropical spastic paraparesis human T-lymphotropic virus 1 (HTLV-1) associated myelopathy, west nile virus encephalopathy, La Crosse virus encephalitis, Bunyavirus encephalitis, pediatric viral encephalitis, essential tremor, Charcot-Marie-Tooth disease, motomeuron disease, spinal muscular atrophy (SMA), hereditary sensory and autonomic neuropathy (HSAN), adrenomyeloneuropathy, progressive supra nuclear palsy (PSP), Friedrich's ataxia, hereditary ataxias, noise induced hearing loss, congenital hearing loss.

In some embodiments, a neuropathy or axonopathy associated with axonal degeneration can be any of a number of neuropathies or axonopathys such as, for example, those that are hereditary or congenital or associated with Parkinson's disease, Alzheimer's disease, Herpes infection, diabetes, amyotrophic lateral sclerosis, a demyelinating disease, ischemia or stroke, chemical injury, thermal injury, and AIDS. In addition, neurodegenerative diseases not mentioned above as well as a subset of the above mentioned diseases can also be treated with the methods of the present disclosure. Such subsets of diseases can include Parkinson's disease or non-Parkinson's diseases, or Alzheimer's disease.

Neuropathies and axonopathies can include any disease or condition involving neurons and/or supporting cells, such as for example, glia, muscle cells or fibroblasts, and, in particular, those diseases or conditions involving axonal damage. Axonal damage can be caused by traumatic injury or by non-mechanical injury due to diseases, conditions, or exposure to toxic molecules or drugs. The result of such damage can be degeneration or dysfunction of the axon and loss of functional neuronal activity. Disease and conditions producing or associated with such axonal damage are among a large number of neuropathic diseases and conditions. Such neuropathies can include peripheral neuropathies, central neuropathies, and combinations thereof. Furthermore, peripheral neuropathic manifestations can be produced by diseases focused primarily in the central nervous systems and central nervous system manifestations can be produced by essentially peripheral or systemic diseases.

Peripheral neuropathies can involve damage to the peripheral nerves, and can be caused by diseases of the nerves or as the result of systemic illnesses. Some such diseases can include diabetes, uremia, infectious diseases such as AIDs or leprosy, nutritional deficiencies, vascular or collagen disorders such as atherosclerosis, and autoimmune diseases such as systemic lupus erythematosus, scleroderma, sarcoidosis, rheumatoid arthritis, and polyarteritis nodosa. Peripheral nerve degeneration can also result from traumatic (mechanical) damage to nerves as well as chemical or thermal damage to nerves. Such conditions that injure peripheral nerves include compression or entrapment injuries such as glaucoma, carpal tunnel syndrome, direct trauma, penetrating injuries, contusions, fracture or dislocated bones; pressure involving superficial nerves (ulna, radial, or peroneal) which can result from prolonged use of crutches or staying in one position for too long, or from a tumor; intraneural hemorrhage; ischemia; exposure to cold or radiation or certain medicines or toxic substances such as herbicides or pesticides. In particular, the nerve damage can result from chemical injury due to a cytotoxic anticancer agent such as, for example, taxol, cisplatinin, a proteasome inhibitor, or a vinca alkaloid such as vincristine. Typical symptoms of such peripheral neuropathies include weakness, numbness, paresthesia (abnormal sensations such as burning, tickling, pricking or tingling) and pain in the arms, hands, legs and/or feet. The neuropathy can also be associated with mitochondrial dysfunction. Such neuropathies can exhibit decreased energy levels, i.e., decreased levels of NAD and ATP.

A peripheral neuropathy can also be a metabolic and endocrine neuropathy which includes a wide spectrum of peripheral nerve disorders associated with systemic diseases of metabolic origin. These diseases include, for example, diabetes mellitus, hypoglycemia, uremia, hypothyroidism, hepatic failure, polycythemia, amyloidosis, acromegaly, porphyria, disorders of lipid/glycolipid metabolism, nutritional/vitamin deficiencies, and mitochondrial disorders, among others. The common hallmark of these diseases is involvement of peripheral nerves by alteration of the structure or function of myelin and axons due to metabolic pathway dysregulation.

Neuropathies can also include optic neuropathies such as glaucoma; retinal ganglion degeneration such as those associated with retinitis pigmentosa and outer retinal neuropathies; optic nerve neuritis and/or degeneration including that associated with multiple sclerosis; traumatic injury to the optic nerve which can include, for example, injury during tumor removal; hereditary optic neuropathies such as Kijer's disease and Leber's hereditary optic neuropathy; ischemic optic neuropathies, such as those secondary to giant cell arteritis; metabolic optic neuropathies such as neurodegenerative diseases including Leber's neuropathy mentioned earlier, nutritional deficiencies such as deficiencies in vitamins B₁₂ or folic acid, and toxicities such as due to ethambutol or cyanide; neuropathies caused by adverse drug reactions and neuropathies caused by vitamin deficiency. Ischemic optic neuropathies also include non-arteritic anterior ischemic optic neuropathy.

Neurodegenerative diseases that are associated with neuropathy or axonopathy in the central nervous system include a variety of diseases. Such diseases include those involving progressive dementia such as, for example, Alzheimer's disease, senile dementia, Pick's disease, and Huntington's disease; central nervous system diseases affecting muscle function such as, for example, Parkinson's disease, motor neuron diseases and progressive ataxias such as amyotrophic lateral sclerosis; demyelinating diseases such as, for example multiple sclerosis; viral encephalitides such as, for example, those caused by enteroviruses, arboviruses, and herpes simplex virus; and prion diseases. Mechanical injuries such as glaucoma or traumatic injuries to the head and spine can also cause nerve injury and degeneration in the brain and spinal cord. In addition, ischemia and stroke as well as conditions such as nutritional deficiency and chemical toxicity such as with chemotherapeutic agents can cause central nervous system neuropathies.

As used herein, the terms “treatment,” “treat,” and “treating” refer to reversing, alleviating, delaying the onset of, or inhibiting the progress of a disease or disorder, or one or more symptoms thereof, as described herein. In some embodiments, treatment may be administered after one or more symptoms have developed. In other embodiments, treatment may be administered in the absence of symptoms. For example, treatment may be administered to a susceptible individual prior to the onset of symptoms (e.g., in light of a history of symptoms and/or in light of genetic or other susceptibility factors). Treatment may also be continued after symptoms have resolved, for example to prevent or delay their recurrence.

The exact amount required will vary from subject to subject, depending on the species, age, and general condition of the subject, the severity of the infection, the particular agent, its mode of administration, and the like. A provided compound or composition of the present disclosure is preferably formulated in dosage unit form for ease of administration and uniformity of dosage. The expression “dosage unit form” as used herein refers to a physically discrete unit of agent appropriate for the patient to be treated. It will be understood, however, that the total daily usage of a provided compound or composition of the present disclosure will be decided by the attending physician within the scope of sound medical judgment. The specific effective dose level for any particular patient or organism will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed, and like factors well known in the medical arts.

A pharmaceutically acceptable composition of this disclosure can be administered to humans and other animals orally, rectally, intravenously, parenterally, intracisternally, intravaginally, intraperitoneally, topically (as by powders, ointments, or drops), bucally, as an oral or nasal spray, or the like, depending on the severity of the infection being treated. In certain embodiments, a provided compound of the present disclosure may be administered orally or parenterally at dosage levels of about 0.01 mg/kg to about 50 mg/kg and preferably from about 1 mg/kg to about 25 mg/kg, of subject body weight per day, one or more times a day, to obtain the desired therapeutic effect.

Liquid dosage forms for oral administration include, but are not limited to, pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a provided compound, it is often desirable to slow the absorption of a compound from subcutaneous or intramuscular injection. This may be accomplished by the use of a liquid suspension of crystalline or amorphous material with poor water solubility. The rate of absorption of the compound then depends upon its rate of dissolution that, in turn, may depend upon crystal size and crystalline form. Alternatively, delayed absorption of a parenterally administered compound form is accomplished by dissolving or suspending a compound in an oil vehicle. Injectable depot forms are made by forming microencapsule matrices of a compound in biodegradable polymers such as polylactide-polyglycolide. Depending upon the ratio of compound to polymer and the nature of the particular polymer employed, the rate of compound release can be controlled. Examples of other biodegradable polymers include poly(orthoesters) and poly(anhydrides). Depot injectable formulations are also prepared by entrapping a compound in liposomes or microemulsions that are compatible with body tissues.

Compositions for rectal or vaginal administration are preferably suppositories which can be prepared by mixing the compounds of this disclosure with suitable non-irritating excipients or carriers such as cocoa butter, polyethylene glycol or a suppository wax which are solid at ambient temperature but liquid at body temperature and therefore melt in the rectum or vaginal cavity and release the active compound.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes. Solid compositions of a similar type may also be employed as fillers in soft and hard-filled gelatin capsules using such excipients as lactose or milk sugar as well as high molecular weight polyethylene glycols and the like.

A provided compound can also be in micro-encapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active compound may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

Dosage forms for topical or transdermal administration of a compound of this disclosure include ointments, pastes, creams, lotions, gels, powders, solutions, sprays, inhalants or patches. The active component is admixed under sterile conditions with a pharmaceutically acceptable carrier and any needed preservatives or buffers as may be required. Ophthalmic formulation, ear drops, and eye drops are also contemplated as being within the scope of this disclosure. Additionally, the present disclosure contemplates the use of transdermal patches, which have the added advantage of providing controlled delivery of a compound to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

According to one embodiment, the present disclosure relates to a method of inhibiting SARM1 NADase activity in a biological sample comprising the step of contacting said biological sample with a provided compound, or a composition comprising said compound.

In certain embodiments, the present disclosure relates to a method of treating axonal degeneration in a biological sample comprising the step of contacting said biological sample with a a provided compound, or a composition comprising said compound.

The term “biological sample”, as used herein, includes, without limitation, cell cultures or extracts thereof; biopsied material obtained from a mammal or extracts thereof; and blood, saliva, urine, feces, semen, tears, or other body fluids or extracts thereof.

Inhibition of enzymes in a biological sample is useful for a variety of purposes that are known to one of skill in the art. Examples of such purposes include, but are not limited to biological assays, gene expression studies, and biological target identification.

Another embodiment of the present disclosure relates to a method of inhibiting SARM1 NADase activity in a patient comprising the step of administering to said patient a provided compound, or a composition comprising said compound.

Those additional agents may be administered separately from a provided compound or composition thereof, as part of a multiple dosage regimen. Alternatively, those agents may be part of a single dosage form, mixed together with a provided compound in a single composition. If administered as part of a multiple dosage regime, the two active agents may be submitted simultaneously, sequentially or within a period of time from one another, normally within five hours from one another.

As used herein, the term “combination,” “combined,” and related terms refers to the simultaneous or sequential administration of therapeutic agents in accordance with this disclosure. For example, a provided compound may be administered with another therapeutic agent simultaneously or sequentially in separate unit dosage forms or together in a single unit dosage form. Accordingly, the present disclosure provides a single unit dosage form comprising a provided compound, an additional therapeutic agent, and a pharmaceutically acceptable carrier, adjuvant, or vehicle.

The amount of both, a provided compound and additional therapeutic agent (in those compositions which comprise an additional therapeutic agent as described above) that may be combined with the carrier materials to produce a single dosage form will vary depending upon the host treated and the particular mode of administration. Preferably, compositions of this disclosure should be formulated so that a dosage of between 0.01-100 mg/kg body weight/day of a provided compound can be administered.

In those compositions which comprise an additional therapeutic agent, that additional therapeutic agent and a provided compound may act synergistically. Therefore, the amount of additional therapeutic agent in such compositions will be less than that required in a monotherapy utilizing only that therapeutic agent. In such compositions a dosage of between 0.01-100 μg/kg body weight/day of the additional therapeutic agent can be administered.

The amount of additional therapeutic agent present in a composition comprising a provided compound will be no more than the amount that would normally be administered in a composition comprising that therapeutic agent as the only active agent. Preferably the amount of additional therapeutic agent in a provided composition will range from about 50% to 100% of the amount normally present in a composition comprising that agent as the only therapeutically active agent.

### EXEMPLIFICATION

The present teachings including descriptions provided in the Examples that are not intended to limit the scope of any claim. Unless specifically presented in the past tense, inclusion in the Examples is not intended to imply that the experiments were actually performed. The following non-limiting examples are provided to further illustrate the present teachings. Those of skill in the art, in light of the present disclosure, will appreciate that many changes can be made in the specific embodiments that are disclosed and still obtain a like or similar result without departing from the spirit and scope of the present teachings.

### Materials and Methods for Examples 1-10

### NRK1-HEK293T Cell Lines.

A clonal HEK293T cell line (NRK1-HEK293T) that expresses Nicotinamide Riboside Kinase 1 (NRK1) was developed so that supplementation with NR during protein expression would significantly augment cellular NAD+ levels and maintain cell viability adequate for protein purification (FIG. 3). FIG. 3 illustrates that a NRK1-HEK293T stable line with NR supplementation maintains higher NAD+ levels upon SARM1-TIR expression. Data was generated from three independent NAD+ measurements from three independent transfection experiments, and normalized to data from a non-transfected experiment run concurrently. Data are presented as mean±SEM; Error bars: SEM; *** P<0.001 two tailed student's t-test.

### Methods.

Some methods and compositions described herein utilize laboratory techniques well known to skilled artisans, and can be found in laboratory manuals such as Sambrook, J., et al., Molecular Cloning: A Laboratory Manual, 3rd ed. Cold Spring Harbor Laboratory Press, Cold Spring Harbor, N.Y., 2001; Methods In Molecular Biology, ed. RiChard, Humana Press, N J, 1995; Spector, D. L. et al., Cells: A Laboratory Manual, Cold Spring Harbor Laboratory Press, Cold Spring Harbor, N.Y., 1998; and Harlow, E., Using Antibodies: A Laboratory Manual, Cold Spring Harbor Laboratory Press, Cold Spring Harbor, N.Y., 1999. Methods of administration of pharmaceuticals and dosage regimes, can be determined according to standard principles of pharmacology, using methods provided by standard reference texts such as Remington: the Science and Practice of Pharmacy (Alfonso R. Gennaro ed. 19th ed. 1995); Hardman, J. G., et al., Goodman & Gilman's The Pharmacological Basis of Therapeutics, Ninth Edition, McGraw-Hill, 1996; and Rowe, R. C., et al., Handbook of Pharmaceutical Excipients, Fourth Edition, Pharmaceutical Press, 2003.

### Reagents.

MagStrep (Strep-Tactin) type 3 XT beads (IBA-Lifesciences, 2-4090-002). Dynabeads HisTag Isolation and Pulldown (ThermoFisher, 10103D). Biotin (Sigma, B4501). β-Nicotinamide Adenine Dinucleotide (Sigma), Nicotinic Acid Adenine Dinucleotide (Sigma), SYPRO Ruby Protein Gel stain (ThermoFisher, S12000), X-tremeGENE 9 DNA transfection reagent (Roche), Shuffle T7 Express Competent E-coli (New England BioLabs) Cell Culture.

HEK293T and NRK1-HEK293T cells were maintained in 10% FBS in DMEM, supplemented with penicillin/streptomycin and glutamine, and passaged by suspending in 0.05% trypsin. Cell lines were continuously monitored for contamination. A batch of HEK293T was tested for Mycoplasma contamination. HEK293T was obtained from ATCC. NRK1-HEK293T is a cell line developed that stably expresses Nicotinamide Riboside Kinase 1 (NRK1) so that supplementation with Nicotinamide Riboside (NR), an NAD+ biosynthetic precursor, during protein expression would significantly augment cellular NAD+ levels and maintain cell viability adequate for protein purification.

### Recombinant DNA.

Mammalian Expression constructs were cloned into FCIV lentiviral vector: StrepTag-hSARM1-TIR-Venus, StrepTag-hSARM1-TIR(E596K)-Venus, StrepTag-GST-MyD88-TIR, StrepTag-GST-TLR4-TIR, StrepTag-hSARM1-TIR-Venus-HisTag, StrepTag-hSARM1-TIR(E596K)-Venus-HisTag.

Bacterial expression constructs were cloned into pET30a+: StrepTag-hSARM1-TIR-HisTag, StrepTag-mSARM1-TIR-HisTag, StrepTag-zfSARM1TIR-HisTag.

### TIR Domain Residues:

hSARM1-TIR (561-724), mSARM1-TIR (561-724), zfSARM1-TIR (554-713), MyD88-TIR (148-296), TLR4-TIR (670-839).

### Mouse Embryonic Dorsal Root Ganglion (DRG) Neuronal Culture.

DRG neurons were isolated from SARM1−/− E13.5 mouse embryos as previously described (Gerdts et al., 2015, Science 348, 453-457) and seeded on plates pre-coated with poly-D-Lysine (Sigma-Aldrich) and laminin (Life Technologies). DRG neurons were maintained in neurobasal medium supplemented with L-glutamine, 2% B27 (Gibco), 50 ng/mL nerve growth factor (Envigo Bioproducts), and 1 μM 5-fluoro-2′deoxyuridine plus 1 μM uridine (Sigma-Aldrich). On DIV 1, neurons were transduced with lentiviral particles generated from HEK293T cells as previously described (Sasaki et al., 2009, J. Neurosci., 29, 5525-5535) expressing Venus alone or the indicated SARM1 construct fused to Venus at the C-terminus. Axons from SARM1−/− DRGs expressing the indicated construct were severed with a razor blade or treated with 40 nM vincristine on DIV 7. SARM1−/− mice (C57/BL6) were housed (12 hr dark/light cycle and less than 5 mice per cage) and used under the direction of institutional animal study guidelines at Washington University in St. Louis.

Protein Expression and Purification from NRK1-HEK293T Stable Line.

Approximately 10 million cells were plated and transfected the next day with 15 μg of StrepTag SARM1-TIR construct DNA using X-tremeGENE™ 9 reagent (Sigma-Aldrich, St. Louis MO). Nicotinamide Riboside (NR) was added at a final concentration of 1 mM to improve cell viability. After 2 days the cells were harvested and lysed by sonication in binding buffer (50 mM Sodium Phosphate buffer pH 8, 300 mM Sodium Chloride, 0.01% Tween-20, protease inhibitor tablets). For single step affinity purification, the whole cell lysates were incubated with 20 μL MagStrep (Strep-Tactin) type 3 XT beads suspension (IBA Lifesciences) for 30 min. The beads were then washed three times with binding buffer and resuspended in 100 μL of binding buffer for enzymatic assays and other downstream applications.

Tandem Affinity Purification (TAP) from NRK1-HEK293T Stable Line.

Dual tagged (Strep-tag and His tag SARM1-TIR) proteins were first purified by Strep Tag affinity methods as described above. For tandem affinity purification, the proteins were then eluted from MagStrep type 3 XT beads with 22.5 mM biotin for 25 min. Supernatant containing the eluted protein was separated from MagStrep beads, and then incubated with 10 μL Co2+ Dynabead suspension for 30 min to bind SARM1-TIR proteins via the His tag. The beads were then washed at least two times with binding buffer and resuspended in 100 μL of binding buffer for downstream applications.

### Bacterial Protein Expression and Tandem Affinity Purification (TAP).

The appropriate dual tag (StrepTag and HisTag) SARM1-TIR was cloned into a pET30a+ plasmid. These constructs as well as non-recombinant pET30a+ were transformed into Shuffle T7 Express Competent E.-coli (New England BioLabs). Single colonies were grown overnight and the next day, cultures were diluted in LB media, grown at 30° C. until they reached A600=0.4-0.8, when IPTG (0.5 mM final concentration) was added. The bacteria were grown for an additional 4 h, pelleted by centrifugation, washed with PBS and stored at −80° C. For protein purification, the frozen bacterial pellet was thawed on ice, resuspended in binding buffer (without protease inhibitors) and incubated with 100 μg/mL lysozyme for 15 min on ice.

Protease inhibitor cocktail was then added and the cells were lysed by sonication. Tandem affinity purification was carried out as described above.

### Preparation of Peptides for LC-MS.

Purified TAP complexes were eluted by boiling the cobalt magnetic beads for 15 min in Tris-HCl buffer (pH 7.6, 100 mM) (40 μL) containing 4% SDS and dithiothreitol (100 mM). The beads were spun at 16,000×g for 5 min and the eluted proteins were mixed with 300 μL of Tris-HCl buffer (pH 8.5, 100 mM) containing 8M urea. The SDS was removed using a filter-aided-sample-preparation (FASP) method (Wisniewski et al., Nat. Methods, 2009, 6, 359-362.). After buffer exchange, 100 μL of buffer (ammonium bicarbonate, pH 7.8, 50 mM) was pipetted into the Microcon® filtration unit (YM-30) and trypsin was added (1 μg in 1 μL). The digest was incubated for 4 h at 37° C. and then overnight in a humid chamber after the addition of another aliquot of trypsin. The digest was acidified (5 μL of neat formic acid) and the peptides were recovered by centrifugation to the lower chamber. The acidified peptides were treated with ethyl acetate as previously described (Erde et al., J. Proteome Res., 2014, 13, 1885-1895). The peptides were desalted by solid phase extraction on a Beckman BioMek NxP robot with C₄ and porous graphite carbon Nutips (Glygen) (Chen et al., Mol. Cell. Proteomics, 2012, 11, M111.011445). The peptides that eluted with acetonitrile (60% in 1% formic acid) were combined, dried in a vacuum centrifuge, dissolved in acetonitrile/formic acid (1%/0.1%) (16 μL). An aliquot (2 μL) was taken for analysis using a fluorescent assay (ThermoFisher Scientific) and the remainder was pipetted into autosampler vials (SUN-SRi), concentrated by vacuum centrifugation and dissolved in aqueous TFA (0.1%) (0.6/μg) for LC-MS analysis (see below).

### NADase Assay and Metabolite Extraction.

Ten microliters of beads incubated with the indicated cell lysate were incubated with 5 μM NAD+ in reaction buffer (92.4 mM NaCl and 0.64×PBS). Reactions were carried out at 25° C. for the indicated amount of time and stopped by addition of 1M of perchloric acid (HClO₄) and placing the tube on ice. NAD+ metabolites were extracted using HClO₄/K₂CO₃ method and quantified by HPLC (see HPLC for metabolite measurement). For LC-MS/MS analysis, the extraction was performed using 50% Methanol in distilled water and chloroform (see LC-MS/MS metabolite measurement for further details).

### HPLC Metabolite Measurement.

Metabolites were isolated from enzyme reaction mixture by extracting with 1M HClO₄, then neutralized with 3M K₂CO₃, and followed by separation by centrifugation. The supernatant (90 μL) containing the extracted metabolites was mixed with 0.5M Potassium Phosphate buffer (10 μL) and metabolites were analyzed by HPLC (Nexera X2) with Kinetex (100×3 mm, 2.6 μm; Phenomenex) column. Internal standards for NAD+, Nicotinamide (Nam), Nicotinic Acid Adenine Dinucleotide (NaAD), ADP Ribose (ADPR) or cADPR were used to generate standard curves for quantification of the respective compounds. The levels for each compound in each experimental sample were normalized to the 0 min time point that was analyzed concurrently.

### LC-MS/MS Metabolite Measurement.

Samples were prepared by mixing the reactions with 50% methanol in distilled water. The samples were placed on ice, and centrifuged.

Soluble metabolites in the supernatant were extracted with chloroform, and the aqueous phase was lyophilized and stored at −20° C. until LC-MS/MS analysis.

For LC-MS/MS, the metabolite samples were reconstituted with 5 mM ammonium formate, centrifuged 12,000×g for 10 min, and the cleared supernatant was applied to the LC-MS/MS for metabolite identification and quantification. Liquid chromatography was performed using an HPLC system (1290; Agilent) with a Synergi Fusion-RP (4.6×150 mm, 4 μm; Phenomenex) column. Samples (10 μl) were injected at a flow rate of 0.55 ml/min with 5 mM ammonium formate for mobile phase A and 100% methanol for mobile phase B. Metabolites were eluted with gradients of 0-7 min, 0-70% B; 7-8 min, 70% B; 9-12 min, 0% B. The metabolites were detected with a Triple Quad mass spectrometer (6460 MassHunter; Agilent) under positive ESI multiple reaction monitoring (MRM). Metabolites were quantified with the aid of a MassHunter quantitative analysis tool (Agilent) with standard curves. Standard curves for each compound were generated by analyzing NAD+, ADPR, and Nam reconstituted in 5 mM ammonium formate. The levels for each compound in each experimental sample were normalized to the 0 min time point that was analyzed concurrently. Sample identity was blinded to individual performing experiment.

### Endogenous Bacterial and Mammalian Cell NAD+ Quantification.

Overnight cultures of E. coli harboring a SARM1-TIR construct were diluted and grown at 30° C. until they reached A600=0.4-0.8. IPTG (0.1 mM final concentration) was added to induce protein expression and the cultures were harvested 60 min later. The cultures were normalized to A600=0.5±0.05 and the pellet from 500 μl of culture suspension was lysed by adding 0.5M HClO₄. NAD+ metabolites were extracted using HClO₄/K₂CO₃ method and measured by HPLC. Two hundred thousand NRK1-HEK293T cells grown in presence of NR were transfected with 1 μg SARM1-TIR expression construct. After two days, the NAD+ metabolites were extracted with 0.5M HClO₄ and 3M K₂CO₃ and measured by HPLC.

### SYPRO Ruby Gel Staining.

Purified bead-SARM1-TIR protein complexes were boiled in Laemmli buffer for 10 min and separated on a 10% Bis-Tris Plus gel. After electrophoresis, the gel was fixed in 50% Methanol/7% acetic acid for 30 min×2, then incubated overnight in SYPRO Ruby Protein Gel stain (Thermo Fisher). The next day, the gel was washed with 10% methanol/7% acetic acid solution for 30 min, rinsed in distilled water for 5 minutes×2, and stained proteins were visualized with a UV transilluminator.

### Enzyme Kinetics Studies.

Vmax, Km, kcat were determined from the reaction velocity of NAD+ consumption in the first 60 seconds of reaction for increasing substrate (NAD+) concentration, and fitting the data to the Michaelis-Menten equation using nonlinear curve fit in GraphPad Prism 7 (GraphPad Software, Inc., La Jolla, CA). kcat was calculated per dimer of purified hSARM1-TIR. Data are presented as Mean±SEM from three independents biological samples and reaction measurements. Enzyme concentration was determined via densitometry analysis on a SYPRO Ruby gel of purified protein, with carbonic anhydrase used as a standard.

### Enzyme Inhibition Studies.

Purified bacterial hSARM1-TIR was tested in the NADase assay with the addition of 1 mM Nam or 1 mM ADPR in the reaction mixture. For dose-response inhibition experiments, varying concentrations of Nam (1, 10, 102, 103, 104 μM) were added to the reaction mixture. The reaction was stopped after 5 min and NAD+ metabolites were extracted by the perchloric acid method and measured by HPLC as indicated above.

### Axonal NAD+ Measurement.

SARM1−/− DRGs were transduced with lentivirus as described above. Cells were supplemented with fresh media every 2 days. On DIV 7, axons were severed with a razor blade. At the indicated timepoint, cell bodies were removed then axonal NAD+ was extracted using perchloric acid/sodium carbonate method and separated with high performance liquid chromatography as previously described (Sasaki et al., J. Neurosci., 2009, 29, 5525-5535).

### Modeling SARM1-TIR Domain.

The human SARM1 TIR domain (aa559-724) was analyzed for structural homologs in the protein data bank (PDB) using HHpred (Söding, J. et al., Nucleic Acids Res., 2005, 33, W244-248) and PHYRE2 (Kelley, L. A., et al., Nat. Protoc., 2015, 10, 845-858.). Protein sequence alignments were generated by HHpred and formatted with JalView. Hits with an E-value greater than 0.1 and score below 40 have a reduced probability of accurate prediction and were excluded. PHYRE2 and SWISS-MODEL (Arnold, K., et al., Bioinformatics, 2006, 22, 195-201) were used to generate 3D structural models of the SARM1 TIR domain using MilB CMP-glycosidase as a template (PDB: 4JEM) or nucleoside 2-deoxyribsoyltransrferase (PDB: 1F8Y). These structures were visualized and superimposed with Chimera (Pettersen, E. F., et al., J. Comput. Chem., 2004, 25, (1605-12)

### Statistical Analyses.

Statistical methods were not used to predetermine sample size. Number and description of n is indicated in each figure legend or appropriate method section. One-way analysis of variance (ANOVA) comparisons were performed for multiple groups and unpaired t-tests or unpaired two-tailed t-tests were used for individual comparisons. Data meets the assumptions of all statistical tests performed with similar variance between groups. All error bars represent SEM and are an estimate of variation within sample groups. Samples from NADase mini-timecourse (1-4 min) experiments that were performed later than initial 5, 10 min reactions and kinetic assays, that had enzymatic activities that were partially reduced either due to increasing storage of bacteria pellets or other technical/biological phenomenon, were excluded from analysis. Fresh bacteria preparations were subsequently prepared. For quantification of Venus expression, DRGs were fixed in paraformaldyhyde and Venus fluorescence visualized by microscopy from multiple fields of axons for each experiment.

DRGs were co-stained for beta tubulin (Mouse anti-beta3 tubulin (TUJ1); from Biolegend) to assess total axon area for each field. Axon degeneration was quantified in distal axons from brightfield images using an ImageJ macro (Sasaki, Y., et al., J. Neurosci., 2009, 29, 5525-5535) that measures the ratio of fragmented axon area to total axon area. For an individual experiment, six fields were analyzed from 2-3 wells per condition. Other data analyses were done with Graph Pad Prism 7, Image J macro, Microsoft Excel, Adobe Illustrator and Photoshop.

### Data and Software Availability

Recombinant DNA sequences have been deposited in BankIt with Accession numbers: KY584388-KY584401.

### Example 1

This example illustrates a SAM-TIR assay for NADase activity and use of the assay to identify and/or characterize compounds that block SARM1-mediated NAD+ cleavage, a crucial step in the elimination of damaged or unhealthy axons. This assay can be utilized, for example, to identify and/or characterize compounds that inhibit TIR domain catalyzed NAD+ cleavage and potentially those that disrupt SAM-mediated multimerization. This assay makes use of a fragment of the SARM1 molecule encompassing the SAM and TIR domains. As demonstrated herein, expression of this fragment without the autoinhibitory N-terminal domain generates an active enzyme that cleaves NAD+.

### Preparation of SARM1 SAM-TIR Lysate (STL)

NRK1-HEK293T cells represent a cell line that has been stably transfected with an FCIV expression vector that expresses human Nicotinamide Riboside Kinase 1 (NRK1), an enzyme that converts the NAD+ biosynthetic precursor nicotinamide riboside (NR) to NMN, the immediate precursor of NAD+. This expression vector has the DNA sequence:

(SEQ ID NO: 7)
gtcgacggatcgggagatctcccgatcccctatggtgcactctcagtacaatctgctctgatgccgcatagttaagccagtatctgctccc
tgcttgtgtgttggaggtcgctgagtagtgcgcgagcaaaatttaagctacaacaaggcaaggcttgaccgacaattgcatgaagaatct
gcttagggttaggcgttttgcgctgcttcgcgatgtacgggccagatatacgcgttgacattgattattgactagttattaatagtaatcaatt
acggggtcattagttcatagcccatatatggagttccgcgttacataacttacggtaaatggcccgcctggctgaccgcccaacgacccc
cgcccattgacgtcaataatgacgtatgttcccatagtaacgccaatagggactttccattgacgtcaatgggtggagtatttacggtaaac
tgcccacttggcagtacatcaagtgtatcatatgccaagtacgccccctattgacgtcaatgacggtaaatggcccgcctggcattatgcc
cagtacatgaccttatgggactttcctacttggcagtacatctacgtattagtcatcgctattaccatggtgatgcggttttggcagtacatca
atgggcgtggatagcggtttgactcacggggatttccaagtctccaccccattgacgtcaatgggagtttgttttggcaccaaaatcaacg
ggactttccaaaatgtcgtaacaactccgccccattgacgcaaatgggcggtaggcgtgtacggtgggaggtctatataagcagcgcgt
tttgcctgtactgggtctctctggttagaccagatctgagcctgggagctctctggctaactagggaacccactgcttaagcctcaataaag
cttgccttgagtgcttcaagtagtgtgtgcccgtctgttgtgtgactctggtaactagagatccctcagacccttttagtcagtgtggaaaatc
tctagcagtggcgcccgaacagggacttgaaagcgaaagggaaaccagaggagctctctcgacgcaggactcggcttgctgaagcg
cgcacggcaagaggcgaggggcggcgactggtgagtacgccaaaaattttgactagcggaggctagaaggagagagatgggtgcg
agagcgtcagtattaagcgggggagaattagatcgcgatgggaaaaaattcggttaaggccagggggaaagaaaaaatataaattaaa
acatatagtatgggcaagcagggagctagaacgattcgcagttaatcctggcctgttagaaacatcagaaggctgtagacaaatactgg
gacagctacaaccatcccttcagacaggatcagaagaacttagatcattatataatacagtagcaaccctctattgtgtgcatcaaaggat
agagataaaagacaccaaggaagctttagacaagatagaggaagagcaaaacaaaagtaagaccaccgcacagcaagcggccgct
gatcttcagacctggaggaggagatatgagggacaattggagaagtgaattatataaatataaagtagtaaaaattgaaccattaggagt
agcacccaccaaggcaaagagaagagtggtgcagagagaaaaaagagcagtgggaataggagctttgttccttgggttcttgggagc
agcaggaagcactatgggcgcagcgtcaatgacgctgacggtacaggccagacaattattgtctggtatagtgcagcagcagaacaat
ttgctgagggctattgaggcgcaacagcatctgttgcaactcacagtctggggcatcaagcagctccaggcaagaatcctggctgtgga
aagatacctaaaggatcaacagctcctggggatttggggttgctctggaaaactcatttgcaccactgctgtgccttggaatgctagttgg
agtaataaatctctggaacagatttggaatcacacgacctggatggagtgggacagagaaattaacaattacacaagcttaatacactcct
taattgaagaatcgcaaaaccagcaagaaaagaatgaacaagaattattggaattagataaatgggcaagtttgtggaattggtttaacat
aacaaattggctgtggtatataaaattattcataatgatagtaggaggcttggtaggtttaagaatagtttttgctgtactttctatagtgaatag
agttaggcagggatattcaccattatcgtttcagacccacctcccaaccccgaggggacccgacaggcccgaaggaatagaagaaga
aggtggagagagagacagagacagatccattcgattagtgaacggatcggcactgcgtgcgccaattctgcagacaaatggcagtatt
catccacaattttaaaagaaaaggggggattggggggtacagtgcaggggaaagaatagtagacataatagcaacagacatacaaact
aaagaattacaaaaacaaattacaaaaattcaaaattttcgggtttattacagggacagcagagatccagtttggttaattaagggtgcagc
ggcctccgcgccgggttttggcgcctcccgcgggcgcccccctcctcacggcgagcgctgccacgtcagacgaagggcgcaggag
cgttcctgatccttccgcccggacgctcaggacagcggcccgctgctcataagactcggccttagaaccccagtatcagcagaaggac
attttaggacgggacttgggtgactctagggcactggttttctttccagagagcggaacaggcgaggaaaagtagtcccttctcggcgat
tctgcggagggatctccgtggggcggtgaacgccgatgattatataaggacgcgccgggtgtggcacagctagttccgtcgcagccg
ggatttgggtcgcggttcttgtttgtggatcgctgtgatcgtcacttggtgagttgcgggctgctgggctggccggggctttcgtggccgc
cgggccgctcggtgggacggaagcgtgtggagagaccgccaagggctgtagtctgggtccgcgagcaaggttgccctgaactggg
ggttggggggagcgcacaaaatggcggctgttcccgagtcttgaatggaagacgcttgtaaggcgggctgtgaggtcgttgaaacaa
ggtggggggcatggtgggcggcaagaacccaaggtcttgaggccttcgctaatgcgggaaagctcttattcgggtgagatgggctgg
ggcaccatctggggaccctgacgtgaagtttgtcactgactggagaactcgggtttgtcgtctggttgcgggggcggcagttatgcggt
gccgttgggcagtgcacccgtacctttgggagcgcgcgcctcgtcgtgtcgtgacgtcacccgttctgttggcttataatgcagggggg
gccacctgccggtaggtgtgcggtaggcttttctccgtcgcaggacgcagggttcgggcctagggtaggctctcctgaatcgacaggc
gccggacctctggtgaggggagggataagtgaggcgtcagtttctttggtcggttttatgtacctatcttcttaagtagctgaagctccggtt
ttgaactatgcgctcggggttggcgagtgtgttttgtgaagttttttaggcaccttttgaaatgtaatcatttgggtcaatatgtaattttcagtgt
tagactagtaaagcttctgcaggtcgactctagaaaattgtccgctaaattctggccgtttttggcttttttgttagacgaagcttgggctgca
ggtcgactctagaggatcatgaagagatttgtcattggaattggtggtgtgacaaacggagggaagacgacactggctaagagcttgca
gaagcaccttcccaactgcagcgtcatatctcaggatgacttcttcaagccagagtctgagatagacatagatgaaaatggttttttgcagt
atgatgtgcttgaagcgctaaatatggaaaaaatgatgtcagcagtttcctgttggatggaaaacccaggaagctctgcgggaccagca
gccttggaaagtgctcaaggggttcccattttaattattgaaggtttccttctctttaattataagcctctggacaccatatggaacagaagtta
cttcctgaccgttccatatgaagaatgtaagaggagaaggagtaccagagtatatgagcctccagaccctccagggtacttcgatggcc
acgtgtggcccatgtacctaaagcacagacaggaaatgagctccatcacctgggacattgtttacctggatggaacaaggtctgaagag
gacctcttctctcaggtgtatgaagatgtcaagcaggaactagagaagcaaaatggtttgGACTATAAAGATGATGAT
GATAAGTAAgctagctaccggtgatccgcccctctccctcccccccccctaacgttactggccgaagccgcttggaataaggc
cggtgtgcgtttgtctatatgttattttccaccatattgccgtcttttggcaatgtgagggcccggaaacctggccctgtcttcttgacgagca
ttcctaggggtctttcccctctcgccaaaggaatgcaaggtctgttgaatgtcgtgaaggaagcagttcctctggaagcttcttgaagaca
aacaacgtctgtagcgaccctttgcaggcagcggaaccccccacctggcgacaggtgcctctgcggccaaaagccacgtgtataaga
tacacctgcaaaggcggcacaaccccagtgccacgttgtgagttggatagttgtggaaagagtcaaatggctctcctcaagcgtattcaa
caaggggctgaaggatgcccagaaggtaccccattgtatgggatctgatctggggcctcggtgcacatgctttacatgtgtttagtcgag
gttaaaaaaacgtctaggccccccgaaccacggggacgtggttttcctttgaaaaacacgatgataatatggccacaaccATGGatg
gccaagttgaccagtgccgttccggtgctcaccgcgcgcgacgtcgccggagcggtcgagttctggaccgaccggctcgggttctcc
cgggacttcgtggaggacgacttcgccggtgtggtccgggacgacgtgaccctgttcatcagcgcggtccaggaccaggtggtgccg
gacaacaccctggcctgggtgtgggtgcgcggcctggacgagctgtacgccgagtggtcggaggtcgtgtccacgaacttccggga
cgcctccgggccggccatgaccgagatcggcgagcagccgtggggggggagttcgccctgcgcgacccggccggcaactgcgt
gcacttcgtggccgaggagcaggactgagaattcgatatcaagcttatcgataatcaacctctggattacaaaatttgtgaaagattgact
ggtattcttaactatgttgctccttttacgctatgtggatacgctgctttaatgcctttgtatcatgctattgcttcccgtatggctttcattttctcct
ccttgtataaatcctggttgctgtctctttatgaggagttgtggcccgttgtcaggcaacgtggcgtggtgtgcactgtgtttgctgacgcaa
cccccactggttggggcattgccaccacctgtcagctcctttccgggactttcgctttccccctccctattgccacggcggaactcategc
cgcctgccttgcccgctgctggacaggggctcggctgttgggcactgacaattccgtggtgttgtcggggaaatcatcgtcctttccttgg
ctgctcgcctgtgttgccacctggattctgcgcgggacgtccttctgctacgtcccttcggccctcaatccagcggaccttccttcccgcg
gcctgctgccggctctgcggcctcttccgcgtcttcgccttcgccctcagacgagtcggatctccctttgggccgcctccccgcatcgat
accgtcgacctcgagacctagaaaaacatggagcaatcacaagtagcaatacagcagctaccaatgctgattgtgcctggctagaagc
acaagaggaggaggaggtgggttttccagtcacacctcaggtacctttaagaccaatgacttacaaggcagctgtagatcttagccactt
tttaaaagaaaaggggggactggaagggctaattcactcccaacgaagacaagatatccttgatctgtggatctaccacacacaaggct
acttccctgattggcagaactacacaccagggccagggatcagatatccactgacctttggatggtgctacaagctagtaccagttgagc
aagagaaggtagaagaagccaatgaaggagagaacacccgcttgttacaccctgtgagcctgcatgggatggatgacccggagaga
gaagtattagagtggaggtttgacagccgcctagcatttcatcacatggcccgagagctgcatccggactgtactgggtctctctggttag
accagatctgagcctgggagctctctggctaactagggaacccactgcttaagcctcaataaagcttgccttgagtgcttcaagtagtgtg
tgcccgtctgttgtgtgactctggtaactagagatccctcagacccttttagtcagtgtggaaaatctctagcagggcccgtttaaacccgc
tgatcagcctcgactgtgccttctagttgccagccatctgttgtttgcccctcccccgtgccttccttgaccctggaaggtgccactcccact
gtcctttcctaataaaatgaggaaattgcatcgcattgtctgagtaggtgtcattctattctggggggtggggggggcaggacagcaagg
gggaggattgggaagacaatagcaggcatgctggggatgcggtgggctctatggcttctgaggcggaaagaaccagctggggctcta
gggggtatccccacgcgccctgtagcggcgcattaagcgcggcgggtgtggtggttacgcgcagcgtgaccgctacacttgccagcg
ccctagcgcccgctcctttcgctttcttcccttcctttctcgccacgttcgccggctttccccgtcaagctctaaatcgggggctccctttagg
gttccgatttagtgctttacggcacctcgaccccaaaaaacttgattagggtgatggttcacgtagtgggccatcgccctgatagacggttt
ttcgccctttgacgttggagtccacgttctttaatagtggactcttgttccaaactggaacaacactcaaccctatctcggtctattcttttgattt
ataagggattttgccgatttcggcctattggttaaaaaatgagctgatttaacaaaaatttaacgcgaattaattctgtggaatgtgtgtcagtt
agggtgtggaaagtccccaggctccccagcaggcagaagtatgcaaagcatgcatctcaattagtcagcaaccaggtgtggaaagtcc
ccaggctccccagcaggcagaagtatgcaaagcatgcatctcaattagtcagcaaccatagtcccgcccctaactccgcccatcccgc
ccctaactccgcccagttccgcccattctccgccccatggctgactaattttttttatttatgcagaggccgaggccgcctctgcctctgagc
tattccagaagtagtgaggaggcttttttggaggcctaggcttttgcaaaaagctcccgggagcttgtatatccattttcggatctgatcagc
acgtgttgacaattaatcatcggcatagtatatcggcatagtataatacgacaaggtgaggaactaaaccatggccaagttgaccagtgc
cgttccggtgctcaccgcgcgcgacgtcgccggagcggtcgagttctggaccgaccggctcgggttctcccgggacttcgtggagga
cgacttcgccggtgtggtccgggacgacgtgaccctgttcatcagcgcggtccaggaccaggtggtgccggacaacaccctggcctg
ggtgtgggtgcgcggcctggacgagctgtacgccgagtggtcggaggtcgtgtccacgaacttccgggacgcctccgggccggcca
tgaccgagatcggcgagcagccgtggggggggagttcgccctgcgcgacccggccggcaactgcgtgcacttcgtggccgagga
gcaggactgacacgtgctacgagatttcgattccaccgccgccttctatgaaaggttgggcttcggaatcgttttccgggacgccggctg
gatgatcctccagcgcggggatctcatgctggagttcttcgcccaccccaacttgtttattgcagcttataatggttacaaataaagcaata
gcatcacaaatttcacaaataaagcatttttttcactgcattctagttgtggtttgtccaaactcatcaatgtatcttatcatgtctgtataccgtc
gacctctagctagagcttggcgtaatcatggtcatagctgtttcctgtgtgaaattgttatccgctcacaattccacacaacatacgagccg
gaagcataaagtgtaaagcctggggtgcctaatgagtgagctaactcacattaattgcgttgcgctcactgcccgctttccagtcgggaa
acctgtcgtgccagctgcattaatgaatcggccaacgcgcggggagaggcggtttgcgtattgggcgctcttccgcttcctcgctcactg
actcgctgcgctcggtcgttcggctgcggcgagcggtatcagctcactcaaaggcggtaatacggttatccacagaatcaggggataa
cgcaggaaagaacatgtgagcaaaaggccagcaaaaggccaggaaccgtaaaaaggccgcgttgctggcgtttttccataggctccg
cccccctgacgagcatcacaaaaatcgacgctcaagtcagaggtggcgaaacccgacaggactataaagataccaggcgtttccccct
ggaagctccctcgtgcgctctcctgttccgaccctgccgcttaccggatacctgtccgcctttctcccttcgggaagcgtggcgctttctca
tagctcacgctgtaggtatctcagttcggtgtaggtcgttcgctccaagctgggctgtgtgcacgaaccccccgttcagcccgaccgctg
cgccttatccggtaactatcgtcttgagtccaacccggtaagacacgacttatcgccactggcagcagccactggtaacaggattagcag
agcgaggtatgtaggcggtgctacagagttcttgaagtggtggcctaactacggctacactagaagaacagtatttggtatctgcgctctg
ctgaagccagttaccttcggaaaaagagttggtagctcttgatccggcaaacaaaccaccgctggtagcggtggtttttttgtttgcaagc
agcagattacgcgcagaaaaaaaggatctcaagaagatcctttgatcttttctacggggtctgacgctcagtggaacgaaaactcacgtt
aagggattttggtcatgagattatcaaaaaggatcttcacctagatccttttaaattaaaaatgaagttttaaatcaatctaaagtatatatgagt
aaacttggtctgacagttaccaatgcttaatcagtgaggcacctatctcagcgatctgtctatttcgttcatccatagttgcctgactccccgt
cgtgtagataactacgatacgggagggcttaccatctggccccagtgctgcaatgataccgcgagacccacgctcaccggctccagatt
tatcagcaataaaccagccagccggaagggccgagcgcagaagtggtcctgcaactttatccgcctccatccagtctattaattgttgcc
gggaagctagagtaagtagttcgccagttaatagtttgcgcaacgttgttgccattgctacaggcatcgtggtgtcacgctcgtcgtttggt
atggcttcattcagctccggttcccaacgatcaaggcgagttacatgatcccccatgttgtgcaaaaaageggttagctcetteggtcctcc
gatcgttgtcagaagtaagttggccgcagtgttatcactcatggttatggcagcactgcataattctcttactgtcatgccatccgtaagatg
cttttctgtgactggtgagtactcaaccaagtcattctgagaatagtgtatgcggcgaccgagttgctcttgcccggcgtcaatacgggata
ataccgcgccacatagcagaactttaaaagtgctcatcattggaaaacgttcttcggggcgaaaactctcaaggatcttaccgctgttgag
atccagttcgatgtaacccactcgtgcacccaactgatcttcagcatcttttactttcaccagcgtttctgggtgagcaaaaacaggaaggc
aaaatgccgcaaaaaagggaataagggcgacacggaaatgttgaatactcatactcttcctttttcaatattattgaagcatttatcagggtt
attgtctcatgagcggatacatatttgaatgtatttagaaaaataaacaaataggggttccgcgcacatttccccgaaaagtgccacctgac.

When these NRK1-expressing cells are supplemented with NR, NAD⁺ levels augmented and cell viability is enhanced to enable efficient production and purification of the constitutively active human SARM1 SAM-TIR (SEQ ID NO: 1) protein fragment.

To express SARM1 SAM-TIR, the SARM1 N-terminal auto-inhibitory domain was deleted, keeping only the initiator Met.

Downstream from this initiator Met, the resulting protein has an N-terminal STREP-TAG® and is composed of human SARM1 residues 410 to 721: MSAWSHPQFEKGGGSGGGSGGSAWSHPQFEKGGGSSGGGGGGSSGGGASVPSWKE AEVQTWLQQIGFSKYCESFREQQVDGDLLLRLTEEELQTDLGMKSGITRKRFFRELTE LKTFANYSTCDRSNLADWLGSLDPRFRQYTYGLVSCGLDRSLLHRVSEQQLLEDCGI HLGVHRARILTAAREMLHSPLPCTGGKPSGDTPDVFISYRRNSGSQLASLLKVHLQLH GFSVFIDVEKLEAGKFEDKLIQSVMGARNFVLVLSPGALDKCMQDHDCKDWVHKEI VTALSCGKNIVPIIDGFEWPEPQVLPEDMQAVLTFNGIKWSHEYQEATIEKIIRFLQGR SSRDSSAGSDTSLEGAAPMGPT (SEQ ID NO: 8). The fragment encoding the SARM1 SAM-TIR protein was cloned into the FCIV expression construct by standard methods to generate the FCIV-SST vector. The resultant vector has the following sequence:

(SEQ ID NO: 9)
gtcgacggatcgggagatctcccgatcccctatggtgcactctcagtaca
atctgctctgatgccgcatagttaagccagtatctgctccctgcttgtgt
gttggaggtcgctgagtagtgcgcgagcaaaatttaagctacaacaaggc
aaggcttgaccgacaattgcatgaagaatctgcttagggttaggcgtttt
gcgctgcttcgcgatgtacgggccagatatacgcgttgacattgattatt
gactagttattaatagtaatcaattacggggtcattagttcatagcccat
atatggagttccgcgttacataacttacggtaaatggcccgcctggctga
ccgcccaacgacccccgcccattgacgtcaataatgacgtatgttcccat
agtaacgccaatagggactttccattgacgtcaatgggtggagtatttac
ggtaaactgcccacttggcagtacatcaagtgtatcatatgccaagtacg
ccccctattgacgtcaatgacggtaaatggcccgcctggcattatgccca
gtacatgaccttatgggactttcctacttggcagtacatctacgtattag
tcatcgctattaccatggtgatgcggttttggcagtacatcaatgggcgt
ggatagcggtttgactcacggggatttccaagtctccaccccattgacgt
caatgggagtttgttttggcaccaaaatcaacgggactttccaaaatgtc
gtaacaactccgccccattgacgcaaatgggcggtaggcgtgtacggtgg
gaggtctatataagcagcgcgttttgcctgtactgggtctctctggttag
accagatctgagcctgggagctctctggctaactagggaacccactgctt
aagcctcaataaagcttgccttgagtgcttcaagtagtgtgtgcccgtct
gttgtgtgactctggtaactagagatccctcagacccttttagtcagtgt
ggaaaatctctagcagtggcgcccgaacagggacttgaaagcgaaaggga
aaccagaggagctctctcgacgcaggactcggcttgctgaagcgcgcacg
gcaagaggcgaggggcggcgactggtgagtacgccaaaaattttgactag
cggaggctagaaggagagagatgggtgcgagagcgtcagtattaagcggg
ggagaattagatcgcgatgggaaaaaattcggttaaggccagggggaaag
aaaaaatataaattaaaacatatagtatgggcaagcagggagctagaacg
attcgcagttaatcctggcctgttagaaacatcagaaggctgtagacaaa
tactgggacagctacaaccatcccttcagacaggatcagaagaacttaga
tcattatataatacagtagcaaccctctattgtgtgcatcaaaggataga
gataaaagacaccaaggaagctttagacaagatagaggaagagcaaaaca
aaagtaagaccaccgcacagcaagcggccgctgatcttcagacctggagg
aggagatatgagggacaattggagaagtgaattatataaatataaagtag
taaaaattgaaccattaggagtagcacccaccaaggcaaagagaagagtg
gtgcagagagaaaaaagagcagtgggaataggagctttgttccttgggtt
cttgggagcagcaggaagcactatgggcgcagcgtcaatgacgctgacgg
tacaggccagacaattattgtctggtatagtgcagcagcagaacaatttg
ctgagggctattgaggcgcaacagcatctgttgcaactcacagtctgggg
catcaagcagctccaggcaagaatcctggctgtggaaagatacctaaagg
atcaacagctcctggggatttggggttgctctggaaaactcatttgcacc
actgctgtgccttggaatgctagttggagtaataaatctctggaacagat
ttggaatcacacgacctggatggagtgggacagagaaattaacaattaca
caagcttaatacactccttaattgaagaatcgcaaaaccagcaagaaaag
aatgaacaagaattattggaattagataaatgggcaagtttgtggaattg
gtttaacataacaaattggctgtggtatataaaattattcataatgatag
taggaggcttggtaggtttaagaatagtttttgctgtactttctatagtg
aatagagttaggcagggatattcaccattatcgtttcagacccacctccc
aaccccgaggggacccgacaggcccgaaggaatagaagaagaaggtggag
agagagacagagacagatccattcgattagtgaacggatcggcactgcgt
gcgccaattctgcagacaaatggcagtattcatccacaattttaaaagaa
aaggggggattggggggtacagtgcaggggaaagaatagtagacataata
gcaacagacatacaaactaaagaattacaaaaacaaattacaaaaattca
aaattttcgggtttattacagggacagcagagatccagtttggttaatta
agggtgcagcggcctccgcgccgggttttggcgcctcccgcgggcgcccc
cctcctcacggcgagcgctgccacgtcagacgaagggcgcaggagcgttc
ctgatccttccgcccggacgctcaggacagcggcccgctgctcataagac
tcggccttagaaccccagtatcagcagaaggacattttaggacgggactt
gggtgactctagggcactggttttctttccagagagcggaacaggcgagg
aaaagtagtcccttctcggcgattctgcggagggatctccgtggggcggt
gaacgccgatgattatataaggacgcgccgggtgtggcacagctagttcc
gtcgcagccgggatttgggtcgcggttcttgtttgtggatcgctgtgatc
gtcacttggtgagttgcgggctgctgggctggccggggctttcgtggccg
ccgggccgctcggtgggacggaagcgtgtggagagaccgccaagggctgt
agtctgggtccgcgagcaaggttgccctgaactgggggttggggggagcg
cacaaaatggcggctgttcccgagtcttgaatggaagacgcttgtaaggc
gggctgtgaggtcgttgaaacaaggtggggggcatggtgggcggcaagaa
cccaaggtcttgaggccttcgctaatgcgggaaagctcttattcgggtga
gatgggctggggcaccatctggggaccctgacgtgaagtttgtcactgac
tggagaactcgggtttgtcgtctggttgcgggggcggcagttatgcggtg
ccgttgggcagtgcacccgtacctttgggagcgcgcgcctcgtcgtgtcg
tgacgtcacccgttctgttggcttataatgcagggtggggccacctgccg
gtaggtgtgcggtaggcttttctccgtcgcaggacgcagggttcgggcct
agggtaggctctcctgaatcgacaggcgccggacctctggtgaggggagg
gataagtgaggcgtcagtttctttggtcggttttatgtacctatcttctt
aagtagctgaagctccggttttgaactatgcgctcggggttggcgagtgt
gttttgtgaagttttttaggcaccttttgaaatgtaatcatttgggtcaa
tatgtaattttcagtgttagactagtaaagcttctgcaggtcgactctag
aaaattgtccgctaaattctggccgtttttggcttttttgttagacgaag
cttgggctgcaggtcgactctagaggatccGGATCCGCCACCATGTCAgc
tTGGAGCCACCCACAATTCGAAAAAGGCGGTGGCTCAGGCGGTGGCTCAG
GTGGCTCAGCTTGGAGCCACCCACAATTCGAAAAAGGCGGTGGCTCATCT
GGCGGAGGTGGCGGTGGCTCATCTGGCGGAGGTGCTAGCgtgcccagctg
gaaggaggccgaggttcagacgtggctgcagcagatcggtttctccaagt
actgcgagagcttccgggagcagcaggtggatggcgacctgcttctgcgg
ctcacggaggaggaactccagaccgacctgggcatgaaatcgggcatcac
ccgcaagaggttctttagggagctcacggagctcaagaccttcgccaact
attctacgtgcgaccgcagcaacctggcggactggctgggcagcctggac
ccgcgcttccgccagtacacctacggcctggtcagctgcggcctggaccg
ctccctgctgcaccgcgtgtctgagcagcagctgctggaagactgcggca
tccacctgggcgtgcaccgcgcccgcatcctcacggcggccagagaaatg
ctacactccccgctgccctgtactggtggcaaacccagtggggacactcc
agatgtcttcatcagctaccgccggaactcaggttcccagctggccagtc
tcctgaaggtgcacctgcagctgcatggcttcagtgtcttcattgatgtg
gagaagctggaagcaggcaagttcgaggacaaactcatccagagtgtcat
gggtgcccgcaactttgtgttggtgctatcacctggagcactggacaagt
gcatgcaagaccatgactgcaaggattgggtgcataaggagattgtgact
gctttaagctgcggcaagaacattgtgcccatcattgatggcttcgagtg
gcctgagccccaggtcctgcctgaggacatgcaggctgtgcttactttca
acggtatcaagtggtcccacgaataccaggaggccaccattgagaagatc
atccgcttcctgcagggccgctcctcccgggactcatctgcaggctctga
caccagtttggagggtgctgcacccatgggtccaacctaaactctagaat
tcgatatcaagcttatcgataatcaacctctggattacaaaatttgtgaa
agattgactggtattcttaactatgttgctccttttacgctatgtggata
cgctgattaatgcctttgtatcatgctattgcttcccgtatggctttcat
tttctcctccttgtataaatcctggttgctgtctctttatgaggagttgt
ggcccgttgtcaggcaacgtggcgtggtgtgcactgtgtttgctgacgca
acccccactggttggggcattgccaccacctgtcagctcctttccgggac
tttcgctttccccctccctattgccacggcggaactcatcgccgcctgcc
ttgcccgctgctggacaggggctcggctgttgggcactgacaattccgtg
gtgttgtcggggaaatcatcgtcctttccttggctgctcgcctgtgttgc
cacctggattctgcgcgggacgtccttctgctacgtcccttcggccctca
atccagcggaccttccttcccgcggcctgctgccggctctgcggcctctt
ccgcgtcttcgccttcgccctcagacgagtcggatctccctttgggccgc
ctccccgcatcgataccgtcgacctcgagacctagaaaaacatggagcaa
tcacaagtagcaatacagcagctaccaatgctgattgtgcctggctagaa
gcacaagaggaggaggaggtgggttttccagtcacacctcaggtaccttt
aagaccaatgacttacaaggcagctgtagatcttagccactattaaaaga
aaaggggggactggaagggctaattcactcccaacgaagacaagatatcc
ttgatctgtggatctaccacacacaaggctacttccctgattggcagaac
tacacaccagggccagggatcagatatccactgacctttggatggtgcta
caagctagtaccagttgagcaagagaaggtagaagaagccaatgaaggag
agaacacccgcttgttacaccctgtgagcctgcatgggatggatgacccg
gagagagaagtattagagtggaggtttgacagccgcctagcatttcatca
catggcccgagagctgcatccggactgtactgggtctctctggttagacc
agatctgagcctgggagctctctggctaactagggaacccactgcttaag
cctcaataaagcttgccttgagtgcttcaagtagtgtgtgcccgtctgtt
gtgtgactctggtaactagagatccctcagacccttttagtcagtgtgga
aaatctctagcagggcccgtttaaacccgctgatcagcctcgactgtgcc
ttctagttgccagccatctgttgtttgcccctcccccgtgccttccttga
ccctggaaggtgccactcccactgtcctttcctaataaaatgaggaaatt
gcatcgcattgtctgagtaggtgtcattctattctggggggtggggtggg
gcaggacagcaagggggaggattgggaagacaatagcaggcatgctgggg
atgcggtgggctctatggcttctgaggcggaaagaaccagctggggctct
agggggtatccccacgcgccctgtagcggcgcattaagcgcggcgggtgt
ggtggttacgcgcagcgtgaccgctacacttgccagcgccctagcgcccg
ctcctttcgctttcttcccttcctttctcgccacgttcgccggctttccc
cgtcaagctctaaatcgggggctccctttagggttccgatttagtgcttt
acggcacctcgaccccaaaaaacttgattagggtgatggttcacgtagtg
ggccatcgccctgatagacggtttttcgccctttgacgttggagtccacg
ttctttaatagtggactcttgttccaaactggaacaacactcaaccctat
ctcggtctattcttttgatttataagggattttgccgatttcggcctatt
ggttaaaaaatgagctgatttaacaaaaatttaacgcgaattaattctgt
ggaatgtgtgtcagttagggtgtggaaagtccccaggctccccagcaggc
agaagtatgcaaagcatgcatctcaattagtcagcaaccaggtgtggaaa
gtccccaggctccccagcaggcagaagtatgcaaagcatgcatctcaatt
agtcagcaaccatagtcccgcccctaactccgcccatcccgcccctaact
ccgcccagttccgcccattctccgccccatggctgactaatttatttatt
tatgcagaggccgaggccgcctctgcctctgagctattccagaagtagtg
aggaggctataggaggcctaggcttttgcaaaaagctcccgggagcttgt
atatccattttcggatctgatcagcacgtgttgacaattaatcatcggca
tagtatatcggcatagtataatacgacaaggtgaggaactaaaccatggc
caagttgaccagtgccgttccggtgctcaccgcgcgcgacgtcgccggag
cggtcgagttctggaccgaccggctcgggttctcccgggacttcgtggag
gacgacttcgccggtgtggtccgggacgacgtgaccctgttcatcagcgc
ggtccaggaccaggtggtgccggacaacaccctggcctgggtgtgggtgc
gcggcctggacgagctgtacgccgagtggtcggaggtcgtgtccacgaac
ttccgggacgcctccgggccggccatgaccgagatcggcgagcagccgtg
ggggcgggagttcgccctgcgcgacccggccggcaactgcgtgcacttcg
tggccgaggagcaggactgacacgtgctacgagatttcgattccaccgcc
gccttctatgaaaggttgggcttcggaatcgttttccgggacgccggctg
gatgatcctccagcgcggggatctcatgctggagttcttcgcccacccca
acttgtttattgcagcttataatggttacaaataaagcaatagcatcaca
aatttcacaaataaagcatttttttcactgcattctagttgtggtttgtc
caaactcatcaatgtatcttatcatgtctgtataccgtcgacctctagct
agagcttggcgtaatcatggtcatagctgtttcctgtgtgaaattgttat
ccgctcacaattccacacaacatacgagccggaagcataaagtgtaaagc
ctggggtgcctaatgagtgagctaactcacattaattgcgttgcgctcac
tgcccgctttccagtcgggaaacctgtcgtgccagctgcattaatgaatc
ggccaacgcgcggggagaggcggtttgcgtattgggcgctcttccgcttc
ctcgctcactgactcgctgcgctcggtcgttcggctgcggcgagcggtat
cagctcactcaaaggcggtaatacggttatccacagaatcaggggataac
gcaggaaagaacatgtgagcaaaaggccagcaaaaggccaggaaccgtaa
aaaggccgcgttgctggcgtttttccataggctccgcccccctgacgagc
atcacaaaaatcgacgctcaagtcagaggtggcgaaacccgacaggacta
taaagataccaggcgtttccccctggaagctccctcgtgcgctctcctgt
tccgaccctgccgcttaccggatacctgtccgcctttctcccttcgggaa
gcgtggcgctttctcatagctcacgctgtaggtatctcagttcggtgtag
gtcgttcgctccaagctgggctgtgtgcacgaaccccccgttcagcccga
ccgctgcgccttatccggtaactatcgtcttgagtccaacccggtaagac
acgacttatcgccactggcagcagccactggtaacaggattagcagagcg
aggtatgtaggcggtgctacagagttcttgaagtggtggcctaactacgg
ctacactagaagaacagtatttggtatctgcgctctgctgaagccagtta
ccttcggaaaaagagttggtagctcttgatccggcaaacaaaccaccgct
ggtagcggtggtttttttgtttgcaagcagcagattacgcgcagaaaaaa
aggatctcaagaagatcctttgatcttttctacggggtctgacgctcagt
ggaacgaaaactcacgttaagggattttggtcatgagattatcaaaaagg
atcttcacctagatccttttaaattaaaaatgaagttttaaatcaatcta
aagtatatatgagtaaacttggtctgacagttaccaatgcttaatcagtg
aggcacctatctcagcgatctgtctatttcgttcatccatagttgcctga
ctccccgtcgtgtagataactacgatacgggagggcttaccatctggccc
cagtgctgcaatgataccgcgagacccacgctcaccggctccagatttat
cagcaataaaccagccagccggaagggccgagcgcagaagtggtcctgca
actttatccgcctccatccagtctattaattgttgccgggaagctagagt
aagtagttcgccagttaatagtttgcgcaacgttgttgccattgctacag
gcatcgtggtgtcacgctcgtcgtttggtatggcttcattcagctccggt
tcccaacgatcaaggcgagttacatgatcccccatgttgtgcaaaaaagc
ggttagctccttcggtcctccgatcgttgtcagaagtaagttggccgcag
tgttatcactcatggttatggcagcactgcataattctcttactgtcatg
ccatccgtaagatgcttttctgtgactggtgagtactcaaccaagtcatt
ctgagaatagtgtatgcggcgaccgagttgctcttgcccggcgtcaatac
gggataataccgcgccacatagcagaactttaaaagtgctcatcattgga
aaacgttcttcggggcgaaaactctcaaggatcttaccgctgttgagatc
cagttcgatgtaacccactcgtgcacccaactgatcttcagcatctttta
ctttcaccagcgtttctgggtgagcaaaaacaggaaggcaaaatgccgca
aaaaagggaataagggcgacacggaaatgttgaatactcatactcttcca
tttcaatattattgaagcatttatcagggttattgtctcatgagcggata
catatttgaatgtatttagaaaaataaacaaataggggttccgcgcacat
ttccccgaaaagtgccacctgac.

NRK1-HEK293T cells were seeded onto 150 cm² plates at 20×10⁶ cells per plate. The next day, the cells were transfected with 15 μg FCIV-SST (SAM-TIR expression plasmid, SEQ ID NO: 9) using X-TREMEGENE™ 9 DNA Transfection Reagent (Roche product #06365787001). The cultures were supplemented with 1 mM NR at time of transfection to minimize toxicity from SAM-TIR overexpression. Forty-eight hours after transfection, cells were harvested, pelleted by centrifugation at 1,000 rpm (Sorvall ST 16R centrifuge, Thermo Fisher), and washed once with cold PBS (0.01 M phosphate buffered saline NaCl 0.138 M; KCl 0.0027 M; pH 7.4). The cells were resuspended in PBS with protease inhibitors (cOmplete™ protease inhibitor cocktail, Roche product #11873580001) and cell lysates were prepared by sonication (Branson Sonifer 450, output=3, 20 episodes of stroke). The lysates were centrifuged (12,000×g for 10 min at 4° C.) to remove cell debris and the supernatants (containing SARM1 SAM-TIR protein) were stored at −80° C. for later use in the in vitro SARM1 SAM-TIR NADase assay (see below). Protein concentration was determined by the Bicinchoninic (BCA) method and used to normalize lysate concentrations.

### Compound Library

The NCI Diversity IV compound library and the Pharmacon 1600 compound library were screened for SARM1 SAM-TIR inhibitors. The stock concentration for each compound is 10 mM (in DMSO). The compounds were first diluted 10-fold to produce a 1 mM stock (in DMSO). This stock was further diluted 20-fold into 20% DMSO/80% water to produce 50 μM working stocks of each compound.

### In Vitro SARM1 SAM-TIR NADase Assays and Inhibitor Screen

### HPLC-Based Assay 1.

Reaction mixtures were prepared on ice by mixing SARM1 SAM-TIR cell lysate (0.14 μg total protein), compound stock (5 μM final concentration), and PBS (pH 7.4) to a final volume of 12 μl. NAD+(5 μM final concentration) was then added for a final reaction volume of 20 μl. The mixture was incubated at 37° C. for 60 min; reaction was then stopped by addition of 180 μl of 0.55 M perchloric acid (HClO₄). The reactions were then placed on ice for 10 min, and the reaction plates were centrifuged for 10 min at 4,000 rpm (Sorvall ST 16R centrifuge). The supernatant (120 μl) was transferred to a new plate and 10 μl of 3M K₂CO₃ was added to neutralize the solution. Precipitated salts were removed by centrifugation 10 min at 4,000 rpm (Sorvall ST 16R centrifuge). The supernatant was transferred and analyzed by HPLC (Shimadzu Nexera X2) with KINETEX® (100×3 mm, 2.6 μm; PHENOMENEX®) column and metabolites were monitored with absorbance at 254 nm.

### Results.

### SARM1 SAM-TIR Lysate Cleaves NAD+

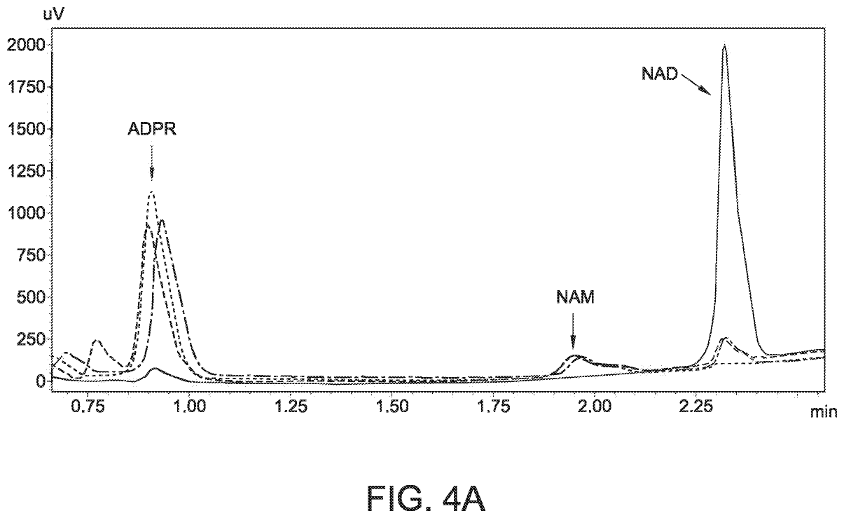

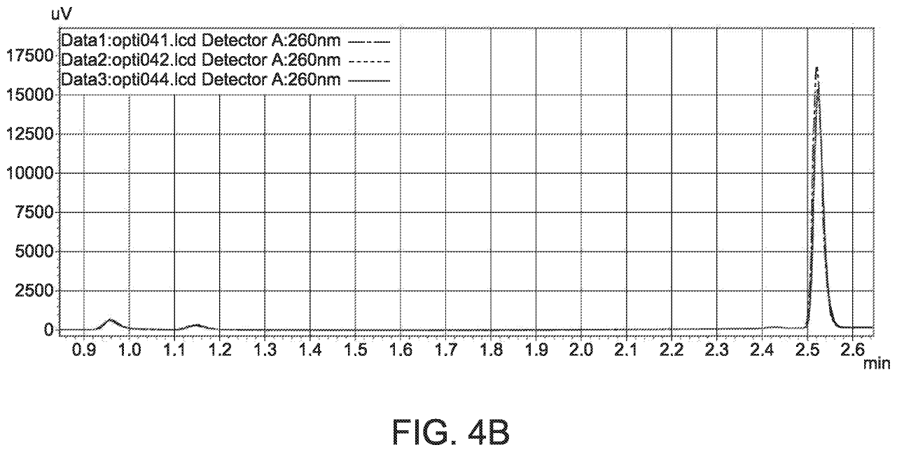

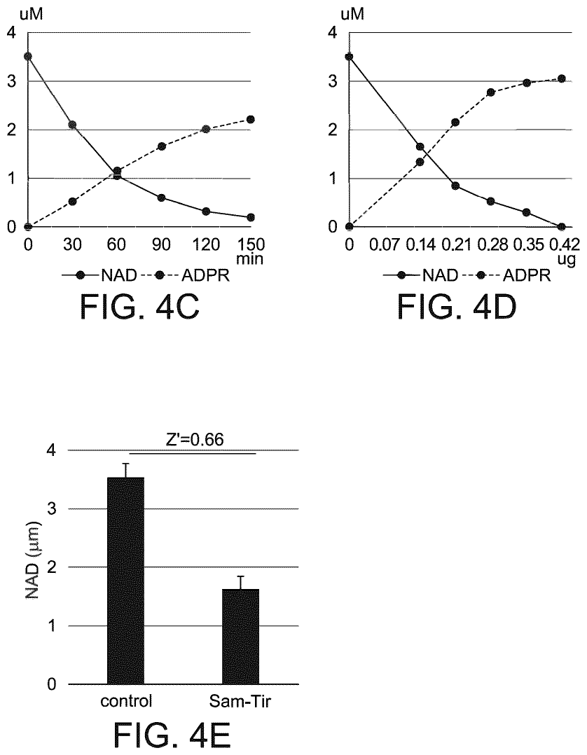

Using HPLC-based assay 1, the SARM1 SAM-TIR lysate cleaved NAD+ in a dose- and time-dependent manner (FIG. 4A, C, D), whereas control lysate prepared from non-transfected NRK1-HEK293T cells showed no NAD+ cleavage (FIG. 4B). Loss of NAD+ was accompanied by an increase in nicotinamide (Nam) and ADP ribose (ADPR), indicating that cleavage of the nicotinamide-ribosyl bond of NAD+(FIG. 4A). SARM1 SAM-TIR lysate was incubated with NAD+(5 μM) for indicated times. The NAD+ levels are shown in FIG. 4A (peak at 2.52 min in HPLC traces) were reduced and ADPR levels were increased (peak at 1.15 min) with time. Trace color: black—NAD alone; green—lysate; blue—on beads, green in eluate.

For FIG. 4A, the SARM1 Sam-TIR protein was purified by Strep Tag affinity methods. HEK-NRK1 lysate (100 ul) was incubated with 20 μL MagStrep (Strep-Tactin) type 3 XT beads suspension (IBA Lifesciences) for 30 min (in buffer W: 100 mM Tris/HCl, pH 8.0; 150 mM NaCl; 1 mM EDTA). The beads were then washed three times with buffer W, and bound proteins were eluted from MagStrep type 3 XT beads with 25 mM biotin in buffer W for 30 min. Supernatant containing the eluted protein can be used for NADase activity assay. Pierce protease inhibitors (ThermoFisher cat #88266) were added into all buffers. FIG. 4A shows HPLC traces for the starting substrate, NAD, and the cleavage products, ADPR and Nicotinamide (NAM), that are generated by active SARM1 TIR NADase. The black trace shows NAD without added enzyme. The red trace shows that SAM-TIR-containing lysate has potent NADase activity (NAD is lost and the products, ADPR and NAM, are generated). The Blue trace shows that the SAM-TIR enzyme can be purified on beads as described above and this enzyme is active (again, loss of NAD and generation of ADPR and NAM). Finally, the green trace shows that active SAM-TIR enzyme can be eluted from the beads and remains active (loss of NAD, generation of ADPR and NAM). FIG. 4B shows that control lysate didn't consume NAD+ after the same period of incubation. FIG. 4C shows quantitative values of NAD+ and ADPR of HPLC traces in FIG. 4A. FIG. 4D shows that cleavage of NAD+ by SARM1 SAM-TIR lysate is dose-dependent. The indicated amount of SARM1 SAM-TIR lysate was incubated with NAD+(5 μM) at 37° C. for 60 min and conversion of NAD+ to ADPR was monitored. FIG. 4E shows that quantitation of NAD+/ADPR ratio after 60 min reaction using 0.14 μg protein of either control and SAM-TIR lysate. These results are consistent with the NADase activity observed using TIR Assay (see FIG. 8).

In summary, the present Example demonstrates that a lysate containing the SARM1-TIR domain contains NADase activity.

Identification and/or Characterization of SARM1 SAM-TIR NADase Inhibitors.

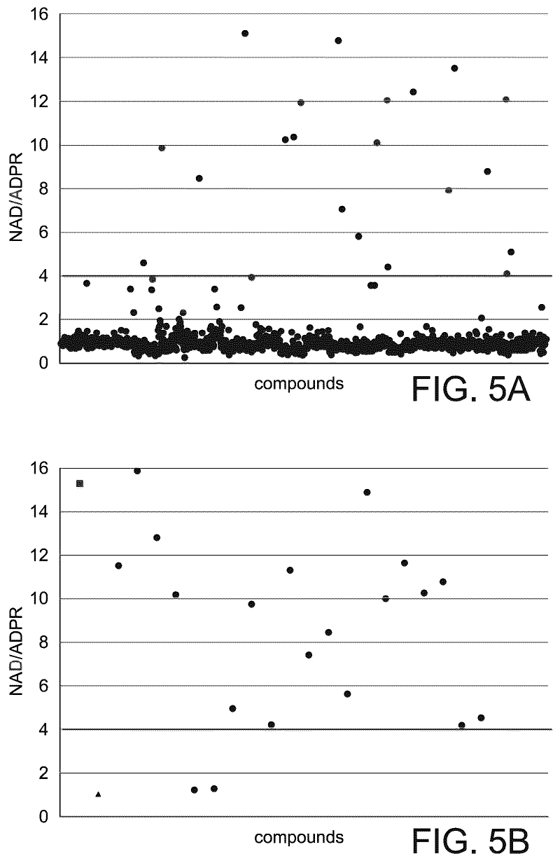

To identify inhibitors of SARM1 NADase activity, the levels of NAD+ and the enzymatic cleavage product ADPR in the reactions were quantified by HPLC. From these values, the NAD+/ADPR ratio for each compound was calculated and the ratio used as a measure of NAD+ cleavage activity (Note: there is a small residual but detectable ADPR signal in control samples derived from the HEK293 lysate). This ratio was compared to the ratio generated in the absence of compound inhibitors. A significant reduction of NADase activity (defined as NAD+/ADPR ratio >4) was used to identify compounds that inhibited SAM-TIR catalyzed NAD+ cleavage (FIG. 5A-B). FIG. 5A illustrates a primary screen of all 1600 compounds from the library (5 μM compound with 5 μM NAD+). In FIG. 5B, the 20 positive hits (NAD+/ADPR>4 from the top panel were re-tested. Eighteen of the 20 original ‘positive hits’ were again identified as inhibitors in the secondary screen (controls: square, no reaction time; triangle: DMSO control).

Identification and Characterization of Compounds that Inhibit SARM1 SAM-TIR NADase Activity

The NAD+/ADPR ratio was used to determine the NAD+ cleavage activity of the SARM1 SAM-TIR lysate using the HPLC based assay 1. It will be appreciated that any precise, quantitative method of measuring NAD+ levels could be used for the detection of SARM1 NADase activity. An NAD+/ADPR ratio=˜1 was established as a baseline control (without inhibitor). The assay was robust (Z′=0.537, control lysate (n=14) NAD+/ADPR=19.52±2.25; SAM-TIR lysate (n=14) NAD+/ADPR=1.186±0.607 (mean±SD). In the control condition, a small amount of ADPR is detected by HPLC) (FIG. 4A-B). An empirically generated (NAD+/ADPR) cutoff value of 4:1 was used, where NAD+/ADPR>4 represents significant suppression of SARM1 SAM-TIR lysate NADase activity.

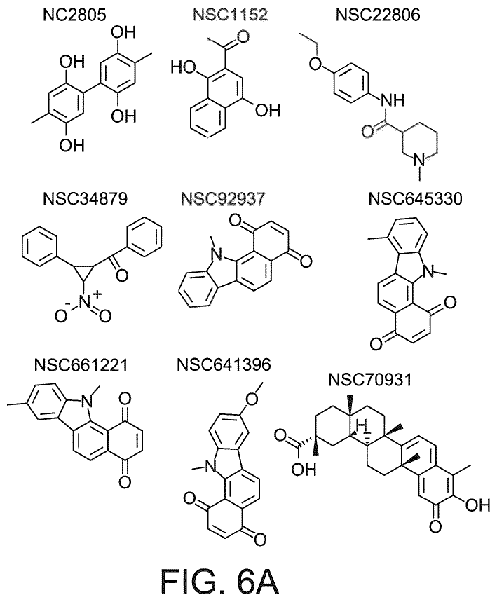

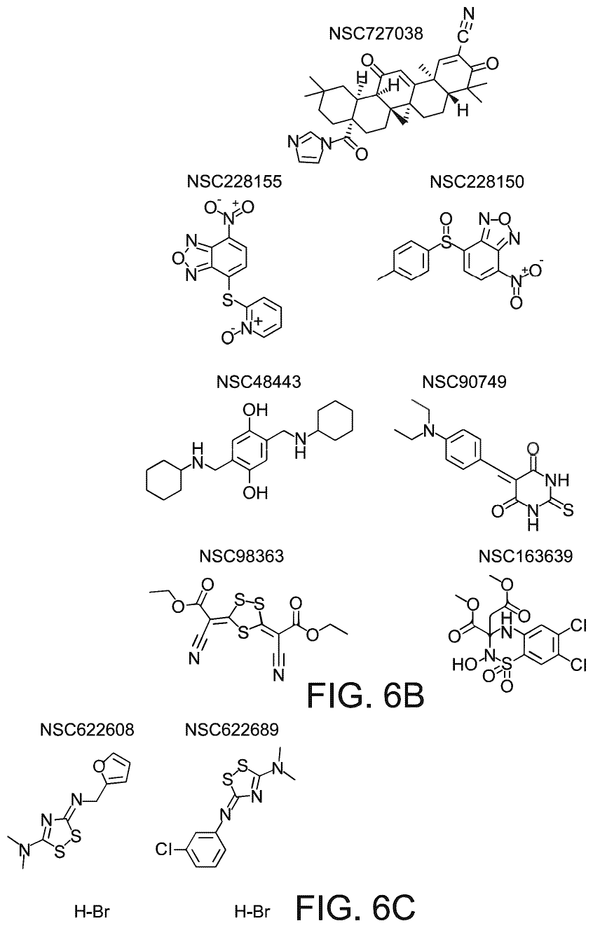

Twenty compounds out of 1600 from the NCI Diversity IV compound library were identified as inhibitors in the primary screen (FIG. 5A). Eighteen of these were identified as positive ‘hits’ in a secondary screen, with 10 of them showing robust inhibition of SARM1 SAM-TIR activity (i.e., NAD+/ADPR>10 (FIG. 5B; FIG. 6A-C).

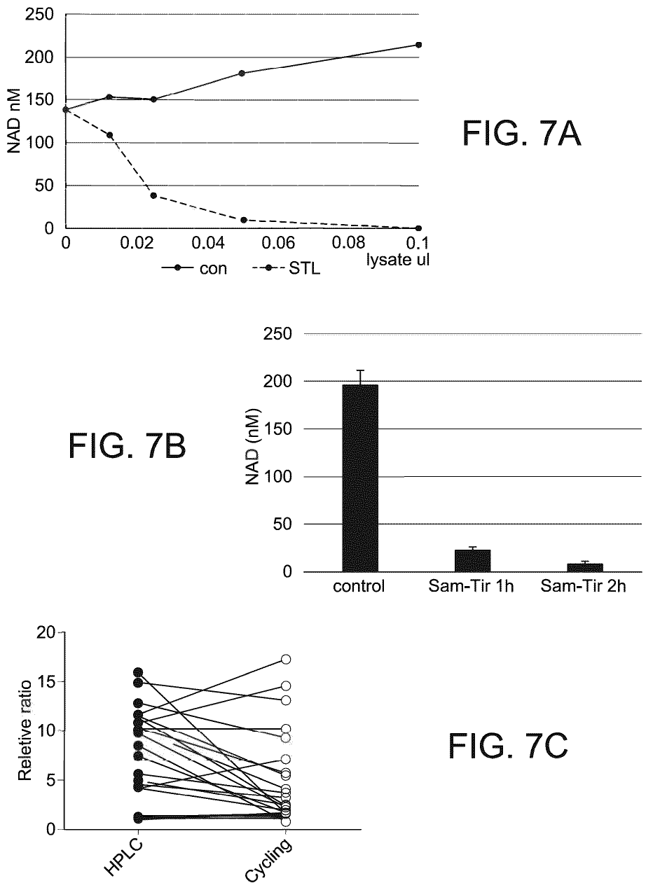

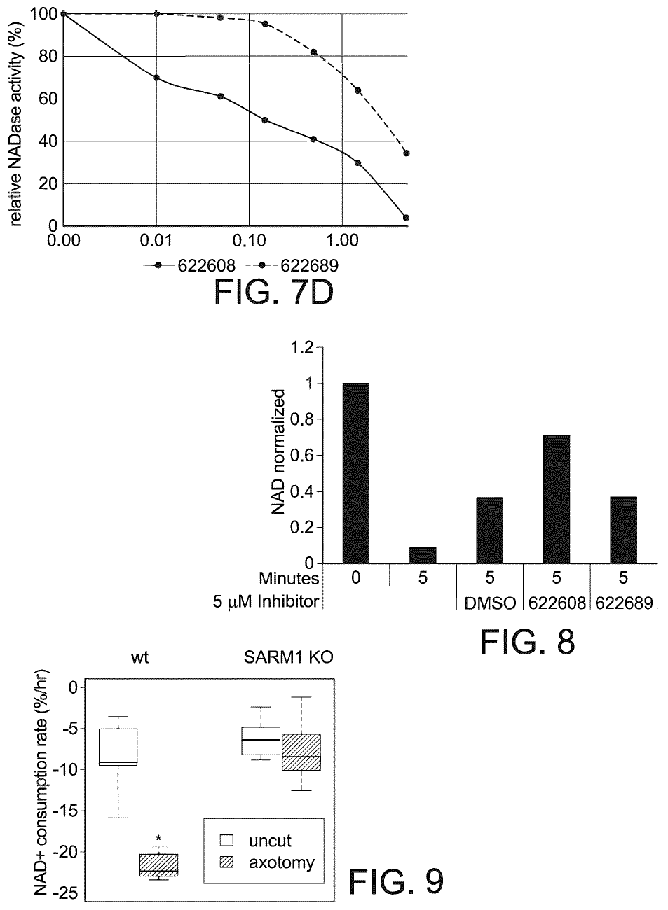

Inhibitors identified in the initial screen were then tested in the NAD+ Glo assay (see section infra), which employ an enzymatic cycling reaction to determine NAD+ concentration. The assay itself is highly reproducible (FIG. 7A-B). FIG. 7A illustrates SAM-TIR lysate (STL) but not control (con) lysate decreased NAD+ determined by NAD+ Glo assay. The elevated NAD+ levels in high dose lysate conditions are mostly likely derived from lysate itself. FIG. 7B illustrates that the assay is very robust (Z′=0.66 control 2 h reaction time vs SAM-TIR 1 h reaction time; Z′=0.71 control 2 h reaction time vs SAM-TIR 2 h reaction time). Control 2 h NAD+=196.20±15.66 nM; SAM-TIR 1 h NAD+=22.48±3.98 nM; SAM-TIR 2 h NAD+=8.18 2.79 nM. Most hits identified in the initial HPLC assay (14/18) showed significant inhibition of SAM-TIR NADase activity in NAD+-Glo assay (FIG. 7C). Cycling assay is highly correlated with HPLC assay. 14 out of 18 hits from HPLC also blocked NADase activity significantly (2 fold increase of luminescence intensity). Relative ratio for HPLC assay 1 represents the NAD+/ADPR ratio, while for the cycling assay, it represents the ratio of IC50≈150 nM (FIG. 7D). Two compounds showed the best inhibition in NAD+ Glo assay. IC50 for NSC622608≈150 nM.

Luminescence-based assay. This assay can complement the results obtained by HPLC, and can permit a higher throughput of compound library screening than is possible with HPLC methods. This assay is an adaptation of the NAD+/NADH-GLO™ assay (Promega G9071, Promega Corporation, Madison, WI). In this assay, NAD+ cycling enzymes convert NAD+ into NADH. In the presence of NADH, the reductase enzymatically converts a pro-luciferin reductase substrate into luciferin. Luciferin is detected using ULTRA-GLO™ rLuciferase, and the chemiluminescence intensity is proportional to the amount of NAD+ and NADH in the sample. Under the present assay conditions, the amount of NAD+ and NADH present in the lysate is undetectable with this assay, precluding any endogenous contribution to the final NAD+ detected. The assay was set up as follows: 2 μl candidate inhibitor (final concentration 1 μM, 2% DMSO), 0.07 μg lysate (2 μl), and 2 μl of 400 nM NAD+. The reaction was incubated at 37° C. for 60 min, then 6 μl NAD+/NADH-GLO™ detection reagent was added. After 30 min at room temperature, the luminescent signals were quantified using a CYTATION™ 5 imaging reader (BIOTEK®). The SARM1 SAM-TIR lysate catalyzed a dose-dependent depletion of NAD+, whereas NAD+ levels did not decline when reactions were performed with lysate prepared from control NRK1-HEK293T cells (FIG. 7A-D).

### Example 2

The present Example describes a SARM1 TIR-based Assay. This assay is similar to the assay described in Example 1, but allows for the identification and/or characterization of compounds that directly interact with the TIR domain, whereas the assay described in Example 1 can also identify compounds that disrupt SAM domain interactions. This assay makes use of the bacterial expression of a tagged version of the SARM1 TIR fragment that can be affinity purified. Displaying this artificial SARM1 TIR domain on a solid surface (i.e. affinity beads) generates an active NAD+ cleavage enzyme.

### Materials and Methods

Tagged proteins included the following:
StrepTag-humanSARM1-TIR-6xHisTag
(SEQ ID NO: 10)
MSAWSHPQFEKGGGSGGGSGGSAWSHPQFEKGGGSSGGGASTPDVFISYR
RNSGSQLASLLKVHLQLHGFSVFIDVEKLEAGKFEDKLIQSVMGARNFVL
VLSPGALDKCMQDHDCKDWVHKEIVTALSCGKNIVPIIDGFEWPEPQVLP
EDMQAVLTFNGIKWSHEYQEATIEKIIRFLQGRSSRDSSAGSDTSLEGAA
PMGPTHHHHHH
StrepTag-mouseSARM1-TIR-6xHisTag
(SEQ ID NO: 11)
MSAWSHPQFEKGGGSGGGSGGSAWSHPQFEKGGGSSGGGASTPDVFISYR
RNSGSQLASLLKVHLQLHGFSVFIDVEKLEAGKFEDKLIQSVIAARNFVL
VLSAGALDKCMQDHDCKDWVHKEIVTALSCGKNIVPIIDGFEWPEPQALP
EDMQAVLTFNGIKWSHEYQEATIEKIIRFLQGRPSQDSSAGSDTSLEGAT
PMGLPHHHHHH
StrepTag-zebrafishSARM1-TIR-6xHisTag
(SEQ ID NO: 12)
MSAWSHPQFEKGGGSGGGSGGSAWSHPQFEKGGGSSGGGASPDVFISYRR
TTGSQLASLLKVHLQLRGFSVFIDVEKLEAGRFEEKLITSVQRARNFILV
LSANALDKCMGDVAMKDWVHKEIVTALNGKKNIVPVTDNFVWPDPTSLPE
DMSTILKFNGIKWSHEYQEATIEKILRFLEGCPSQEKPDGAKTDKKEPQK
KHHHHHH

### Bacterial Protein Expression and Tandem Affinity Purification (TAP).

The TIR domain of SARM1 was tagged with a tandem STREP-TAG® at the N-terminus, and a polyhistidine tag at the C-terminus, and was cloned into a pET30a+ plasmid. The construct was then transformed into SHuffle® T7 Express Competent E-coli (New England BioLabs, Ipswich, MA) and single colonies were grown overnight. The next day, cultures were diluted in LB media, grown at 30° C. until they reached A600=0.4-0.8, when IPTG (0.5 mM final concentration) was added. The bacteria were grown for an additional 4 h, pelleted by centrifugation, washed with PBS and stored at −80° C. For protein purification, the frozen bacterial pellet was thawed on ice, resuspended in binding buffer (without protease inhibitors) and incubated with 100 μg/mL lysozyme for 15 min on ice. Protease inhibitor cocktail was then added and the cells were lysed by sonication.

The SARM1 TIR protein was first purified by Strep Tag affinity methods where bacterial lysates were incubated with 20 μL MagStrep (STREP-TACTIN®, IBA GmBH, Gottingen Germany) type 3 XT beads suspension (IBA Lifesciences) for 30 min. The beads were then washed three times with binding buffer, and bound proteins were eluted from MagStrep type 3 XT beads with 22.5 mM biotin for 25 min. Supernatant containing the eluted protein was separated from MagStrep beads, and incubated with 10 μL Co2+ DYNABEAD® (ThermoFisher Scientific, Waltham, MA) suspension for 30 min to bind SARM1-TIR proteins via the His tag. The beads were then washed at least two times with binding buffer and resuspended in 100 μL of binding buffer for NADase assay.

Ten microliters of purified SARM1-TIR laden beads were incubated with 5 μM NAD+ in reaction buffer (92.4 mM NaCl and 0.64×PBS). Reactions were carried out at 25° C. for the indicated amount of time and stopped by addition of 1M of perchloric acid (HClO₄) and placing the tube on ice. NAD+ metabolites were extracted using HClO₄/K₂CO₃ method and quantified by HPLC (see metabolite measurement below). For LC-MS/MS analysis, the extraction was performed using 50% methanol in distilled water and chloroform (see LC-MS/MS metabolite measurement below). FIG. 8 shows that some compounds identified as inhibitors in the SARM1 SAM-TIR assay also inhibit NADase activity of purified SARM1 TIR in the in vitro assay. Select potent inhibitors (622608, 622689) identified from SARM1 SAM-TIR lysate screen were added to the reaction at 5 μM. NAD normalized to control at 0 min.

### HPLC Metabolite Measurement.

Metabolites were isolated from enzyme reaction mixture by extracting with 1M HClO4, then neutralized with 3M K2CO3, and followed by separation by centrifugation. The supernatant (90 μL) containing the extracted metabolites was mixed with 0.5M Potassium Phosphate buffer (10 μL) and metabolites were analyzed by HPLC (Nexera X2) with KINETEX® (100×3 mm, 2.6 μm; PHENOMENEX®) column and metabolites are monitored with absorbance at 254 nm. Internal standards for NAD+, Nicotinamide (Nam), ADP Ribose (ADPR) were used to generate standard curves for quantification of the respective compounds. The levels for each compound in each experimental sample was normalized to the 0 min time point that was analyzed concurrently.

### LC-MS/MS Metabolite Measurement.

Samples were prepared by mixing the reactions with 50% methanol in distilled water. The samples were placed on ice, centrifuged, soluble metabolites in the supernatant were extracted with chloroform, and the aqueous phase was lyophilized and stored at −20° C. until LC-MS/MS analysis. For LC-MS/MS, the metabolite samples were reconstituted with 5 mM ammonium formate, centrifuged 12,000×g for 10 min, and the cleared supernatant was applied to the LC-MS/MS for metabolite identification and quantification. Liquid chromatography was performed by HPLC system (1290; Agilent) with SYNERGI™ Fusion-RP (4.6×150 mm, 4 m; PHENOMENEX®, Phenomenex, Torance, CA) column. Samples (10 l) were injected at a flow rate of 0.55 ml/min with 5 mM ammonium formate for mobile phase A and 100% methanol for mobile phase B and metabolites were eluted with gradients of 0-7 min, 0-70% B; 7-8 min, 70% B; 9-12 min, 0% B. Metabolites were detected with Triple Quad mass spectrometer (6460 MassHunter; AGILENT®) under positive ESI multiple reaction monitoring (NAD+:664>428 with 160V (fragmentation), 22V (collision), 7V (post-acceleration)). Metabolites were quantified by MassHunter quantitative analysis tool (AGILENT®) with standard curves. Standard curves for each compound were generated by analyzing NAD+, ADPR, and Nam reconstituted in 5 mM ammonium formate. The levels for each compound in each experimental sample were normalized to the 0 min time point that was analyzed concurrently. Sample identity was blinded to individual performing experiment.

### Example 3

This example illustrates an NAD flux assay which allows for the identification and/or characterization of compounds that inhibit SARM1-mediated NAD consumption in axons of cultured neurons. This assay utilizes the full-length SARM1 protein activated by a neuronal injury in neurons. This assay measures the injury-activated SARM1-dependent degradation of NAD+ in axons. This method allows for the independent assessment of NAD+ synthesis and NAD+ consumption.

### DRG Neuronal Culture.

Mouse dorsal root ganglion (DRG) were dissected from embryonic days 13.5 CD1 mouse embryo (˜50 ganglion per embryo) and incubated with 0.05% Trypsin solution containing 0.02% EDTA (Gibco) at 37° C. for 15 min. Then cell suspensions are triturated by gentle pipetting and washed 3 times with DRG growth medium (Neurobasal medium (Gibco) containing 2% B27 (Invitrogen), 100 ng/ml 2.55 NGF (Harlan Bioproduts), 1 μM uridine (Sigma), 1 μM 5-fluoro-2′-deoxyuridine (Sigma), penicillin, and streptomycin). Cells were suspended in DRG growth medium at a ratio of 100 μl medium/50 DRGs. The cell density of these suspensions was −7×106 cells/ml. Cell suspension (10 μl) was placed in the center of the well using 24-well tissue culture plates (Corning) coated with poly-D-Lysine (0.1 mg/ml; Sigma) and laminin (3 μg/ml; Invitrogen). Cells were allowed to adhere in humidified tissue culture incubator (5% CO2) for 15 min and then DRG growth medium was gently added (500 μl).

### Axonal Metabolite Collection.

At DIV6, neuronal cell bodies and axons were separated using a microsurgical blade under the microscope at 0 (for control NAD+ consumption) or 4 (for axotomized axonal NAD+ consumption) hours prior to metabolite collection. Then the DRG cultures were placed on ice, culture medium was replaced with ice-cold 0.9% NaCl solution (0.5 μl), and the DRG cell bodies were removed using a pipet. The 0.9% NaCl solution was removed, and the axonal metabolites were extracted by incubation with ice-cold 1:1 mixture of MeOH and water (150 μl per well) on ice for 10 min. The metabolite containing solutions were transferred into test tubes and extracted twice with chloroform (100 μl per sample). The aqueous phase (120 l) was lyophilized and reconstituted with 50 μl of 5 mM ammonium formate and cleared supernatants after centrifugation at 12,000×g for 10 min were transferred to sample vials and measured.

NAD+ Measurement using LC-MS/MS.

Serial dilutions of NAD+(25 μM to 320 μM, Sigma) in 5 mM ammonium formate were used for calibration. Liquid chromatography was performed with 10 μl of each sample injected at a flow rate of 0.55 ml/min with 5 mM ammonium formate for mobile phase A and 100% methanol for mobile phase B (HPLC:1290; Agilent with Synergi Fusion-RP (4.6×150 mm, 4 m; Phenomenex)). Metabolites were eluted with gradients of 0-7 min, 0-70% B; 7-8 min, 70% B; 9-12 min, 0% B. The metabolites were detected with a Triple Quad mass spectrometer (6460 MassHunter; Agilent) under positive ESI multiple reaction monitoring (MRM) (D4-NAD+:668>428, D3-NAD+:667>428, NAD+:664>428 with 160V (fragmentation), 22V (collision), 7V (post-acceleration)). Metabolites were quantified by MassHunter quantitative analysis tool (Agilent) with standard curves.

### NAD+ Consumption Measurement.

For NAD+ consumption measurements, DRG neurons were incubated with D4-Nam (300 μM: 2,3,4,5 deuterium Nam; C/D/N Isotopes Inc., D-3457) for 4 hours and axonal metabolites were collected as described above. For NAD+ flux measurements after axonal injury, D4-Nam was added at the same time as axotomy. Labeled (heavy) or non-labeled (light) NAD+ was quantified by LC-MS/MS. For heavy-labeled NAD+, D3-NAD+ as well as D4-NAD+ was observed. This is due to the replacement of deuterium at C₄ position with non-labeled proton during NAD+-NADH cycling. The values of D3-NAD+ and D4-NAD+ were added and used this combined value as the amount of heavy NAD+. The net rate of NAD+ consumption were calculated by % decrease of light NAD+ over total NAD+(sum of heavy and light NAD+) at 4 hours after D4-Nam application and expressed %/hr. Axonal NAD+ consumption was −8.5±3.8%/hr without axotomy and increased to −21.7±1.6%/hr in axotomized axons. This acceleration of NAD+ consumption is completely blocked in SARM1 KO axons (−6.3±2.4%/hr uninjured vs. −7.9±3.7%/hr after axotomy) and can be used for a read out of SARM1 activation after injury (FIG. 9). FIG. 9 shows that the depletion of SARM1 completely blocked the increase of NAD+ consumption. Thus the increased NAD+ consumption can be used as a read out of SARM1 activation in injured neurons. one-way ANOVA F(3,32)=50.6, p=3×10-12. * p<0.005 denotes significant difference from control uncut with Holm-Bonferroni multiple comparison (n=9).

### NAD+ Consumption Assay for Assessing the Efficacy of SARM1 Inhibitors in Neurons.

Selected chemical compounds (final concentration 5 μM at 30 min prior to D4-Nam addition) as well as 300 μM D4-Nam were added to DRG culture medium and axons were immediately transected (3 wells) or keep intact (3 wells). Axonal metabolites were collected at 4 hours post D4-Nam addition and metabolites can be analyzed as described above. NAD+ consumption rate before and after axotomy can be calculated. Shown here is a demonstration that in the absence of SARM1 (SARM1 knockout, KO), there is no axotomy-induced increase in NAD+ consumption rate (FIG. 9). Thus the inhibitory effects of compounds on SARM1 activation can be assessed by a decrease in the post-injury NAD+ consumption rate. This assay tests the efficacy of SARM1 inhibitors in axons of cultured neurons.

### Example 4

This example illustrates an in vitro axon degeneration assay and application of this assay to characterize compounds. In this example, this assay was used to test whether inhibitors of SARM1 NADase activity can inhibit axon degeneration that rapidly follows axonal NAD+ loss after injury.

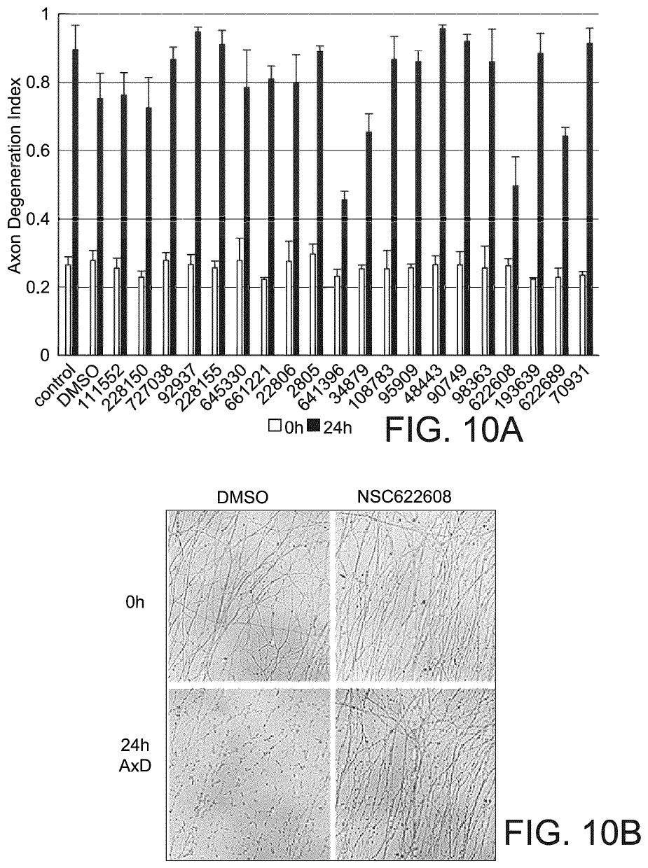

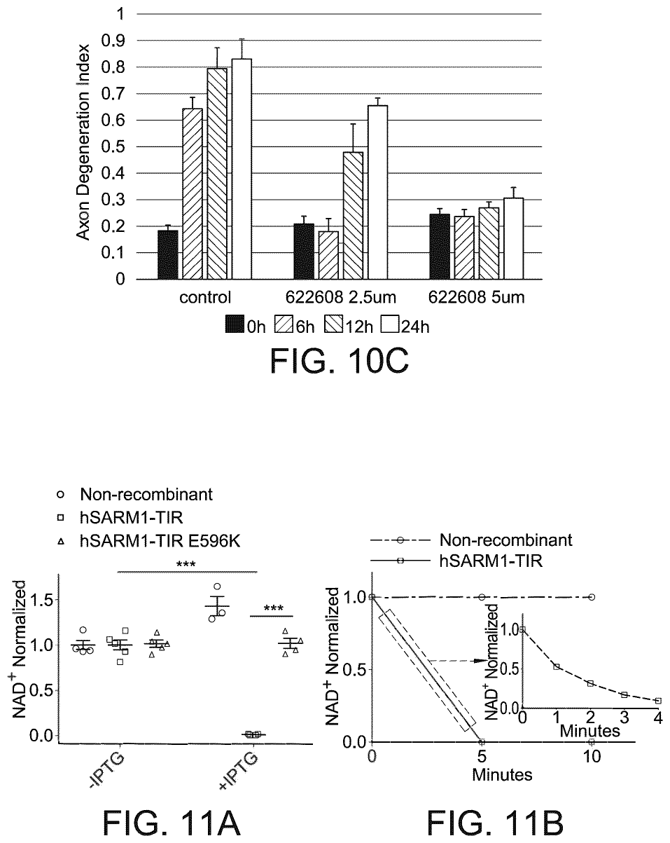

Axonal degeneration was induced by axotomy or by the addition of vincristine (0.04 μM) using DRG drop cultures in 96 well at DIV 6. Axotomy was performed by separating cell bodies and axons using a micro surgical blade under the microscope. Bright field images of axons (6 fields per well) were taken at 0-72 hours after axotomy using a high content imager (Operetta; Perkin-Elmer) with a 20× objective. Axon degeneration was quantified using degeneration index (DI) calculated using ImageJ (NIH, Sasaki et al., 2009, J. Neurosci., 19(17): 5525-5535). The average DI from 6 fields per well was obtained and averaged for each independent well. The DI was calculated from axon images from the same fields before (0 hour) and after (9-72 hours) axotomy. Compounds (in FIG. 10A) with a significant blockade of SARM1 NADase activity from the assays in Examples 1-3 were tested for their effects on axon degeneration in cultures of DRG neurons as described above. All 18 positive hits from HPLC screen were tested (at 5 μM) for their ability to inhibit axon degeneration. The candidate compounds are added to the culture medium at the concentration of 0.05 to 5 μM 30 min before axotomy. Axon degeneration was monitored by imaging axons before injury, and various time points after axotomy.

### Results

FIG. 10A illustrates the axon degeneration indices before injury and 24 h after injury (axotomy). A higher degeneration index indicates more axon degeneration (i.e. less inhibition). FIG. 10B illustrates a representative compound showing significant protection (NSC622608). The representative images before and after axotomy are shown. FIG. 10C illustrates dose dependent inhibition of axon degeneration by compound NSC622608.

Thus, the present Example demonstrates successful development of an axon degeneration assay to characterize compounds. Moreover, the present Example demonstrates that a compound identified in the present disclosure as an inhibitor of SARM1-TIR NADase activity also inhibits axon degeneration in a dose-dependent manner.

### Example 5

The present Example demonstrates that a SARM1-TIR complex purified from mammalian cells cleaves NAD+.

This example also illustrates application of an NAD+ depletion assay.

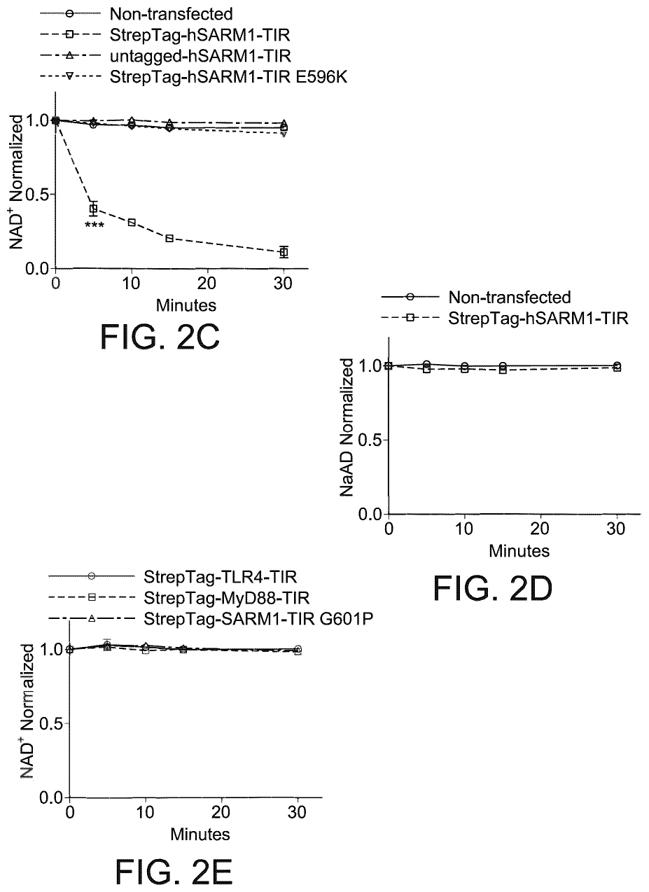

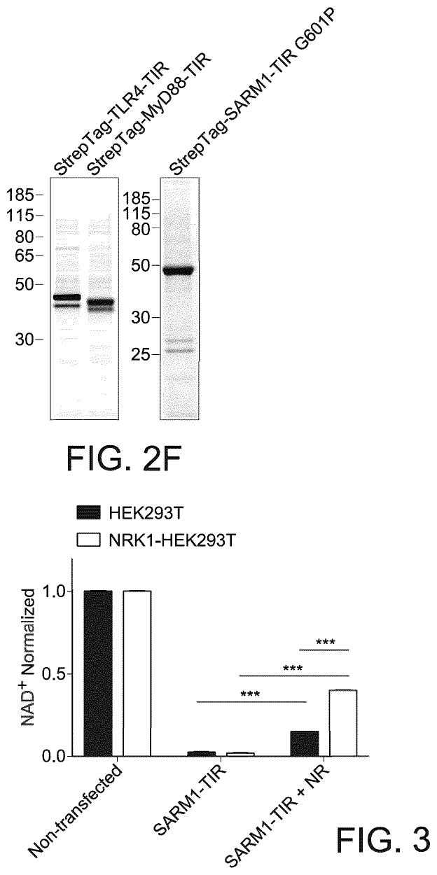

The human SARM1-TIR domain was engineered with a tandem StrepTag II at the N-terminus, a Venus fluorescent tag at the C-terminus, and expressed it transiently in NRK1-HEK293T cells supplemented with NR. Cell lysates were subsequently prepared by lysing cells under native conditions by sonication, and the recombinant SARM1-TIR protein complexes were affinity purified using MagStrep (Strep-Tactin) magnetic beads. Beads with SARM1-TIR complexes were incubated with NAD+(5 μM) for up to 30 minutes, metabolites were extracted, and then NAD+ levels were measured using HPLC (FIG. 2B). NAD+ levels dropped precipitously, within 5 minutes, when beads loaded with SARM1-TIR complexes were tested (FIG. 2C). In contrast, no decrease in NAD+ was observed if beads exposed to lysates were prepared from either non-transfected NRK1-HEK293T cells or from NRK1-HEK293T cells expressing SARM1-TIR lacking the StrepTag II (FIG. 2C). A TIR domain mutant [SARM1(E596K)] that is incapable of supporting injury-induced axonal NAD+ depletion and degeneration was also tested. Magnetic beads loaded with complexes assembled on this SARM1(E596K) mutant failed to degrade NAD+ in this in vitro assay (FIG. 2C).

The substrate specificity of the SARM1-TIR in vitro NADase reaction was examined.

Gerdts, J., et al. (Science, 2015, 348, 453-457) previously showed that Nicotinic Acid Adenine Dinucleotide (NaAD), a closely related analog of NAD+, was not cleaved after SARM1 activation. Using this in vitro assay, it was found that wild type SARM1-TIR complexes do not degrade NaAD (FIG. 2D). Together, these results show that the purified SARM1-TIR complex actively degrades NAD+ in a manner consistent with previous characterization of the axon degeneration process.

Whether the enzymatic activity was unique to complexes associated with the SARM1-TIR domain or whether TIR domains from other proteins could also assemble complexes that exhibit NADase activity was then explored. The TIR domains of TLR4, a Toll-like receptor, and MyD88, another member of the TIR adaptor family, were expressed and purified from NRK1-HEK293T cells and tested them in the in vitro NAD+ depletion assay. Both TLR4 and MyD88 TIR containing complexes showed no NADase activity (FIGS. 2E and 2F). These results support the previously reported unique roles of SARM1 among TIR adaptor proteins (Gerdts, J, et al., Science, 2015, 348, 453-457; O'Neill, L. A., et al., Nat. Rev. Immunol., 2013, 13, 453-460, Summers, D. W., et al., Proc. Natl. Acad. Sci. USA., 2016, 113, E6271-E6280) in promoting axonal degeneration and neuronal NAD+ depletion.

### Example 6

The present Example demonstrates that NAD+ cleavage activity observed in other experiments described herein is not due to other proteins that co-purify with SARM1-TIR and that therefore the SARM1-TIR domain possesses intrinsic NAD+ cleavage activity. Moreover, the present Example describes characterizations of this NAD+ cleavage activity and that the SARM1-TIR enzymatic reaction comprises both cyclase and glycohydrolase activities.

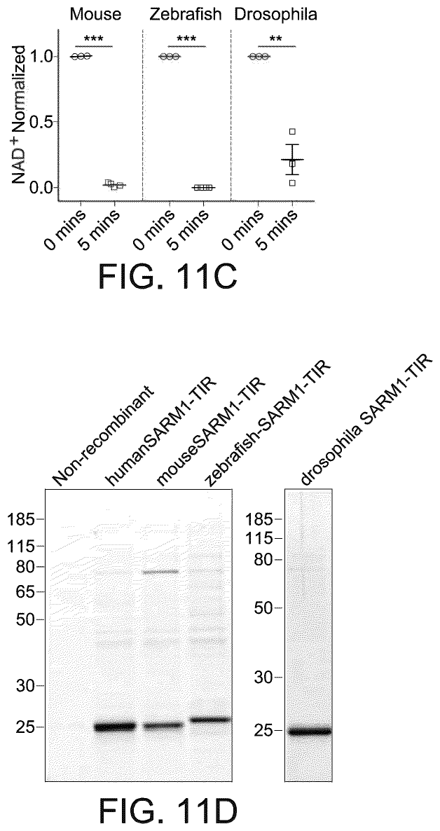

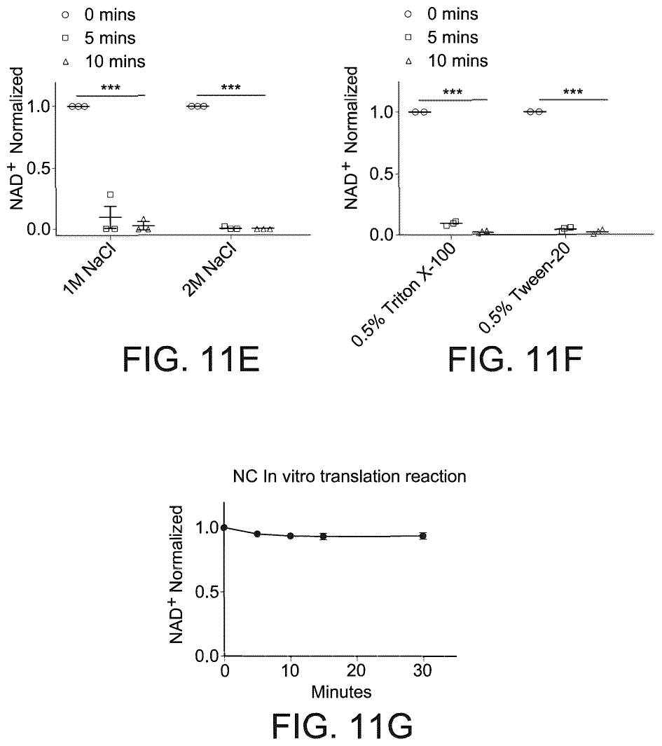

Human SARM1-TIR was expressed in E. coli so that proteins with NADase activity would not be co-purified. SARM1-TIR expression in E. coli was induced by IPTG addition, endogenous metabolites were extracted, and NAD+ levels were assessed by HPLC. Bacteria producing wild type SARM1-TIR had remarkably low (almost undetectable) levels of endogenous NAD+ within 60 minutes after IPTG addition when compared to bacteria harboring non-recombinant vector. Further, bacteria harboring mutant SARM1-TIR (E596K) had NAD+ levels comparable to bacteria harboring non-recombinant vector or to bacteria in which wild type SARM1 was not induced (FIG. 11A). FIG. 11A illustrates endogenous NAD+ levels in bacteria after IPTG induction of human SARM1-TIR. The bacterially expressed SARM1-TIR was purified using TAP and tested for NADase activity. Consistent with the results using SARM1-TIR complexes isolated from mammalian cells in example 5, NAD+ was rapidly consumed by bacterially produced SARM1-TIR protein (FIG. 11B). FIG. 11B illustrates in vitro NAD+ cleavage reaction by human SARM1-TIR protein expressed and purified from bacteria. Although it is highly unlikely that human SARM1-TIR would associate with an E. coli NADase, the intrinsic nature of the SARM1 NADase activity was tested by stringently washing the SARM1 TIR purified complexes with either high salt or detergents to remove potential associated proteins. Using these washed SARM1 TIR beads, they found no decrease in NAD+ cleavage activity, indicating that SARM1 itself has NADase activity (FIGS. 1E and 11F).

Mouse, zebrafish and Drosophila SARM1-TIR domains were expressed and purified in E. coli. The purified proteins were then tested for their ability to cleave NAD+. Similar to human SARM1-TIR domain, bacterially-expressed mouse, zebrafish and Drosophila SARM1-TIR domains also rapidly degrade NAD+ in vitro (FIG. 11C-D). FIG. 11C illustrates that bacterially expressed mouse, zebrafish and Drosophila SARM1-TIR proteins cleave NAD+ in the in vitro NADase assay. FIG. TTD illustrates a SYPRO Ruby gel of SARM1-TIR laden beads purified from bacteria used in NADase assay; representative of three independent experiments. These bacterially expressed proteins lack the Venus fluorescent tag and thus run at a different size than the proteins expressed in NRK1-HEK293T cells. Data were generated from at least three independent reaction experiments using purified protein from at least three independent bacteria clones. Data are presented as mean±SEM; Error bars: SEM; *** P<0.001 unpaired two tailed Student's t-test.

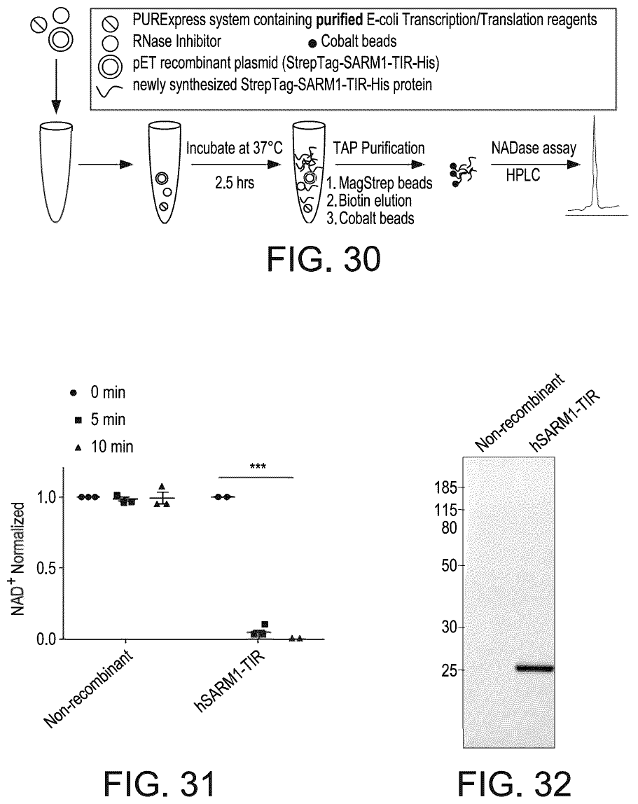

To demonstrate definitively that SARM1-TIR itself possessed the enzymatic activity, human SARM1-TIR was synthesized in a cell-free protein expression system that utilizes purified E. coli components for transcription and translation. None of the purified E-coli transcription/translation components are known NADases (Shimizu et al., Nat. Biotechnol., 2001, 19, 751-755), and these experiments confirmed that these purified components do not exhibit NADase activity (FIG. 11G). To test if SARM1-TIR purified from this in vitro translation system could cleave NAD+, the human SARM1-TIR plasmid DNA was first incubated with the purified transcription and translation reagents and RNase inhibitor for 2.5 hours at 37° C. Next, they purified the newly synthesized protein from the reaction by TAP, and tested for NADase activity in the assay (FIG. 30). The purified SARM1-TIR from this cell-free protein translation system rapidly cleaved NAD+, consistent with prior findings with SARM1-TIR purified from both mammalian cells and bacteria (FIGS. 31 and 32). Without being limited by theory, the finding that the SARM1-TIR domain depletes NAD+ in bacteria and that bacterially synthesized SARM1-TIR from multiple species cleaves NAD+ in vitro demonstrates that the SARM1-TIR domain has intrinsic NADase activity, and shows that SARM1 itself is responsible for the NAD+ depletion observed after axon injury. Moreover, these findings reveal for the first time that a TIR domain, previously demonstrated to function as a protein interaction domain, can also harbor enzymatic activity.

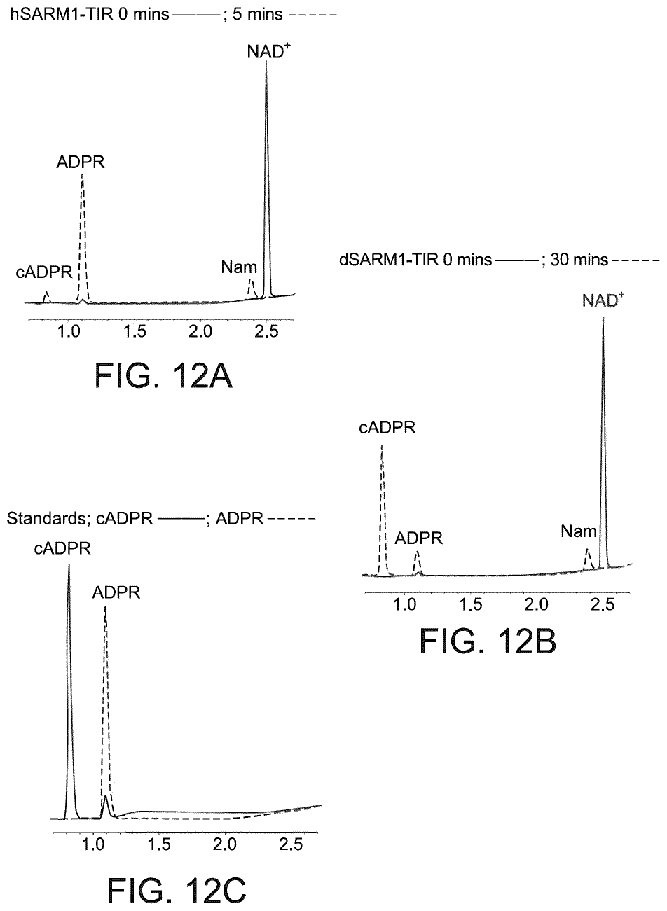

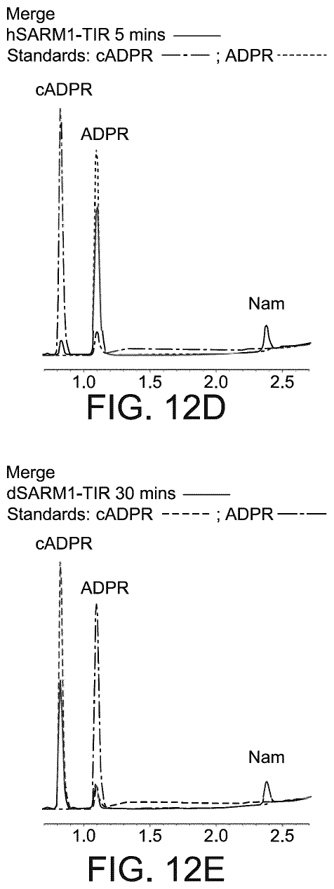

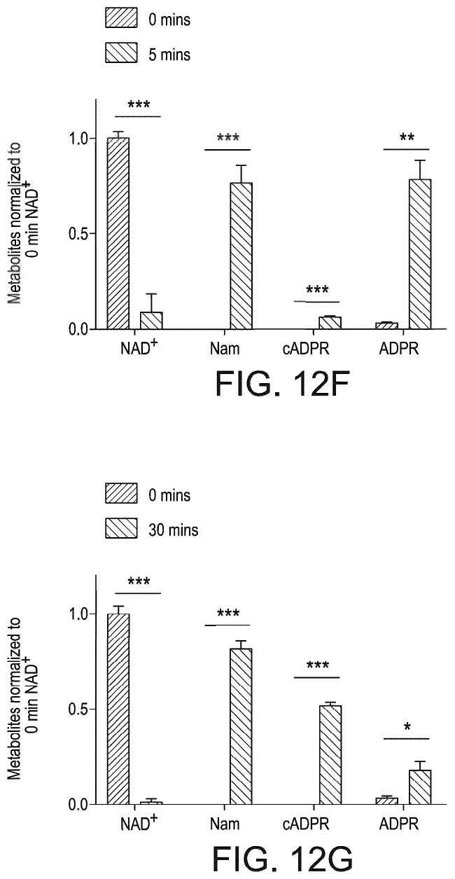

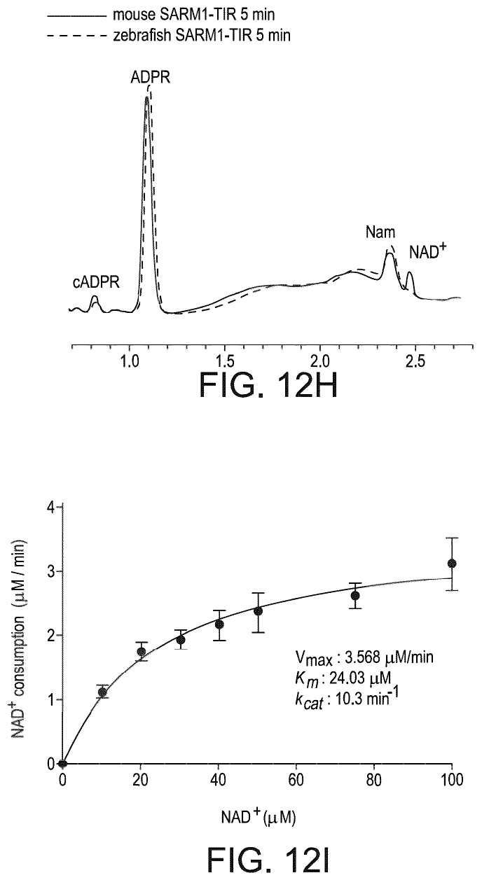

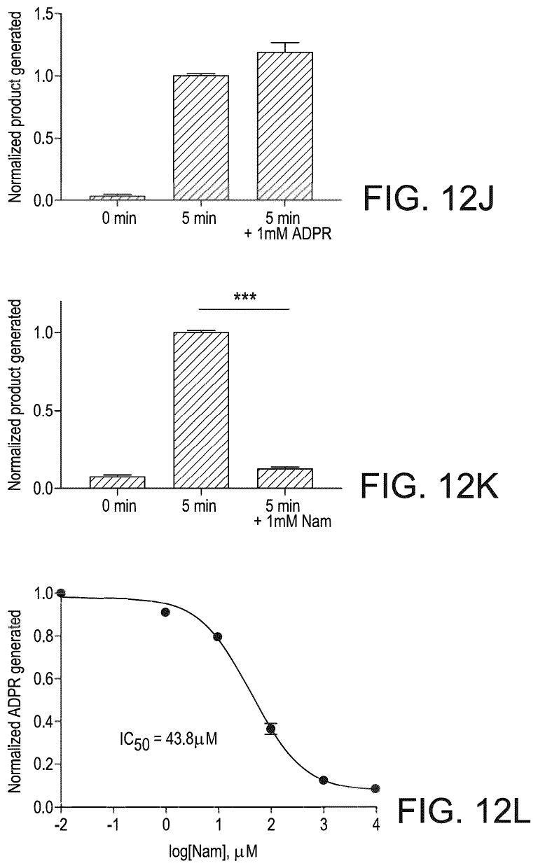

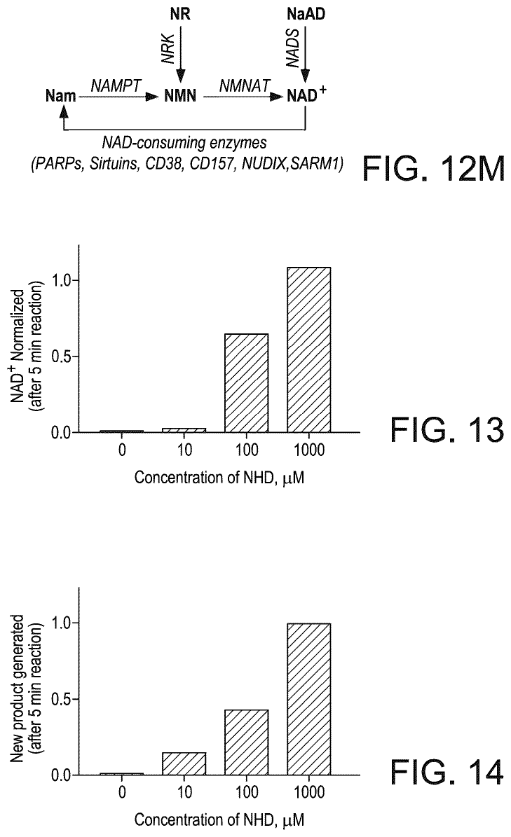

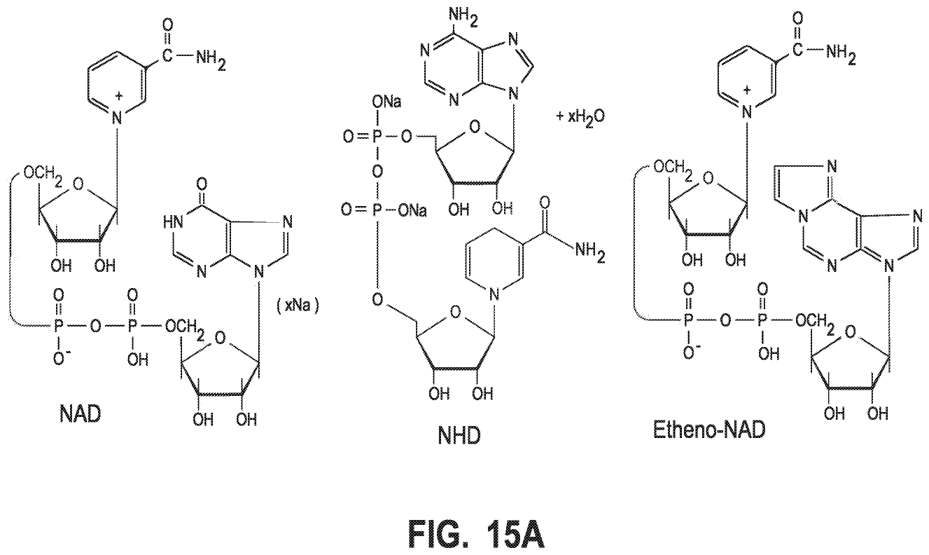

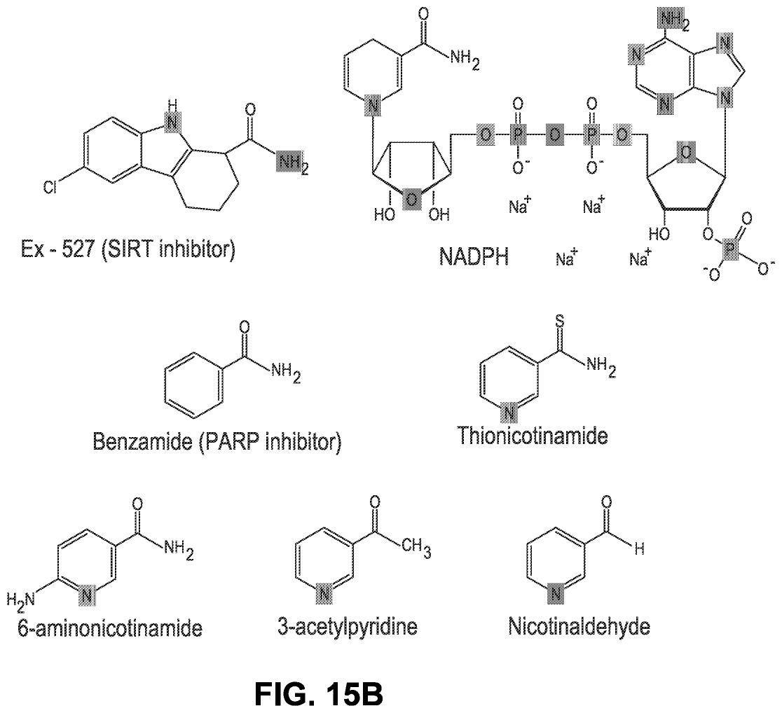

To further characterize the SARM1-TIR NADase activity, the NAD+ cleavage products of this enzymatic reaction were identified and reaction parameters were established. HPLC and LC-MS/MS analysis of the metabolites produced by human SARM1-TIR was performed; Nam and ADP Ribose (ADPR) were identified as major products, and cyclic ADPR (cADPR) as a minor product (FIG. 12A-G). While the mouse and zebrafish orthologs generated a similar ratio of reaction products as the human enzyme (FIG. 12H), the Drosophila SARM1-TIR purified either from bacteria or NRK1-293T cells generated more cADPR than ADPR (FIG. 12A-G). This finding is similar to results with the ADPRibosyl cyclase family of NADases (Liu et al., J. Biol. Chem., 2009, 284, 27637-27645), in which the mammalian ADP Ribosyl Cyclase CD38 cleaves NAD+ to generate ADPR as the major product, with minor amounts of cADPR; while the ADP Ribosyl Cyclase isolated from the sea mollusk Aplysia californica cleaves NAD+ into cADPR (Liu et al., J. Biol. Chem., 2009, 284, 27637-27645). This difference in reaction products between the Drosophila and vertebrate SARM1-TIR NADase may provide insights into the divergent enzymatic activities of the ADP Ribosyl cyclase family of enzymes.

Furthermore, kinetic assays of the SARM1-TIR enzyme revealed saturation kinetics (FIG. 12I), a distinguishing feature of enzyme catalysts, with an estimated Michaelis constant (Km) of 24 μM, maximum velocity (Vmax) of 3.6 μM/min, and turnover number (kcat) of 10.3 min-1 (FIG. 12I). Kinetic parameters for SARM1-TIR cleavage reaction. Vmax, Km, kcat were determined by fitting the data to the Michaelis-Menten equation and are presented as mean±SEM for three independent biological samples and experiment. Although the estimated kcat is lower than the reported values for other ADP-Ribosyl cyclases and NAD+ glycohydrolases (Ghosh et al., J. Biol. Chem., 2010, 285, 5683-5694), the estimated Km values are similar (Cantó et al., Cell Metab., 2015, 22, 31-53).

The reaction products were tested to determine whether they could inhibit the enzymatic activity of SARM1-TIR. While ADPR did not inhibit SARM1-TIR NADase activity (FIG. 12J; activity normalized to Nam generated at 5 min), Nam could inhibit the enzymatic activity with an IC50 of 43.8 μM, which is about 9-fold higher than the starting reaction NAD+ concentration (FIG. 12K-L). FIG. 12K illustrates that Nam inhibits SARM1-TIR enzymatic activity (normalized to ADPR generated at 5 min). FIG. 12L illustrates Nam dose response inhibition of SARM1-TIR enzymatic activity. Inhibitors of the SARM1-TIR domain modeled after nicotinamide can be useful in preventing the early stages of axon degeneration (Gerdts, J et al., Neuron, 2016, 89, 449-460; Fliegert, R., et al., Biochem. Soc. Trans., 2007, 35, 109-114).

These data demonstrated that the TIR domain of SARM1 cleaves NAD+ into Nam and ADPR. SARM1-TIR appears to be unique in this regard, as other tested TIR domains do not have this activity. A crystal structure of the SARM1-TIR domain can be important in identifying the NAD+ binding pocket as well as other key residues involved in NAD+ cleavage.

In summary, these results describe the first enzymatic activity intrinsic to a TIR domain. These data establish that NADase activity is integral to a conserved axon death program. The discovery that SARM1 is the axonal NADase (FIG. 12J) now provides an identified target for the design of inhibitors as novel therapeutic candidates for the treatment of neurodegenerative diseases.

### Example 7

This example describes characterization of analogs of nicotinamide (a known SARM1 NADase inhibitor) and analogs of NAD+ with respect to activity as inhibitors of SARM1 enzymatic activity and/or as substrates for the cleavage reaction. These analogs were tested using an assay that makes use of a bacterially-expressed tagged version of the SARM1 TIR fragment, as described in Example 2. Displaying this artificial SARM1 TIR domain on a solid surface (i.e. affinity beads) generates an active NAD+ cleavage enzyme.

TABLE 4
Substrates and Inhibitors of the SARM1 TIR NAD cleavage
activity as determined by an assay which uses a bacterially expressed,
tagged version of the SARM1 TIR fragment. Nicotinamide
Hypoxanthine dinucleotide (NHD) was both a substrate and an
inhibitor.

| # | Candidate Analogs | Inhibitor | Substrate |
|---|---|---|---|
| 1 | Thionicotinamide | No | No |
| 2 | 6-aminonicotinamide | No | No |
| 3 | 3-acetylpyridine | No | No |
| 4 | Nicotinaldehyde | No | No |
| 5 | Benzamide (PARP inhibitor) | No | No |
| 6 | Ex-527 (Sirtuin inhibitor) | Unclear (might | |
| | | inhibit at No | |
| | | very higher | |
| | | doses of | |
| | | 1 mM) | |
| 7 | Nicotinamide adenine | Unclear | Yes (at higher |
| | dinucleotide 2′- | | doses of at |
| | phosphate, reduced | | least 100 μM) |
| | (NADPH) | | |
| 8 | Nicotinamide Hypoxanthine | Yes | Yes |
| | dinucleotide (NHD) | | |
| 9 | Nicotinamide 1,N6- | Unclear (might | |
| | ethenoadenine | inhibit at No | |
| | dinucleotide | high doses | |
| | | of 1 mM) | |

### Example 8

This example illustrates that Glutamic Acid 642 is a catalytic residue in the active site of the SARM1-TIR enzyme.

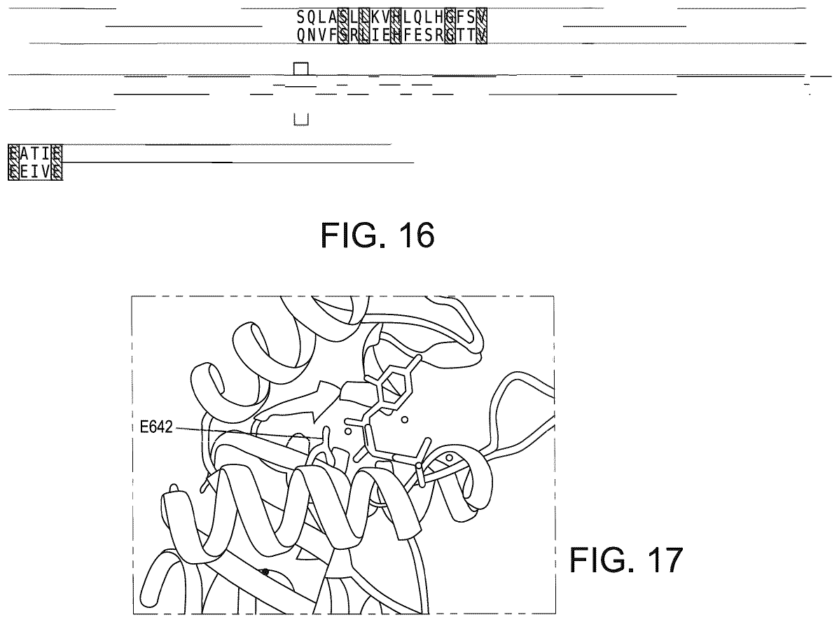

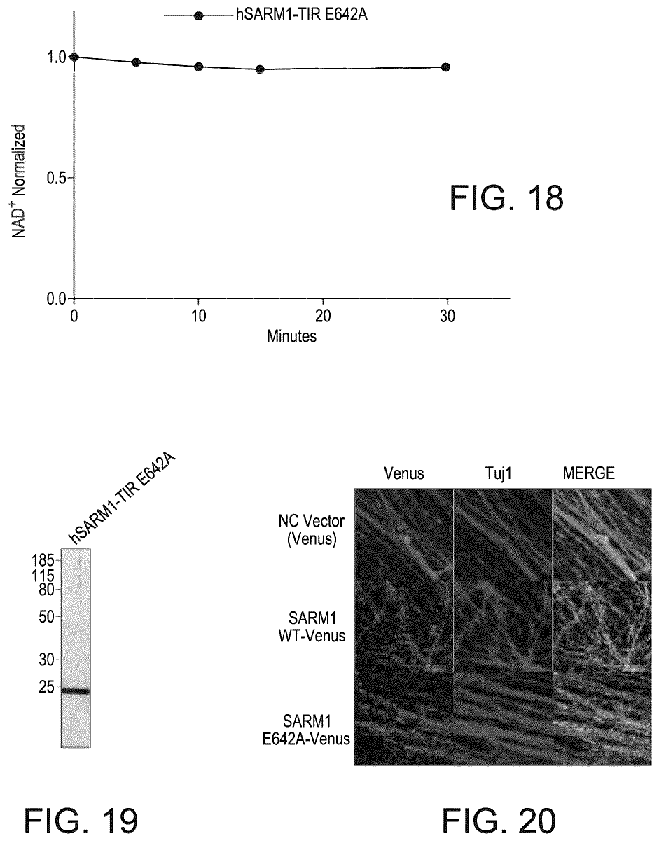

Since there is no reported crystal structure of the SARM1-TIR domain, an unbiased template-based prediction (Soding, J., et al., Nucleic Acids Res., 2005, 33, W244-248) was used to identify protein homologs of SARM1-TIR. A recent bioinformatics study showed that some TIR domains share strong structural similarity to nucleotide/nucleoside hydrolases (Burroughs, A. M., et al., Nucleic Acids Res., 2015, 43, 10633-10654). From domain prediction analysis using SARM1-TIR, other TIR domains were identified as expected. However, in addition to these TIR domains, a number of nucleotide hydrolase/transferase enzymes were also detected. For some of these enzymes, residues that contribute to catalytic activity have been established (Sikowitz, M. D., et al., Biochemistry, 2013, 52, 4037-4047; Armstrong, S. R., et al., Structure, 1995, 4, 97-107). Structural modeling and sequence alignments were used to identify putative residues in the SARM1-TIR domain that might contribute to enzymatic activity (FIG. 16 and FIG. 17). The SARM1-TIR domain was modeled using the crystal structure of two enzymes identified from the prediction: MilB Cytidine 5′ monophosphate (CMP) Hydrolase (PDB: 4JEM) (FIG. 17) and Nucleoside 2-deoxyribosyltransferase (PDB: 1F8Y). A glutamic acid E642 in the SARM1-TIR domain aligned with both the key catalytic glutamic acid residue in CMP hydrolase (Sikowitz, M. D., et al., Biochemistry, 2013, 52, 4037-4047) and the proposed nucleophilic glutamic acid in the active site of nucleoside 2-deoxyribosyltransferase (Armstrong, S. R., et al., Structure, 1995, 4, 97-107) (FIG. 16 and FIG. 17). Moreover, glutamic acid residues are also known catalytic residues in other NADases (Ghosh, J., et al., J. Biol. Chem., 2010, 285, 5683-5694). To test if SARM1 TIR E642 had similar catalytic properties, this residue was mutated to an Alanine (E642A) in SARM1-TIR, purified the protein from the cell-free protein translation system, and tested it for NAD+ cleavage activity. Purified SARM1-TIR E642A failed to cleave NAD+ in the NADase assay (FIG. 18 and FIG. 19). E642 in the SARM1-TIR domain is a key catalytic residue within the active site that is responsible for NAD+ cleavage.

### Example 9

This example illustrates that SARM1 enzymatic activity functions in axons to promote pathological axonal degeneration.

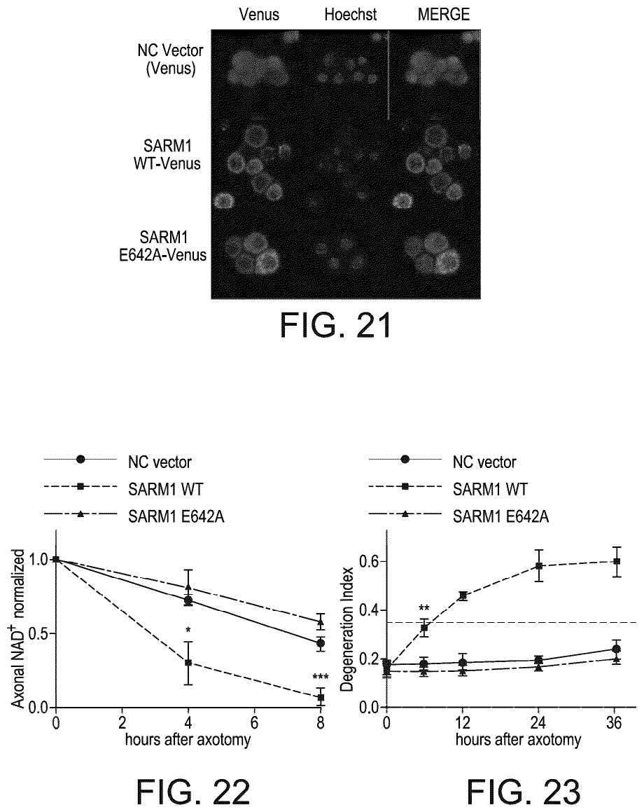

Having demonstrated that the SARM1 TIR domain is an enzyme and having identified its catalytic residue, enzymatic activity of the SARM1-TIR domain and, in particular, the identified glutamate, were investigated to determine whether either are required for the pro-degenerative functions of full-length SARM1 in neurons. In wild type neurons, axotomy triggers rapid depletion of axonal NAD+ and axonal degeneration, while in SARM1-deficient neurons axonal degeneration is blocked and NAD+ levels remain significantly higher than in injured wild type axons (Gerdts et al., Science, 2015, 348, 453-457). First, the SARM1 NADase activity was tested to determine whether such activity is necessary for injury-induced axonal NAD+ depletion and subsequent axonal degeneration. In these experiments, either wild type (enzymatically active) full-length SARM1 or mutant (enzymatically disabled) SARM1(E642A) were expressed in cultured SARM1-deficient DRG neurons. FIG. 20 and FIG. 21 illustrate that both were well expressed in axons. Following axotomy, axonal NAD+ levels and axonal degeneration were measured.

Expression of enzymatically active, wild type SARM1 in SARM1-deficient DRG neurons promotes both axonal NAD+ depletion and axonal degeneration after axotomy.

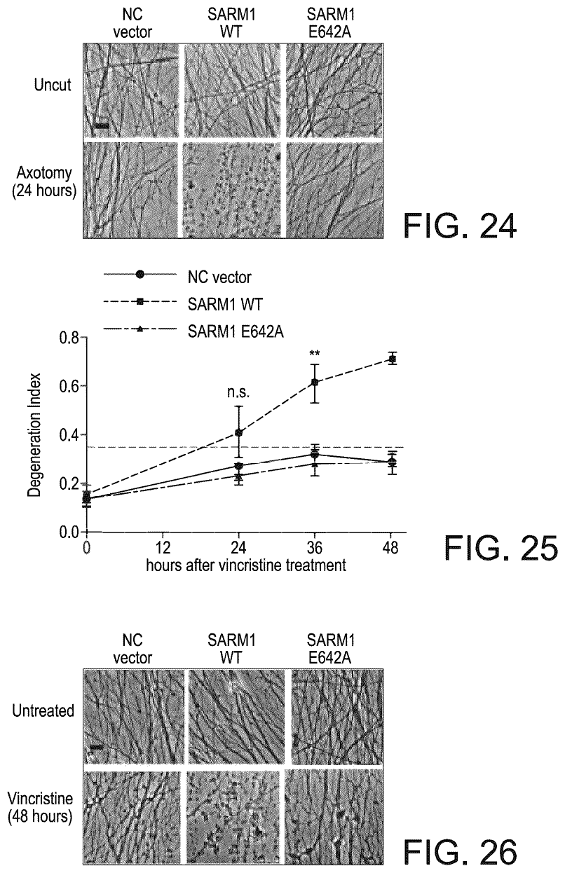

In contrast to wild type SARM1, when the enzymatically disabled SARM1(E642A) mutant is expressed in these neurons, axotomy did not induce axonal degeneration or rapid NAD+ depletion (FIG. 22-24).

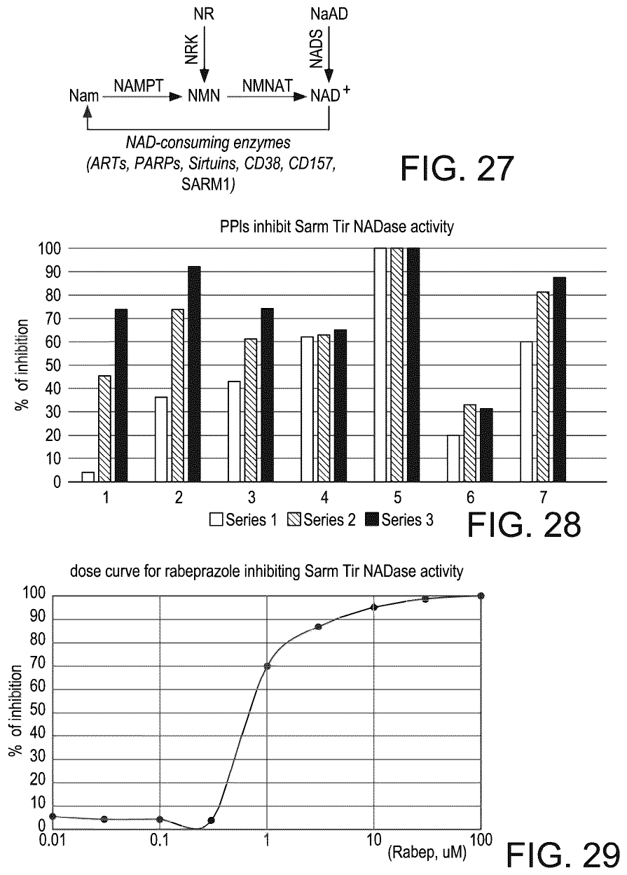

The requirement for SARM1 enzyme activity was also tested in another injury model—vincristine-induced neurotoxicity. Cultured SARM1-deficient DRG axons are protected from vincristine-induced axonal degeneration (Gerdts, J., et al., J. Neurosci., 2013, 33, 13569-13580). Moreover, SARM1 is required in mice for the development of vincristine-induced peripheral neuropathy (Geisler et al., 2016, Brain, 139, 3092-3108). As with axotomy, either wild type (enzymatically active) full-length SARM1 or mutant (enzymatically disabled) SARM1(E642A) was expressed in cultured SARM1-deficient DRG neurons. Enzymatically active SARM1 mediates axon loss in response to the chemotherapeutic vincristine, while enzymatically disabled SARM1 does not promote axon loss following vincristine administration (FIG. 25, and FIG. 26). Altogether, these findings demonstrate that the intrinsic NADase activity of SARM1 (FIG. 27) is necessary to promote axonal degeneration after both traumatic and neurotoxic injuries, and suggest that inhibitors of the SARM1 NADase could block pathological axonal degeneration.

### Example 10

This example illustrates the identification and characterization of a family of small molecules that effectively inhibit SARM1 NADase activity.

Initial screening using methods of the present teachings identified dexlansoprazole and tenatorprazole as SARM1 NADase inhibitors. These molecules are both members of a class of molecules referred to as protein pump inhibitors. The rest of the drug class was screened using the HPLC-based SARM1 SAM-TIR NADase assay described in detail in Example 1, with 5 μM NAD. FIG. 28 illustrates testing at 5 μM (Series 1), 15 μM (Series 2), and 50 μM (Series 3): omeprazole (1), lansoprazole (2), esomeprazole magnesium hydrate (3), pantoprazole sodium sesquihydrate (4), rabeprazole sodium (5), dexlansoprazole (6) and tenatoprazole (7). Each member of the family exhibited at least some inhibitory activity (FIG. 28). Based on these results, a dose-response analysis of rabeprozole (FIG. 29) was performed. This molecule showed 95% inhibition at 10 μM and 98.8% inhibition at 30 μM. These results indicate that this family of molecules has SARM1 NADase activity.

| GenBankSend: | |
|---|---|
| Homo sapiens sterile alpha and TIR motif containing protein 1 | |
| isoform a (SARM1) mRNA, complete cds | |
| GenBank: AY444166.1 | |
| LOCUS AY444166 2193 bp mRNA linear PRI Jan. 16, 2004 | |
| DEFINITION Homo sapiens sterile alpha and TIR motif containing | |
| protein 1 | |
| isoform a (SARM1) mRNA, complete cds. ACCESSION AY444166 | |
| VERSION AY444166.1 GI: 38326778 | |
| SOURCE Homo sapiens (human) ORGANISM Homo sapiens | |
| REFERENCE 1 (bases 1 to 2193) | |
| AUTHORS Bousson, J. -C., Casteran, C. and Tiraby, G. TITLE SARM1 | |
| isoforms nucleotide sequence | |
| JOURNAL Unpublished REFERENCE 2 (bases 1 to 2193) | |
| AUTHORS Bousson, J. -C., Casteran, C. and Tiraby, G. TITLE Direct | |
| Submission | |
| JOURNAL Submitted (Oct. 21, 2003) CAYLA, BP4437, 5 rue Jean | |
| Rodier, Toulouse | |
| cedex 4 31405, France FEATURES Location/Qualifiers | |
| source 1 . . . 2193 | |
| /organism = “Homo sapiens” | |
| /mol_type = “mRNA” | |
| /db_xref = “taxon: 9606” | |
| /chromosome = “17” | |
| /map = “17q11” gene 1 . . . 2193 | |
| /gene = “SARM1” | |
| /gene_synonym = “KIAA0524” | |
| /gene_synonym = “SAMD2” | |
| /gene_synonym = “SARM” CDS 1 . . . 2175 | |
| /gene = “SARM1″ | |
| /gene_synonym = “KIAA0524” | |
| /gene_synonym = “SAMD2” | |
| /gene_synonym = “SARM” | |
| /note = “SARM1a; receptor” | |
| /codon_start = 1 | |
| /product = “sterile alpha and TIR motif containing protein 1 | |
| isoform a” | |
| /protein_id = “AAR17520.1” | |
| /db_xref = “GI: 38326779” | |
| /translation = “MVLTLLLSAYKLCRFFAMSGPRPGAERLAVPGPDGGGGTGPWWAAGGRG | |
| PREVSPGAGTEVQDALERALPELQQALSALKQAGGARAVGAGLAEVFQLVEEAWLLPAVGREV | |
| AQGLCDAIRLDGGLDLLLRLLQAPELETRVQAARLLEQILVAENRDRVARIGLGVILNLAKER | |
| EPVELARSVAGILEHMFKHSEETCQRLVAAGGLDAVLYWCRRTDPALLRHCALALGNCALHGG | |
| QAVQRRMVEKRAAEWLFPLAFSKEDELLRLHACLAVAVLATNKEVEREVERSGTLALVEPLVA | |
| SLDPGRFARCLVDASDTSQGRGPDDLQRLVPLLDSNRLEAQCIGAFYLCAEAAIKSLQGKTKV | |
| FSDIGAIQSLKRLVSYSTNGTKSALAKRALRLLGEEVPRPILPSVPSWKEAEVQTWLQQIGFS | |
| KYCESFREQQVDGDLLLRLTEEELQTDLGMKSGITRKRFFRELTELKTFANYSTCDRSNLADW | |
| LGSLDPRFRQYTYGLVSCGLDRSLLHRVSEQQLLEDCGIHLGVHRARILTAAREMLHSPLPCT | |
| GGKPSGDTPDVFISYRRNSGSQLASLLKVHLQLHGFSVFIDVEKLEAGKFEDKLIQSVMGARN | |
| FVLVLSPGALDKCMQDHDCKDWVHKEIVTALSCGKNIVPIIDGFEWPEPQVLPEDMQAVLTFN | |
| GIKWSHEYQEATIEKIIRFLQGRSSRDSSAGSDTSLEGAAPMGPT” ORIGIN | |
| 1 | atggtcctga cgctgcttct ctccgcctac aagctgtgtc gcttcttcgc catgtcgggc |
| 61 | ccacggccgg gcgccgagcg gctggcggtg cctgggccag atgggggcgg tggcacgggc |
| 121 | ccatggtggg ctgcgggtgg ccgcgggccc cgcgaagtgt cgccgggggc aggcaccgag |
| 181 | gtgcaggacg ccctggagcg cgcgctgccg gagctgcagc aggccttgtc cgcgctgaag |
| 241 | caggcgggcg gcgcgcgggc cgtgggcgcc ggcctggccg aggtcttcca actggtggag |
| 301 | gaggcctggc tgctgccggc cgtgggccgc gaggtagccc agggtctgtg cgacgccatc |
| 361 | cgcctcgatg gcggcctcga cctgctgttg cggctgctgc aggcgccgga gttggagacg |
| 421 | cgtgtgcagg ccgcgcgcct gctggagcag atcctggtgg ctgagaaccg agaccgcgtg |
| 481 | gcgcgcattg ggctgggcgt gatcctgaac ctggcgaagg aacgcgaacc cgtagagctg |
| 541 | gcgcggagcg tggcaggcat cttggagcac atgttcaagc attcggagga gacatgccag |
| 601 | aggctggtgg cggccggcgg cctggacgcg gtgctgtatt ggtgccgccg cacggacccc |
| 661 | gcgctgctgc gccactgcgc gctggcgctg ggcaactgcg cgctgcacgg gggccaggcg |
| 721 | gtgcagcgac gcatggtaga gaagcgcgca gccgagtggc tcttcccgct cgccttctcc |
| 781 | aaggaggacg agctgcttcg gctgcacgcc tgcctcgcag tagcggtgtt ggcgactaac |
| 841 | aaggaggtgg agcgcgaggt ggagcgctcg ggcacgctgg cgctcgtgga gccgcttgtg |
| 901 | gcctcgctgg accctggccg cttcgcccgc tgtctggtgg acgccagcga cacaagccag |
| 961 | ggccgcgggc ccgacgacct gcagcgcctc gtgccgttgc tcgactctaa ccgcttggag |
| 1021 | gcgcagtgca tcggggcttt ctacctctgc gccgaggctg ccatcaagag cctgcaaggc |
| 1081 | aagaccaagg tgttcagcga catcggcgcc atccagagcc tgaaacgcct ggtttcctac |
| 1141 | tctaccaatg gcactaagtc ggcgctggcc aagcgcgcgc tgcgcctgct gggcgaggag |
| 1201 | gtgccacggc ccatcctgcc ctccgtgccc agctggaagg aggccgaggt tcagacgtgg |
| 1261 | ctgcagcaga tcggtttctc caagtactgc gagagcttcc gggagcagca ggtggatggc |
| 1321 | gacctgcttc tgcggctcac ggaggaggaa ctccagaccg acctgggcat gaaatcgggc |
| 1381 | atcacccgca agaggttctt tagggagctc acggagctca agaccttcgc caactattct |
| 1441 | acgtgcgacc gcagcaacct ggcggactgg ctgggcagcc tggacccgcg cttccgccag |
| 1501 | tacacctacg gcctggtcag ctgcggcctg gaccgctccc tgctgcaccg cgtgtctgag |
| 1561 | cagcagctgc tggaagactg cggcatccac ctgggcgtgc accgcgcccg catcctcacg |
| 1621 | gcggccagag aaatgctaca ctccccgctg ccctgtactg gtggcaaacc cagtggggac |
| 1681 | actccagatg tcttcatcag ctaccgccgg aactcaggtt cccagctggc cagtctcctg |
| 1741 | aaggtgcacc tgcagctgca tggcttcagt gtcttcattg atgtggagaa gctggaagca |
| 1801 | ggcaagttcg aggacaaact catccagagt gtcatgggtg cccgcaactt tgtgttggtg |
| 1861 | ctatcacctg gagcactgga caagtgcatg caagaccatg actgcaagga ttgggtgcat |
| 1921 | aaggagattg tgactgcttt aagctgcggc aagaacattg tgcccatcat tgatggcttc |
| 1981 | gagtggcctg agccccaggt cctgcctgag gacatgcagg ctgtgcttac tttcaacggt |
| 2041 | atcaagtggt cccacgaata ccaggaggcc accattgaga agatcatccg cttcctgcag |
| 2101 | ggccgctcct cccgggactc atctgcaggc tctgacacca gtttggaggg tgctgcaccc |
| 2161 | atgggtccaa cctaaccagt ccccagttcc cca |

Also Known As:
MyD88-5, SAMD2, SARM Homologs of the SARM1 gene
The SARM1 gene is conserved in chimpanzee, Rhesus monkey, dog,
mouse, rat, chicken, zebrafish, fruit fly, mosquito,
C. elegans, and frog.

### Examples 11-16

As depicted in the Examples below, in certain exemplary embodiments, compounds are prepared according to the following general procedures. It will be appreciated that, although the general methods depict the synthesis of certain compounds of the present disclosure, the following general methods, and other methods known to one of ordinary skill in the art, can be applied to all compounds and subclasses and species of each of these compounds, as described herein.

### General Procedures

The following is a description of the assays used to determine SARM1 NADase activity for the compounds of formula I^{A} and formula I^{B}.

### Assay 1. Preparation of SARM1 SAM-TIR Lysate (STL)

NRK1-HEK293T cells represent a polyclonal cell line that has been stably transfected with an FCIV expression vector that expresses human Nicotinamide Riboside Kinase 1 (NRK1), an enzyme that converts the NAD+ biosynthetic precursor nicotinamide riboside (NR) to NMN, the immediate precursor of NAD+. This expression vector has the DNA sequence: gtcgacggatcgggagatctcccgatcccctatggtgcactctcagtacaatctgctctgatgccgcatagttaagccagtatctgctcc ctgcttgtgtgttggaggtcgctgagtagtgcgcgagcaaaatttaagctacaacaaggcaaggcttgaccgacaattgcatgaagaat ctgcttagggttaggcgttttgcgctgcttcgcgatgtacgggccagatatacgcgttgacattgattattgactagttattaatagtaatca attacggggtcattagttcatagcccatatatggagttccgcgttacataacttacggtaaatggcccgcctggctgaccgcccaacgac ccccgcccattgacgtcaataatgacgtatgttcccatagtaacgccaatagggactttccattgacgtcaatgggtggagtatttacggt aaactgcccacttggcagtacatcaagtgtatcatatgccaagtacgccccctattgacgtcaatgacggtaaatggcccgcctggcatt atgcccagtacatgaccttatgggactttcctacttggcagtacatctacgtattagtcatcgctattaccatggtgatgcggttttggcagt acatcaatgggcgtggatagcggtttgactcacggggatttccaagtctccaccccattgacgtcaatgggagtttgttttggcaccaaa atcaacgggactttccaaaatgtcgtaacaactccgccccattgacgcaaatgggcggtaggcgtgtacggtgggaggtctatataag cagcgcgttttgcctgtactgggtctctctggttagaccagatctgagcctgggagctctctggctaactagggaacccactgcttaagc ctcaataaagcttgccttgagtgcttcaagtagtgtgtgcccgtctgttgtgtgactctggtaactagagatccctcagaccctttagtcag tgtggaaaatctctagcagtggcgcccgaacagggacttgaaagcgaaagggaaaccagaggagctctctcgacgcaggactcgg cttgctgaagcgcgcacggcaagaggcgaggggcggcgactggtgagtacgccaaaaattttgactagcggaggctagaaggaga gagatgggtgcgagagcgtcagtattaagcgggggagaattagatcgcgatgggaaaaaattcggttaaggccagggggaaagaa aaaatataaattaaaacatatagtatgggcaagcagggagctagaacgattcgcagttaatcctggcctgttagaaacatcagaaggct gtagacaaatactgggacagctacaaccatcccttcagacaggatcagaagaacttagatcattatataatacagtagcaaccctctatt gtgtgcatcaaaggatagagataaaagacaccaaggaagctttagacaagatagaggaagagcaaaacaaaagtaagaccaccgc acagcaagcggccgctgatcttcagacctggaggaggagatatgagggacaattggagaagtgaattatataaatataaagtagtaaa aattgaaccattaggagtagcacccaccaaggcaaagagaagagtggtgcagagagaaaaaagagcagtgggaataggagctttgt tccttgggttcttgggagcagcaggaagcactatgggcgcagcgtcaatgacgctgacggtacaggccagacaattattgtctggtata gtgcagcagcagaacaatttgctgagggctattgaggcgcaacagcatctgttgcaactcacagtctggggcatcaagcagctccag gcaagaatcctggctgtggaaagatacctaaaggatcaacagctcctggggatttggggttgctctggaaaactcatttgcaccactgc tgtgccttggaatgctagttggagtaataaatctctggaacagatttggaatcacacgacctggatggagtgggacagagaaattaaca attacacaagcttaatacactccttaattgaagaatcgcaaaaccagcaagaaaagaatgaacaagaattattggaattagataaatggg caagtttgtggaattggtttaacataacaaattggctgtggtatataaaattattcataatgatagtaggaggcttggtaggtttaagaatagt ttttgctgtactttctatagtgaatagagttaggcagggatattcaccattatcgtttcagacccacctcccaaccccgaggggacccgac aggcccgaaggaatagaagaagaaggtggagagagagacagagacagatccattcgattagtgaacggatcggcactgcgtgcgc caattctgcagacaaatggcagtattcatccacaattttaaaagaaaaggggggattggggggtacagtgcaggggaaagaatagtag acataatagcaacagacatacaaactaaagaattacaaaaacaaattacaaaaattcaaaattttcgggtttattacagggacagcagag atccagtttggttaattaagggtgcagcggcctccgcgccgggttttggcgcctcccgcgggcgcccccctcctcacggcgagcgct gccacgtcagacgaagggcgcaggagcgttcctgatccttccgcccggacgctcaggacagcggcccgctgctcataagactcgg ccttagaaccccagtatcagcagaaggacattttaggacgggacttgggtgactctagggcactggttttctttccagagagcggaaca ggcgaggaaaagtagtcccttctcggcgattctgcggagggatctccgtggggcggtgaacgccgatgattatataaggacgcgccg ggtgtggcacagctagttccgtcgcagccgggatttgggtcgcggttcttgtttgtggatcgctgtgatcgtcacttggtgagttgcggg ctgctgggctggccggggcttcgtggccgccgggccgctcggtgggacggaagcgtgtggagagaccgccaagggctgtagtct gggtccgcgagcaaggttgccctgaactgggggttggggggagcgcacaaaatggcggctgttcccgagtcttgaatggaagacg cttgtaaggcgggctgtgaggtcgttgaaacaaggtggggggcatggtgggcggcaagaacccaaggtcttgaggccttcgctaatg cgggaaagctcttattcgggtgagatgggctggggcaccatctggggaccctgacgtgaagtttgtcactgactggagaactcgggtt tgtcgtctggttgcgggggcggcagttatgcggtgccgttgggcagtgcacccgtacctttgggagcgcgcgcctcgtcgtgtcgtga cgtcacccgttctgttggcttataatgcagggtggggccacctgccggtaggtgtgcggtaggctttctccgtcgcaggacgcagggt tcgggcctagggtaggctctcctgaatcgacaggcgccggacctctggtgaggggagggataagtgaggcgtcagtttctttggtcg gttttatgtacctatcttcttaagtagctgaagctccggttttgaactatgcgctcggggttggcgagtgtgttttgtgaagttttttaggcacc ttttgaaatgtaatcatttgggtcaatatgtaattttcagtgttagactagtaaagcttctgcaggtcgactctagaaaattgtccgctaaattc tggccgtttttggcttttttgttagacgaagcttgggctgcaggtcgactctagaggatcatgaagagatttgtcattggaattggtggtgtg acaaacggagggaagacgacactggctaagagcttgcagaagcaccttcccaactgcagcgtcatatctcaggatgacttcttcaagc cagagtctgagatagacatagatgaaaatggttttttgcagtatgatgtgcttgaagcgctaaatatggaaaaaatgatgtcagcagttc ctgttggatggaaaacccaggaagctctgcgggaccagcagccttggaaagtgctcaaggggttcccattttaattattgaaggttcct tctctttaattataagcctctggacaccatatggaacagaagttacttcctgaccgttccatatgaagaatgtaagaggagaaggagtacc agagtatatgagcctccagaccctccagggtacttcgatggccacgtgtggcccatgtacctaaagcacagacaggaaatgagctcc atcacctgggacattgtttacctggatggaacaaggtctgaagaggacctcttctctcaggtgtatgaagatgtcaagcaggaactaga gaagcaaaatggtttgGACTATAAAGATGATGATGATAAGTAAgctagctaccggtgatccgcccctctccc tcccccccccctaacgttactggccgaagccgcttggaataaggccggtgtgcgtttgtctatatgttattttccaccatattgccgtctttt ggcaatgtgagggcccggaaacctggccctgtcttcttgacgagcattcctaggggtctttcccctctcgccaaaggaatgcaaggtct gttgaatgtcgtgaaggaagcagttcctctggaagcttcttgaagacaaacaacgtctgtagcgaccctttgcaggcagcggaacccc ccacctggcgacaggtgcctctgcggccaaaagccacgtgtataagatacacctgcaaaggcggcacaaccccagtgccacgttgt gagttggatagttgtggaaagagtcaaatggctctcctcaagcgtattcaacaaggggctgaaggatgcccagaaggtaccccattgt atgggatctgatctggggcctcggtgcacatgctttacatgtgtttagtcgaggttaaaaaaacgtctaggccccccgaaccacgggga cgtggttttcctttgaaaaacacgatgataatatggccacaaccATGGatggccaagttgaccagtgccgttccggtgctcaccgcg cgcgacgtcgccggagcggtcgagttctggaccgaccggctcgggttctcccgggacttcgtggaggacgacttcgccggtgtggt ccgggacgacgtgaccctgttcatcagcgcggtccaggaccaggtggtgccggacaacaccctggcctgggtgtgggtgcgcggc ctggacgagctgtacgccgagtggtcggaggtcgtgtccacgaacttccgggacgcctccgggccggccatgaccgagatcggcg agcagccgtgggggcgggagttcgccctgcgcgacccggccggcaactgcgtgcacttcgtggccgaggagcaggactgagaat tcgatatcaagcttatcgataatcaacctctggattacaaaatttgtgaaagattgactggtattcttaactatgttgctccttttacgctatgtg gatacgctgctttaatgcctttgtatcatgctattgcttcccgtatggctttcattttctcctccttgtataaatcctggttgctgtctctttatgag gagttgtggcccgttgtcaggcaacgtggcgtggtgtgcactgtgtttgctgacgcaacccccactggttggggcattgccaccacctg tcagctcctttccgggactttcgcttccccctccctattgccacggcggaactcatcgccgcctgccttgcccgctgctggacaggggc tcggctgttgggcactgacaattccgtggtgttgtcggggaaatcatcgtcctttccttggctgctcgcctgtgttgccacctggattctgc gcgggacgtccttctgctacgtcccttcggccctcaatccagcggaccttccttcccgcggcctgctgccggctctgcggcctcttccg cgtcttcgccttcgccctcagacgagtcggatctccctttgggccgcctccccgcatcgataccgtcgacctcgagacctagaaaaaca tggagcaatcacaagtagcaatacagcagctaccaatgctgattgtgcctggctagaagcacaagaggaggaggaggtgggttttcc agtcacacctcaggtacctttaagaccaatgacttacaaggcagctgtagatcttagccactttttaaaagaaaaggggggactggaag ggctaattcactcccaacgaagacaagatatccttgatctgtggatctaccacacacaaggctacttccctgattggcagaactacacac cagggccagggatcagatatccactgacctttggatggtgctacaagctagtaccagttgagcaagagaaggtagaagaagccaatg aaggagagaacacccgcttgttacaccctgtgagcctgcatgggatggatgacccggagagagaagtattagagtggaggtttgaca gccgcctagcatttcatcacatggcccgagagctgcatccggactgtactgggtctctctggttagaccagatctgagcctgggagctc tctggctaactagggaacccactgcttaagcctcaataaagcttgccttgagtgcttcaagtagtgtgtgcccgtctgttgtgtgactctgg taactagagatccctcagaccctttagtcagtgtggaaaatctctagcagggcccgtttaaacccgctgatcagcctcgactgtgccttc tagttgccagccatctgttgtttgcccctcccccgtgccttccttgaccctggaaggtgccactcccactgtcctttcctaataaaatgagg aaattgcatcgcattgtctgagtaggtgtcattctattctggggggtggggtggggcaggacagcaagggggaggattgggaagaca atagcaggcatgctggggatgcggtgggctctatggcttctgaggcggaaagaaccagctggggctctagggggtatccccacgcg ccctgtagcggcgcattaagcgcggcgggtgtggtggttacgcgcagcgtgaccgctacacttgccagcgccctagcgcccgctcct ttcgcttcttcccttcctttctcgccacgttcgccggctttccccgtcaagctctaaatcgggggctccctttagggttccgatttagtgcttt acggcacctcgaccccaaaaaacttgattagggtgatggttcacgtagtgggccatcgccctgatagacggtttttcgcccttgacgtt ggagtccacgttctttaatagtggactcttgttccaaactggaacaacactcaaccctatctcggtctattcttttgatttataagggattttgc cgatttcggcctattggttaaaaaatgagctgatttaacaaaaatttaacgcgaattaattctgtggaatgtgtgtcagttagggtgtggaaa gtccccaggctccccagcaggcagaagtatgcaaagcatgcatctcaattagtcagcaaccaggtgtggaaagtccccaggctcccc agcaggcagaagtatgcaaagcatgcatctcaattagtcagcaaccatagtcccgcccctaactccgcccatcccgcccctaactccg cccagttccgcccattctccgccccatggctgactaattttttttatttatgcagaggccgaggccgcctctgcctctgagctattccagaa gtagtgaggaggcttttttggaggcctaggcttttgcaaaaagctcccgggagcttgtatatccattttcggatctgatcagcacgtgttga caattaatcatcggcatagtatatcggcatagtataatacgacaaggtgaggaactaaaccatggccaagttgaccagtgccgttccgg tgctcaccgcgcgcgacgtcgccggagcggtcgagttctggaccgaccggctcgggttctcccgggacttcgtggaggacgacttc gccggtgtggtccgggacgacgtgaccctgttcatcagcgcggtccaggaccaggtggtgccggacaacaccctggcctgggtgtg ggtgcgcggcctggacgagctgtacgccgagtggtcggaggtcgtgtccacgaacttccgggacgcctccgggccggccatgacc gagatcggcgagcagccgtgggggcgggagttcgccctgcgcgacccggccggcaactgcgtgcacttcgtggccgaggagca ggactgacacgtgctacgagatttcgattccaccgccgccttctatgaaaggttgggcttcggaatcgttttccgggacgccggctgga tgatcctccagcgcggggatctcatgctggagttcttcgcccaccccaacttgtttattgcagcttataatggttacaaataaagcaatagc atcacaaatttcacaaataaagcatttttttcactgcattctagttgtggtttgtccaaactcatcaatgtatcttatcatgtctgtataccgtcga cctctagctagagcttggcgtaatcatggtcatagctgtttcctgtgtgaaattgttatccgctcacaattccacacaacatacgagccgga agcataaagtgtaaagcctggggtgcctaatgagtgagctaactcacattaattgcgttgcgctcactgcccgctttccagtcgggaaac ctgtcgtgccagctgcattaatgaatcggccaacgcgcggggagaggcggtttgcgtattgggcgctcttccgcttcctcgctcactga ctcgctgcgctcggtcgttcggctgcggcgagcggtatcagctcactcaaaggcggtaatacggttatccacagaatcaggggataac gcaggaaagaacatgtgagcaaaaggccagcaaaaggccaggaaccgtaaaaaggccgcgttgctggcgtttttccataggctccg cccccctgacgagcatcacaaaaatcgacgctcaagtcagaggtggcgaaacccgacaggactataaagataccaggcgtttcccc ctggaagctccctcgtgcgctctcctgttccgaccctgccgcttaccggatacctgtccgccettctcccttcgggaagcgtggcgcettc tcatagctcacgctgtaggtatctcagttcggtgtaggtcgttcgctccaagctgggctgtgtgcacgaaccccccgttcagcccgacc gctgcgccttatccggtaactatcgtcttgagtccaacccggtaagacacgacttatcgccactggcagcagccactggtaacaggatt agcagagcgaggtatgtaggcggtgctacagagttcttgaagtggtggcctaactacggctacactagaagaacagtatttggtatctg cgctctgctgaagccagttaccttcggaaaaagagttggtagctcttgatccggcaaacaaaccaccgctggtagcggtggtttttttgtt tgcaagcagcagattacgcgcagaaaaaaaggatctcaagaagatcctttgatcttttctacggggtctgacgctcagtggaacgaaaa ctcacgttaagggattttggtcatgagattatcaaaaaggatcttcacctagatccttttaaattaaaaatgaagttttaaatcaatctaaagt atatatgagtaaacttggtctgacagttaccaatgcttaatcagtgaggcacctatctcagcgatctgtctatttcgttcatccatagttgcct gactccccgtcgtgtagataactacgatacgggagggcttaccatctggccccagtgctgcaatgataccgcgagacccacgctcac cggctccagatttatcagcaataaaccagccagccggaagggccgagcgcagaagtggtcctgcaactttatccgcctccatccagtc tattaattgttgccgggaagctagagtaagtagttcgccagttaatagtttgcgcaacgttgttgccattgctacaggcatcgtggtgtcac gctcgtcgtttggtatggcttcattcagctccggttcccaacgatcaaggcgagttacatgatcccccatgttgtgcaaaaaagcggttag ctccttcggtcctccgatcgttgtcagaagtaagttggccgcagtgttatcactcatggttatggcagcactgcataattctcttactgtcat gccatccgtaagatgcttttctgtgactggtgagtactcaaccaagtcattctgagaatagtgtatgcggcgaccgagttgctcttgcccg gcgtcaatacgggataataccgcgccacatagcagaactttaaaagtgctcatcattggaaaacgtcttcggggcgaaaactctcaag gatcttaccgctgttgagatccagttcgatgtaacccactcgtgcacccaactgatcttcagcatcttttactttcaccagcgtttctgggtg agcaaaaacaggaaggcaaaatgccgcaaaaaagggaataagggcgacacggaaatgttgaatactcatactcttcctttttcaatatt attgaagcatttatcagggttattgtctcatgagcggatacatatttgaatgtatttagaaaaataaacaaataggggttccgcgcacattc cccgaaaagtgccacctgac (SEQ ID NO: 7). When these NRK1-expressing cells are supplemented with NR, NAD+ levels are augmented and cell viability is enhanced to enable efficient production and purification of the constitutively active human SARM1 SAM-TIR protein fragment. To express SARM1 SAM-TIR, the SARM1 N-terminal auto-inhibitory domain was deleted, keeping only the initiator Met. Downstream from this imitator Met, the resulting protein consists of human SARM1 residues 410 to 721: MSAWSHPQFEKGGGSGGGSGGSAWSHPQFEKGGGSSGGGGGGSSGGGASVPSWKE AEVQTWLQQIGFSKYCESFREQQVDGDLLLRLTEEELQTDLGMKSGITRKRFFRELT ELKTFANYSTCDRSNLADWLGSLDPRFRQYTYGLVSCGLDRSLLHRVSEQQLLEDC GIHLGVHRARILTAAREMLHSPLPCTGGKPSGDTPDVFISYRRNSGSQLASLLKVHLQ LHGFSVFIDVEKLEAGKFEDKLIQSVMGARNFVLVLSPGALDKCMQDHDCKDWVH KEIVTALSCGKNIVPIIDGFEWPEPQVLPEDMQAVLTFNGIKWSHEYQEATIEKIIRFL QGRSSRDSSAGSDTSLEGAAPMGPT (SEQ ID NO: 8). The fragment encoding the SARM1 SAM-TIR protein was cloned into the FCIV expression construct by standard methods to generate the FCIV-SST vector. The resultant vector has the following sequence:

(SEQ ID NO: 9)
gtcgacggatcgggagatctcccgatcccctatggtgcactctcagtaca
atctgctctgatgccgcatagttaagccagtatctgctccctgcttgtgt
gttggaggtcgctgagtagtgcgcgagcaaaatttaagctacaacaaggc
aaggcttgaccgacaattgcatgaagaatctgcttagggttaggcgtttt
gcgctgcttcgcgatgtacgggccagatatacgcgttgacattgattatt
gactagttattaatagtaatcaattacggggtcattagttcatagcccat
atatggagttccgcgttacataacttacggtaaatggcccgcctggctga
ccgcccaacgacccccgcccattgacgtcaataatgacgtatgttcccat
agtaacgccaatagggactttccattgacgtcaatgggtggagtatttac
ggtaaactgcccacttggcagtacatcaagtgtatcatatgccaagtacg
ccccctattgacgtcaatgacggtaaatggcccgcctggcattatgccca
gtacatgaccttatgggactttcctacttggcagtacatctacgtattag
tcatcgctattaccatggtgatgcggttttggcagtacatcaatgggcgt
ggatagcggtttgactcacggggatttccaagtctccaccccattgacgt
caatgggagtttgttttggcaccaaaatcaacgggactttccaaaatgtc
gtaacaactccgccccattgacgcaaatgggcggtaggcgtgtacggtgg
gaggtctatataagcagcgcgttttgcctgtactgggtctctctggttag
accagatctgagcctgggagctctctggctaactagggaacccactgctt
aagcctcaataaagcttgccttgagtgcttcaagtagtgtgtgcccgtct
gttgtgtgactctggtaactagagatccctcagacccttttagtcagtgt
ggaaaatctctagcagtggcgcccgaacagggacttgaaagcgaaaggga
aaccagaggagctctctcgacgcaggactcggcttgctgaagcgcgcacg
gcaagaggcgaggggcggcgactggtgagtacgccaaaaattttgactag
cggaggctagaaggagagagatgggtgcgagagcgtcagtattaagcggg
ggagaattagatcgcgatgggaaaaaattcggttaaggccagggggaaag
aaaaaatataaattaaaacatatagtatgggcaagcagggagctagaacg
attcgcagttaatcctggcctgttagaaacatcagaaggctgtagacaaa
tactgggacagctacaaccatcccttcagacaggatcagaagaacttaga
tcattatataatacagtagcaaccctctattgtgtgcatcaaaggataga
gataaaagacaccaaggaagctttagacaagatagaggaagagcaaaaca
aaagtaagaccaccgcacagcaagcggccgctgatcttcagacctggagg
aggagatatgagggacaattggagaagtgaattatataaatataaagtag
taaaaattgaaccattaggagtagcacccaccaaggcaaagagaagagtg
gtgcagagagaaaaaagagcagtgggaataggagctttgttccttgggtt
cttgggagcagcaggaagcactatgggcgcagcgtcaatgacgctgacgg
tacaggccagacaattattgtctggtatagtgcagcagcagaacaatttg
ctgagggctattgaggcgcaacagcatctgttgcaactcacagtctgggg
catcaagcagctccaggcaagaatcctggctgtggaaagatacctaaagg
atcaacagctcctggggatttggggttgctctggaaaactcatttgcacc
actgctgtgccttggaatgctagttggagtaataaatctctggaacagat
ttggaatcacacgacctggatggagtgggacagagaaattaacaattaca
caagcttaatacactccttaattgaagaatcgcaaaaccagcaagaaaag
aatgaacaagaattattggaattagataaatgggcaagtttgtggaattg
gtttaacataacaaattggctgtggtatataaaattattcataatgatag
taggaggcttggtaggtttaagaatagtttttgctgtactttctatagtg
aatagagttaggcagggatattcaccattatcgtttcagacccacctccc
aaccccgaggggacccgacaggcccgaaggaatagaagaagaaggtggag
agagagacagagacagatccattcgattagtgaacggatcggcactgcgt
gcgccaattctgcagacaaatggcagtattcatccacaattttaaaagaa
aaggggggattggggggtacagtgcaggggaaagaatagtagacataata
gcaacagacatacaaactaaagaattacaaaaacaaattacaaaaattca
aaattttcgggtttattacagggacagcagagatccagtttggttaatta
agggtgcagcggcctccgcgccgggttttggcgcctcccgcgggcgcccc
cctcctcacggcgagcgctgccacgtcagacgaagggcgcaggagcgttc
ctgatccttccgcccggacgctcaggacagcggcccgctgctcataagac
tcggccttagaaccccagtatcagcagaaggacattttaggacgggactt
gggtgactctagggcactggttttctttccagagagcggaacaggcgagg
aaaagtagtcccttctcggcgattctgcggagggatctccgtggggcggt
gaacgccgatgattatataaggacgcgccgggtgtggcacagctagttcc
gtcgcagccgggatttgggtcgcggttcttgtttgtggatcgctgtgatc
gtcacttggtgagttgcgggctgctgggctggccggggctttcgtggccg
ccgggccgctcggtgggacggaagcgtgtggagagaccgccaagggctgt
agtctgggtccgcgagcaaggttgccctgaactgggggttggggggagcg
cacaaaatggcggctgttcccgagtcttgaatggaagacgcttgtaaggc
gggctgtgaggtcgttgaaacaaggtggggggcatggtgggcggcaagaa
cccaaggtcttgaggccttcgctaatgcgggaaagctcttattcgggtga
gatgggctggggcaccatctggggaccctgacgtgaagtttgtcactgac
tggagaactcgggtttgtcgtctggttgcgggggcggcagttatgcggtg
ccgttgggcagtgcacccgtacctttgggagcgcgcgcctcgtcgtgtcg
tgacgtcacccgttctgttggcttataatgcagggtggggccacctgccg
gtaggtgtgcggtaggcttttctccgtcgcaggacgcagggttcgggcct
agggtaggctctcctgaatcgacaggcgccggacctctggtgaggggagg
gataagtgaggcgtcagtttctttggtcggttttatgtacctatcttctt
aagtagctgaagctccggttttgaactatgcgctcggggttggcgagtgt
gttttgtgaagttttttaggcaccttttgaaatgtaatcatttgggtcaa
tatgtaattttcagtgttagactagtaaagcttctgcaggtcgactctag
aaaattgtccgctaaattctggccgtttttggcttttttgttagacgaag
cttgggctgcaggtcgactctagaggatccGGATCCGCCACCATGTCAgc
tTGGAGCCACCCACAATTCGAAAAAGGCGGTGGCTCAGGCGGTGGCTCAG
GTGGCTCAGCTTGGAGCCACCCACAATTCGAAAAAGGCGGTGGCTCATCT
GGCGGAGGTGGCGGTGGCTCATCTGGCGGAGGTGCTAGCgtgcccagctg
gaaggaggccgaggttcagacgtggctgcagcagatcggtttctccaagt
actgcgagagcttccgggagcagcaggtggatggcgacctgcttctgcgg
ctcacggaggaggaactccagaccgacctgggcatgaaatcgggcatcac
ccgcaagaggttctttagggagctcacggagctcaagaccttcgccaact
attctacgtgcgaccgcagcaacctggcggactggctgggcagcctggac
ccgcgcttccgccagtacacctacggcctggtcagctgcggcctggaccg
ctccctgctgcaccgcgtgtctgagcagcagctgctggaagactgcggca
tccacctgggcgtgcaccgcgcccgcatcctcacggcggccagagaaatg
ctacactccccgctgccctgtactggtggcaaacccagtggggacactcc
agatgtcttcatcagctaccgccggaactcaggttcccagctggccagtc
tcctgaaggtgcacctgcagctgcatggcttcagtgtcttcattgatgtg
gagaagctggaagcaggcaagttcgaggacaaactcatccagagtgtcat
gggtgcccgcaactttgtgttggtgctatcacctggagcactggacaagt
gcatgcaagaccatgactgcaaggattgggtgcataaggagattgtgact
gctttaagctgcggcaagaacattgtgcccatcattgatggcttcgagtg
gcctgagccccaggtcctgcctgaggacatgcaggctgtgcttactttca
acggtatcaagtggtcccacgaataccaggaggccaccattgagaagatc
atccgcttcctgcagggccgctcctcccgggactcatctgcaggctctga
caccagtttggagggtgctgcacccatgggtccaacctaaactctagaat
tcgatatcaagcttatcgataatcaacctctggattacaaaatttgtgaa
agattgactggtattcttaactatgttgctccttttacgctatgtggata
cgctgattaatgcctttgtatcatgctattgcttcccgtatggctttcat
tttctcctccttgtataaatcctggttgctgtctctttatgaggagttgt
ggcccgttgtcaggcaacgtggcgtggtgtgcactgtgtttgctgacgca
acccccactggttggggcattgccaccacctgtcagctcctttccgggac
tttcgctttccccctccctattgccacggcggaactcatcgccgcctgcc
ttgcccgctgctggacaggggctcggctgttgggcactgacaattccgtg
gtgttgtcggggaaatcatcgtcctttccttggctgctcgcctgtgttgc
cacctggattctgcgcgggacgtccttctgctacgtcccttcggccctca
atccagcggaccttccttcccgcggcctgctgccggctctgcggcctctt
ccgcgtcttcgccttcgccctcagacgagtcggatctccctttgggccgc
ctccccgcatcgataccgtcgacctcgagacctagaaaaacatggagcaa
tcacaagtagcaatacagcagctaccaatgctgattgtgcctggctagaa
gcacaagaggaggaggaggtgggttttccagtcacacctcaggtaccttt
aagaccaatgacttacaaggcagctgtagatcttagccactattaaaaga
aaaggggggactggaagggctaattcactcccaacgaagacaagatatcc
ttgatctgtggatctaccacacacaaggctacttccctgattggcagaac
tacacaccagggccagggatcagatatccactgacctttggatggtgcta
caagctagtaccagttgagcaagagaaggtagaagaagccaatgaaggag
agaacacccgcttgttacaccctgtgagcctgcatgggatggatgacccg
gagagagaagtattagagtggaggtttgacagccgcctagcatttcatca
catggcccgagagctgcatccggactgtactgggtctctctggttagacc
agatctgagcctgggagctctctggctaactagggaacccactgcttaag
cctcaataaagcttgccttgagtgcttcaagtagtgtgtgcccgtctgtt
gtgtgactctggtaactagagatccctcagacccttttagtcagtgtgga
aaatctctagcagggcccgtttaaacccgctgatcagcctcgactgtgcc
ttctagttgccagccatctgttgtttgcccctcccccgtgccttccttga
ccctggaaggtgccactcccactgtcctttcctaataaaatgaggaaatt
gcatcgcattgtctgagtaggtgtcattctattctggggggtggggtggg
gcaggacagcaagggggaggattgggaagacaatagcaggcatgctgggg
atgcggtgggctctatggcttctgaggcggaaagaaccagctggggctct
agggggtatccccacgcgccctgtagcggcgcattaagcgcggcgggtgt
ggtggttacgcgcagcgtgaccgctacacttgccagcgccctagcgcccg
ctcctttcgctttcttcccttcctttctcgccacgttcgccggctttccc
cgtcaagctctaaatcgggggctccctttagggttccgatttagtgcttt
acggcacctcgaccccaaaaaacttgattagggtgatggttcacgtagtg
ggccatcgccctgatagacggtttttcgccctttgacgttggagtccacg
ttctttaatagtggactcttgttccaaactggaacaacactcaaccctat
ctcggtctattcttttgatttataagggattttgccgatttcggcctatt
ggttaaaaaatgagctgatttaacaaaaatttaacgcgaattaattctgt
ggaatgtgtgtcagttagggtgtggaaagtccccaggctccccagcaggc
agaagtatgcaaagcatgcatctcaattagtcagcaaccaggtgtggaaa
gtccccaggctccccagcaggcagaagtatgcaaagcatgcatctcaatt
agtcagcaaccatagtcccgcccctaactccgcccatcccgcccctaact
ccgcccagttccgcccattctccgccccatggctgactaatttatttatt
tatgcagaggccgaggccgcctctgcctctgagctattccagaagtagtg
aggaggctataggaggcctaggcttttgcaaaaagctcccgggagcttgt
atatccattttcggatctgatcagcacgtgttgacaattaatcatcggca
tagtatatcggcatagtataatacgacaaggtgaggaactaaaccatggc
caagttgaccagtgccgttccggtgctcaccgcgcgcgacgtcgccggag
cggtcgagttctggaccgaccggctcgggttctcccgggacttcgtggag
gacgacttcgccggtgtggtccgggacgacgtgaccctgttcatcagcgc
ggtccaggaccaggtggtgccggacaacaccctggcctgggtgtgggtgc
gcggcctggacgagctgtacgccgagtggtcggaggtcgtgtccacgaac
ttccgggacgcctccgggccggccatgaccgagatcggcgagcagccgtg
ggggcgggagttcgccctgcgcgacccggccggcaactgcgtgcacttcg
tggccgaggagcaggactgacacgtgctacgagatttcgattccaccgcc
gccttctatgaaaggttgggcttcggaatcgttttccgggacgccggctg
gatgatcctccagcgcggggatctcatgctggagttcttcgcccacccca
acttgtttattgcagcttataatggttacaaataaagcaatagcatcaca
aatttcacaaataaagcatttttttcactgcattctagttgtggtttgtc
caaactcatcaatgtatcttatcatgtctgtataccgtcgacctctagct
agagcttggcgtaatcatggtcatagctgtttcctgtgtgaaattgttat
ccgctcacaattccacacaacatacgagccggaagcataaagtgtaaagc
ctggggtgcctaatgagtgagctaactcacattaattgcgttgcgctcac
tgcccgctttccagtcgggaaacctgtcgtgccagctgcattaatgaatc
ggccaacgcgcggggagaggcggtttgcgtattgggcgctcttccgcttc
ctcgctcactgactcgctgcgctcggtcgttcggctgcggcgagcggtat
cagctcactcaaaggcggtaatacggttatccacagaatcaggggataac
gcaggaaagaacatgtgagcaaaaggccagcaaaaggccaggaaccgtaa
aaaggccgcgttgctggcgtttttccataggctccgcccccctgacgagc
atcacaaaaatcgacgctcaagtcagaggtggcgaaacccgacaggacta
taaagataccaggcgtttccccctggaagctccctcgtgcgctctcctgt
tccgaccctgccgcttaccggatacctgtccgcctttctcccttcgggaa
gcgtggcgctttctcatagctcacgctgtaggtatctcagttcggtgtag
gtcgttcgctccaagctgggctgtgtgcacgaaccccccgttcagcccga
ccgctgcgccttatccggtaactatcgtcttgagtccaacccggtaagac
acgacttatcgccactggcagcagccactggtaacaggattagcagagcg
aggtatgtaggcggtgctacagagttcttgaagtggtggcctaactacgg
ctacactagaagaacagtatttggtatctgcgctctgctgaagccagtta
ccttcggaaaaagagttggtagctcttgatccggcaaacaaaccaccgct
ggtagcggtggtttttttgtttgcaagcagcagattacgcgcagaaaaaa
aggatctcaagaagatcctttgatcttttctacggggtctgacgctcagt
ggaacgaaaactcacgttaagggattttggtcatgagattatcaaaaagg
atcttcacctagatccttttaaattaaaaatgaagttttaaatcaatcta
aagtatatatgagtaaacttggtctgacagttaccaatgcttaatcagtg
aggcacctatctcagcgatctgtctatttcgttcatccatagttgcctga
ctccccgtcgtgtagataactacgatacgggagggcttaccatctggccc
cagtgctgcaatgataccgcgagacccacgctcaccggctccagatttat
cagcaataaaccagccagccggaagggccgagcgcagaagtggtcctgca
actttatccgcctccatccagtctattaattgttgccgggaagctagagt
aagtagttcgccagttaatagtttgcgcaacgttgttgccattgctacag
gcatcgtggtgtcacgctcgtcgtttggtatggcttcattcagctccggt
tcccaacgatcaaggcgagttacatgatcccccatgttgtgcaaaaaagc
ggttagctccttcggtcctccgatcgttgtcagaagtaagttggccgcag
tgttatcactcatggttatggcagcactgcataattctcttactgtcatg
ccatccgtaagatgcttttctgtgactggtgagtactcaaccaagtcatt
ctgagaatagtgtatgcggcgaccgagttgctcttgcccggcgtcaatac
gggataataccgcgccacatagcagaactttaaaagtgctcatcattgga
aaacgttcttcggggcgaaaactctcaaggatcttaccgctgttgagatc
cagttcgatgtaacccactcgtgcacccaactgatcttcagcatctttta
ctttcaccagcgtttctgggtgagcaaaaacaggaaggcaaaatgccgca
aaaaagggaataagggcgacacggaaatgttgaatactcatactcttcca
tttcaatattattgaagcatttatcagggttattgtctcatgagcggata
catatttgaatgtatttagaaaaataaacaaataggggttccgcgcacat
ttccccgaaaagtgccacctgac.

NRK1-HEK293T cells were seeded onto 150 cm² plates at 20×10⁶ cells per plate in 25 mL growth medium comprised of 90% DMEM (Gibco 11965-084) and 10% FBS (Sigma F0926). The next day, cells were transfected by first premixing 15 μg FCIV-SST SST (SAM-TIR expression plasmid from Washington University) with 45 ul X-tremeGENE 9 DNA Transfection Reagent (Roche product #06365787001) and 750 ul OptiMEM (Gibco 31985062) and then adding this mix directly to the cells. The cultures were supplemented with 1 mM nicotinamide riboside (Thorne Research THR-00467) at time of transfection to minimize toxicity from SAM-TIR overexpression. Forty-eight hours after transfection, cells were harvested, pelleted by centrifugation at 1,000 rpm (Eppendorf Centrifuge 5804R, 15 Amp Version), and washed once with cold PBS (0.01 M phosphate buffered saline NaCl 0.138 M; KCl 0.0027 M; pH 7.4). The cells were resuspended in 0.5 ml PBS with protease inhibitors (Complete protease inhibitor cocktail, Roche product #11873580001). Cell lysates were prepared by sonication (Misonix Microson Ultrasonic Cell Disruptor, output=3, 20 episodes of stroke). The lysates were centrifuged at 12,000×g for 10 min at 4° C. (Eppendorf Centrifuge 5415C) to remove cell debris and the aliquots of supernatant (containing SARM1 SAM-TIR protein) were stored at −80° C. for later use. Protein concentration was determined by the Bicinchoninic (BCA) method and used to normalize lysate concentrations.

### Assay 2. Luminescence-Based Assay (NAD GLo)

This assay is an adaptation of the NAD+/NADH Glo assay (Promega G9071). In this assay, NAD+ cycling enzymes convert NAD+ into NADH. In the presence of NADH, the reductase enzymatically converts a pro-luciferin reductase substrate into luciferin. Luciferin is detected using Ultra-Glo™ rLuciferase, and the chemiluminescence intensity is proportional to the amount of NAD+ and NADH in the sample. In our assay conditions, the amount of NAD+ and NADH present in the lysate is undetectable with this assay, precluding any endogenous contribution to the final NAD+ detected. The assay was set up as follows: 2 μl inhibitor (final concentration 1 μM, 2% DMSO), 0.07 μg lysate (2 μl), and 2 μl of 400 nM NAD+. The reaction was incubated at 37° C. for 60 min, then 6 μl NAD+/NADH Glo detection reagent was added. After 30 min at room temperature, the luminescent signals were quantified using an Analyst HT reader (LJL Biosystems). The SARM1 SAM-TIR lysate catalyzed a dose-dependent depletion of NAD+, whereas NAD+ levels did not decline when reactions were performed with lysate prepared from control NRK1-HEK293T cells.

### Assay 3. HPLC-Based Assay 2

Reaction mixtures were prepared on ice by mixing 10 ul of SARM1 SAM-TIR cell lysate (320 fold dilution of lysate 11-3-2016, or 80 fold lysate dilution for assessment of time dependence) in PBS (pH 7.4) with 5 ul of compound stock. Compounds were first dissolved DMSO at 10 mM (final stock concentration). A 10 point compound dilution curve was prepared first with a 20 ul to 40 ul serial dilution in DMSO, followed by a 10 fold dilution (12 ul+108 ul) in PBS. Top concentration of compound in the assay is 250 μM. Compound and lysate were then preincubated, in duplicate, for various amounts of time (zero or 120 minutes for analysis of time dependence). 5 ul of 20 μM NAD+(5 μM final concentration) was then added for a final reaction volume of 20 μl. The reaction was incubated at 37° C. for 60 (or 10 minutes @room temp for assessment of time dependence), then stopped by addition of 180 μl of 0.55 M perchloric acid (HClO₄). The reactions were then place on for 10 min, 16.6 μl of 3 M K₂CO₃ was added to neutralize the solution. Precipitated salts were removed by centrifugation 10 min at 4,000 rpm (Sorvall ST 16R centrifuge). 80 ul of the supernatant was analyzed by HPLC (Waters 2695) with Kinetex (50×4.6 mm, 5 μm; Phenomenex). NAD and metabolites were eluted with a 1 ml/min gradient from 100% A: KPO₄ (5.026 g K₂HPO₄ and 2.876 KH₂PO₄ in 1 L H2O) to 3% methanol in 1 minute, followed by a linear gradient to 15% methanol in 1.5 minutes, held for 1 minute before returning to 0% methanol for 2.5 minutes for re-equilibration. NAD (3 minutes) and ADPR (1.5 min) were monitored by absorbance at 260 nm. Percent control conversion was established for each compound concentration. Blank (no lysate NAD only) values for ADPR were subtracted from samples and control (lysate+NAD) and control values from NAD depletion were subtracted from samples and blank to determine maximal ADPR conversion or NAD depletion (lysate dilutions used typically produced about a 50% conversion). Blanks and controls were run in triplicate (or more) then averaged. Duplicate data points from the 10 point dose curves were plotted using Grafit and IC₅₀'s were calculated using a 4 Parameter log fit.

### Example 11

### Synthesis of Compound A-6

Compound I^{A}-6 was prepared in accordance with Scheme 1^{A}, supra. The sidechain was prepared according to Scheme 2^{A}. below.

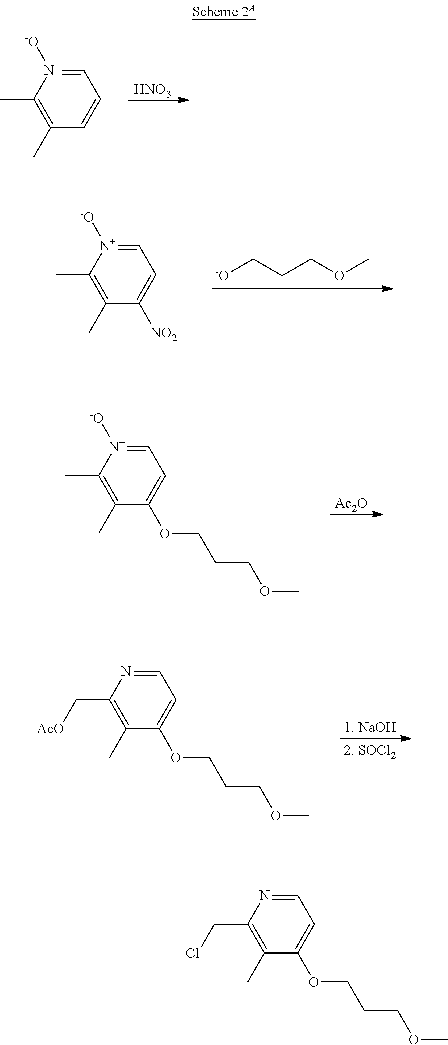

This molecule was then used to prepare Compound I^{A}-6 in accordance with Scheme 1^{A}, supra. The synthetic route is shown below.

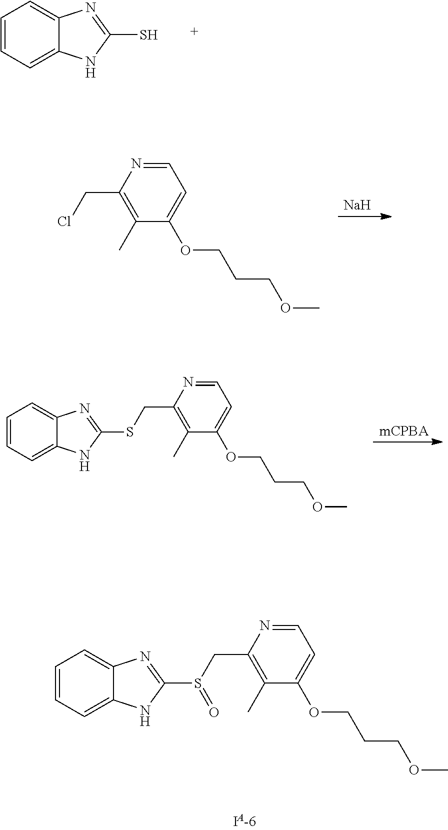

### Example 12

10 Point Dose Curves of SARM1 NADase Activity Inhibition with Compounds I^{A}-2, I^{A}-3, I^{A}-6 and I^{A}-8.

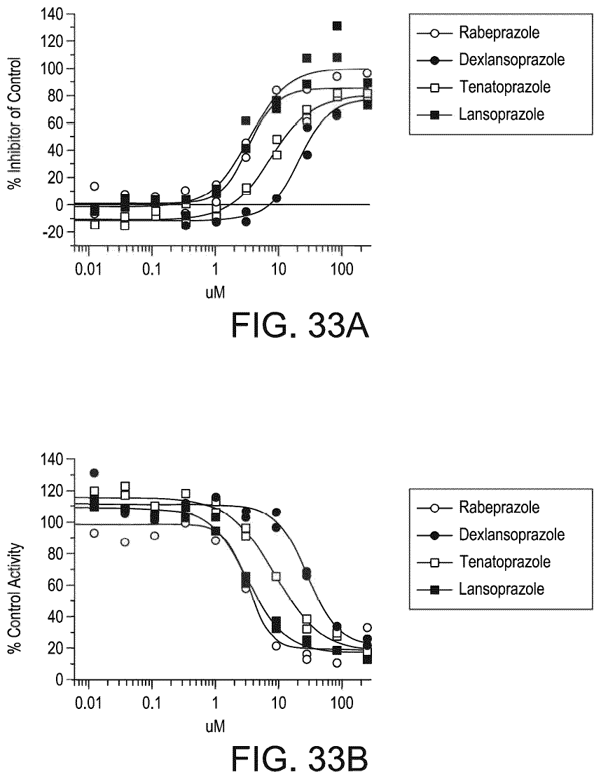

Compounds I^{A}-2, I^{A}-3, I^{A}-6 and I^{A}-8 demonstrate inhibition of SARM1 NADase activity, as shown in FIG. 33. Assay 3 (HPLC-based assay 2), described above, was used to assess NAD consumption and ADPR production from duplicate samples of a 10 point compound curve (average of n=2) ranging from 0.01-250 μM of compounds I^{A}-2, I^{A}-3, I^{A}-6 and I^{A}-8. The results are shown in FIG. 33, whereby FIG. 33A shows NAD consumption as a function of concentration of compounds I^{A}-2, I^{A}-3, I^{A}-6 and I^{A}-8 and FIG. 33B shows ADPR production as a function of concentration of compounds I^{A}-2, I^{A}-3, I^{A}-6 and I^{A}-8. As can be seen, increasing concentration of these compound I^{A}-6 from 0.01-250 μM leads to higher NAD consumption and lower ADPR production. The IC₅₀ for these compounds in Assay 3 are provided below in Table 5^{A}.

TABLE 5^{A}
IC₅₀ Values for SARM1 NADase Activity Inhibition
with Compounds I^{A}-2, I^{A}-3, I^{A}-6 and I^{A}-8.

| | IC₅₀ (μM, NAD | IC₅₀ (μM, ADPR |
|---|---|---|
| Compound | Consumption) | Production) |
| I^{A}-2 | 3.5 | 3.3 |
| I^{A}-3 | 21.4 | 28.3 |
| I^{A}-6 | 3.3 | 3.2 |
| I^{A}-8 | 7.4 | 8.7 |

### Example 13

Screening of SARM1 NADase Activity Inhibition with Compounds I^{A}-3, I^{A}-8, I^{A}-9, I^{A}-10, I^{A}-11 and I^{A}-13.

Compounds I^{A}-3, I^{A}-8, I^{A}-9, I^{A}-10, I^{A}-11 and I^{A}-13 demonstrates inhibition of SARM1 NADase activity, as shown below in Table 6^{A}. Assay 3 (HPLC-based assay), described above, was used to assess NAD consumption and ADPR production from duplicate samples of a single point screening (average of n=2) at 150 μM of each of Compounds I^{A}-3, I^{A}-8, I^{A}-9, I^{A}-10, I^{A}-11 and I^{A}-13. In Table 6^{A}, Compounds I^{A}-3, I^{A}-8, I^{A}-9, I^{A}-10, I^{A}-11 and I^{A}-13 are categorized by their ability to control NAD consumption, with “A” indicating >75%, “B” indicating between 50%-75% and “C” indicating <50%. Compounds I^{A}-3, I^{A}-8, I^{A}-9, I^{A}-10, I^{A}-11 and I^{A}-13 are also categorized by their ability to control ADPR production, with “A” indicating >75%, “B” indicating between 50%-75% and “C” indicating <50%.

TABLE 6^{A}
Single Point Screens of SARM1 NADase Activity Inhibition
with Compounds I^{A}-3, I^{A}-8, I^{A}-9, I^{A}-10, I^{A}-11 and I^{A}-13.

| | NAD Consumption | ADPR Production |
|---|---|---|
| Compound | (%) | (%) |
| I^{A}-3 | A | C |
| I^{A}-8 | B | C |
| I^{A}-9 | A | A |
| I^{A}-10 | A | A |
| I^{A}-11 | A | A |
| I^{A}-13 | A | B |

Without wishing to be bound by any particular theory, it is believed that the compounds of Formula I^{A} described herein may act by a unique mechanism that requires double protonation (in parietal cells which have pH of ˜1), followed by rearrangement to an activated tetracyclic intermediate which rapidly inactivates the H+-K+ ATPase. This is believed to arise from a precise arrangement of the sulfoxide group of the compounds of Formula I^{A} to the two activated rings of the compounds of Formula I^{A}. The data presented herein is indicative of a subtle SAR/discrimination with the benzimidazole-pyridine-sulfoxide scaffold of the compounds of Formula I^{A} and inhibition of SARM1 NADase activity.

### Example 14

### Synthesis of Compound I^{B}-1

Compound I^{B}-1 was prepared in accordance with Scheme 1^{B}, supra. The synthetic route is shown below.

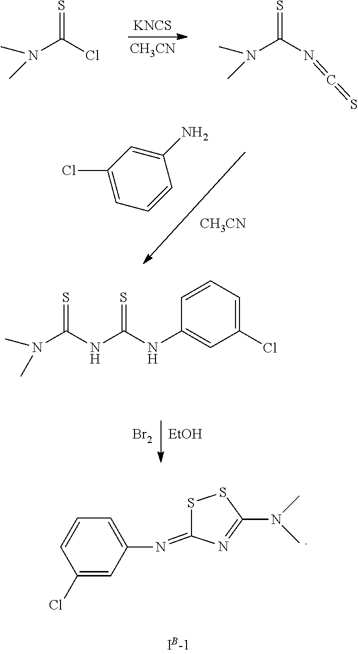

### Example 15

### Synthesis of Compound I^{B}-2

Compound I^{B}-2 was prepared in accordance with Scheme 1^{B}, supra. The synthetic route is shown below.

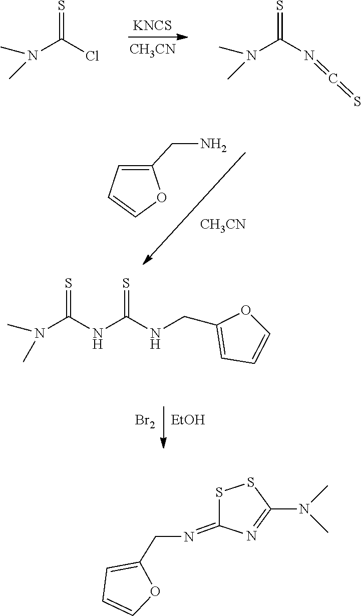

### Example 16

Dose Curves of SARM1 NADase Activity Inhibition with Compounds I^{B}-1 and I^{B}-2

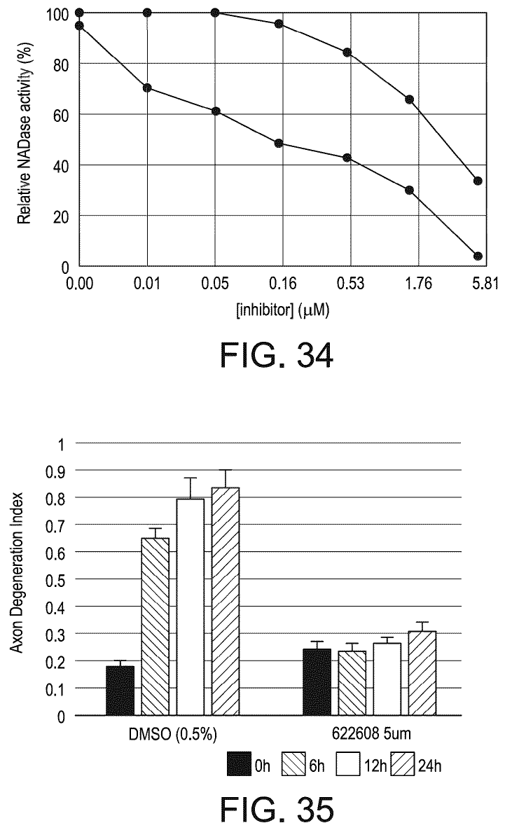

Compounds I^{B}-1 and I^{B}-2 demonstrate inhibition of SARM1 NADase activity, as shown in FIG. 34. Assay 3 (HPLC-based assay), described above, was used to assess NAD consumption from duplicate samples of a 7 point compound curve (average of n=2) ranging from 0-about 6 μM of compounds I^{B}-1 and B-2. The results are shown in FIG. 34, whereby it is shown that NADase activity decreases with increasing concentration of compounds I^{B}-1 and B-2. The upper curve represents compound I^{B}-1 and the lower curve represents B-2. The IC₅₀ for compound I^{B}-2 in Assay 3 was determined to be about 150 nM and the IC₅₀ for compound I^{B}-1 in Assay 3 was determined to be about 0.7 μM.

### Example 17

Prevention of Axonal Degeneration with Compound I^{B}-2

Mouse DRG Drop Culture: Mouse dorsal root ganglion (DRG) was dissected from embryonic days 13.5 CD1 mouse embryo (50 ganglion per embryo) and incubated with 0.05% Trypsin solution containing 0.02% EDTA (Gibco) at 37° C. for 15 min. Then cell suspensions are triturated by gentle pipetting and washed 3 times with the DRG growth medium (Neurobasal medium (Gibco) containing 2% B27 (Invitrogen), 100 ng/ml 2.5S NGF (Harlan Bioproduts), 1 mM uridine (Sigma), 1 mM 5-fluoro-2′-deoxyuridine (Sigma), penicillin, and streptomycin). Cells were suspended in DRG growth medium at a ratio of 100 ml medium/50 DRGs. The cell density of these suspensions was ˜7×10⁶ cells/ml. Cell suspensions (1.5 ml/96 well, 10 ml/24 well) were placed in the center of the well using either 96- or 24-well tissue culture plates (Corning) coated with poly-D-Lysine (0.1 mg/ml; Sigma) and laminin (3 mg/ml; Invitrogen). Cells were allowed to adhere in humidified tissue culture incubator (5% CO₂) for 15 min and then DRG growth medium was gently added (100 ml/96 well, 500 ml/24 well). Lentiviruses were added (1-10×10³ pfu) at 1-2 days in vitro (DIV) and metabolites were extracted or axon degeneration assays were performed at 6-7 DIV. When using 24 well DRG cultures, 50% of the medium was exchanged for a fresh medium at DIV4. NR (100 mM) was added 24 hr before axotomy or metabolite collection.

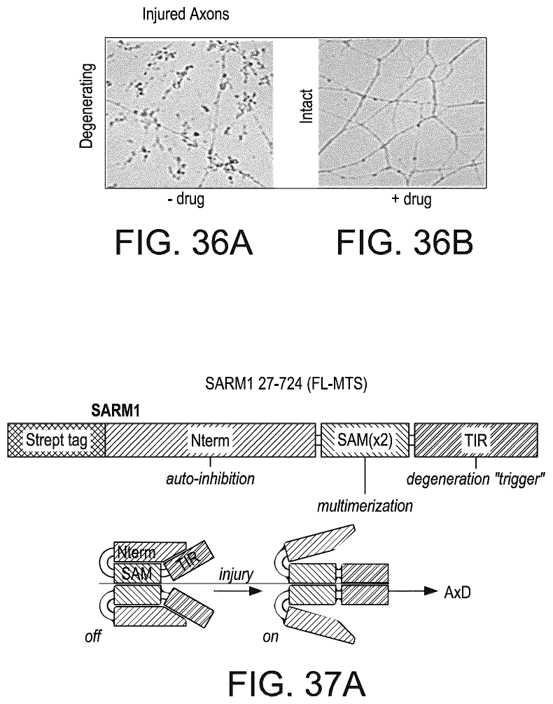

Axon Denegeneration Assay: Axons from DRG drop cultures in 96 well were transected using a micro surgical blade under micro-scope at DIV6. Bright field images of distal axons (6 fields per well) were taken at 0-72 hr after axotomy using a high content imager (Operetta; Perkinelmer) with 20× objective. Axon degeneration was quantified using degeneration index (DI) calculated using ImageJ (NIH). The average DI from 6 fields per well was obtained and averaged for each independent well. The DI was calculated from axon images from the same fields before (0 hr) and after (9-72 hr) axotomy. Experiments were repeated 3 times with 3 independent wells (n=9). For statistical analysis, DI was compared using one-way ANOVA and Holm-Bonferroni multiple comparison using R (RRID:SCR 002394). Data from this experiment is represented below as a bar graph in FIG. 35 and the images are shown in FIG. 36.

Compound I^{B}-2 demonstrates prevention of axonal degeneration in a mouse dorsal root ganglion (DRG) drop culture assay, as described above. FIG. 35 shows a control sample (grouping on left side of FIG. 35) and a sample that includes compound I^{B}-2 (grouping on right side of FIG. 35) and the level of axonal degeneration after exposure to both samples at intervals of 0 hours, 6 hours, 12 hours and 24 hours. As can be seen, axons exposed to compound I^{B}-2 showed a substantial decrease in axonal degeneration compared to the control sample after intervals of 6 hours, 12 hours and 24 hours.

FIG. 36 shows SEM micrographs of injured axons under degenerating conditions with (FIG. 36A) and without (FIG. 36B) exposure to compound B-2. As can be seen, axons exposed to compound I^{B}-2 were intact under degenerating conditions, whereas axons not exposed to compound I^{B}-2 were degenerated.

### Example 18

The present Example demonstrates successful development of an in vitro assay using a full-length SARM1. The assay described in this Example can be used, for example, to identify and/or characterize compounds that inhibit full-length SARM1 in vivo.

Cells expressing SARM1 show decreased expression after extended growth. NAD+ levels are lower in SARM1-expressing cells, but these cells do not die. Moreover, a C-terminal GFP tag decreased SARM1 NADase activity. The presently described assay overcame these challenges.

Full-length SARM1 lacking the mitochondrial targeting sequence (MTS) (FL-MTS SARM1) was produced and tested as described below.

### Production of Affinity Tagged FL-MTS SARM1.

NRK1-HEK293T cells represent a polyclonal cell line that has been stably transfected with an FCIV expression vector that expresses human Nicotinamide Riboside Kinase 1 (NRK1), an enzyme that converts the NAD+ biosynthetic precursor nicotinamide riboside (NR) to NMN, the immediate precursor of NAD+. When these NRK1-expressing cells are supplemented with NR, NAD+ levels are augmented and cell viability is enhanced to enable efficient production and purification of SARM1.

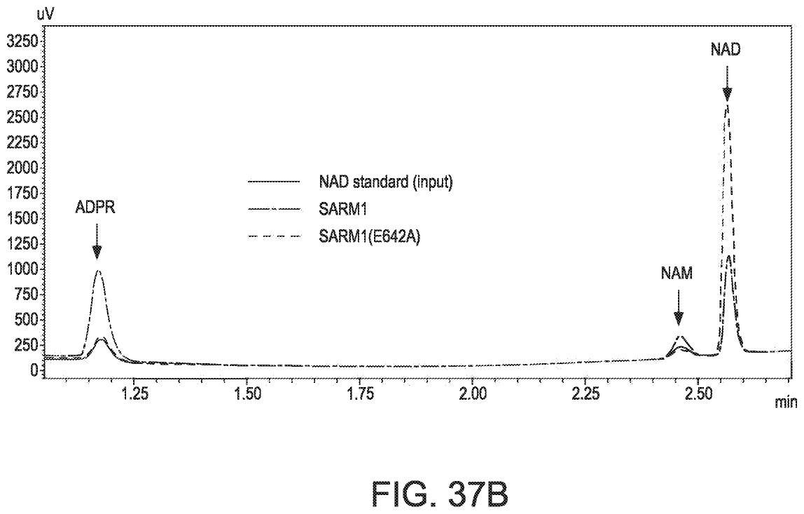

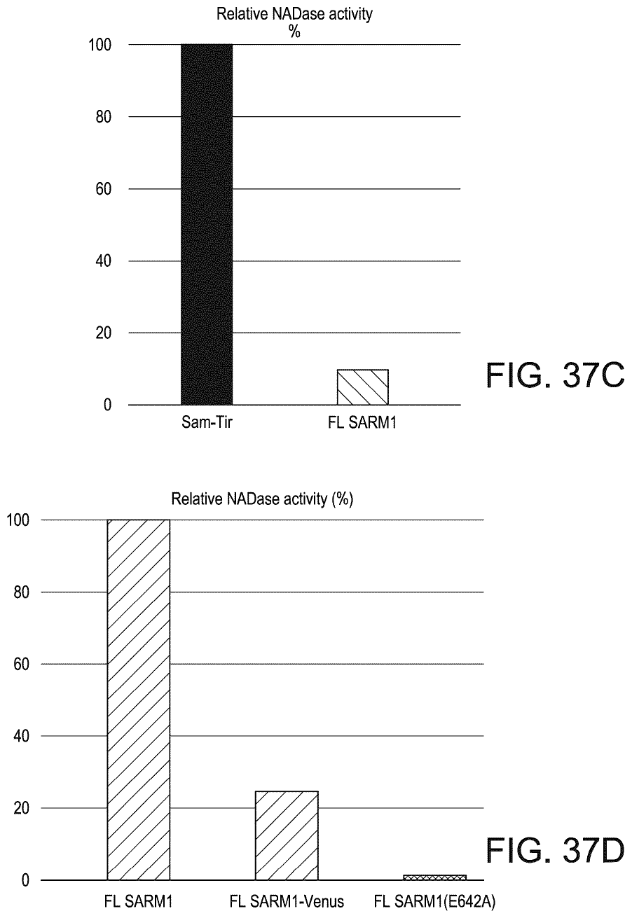

For these experiments, human SARM1 lacking the first 26 residues, which correspond to the SARM1 mitochondrial targeting sequence, was engineered with a StrepTag affinity tag on the N-terminus (referred to as FL-MTS SARM1; see FIG. 37A). This modified SARM1 protein was cloned into the lentiviral vector FCIV to generate an FL-MTS SARM1 mammalian expression vector. A similar construct was generated in which the catalytic Glu residue at SARM1 position 642 was changed to Ala. This inactive mutant FL-MTS SARM1(E642A) was used as a negative control in assays described in this Example. Finally, an active SARM1 mutant (SAM-TIR) was similarly constructed with an N-terminal StrepTag fused to SARM1 residues 409-724 and used as a positive control in these assays.

To produce the StrepTag-FL-MTS SARM1 and StrepTag-FL-MTS SARM1(E642A) or the active SARM1 SAM-TIR protein, NRK1-HEK293T cells were seeded onto 150 cm² plates at 20×10⁶ cells per plate. The next day, the cells were transfected with 15 μg FCIV-FL-MTS SARM1 or FCIV FL-MTS SARM1(E642A) or SARM1 SAM-TIR expression vector using X-tremeGENE 9 DNA Transfection Reagent (Roche product #06365787001). The cultures were supplemented with 1 mM NR at the time of transfection to minimize toxicity from SARM1 protein expression. Forty-eight hours after transfection, cells were harvested, pelleted by centrifugation at 1,000 rpm (Sorvall ST 16R centrifuge, Thermo Fisher), and washed once with cold PBS (0.01 M phosphate buffered saline NaCl 0.138 M; KCl 0.0027 M; pH 7.4). The cells were resuspended in PBS with protease inhibitors (Complete protease inhibitor cocktail, Roche product #11873580001) and cell lysates were prepared by sonication (Branson Sonifer 450, output=3, 20 episodes of stroke). The lysates were centrifuged (12,000×g for 10 min at 4° C.) to remove cell debris and the supernatants containing the affinity-tagged FL-MTS SARM1 or FL-MTS SARM1(E642A) or SARM1 SAM-TIR protein were stored at −80° C. for later use. For affinity purification, the supernatants were incubated with 100 μL MagStrep (Strep-Tactin) type 3 XT beads suspension (IBA Lifesciences) for 30 min. The beads bound with FL-MTS SARM1 or FL-MTS SARM1(E642A) or SARM1 SAM-TIR protein were then washed three times with binding buffer and resuspended in 100 μL of binding buffer for enzymatic assays.

### Assaying for NAD Cleavage Activity

Reaction mixtures were prepared using MagStrep beads laden with affinity tagged FL-MTS SARM1 or FL-MTS SARM1(E642A) protein (1 to 30 ng on 1-4 ul of beads or the active SAM-TIR protein (0.25 ng) and PBS (pH 7.4) to a final volume of 12 μl. NAD (5 μM final concentration) was then added for a final reaction volume of 20 μl. The reaction was incubated at 37° C. for 60 min, and then stopped by addition of 180 μl of 0.55 M perchloric acid (HClO₄) and placed on ice. After 10 min on ice, the reaction plates were centrifuged for 10 min at 4,000 rpm (Sorvall ST 16R centrifuge). The supernatant (120 μl) was transferred to a new plate and 10 μl of 3M K2CO3 was added to neutralize the solution. Precipitated salts were removed by centrifugation for 10 min at 4,000 rpm (Sorvall ST 16R centrifuge). The supernatant (90 μl) containing the extracted metabolites was mixed with 0.5 M Potassium Phosphate buffer (10 μL) and metabolites were analyzed by HPLC (Shimadzu Nexera X2) with C18 reverse phase column (Kinetex 100×3 mm, 2.6 μm; Phenomenex) to quantify the amounts of NAD and ADPR, a product of the NAD cleavage reaction. Internal standards for NAD and ADPR were used to generate standard curves for quantification of the respective compounds. The levels for each compound in each experimental sample were normalized to the 0 min time point that was analyzed concurrently. From these values, the NAD/ADPR ratio was calculated as a measure of NAD cleavage activity.

FIG. 37B shows HPLC traces from an assay, with peaks corresponding to NAD, NAM, and ADPR delineated with arrows. As shown in FIGS. 37C and 37D, full-length SARM1 showed significantly lower NADase activity than SAM-TIR, while the FL SARM1 E642A mutant had significantly lower NADase activity than full-length SARM1.

Thus, the presently described assay successfully measures NADase activity using full-length SARM1.

While we have described a number of embodiments of this disclosure, it is apparent that our basic examples may be altered to provide other embodiments that utilize the compounds and methods of this disclosure. Therefore, it will be appreciated that the scope of this disclosure is to be defined by the appended claims rather than by the specific embodiments that have been represented by way of example.

We claim:

1. A method of inhibiting SARM1 NADase activity and/or treating a neurodegenerative or neurological disease or disorder in a patient in need thereof, comprising administering to said patient the composition according to formula I^{A}: 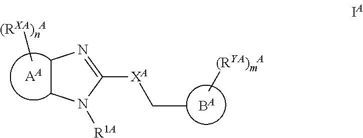
or a pharmaceutically acceptable salt thereof, wherein:
X^{A} is —S—, —SO— or —SO₂—;
R^{1A} is hydrogen, C₁₋₄ aliphatic, alkali metal, alkaline earth metal, ammonium or N⁺(C₁₋₄alkyl)₄;
Ring A^{A} is selected from a benzo fused ring and a 5-6 membered heteroaromatic fused ring having 1-3 heteroatoms independently selected from nitrogen, oxygen, and sulfur;
Ring B^{A} is selected from phenyl, an 8-10 membered bicyclic aromatic carbocyclic ring, a 4-8 membered saturated or partially unsaturated monocyclic heterocyclic ring having 1-2 heteroatoms independently selected from nitrogen, oxygen, or sulfur, a 5-6 membered monocyclic heteroaromatic ring having 1-4 heteroatoms independently selected from nitrogen, oxygen, or sulfur, or an 8-10 membered bicyclic heteroaromatic ring having 1-5 heteroatoms independently selected from nitrogen, oxygen, or sulfur;
R^{XA} and R^{YA} are independently hydrogen, C₁₋₄ aliphatic optionally substituted with 1-4 halogen, —OR^{A}, —SR^{A}, —N(R^{A})₂, —N(R^{A})C(O)R^{A}, —C(O)N(R^{A})₂, —N(R^{A})C(O)N(R^{A})₂, —N(R^{A})C(O)OR^{A}, —OC(O)N(R^{A})₂, —N(R)S(O)₂R^{A}, —S(O)₂N(R^{A})₂, —C(O)R^{A}, —C(O)OR^{A}, —OC(O)R^{A}, —S(O)R^{A}, —S(O)₂R^{A}, phenyl, an 8-10 membered bicyclic aromatic carbocyclic ring, a 4-8 membered saturated or partially unsaturated monocyclic heterocyclic ring having 1-2 heteroatoms independently selected from nitrogen, oxygen, or sulfur, a 5-6 membered monocyclic heteroaromatic ring having 1-4 heteroatoms independently selected from nitrogen, oxygen, or sulfur, or an 8-10 membered bicyclic heteroaromatic ring having 1-5 heteroatoms independently selected from nitrogen, oxygen, or sulfur;
each R^{A} is independently hydrogen or an optionally substituted group selected from C₁₋₆ aliphatic, a 3-8 membered saturated or partially unsaturated monocyclic carbocyclic ring, phenyl, an 8-10 membered bicyclic aromatic carbocyclic ring, a 4-8 membered saturated or partially unsaturated monocyclic heterocyclic ring having 1-2 heteroatoms independently selected from nitrogen, oxygen, or sulfur, a 5-6 membered monocyclic heteroaromatic ring having 1-4 heteroatoms independently selected from nitrogen, oxygen, or sulfur, or an 8-10 membered bicyclic heteroaromatic ring having 1-5 heteroatoms independently selected from nitrogen, oxygen, or sulfur;
m^{A} and n^{A} are independently 0, 1, 2, or 3.

2. The method according to claim 1, wherein the compound of formula I^{A} is selected from the group consisting of:

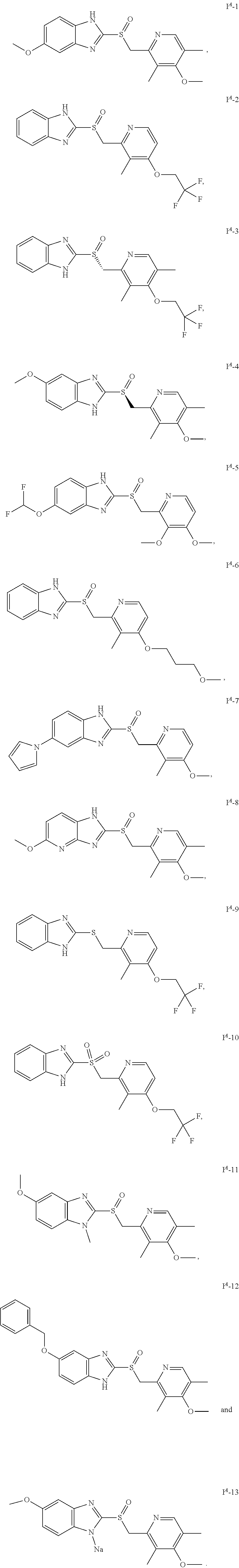

3. The method according to claim 1, wherein X^{A} is —SO—.

4. The method according to claim 1, wherein n^{A} is 0 or 1 and m^{A} is 2 or 3.

5. The method according to claim 1, wherein Ring A^{A} is an arylo fused ring and Ring B^{A} is a heteroaryl ring.

6. The method according to claim 1, wherein Ring A^{A} is a benzo fused ring and Ring B^{A} is a pyridyl ring.

7. The method according to claim 1, wherein Ring A^{A} is a heteroaromatic fused ring and Ring B^{A} is a heteroaryl ring.

8. The method according to claim 1, wherein Ring A^{A} is selected from the group consisting of a pyrido fused ring, a pyrimidino fused ring, a pyridazino fused ring, pyrazino fused ring, a triazino fused ring, a pyrrolo fused ring, a thiopheno fused ring, a furano fused ring, a thiazolofused ring, an isothiazolo fused ring, an imidazolo fused ring, a pyrazolo fused ring, an oxazolo fused ring and an isoxazolo fused ring.

9. The method according to claim 1, wherein Ring B^{A} is selected from the group consisting of phenyl, biphenyl, napthyl, anthracyl, indanyl, phthalimidyl, naphthimidyl, phenanthridinyl, tetrahydronaphthyl, thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, indolizinyl, purinyl, naphthyridinyl and pteridinyl.

10. The method according to claim 1, wherein R^{1A} is hydrogen, C₁₋₄ aliphatic or alkali metal.

11. The method according to claim 1, wherein R^{1A} is hydrogen, methyl or sodium.

12. The method according to claim 1, wherein R^{YA} is hydrogen, C₁₋₄ aliphatic optionally substituted with 1-4 halogen or —OR^{A}; and R^{A} is optionally substituted C₁₋₆ aliphatic.

13. The method according to claim 12, wherein R^{YA} is hydrogen, —CH₃, —OCH₃, —OCH₂CF₃ or —O(CH₂)₃OCH₃.

14. The method according to claim 1, wherein R^{XA} is hydrogen, —OR^{A}, or heteroaryl; and R^{A} is optionally substituted C₁₋₆ aliphatic or benzyl.

15. The method according to claim 14, wherein R^{XA} is hydrogen, —OCH₃, —OCHCF₂, pyrrolyl or —OCH₂-phenyl.

16. The method according to claim 1, wherein the compounds of Formula I^{A} are administered as part of a pharmaceutically acceptable composition.

17. The method according to claim 1, wherein the compounds of Formula I^{A} are administered orally.

18. The method according to claim 1, wherein the compounds of Formula I^{A} are administered in a range of 0.01-100 mg/kg body weight of the patient.

19. The method according to claim 1, wherein the neurodegenerative or neurological disease or disorder is associated with axonal degeneration, axonal damage, axonopathy, a demyelinating disease, a central pontine myelinolysis, a nerve injury disease or disorder, a metabolic disease, a mitochondrial disease, metabolic axonal degeneration, axonal damage resulting from a leukoencephalopathy or a leukodystrophy.

20. The method according to claim 1, wherein the neurodegenerative or neurological disease or disorder is selected from the group consisting of spinal cord injury, stroke, multiple sclerosis, progressive multifocal leukoencephalopathy, congenital hypomyelination, encephalomyelitis, acute disseminated encephalomyelitis, central pontine myelolysis, osmotic hyponatremia, hypoxic demyelination, ischemic demyelination, adrenoleukodystrophy, Alexander's disease, Niemann-Pick disease, Pelizaeus Merzbacher disease, periventricular leukomalacia, globoid cell leukodystrophy (Krabbe's disease), Wallerian degeneration, optic neuritis, transverse myelitis, amyotrophic lateral sclerosis (ALS, Lou Gehrig's disease), Huntington's disease, Alzheimer's disease, Parkinson's disease, Tay-Sacks disease, Gaucher's disease, Hurler Syndrome, traumatic brain injury, post radiation injury, neurologic complications of chemotherapy (chemotherapy induced neuropathy; CIPN), neuropathy, acute ischemic optic neuropathy, vitamin B12 deficiency, isolated vitamin E deficiency syndrome, Bassen-Komzweig syndrome, Glaucoma, Leber's hereditary optic atrophy (neuropathy), Leber congenital amaurosis, neuromyelitis optica, metachromatic leukodystrophy, acute hemorrhagic leukoencephalitis, trigeminal neuralgia, Bell's palsy, cerebral ischemia, multiple system atrophy, traumatic glaucoma, tropical spastic paraparesis human T-lymphotropic virus 1 (HTLV-1) associated myelopathy, west nile virus encephalopathy, La Crosse virus encephalitis, Bunyavirus encephalitis, pediatric viral encephalitis, essential tremor, Charcot-Marie-Tooth disease, motorneuron disease, spinal muscular atrophy (SMA), hereditary sensory and autonomic neuropathy (HSAN), adrenomyeloneuropathy, progressive supra nuclear palsy (PSP), Friedrich's ataxia, hereditary ataxias, noise induced hearing loss, congenital hearing loss, Lewy Body Dementia, frontotemporal dementia, amyloidosis, diabetic neuropathy, HIV neuropathy, enteric neuropathies and axonopathies, Guillain-Barre syndrome, and severe acute motor axonal neuropathy (AMAN).

21. A method of inhibiting SARM1 NADase activity and/or treating a neurodegenerative or neurological disease or disorder in a patient in need thereof, comprising administering to said patient the composition according to formula I^{B}: 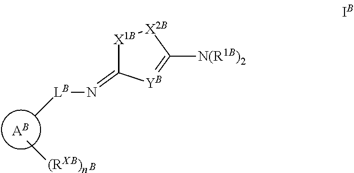
or a pharmaceutically acceptable salt thereof, wherein:
X^{1B} and X^{2B} are independently —O—, —S—, or —NR^{B}—, provided that one of X^{1B} and X^{2B} is —O— or —S— and both of X^{1B} and X^{2B} are not —O—;
Y^{B} is —N— or —CH—;
each R^{1B} is independently hydrogen or optionally substituted C₁₋₄ aliphatic;
Ring A^{B} is selected from phenyl, an 8-10 membered bicyclic aromatic carbocyclic ring, a 4-8 membered saturated or partially unsaturated monocyclic heterocyclic ring having 1-2 heteroatoms independently selected from nitrogen, oxygen, or sulfur, a 5-6 membered monocyclic heteroaromatic ring having 1-4 heteroatoms independently selected from nitrogen, oxygen, or sulfur, or an 8-10 membered bicyclic heteroaromatic ring having 1-5 heteroatoms independently selected from nitrogen, oxygen, or sulfur;
each R^{XB} is independently hydrogen, halogen or an optionally substituted group selected from C₁₋₆ aliphatic, a 3-8 membered saturated or partially unsaturated monocyclic carbocyclic ring, phenyl, an 8-10 membered bicyclic aromatic carbocyclic ring, a 4-8 membered saturated or partially unsaturated monocyclic heterocyclic ring having 1-2 heteroatoms independently selected from nitrogen, oxygen, or sulfur, a 5-6 membered monocyclic heteroaromatic ring having 1-4 heteroatoms independently selected from nitrogen, oxygen, or sulfur, or an 8-10 membered bicyclic heteroaromatic ring having 1-5 heteroatoms independently selected from nitrogen, oxygen, or sulfur;
each R^{B} is independently hydrogen or an optionally substituted group selected from C₁₋₆ aliphatic, a 3-8 membered saturated or partially unsaturated monocyclic carbocyclic ring, phenyl, an 8-10 membered bicyclic aromatic carbocyclic ring, a 4-8 membered saturated or partially unsaturated monocyclic heterocyclic ring having 1-2 heteroatoms independently selected from nitrogen, oxygen, or sulfur, a 5-6 membered monocyclic heteroaromatic ring having 1-4 heteroatoms independently selected from nitrogen, oxygen, or sulfur, or an 8-10 membered bicyclic heteroaromatic ring having 1-5 heteroatoms independently selected from nitrogen, oxygen, or sulfur;
L^{B} is a covalent bond, a C₁₋₆ membered straight or branched bivalent hydrocarbon chain, cyclopropylenyl, cyclobutylenyl, or oxetanylenyl; and
n^{B} is 0, 1, 2, 3 or 4.

22. The method according to claim 21, wherein the compound of formula I^{B} is selected from the group consisting of:

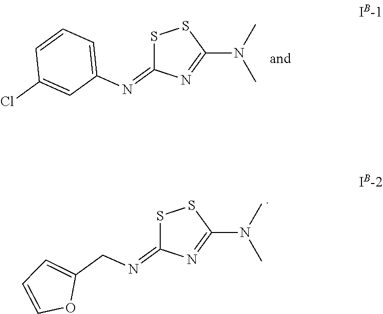 [SMILES: CN(C)c1n/c(=N/c2cccc(Cl)c2)ss1 ; CN(C)c1n/c(=N/Cc2ccco2)ss1]

23. The method according to claim 21, wherein X^{1B} and X^{2B} are —S— and Y^{B} is —N—.

24. The method according to claim 21, wherein Ring A^{B} is aryl or heteroaryl.

25. The method according to claim 21, wherein Ring A^{B} is selected from the group consisting of phenyl, biphenyl, napthyl and anthracyl.

26. The method according to claim 21, wherein Ring A^{B} is selected from the group consisting of indanyl, phthalimidyl, naphthimidyl, phenanthridinyl, tetrahydronaphthyl, thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, indolizinyl, purinyl, naphthyridinyl and pteridinyl.

27. The method according to claim 21, wherein R^{1B} is optionally substituted C₁₋₄ aliphatic.

28. The method according to claim 21, wherein R^{1B} is methyl.

29. The method according to claim 21, wherein L^{B} is a covalent bond or a 1-6 membered straight or branched bivalent hydrocarbon chain.

30. The method according to claim 21, wherein L^{B} is a covalent bond or a methylene group.

31. The method according to claim 21, wherein R^{XB} is hydrogen, halogen or optionally substituted C₁₋₆ aliphatic.

32. The method according to claim 21, wherein R^{XB} is hydrogen or —Cl.

33. The method according to claim 21, wherein the compounds of Formula I^{B} are administered as part of a pharmaceutically acceptable composition.

34. The method according to claim 21, wherein the compounds of Formula I^{B} are administered orally.

35. The method according to claim 21, wherein the compounds of Formula I^{B} are administered in a range of 0.01-100 mg/kg body weight of the patient.

36. The method according to claim 21, wherein the neurodegenerative or neurological disease or disorder is associated with axonal degeneration, axonal damage, axonopathy, a demyelinating disease, a central pontine myelinolysis, a nerve injury disease or disorder, a metabolic disease, a mitochondrial disease, metabolic axonal degeneration, axonal damage resulting from a leukoencephalopathy or a leukodystrophy.

37. The method according to claim 21, wherein the neurodegenerative or neurological disease or disorder is selected from the group consisting of spinal cord injury, stroke, multiple sclerosis, progressive multifocal leukoencephalopathy, congenital hypomyelination, encephalomyelitis, acute disseminated encephalomyelitis, central pontine myelolysis, osmotic hyponatremia, hypoxic demyelination, ischemic demyelination, adrenoleukodystrophy, Alexander's disease, Niemann-Pick disease, Pelizaeus Merzbacher disease, periventricular leukomalacia, globoid cell leukodystrophy (Krabbe's disease), Wallerian degeneration, optic neuritis, transverse myelitis, amyotrophic lateral sclerosis (ALS, Lou Gehrig's disease), Huntington's disease, Alzheimer's disease, Parkinson's disease, Tay-Sacks disease, Gaucher's disease, Hurler Syndrome, traumatic brain injury, post radiation injury, neurologic complications of chemotherapy (chemotherapy induced neuropathy; CIPN), neuropathy, acute ischemic optic neuropathy, vitamin B2 deficiency, isolated vitamin E deficiency syndrome, Bassen-Kornzweig syndrome, Glaucoma, Leber's hereditary optic atrophy, Leber congenital amaurosis, neuromyelitis optica, metachromatic leukodystrophy, acute hemorrhagic leukoencephalitis, trigeminal neuralgia, Bell's palsy, cerebral ischemia, multiple system atrophy, traumatic glaucoma, tropical spastic paraparesis human T-lymphotropic virus 1 (HTLV-1) associated myelopathy, west nile virus encephalopathy, La Crosse virus encephalitis, Bunyavirus encephalitis, pediatric viral encephalitis, essential tremor, Charcot-Marie-Tooth disease, motorneuron disease, spinal muscular atrophy (SMA), hereditary sensory and autonomic neuropathy (HSAN), adrenomyeloneuropathy, progressive supra nuclear palsy (PSP), Friedrich's ataxia, hereditary ataxias, noise induced hearing loss and congenital hearing loss.

38. A method of identifying a SARM1 NADase inhibitor, comprising:
a. providing a mixture comprising i) a mutant or fragment of SARM1, ii) NAD+ and iii) a candidate inhibitor, wherein the mutant or fragment has constitutive NADase activity;
b. incubating the mixture;
c. quantifying NAD+ and ADPR in the mixture after the incubating;
d. determining the molar ratio of NAD+:ADPR; and
e. identifying the candidate inhibitor compound as an NADase inhibitor if the molar ratio is greater than that of a control mixture that does not contain the candidate inhibitor.

39. A method in accordance with claim 38, wherein the quantifying NAD+ and ADPR in the mixture comprises performing an HPLC analysis.

40. A method in accordance with claim 38, wherein the mixture comprises a cell lysate comprising the mutant or fragment of SARM1.

41. A method in accordance with claim 40, wherein cell lysate is a lystate of NRK1-HEK293T cells comprising the mutant or fragment of SARM1.

42. A method in accordance with claim 38, wherein the mutant or fragment of SARM1 is a SARM-TIR fragment.

43. A method in accordance with claim 38, wherein the mutant or fragment of SARM1 consists of human SARM1 residues 410 to 721.

44. A method in accordance with claim 38, wherein the mutant or fragment of SARM1 consists of murine SARM1 residues homologous to human SARM1 residues 410 to 721.

45. A method in accordance with claim 38, wherein the mutant or fragment of SARM1 is a SARM1 polypeptide deleted for an N-terminal auto-inhibitory domain.

46. A method in accordance with claim 38, wherein the candidate inhibitor compound is identified as an NADase inhibitor if the molar ratio of NAD+:ADPR is greater than 4:1.

47. A method in accordance with claim 40, wherein the quantifying NAD+ in the lysate comprises performing a chemiluminescence assay.

48. A polypeptide consisting of a mutant or fragment of SARM1, wherein the mutant or fragment has constitutive NADase activity.

49. A polypeptide consisting of:
a mutant or fragment of SARM1, wherein the mutant or fragment has constitutive NADase activity; and
at least one tag.

50. A polypeptide in accordance with claim 49, wherein the at least one tag is selected from the group consisting of a Strep tag, a His tag, and a combination thereof.

51. A polypeptide in accordance with claim 49, wherein the mutant or fragment of SARM1 is a SARM1-TIR fragment.

52. A polypeptide in accordance with claim 49, consisting of a SARM1-TIR fragment, a His tag, and a streptavidin tag.

53. A polypeptide in accordance with claim 52, wherein the streptavidin tag is a tandem streptavidin tag.

54. A polypeptide in accordance with claim 49, consisting of, in amino-to-carboxy terminal order, a tandem streptavidin tag, a SARM1-TIR fragment, and a His tag.

55. A polypeptide in accordance with claim 49, wherein the mutant or fragment of SARM1 is a SARM1 polypeptide deleted for an N-terminal auto-inhibitory domain.

56. A polypeptide in accordance with claim 49, wherein the mutant or fragment of SARM1 consists of human SARM1 residues 410 to 721.

57. A polypeptide in accordance with claim 49, wherein the mutant or fragment of SARM1 consists of murine SARM1 residues homologous to human SARM1 residues 410 to 721.

58. A polypeptide having constitutive NADase activity and at least 70% sequence identity with a sequence of a fragment of human SARM1 that has constitutive NADase activity.

59. A polypeptide in accordance with claim 58, having at least 80% sequence identity with a sequence of a fragment of human SARM1 that has constitutive NADase activity.

60. A polypeptide in accordance with claim 58, having at least 90% sequence identity with a sequence of a fragment of human SARM1 that has constitutive NADase activity.

61. A polypeptide in accordance with claim 58, having at least 95% sequence identity with a sequence of a fragment of human SARM1 that has constitutive NADase activity.

62. A fragment of human SARM1 that has constitutive NADase activity.

63. A vector encoding the polypeptide of any one of claims 48-62.

64. A composition comprising:
the polypeptide of any one of claims 48-62; and a solid support.

65. A composition in accordance with claim 64, wherein the solid support is a bead.

66. A method of identifying a SARM1 NADase inhibitor, comprising:
a. providing a mixture comprising a solid support to which is bound i) a polypeptide in accordance with any one of claims 48-62 and at least one tag, ii) NAD+, and iii) a candidate inhibitor;
b. incubating the mixture;
c. quantifying the NAD+ after the incubating; and
d. identifying the candidate inhibitor compound as an NADase inhibitor if the concentration of NAD+ is greater than that of a control.

67. A method in accordance with claim 66, wherein the at least one tag is an N-terminal tag.

68. A method in accordance with claim 67, wherein the N-terminal tag is a streptavidin tag.

69. A method in accordance with claim 68, wherein the N-terminal protein tag is a tandem streptavidin tag.

70. A method in accordance with claim 66, wherein the at least one tag is a C-terminal tag.

71. A method in accordance with claim 70, wherein the C-terminal tag is a His tag.

72. A method in accordance with claim 66, wherein the solid support is a His tag purification bead.

73. A method in accordance with claim 66, wherein the at least one tag is at least two tags.

74. A method in accordance with claim 73, wherein the at least two tags are an N-terminal tag and a C-terminal tag.

75. A method in accordance with claim 74, wherein the N-terminal tag is a tandem streptavidin tag and the C-terminal tag is a His tag.

76. A method in accordance with claim 66, wherein the quantifying NAD+ comprises performing an HPLC assay.

77. A method of identifying a SARM1 NADase inhibitor, comprising:
a. providing a mixture comprising i) at least one cultured neuron comprising at least one axon and ii) a candidate SARM1 NADase inhibitor;
b. adding a labeled NAM to the mixture;
c. transecting the at least one axon;
d. quantifying the amount of labeled and unlabeled NAD+ in the mixture; and
e. identifying an inhibitor of SARM1 NADase when the post-injury NAD+ consumption rate is decreased compared to that of a control mixture that does not contain the candidate inhibitor.

78. A method of identifying a SARM1 NADase inhibitor in accordance with claim 77, further comprising determining the net rate of NAD+ consumption.

79. A method of identifying a SARM1 NADase inhibitor in accordance with claim 77, wherein the determining the net rate of NAD+ consumption comprises calculating the % decrease of light NAD+ over heavy NAD+ over time.

80. A method in accordance with claim 77, wherein the labeled NAM is deuterium labeled NAM.

81. A method in accordance with claim 77, wherein the labeled NAM is D4-NAM.

82. A method in accordance with claim 77, wherein the quantifying the labeled and unlabeled NAD+ comprises performing an HPLC assay.

83. A method in accordance with claim 77, wherein the at least one cultured neuron is at least one dorsal root ganglion cultured neuron.

84. A method of identifying an inhibitor of axonal degeneration, comprising:
a. providing a mixture comprising i) at least one cultured neuron comprising an axon and ii) a candidate inhibitor;
b. disrupting the neuron;
c. calculating a degeneration index using at least one microscope image; and
d. identifying an inhibitor of axon degeneration when there is a statistically significant decrease in the degeneration index compared to that of a control.

85. A method in accordance with claim 84, wherein the disrupting the neuron comprises transecting the axon.

86. A method in accordance with claim 84, wherein the disrupting the neuron comprises adding vincristine to the mixture.

87. A method of identifying a SARM1 NADase inhibitor, comprising:
a. providing a mixture comprising i) a mutant or fragment of SARM1, ii) NAD+ and iii) a candidate inhibitor, wherein the mutant or fragment has constitutive NADase activity;
b. incubating the mixture;
c. quantifying NAD+ in the mixture after the incubating; and
d. identifying the candidate inhibitor compound as an NADase inhibitor if the amount of NAD+ is greater than that of a control mixture that does not contain the candidate inhibitor.

88. A method in accordance with claim 87, wherein the quantifying NAD+ in the mixture comprises performing a chemiluminescence assay.

89. A method in accordance with claim 87, wherein the quantifying NAD+ in the mixture comprises performing an HPLC analysis.

90. A method in accordance with claim 87, wherein the mixture comprises a cell lysate comprising the mutant or fragment of SARM1.

91. A method in accordance with claim 87, wherein the cell lysate is a lysate of NRK1-HEK293T cells comprising the mutant or fragment of SARM1.

92. A method in accordance with claim 87, wherein the a mutant or fragment of SARM1 is a SAM-TIR fragment.

93. A method in accordance with claim 87, wherein the mutant or fragment of SARM1 consists of human SARM1 residues 410 to 721.

94. A method in accordance with claim 87, wherein the mutant or fragment of SARM1 consists of murine SARM1 modified to exhibit constitutive NADase activity.

95. A method in accordance with claim 87, wherein the mutant or fragment of SARM1 is a SARM1 polypeptide deleted for an N-terminal auto-inhibitory domain.

96. A method of treating an axonopathy, comprising administering a pharmaceutically effective amount of an inhibitor of SARM1 NADase activity.

97. A method of inhibiting SARM1 NADase activity, comprising contacting SARM1 with a SARM1 NADase inhibitor.

98. A cell in vitro comprising a polypeptide of any one of claims 48-62.

99. A cell in accordance with claim 98, wherein the cell is a eukaryotic cell.

100. A cell in accordance with claim 98, wherein the cell is a mammalian cell.

101. A cell in vitro comprising a nucleic acid encoding a polypeptide of any one of claims 48-62.

102. A cell in accordance with claim 101, wherein the cell is a eukaryotic cell.

103. A cell in accordance with claim 101, wherein the cell is a mammalian cell.

104. A prokaryotic cell comprising a polypeptide of any one of claims 48-62.

105. A prokaryotic cell of claim 104, wherein the cell is an E. coli.

106. A prokaryotic cell comprising a nucleic acid sequence encoding a polypeptide of any one of claims 48-62.

107. A prokaryotic cell of claim 106, wherein the cell is an E. coli.

108. A method of treating an axonopathy, comprising administering to a subject in need thereof a therapeutically effective amount of an inhibitor of SARM1 NADase activity.

109. A method of treating an axonopathy in accordance with claim 108, wherein the inhibitor of SARM1 NADase activity is a proton pump inhibitor.

110. A method of treating an axonopathy in accordance with claim 108, wherein the inhibitor of SARM1 NADase activity is a compound of formula I^{C}: 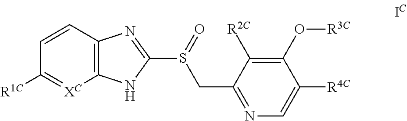
or a pharmaceutically acceptable salt thereof,
wherein:
X^{C} is N or C;
R^{1C} is H, C₁-C₅ alkyl, C₁-C₅ alkoxy, or C₁-C₅ haloalkoxy;
R^{2C} is C₁-C₅ alkyl or C₁-C₅ alkoxy;
R^{3C} is C₁-C₁₀ alkyl, C₁-C₁₀ haloalkyl or an ether; and
R^{4C} is H, C₁-C₅ alkyl or C₁-C₅ alkoxy.

111. A method of treating an axonopathy in accordance with claim 108, wherein the inhibitor of SARM1 NADase activity is selected from the group consisting of

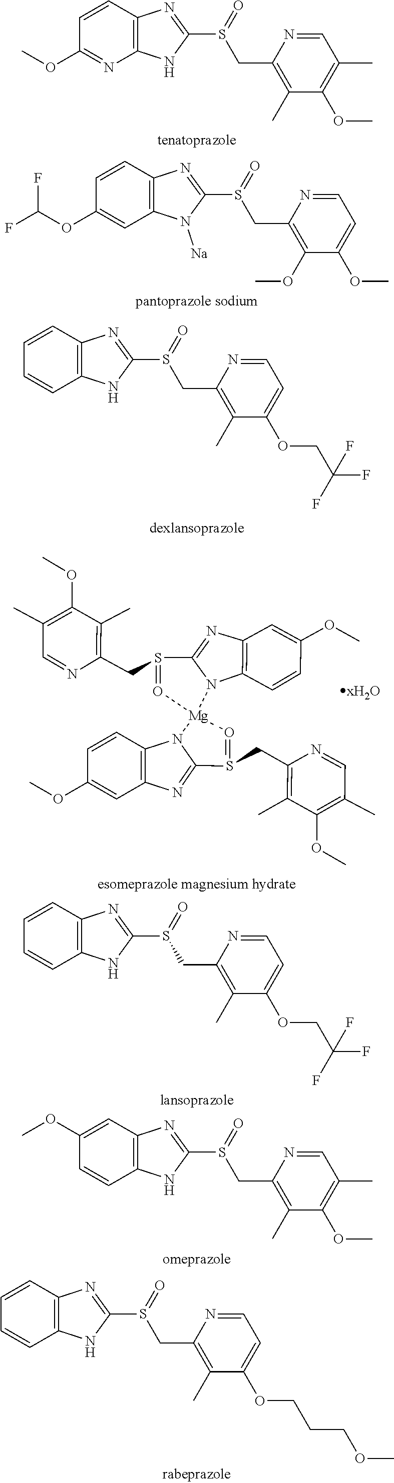

112. A method of treating an axonopathy in accordance with claim 108, wherein the inhibitor of SARM1 NADase activity is selected from the group consisting of:

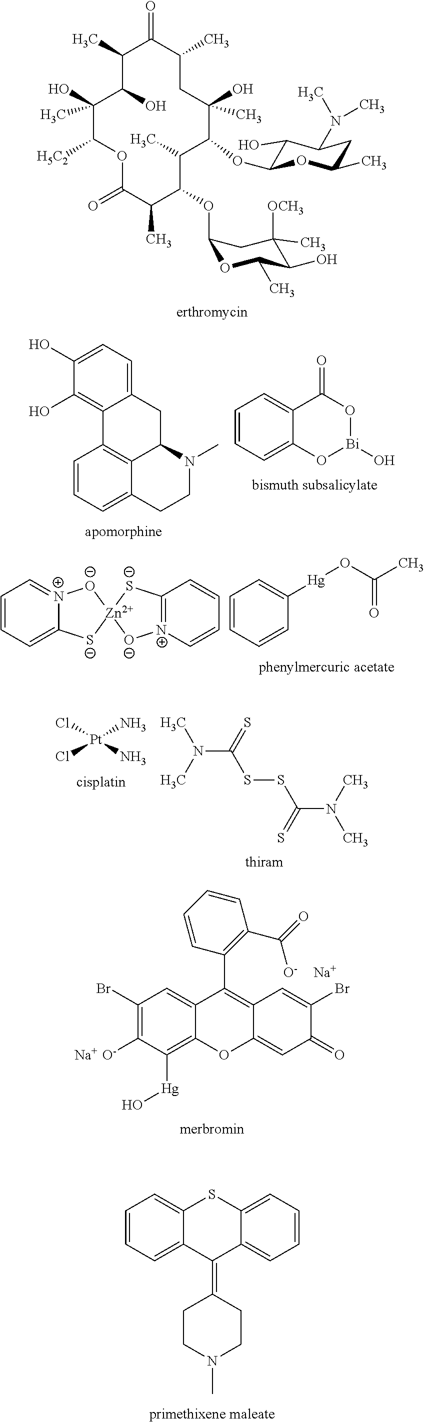

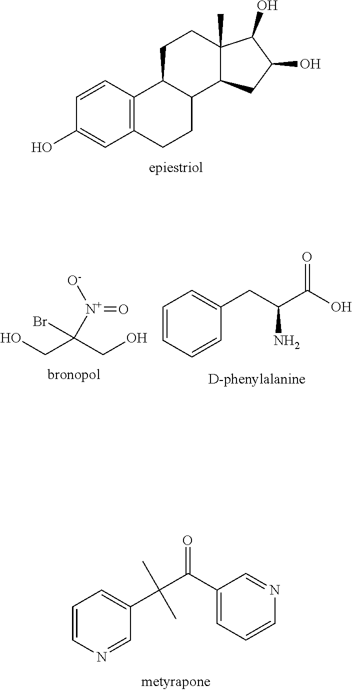

113. A method of treating an axonopathy, comprising administering to a subject in need thereof a therapeutically effective amount of Nicotinamide Hypoxanthine dinucleotide (NHD).

114. A method of identifying a SARM1 NADase inhibitor, comprising:
a) providing a mixture comprising i) a polypeptide that has at least 70% sequence identity with a fragment of human SARM1 that has constitutive NADase activity, ii) NAD+ and iii) a candidate inhibitor, wherein the polypeptide has constitutive NADase activity;
b) incubating the mixture;
c) quantifying NAD+ and at least one NADase cleavage product in the mixture after the incubating; and
d) identifying the candidate inhibitor compound as an NADase inhibitor if the molar ratio of NAD+ to the at least one NADase cleavage product is greater than that of a control mixture that does not contain the candidate inhibitor.

115. A method in accordance with claim 113, wherein the polypeptide has at least 80% sequence identity with a fragment of human SARM1 that has constitutive NADase activity.

116. A method in accordance with claim 113, wherein the polypeptide has at least 90% sequence identity with a fragment of human SARM1 that has constitutive NADase activity.

117. A method in accordance with claim 113, wherein the polypeptide has at least 95% sequence identity with a fragment of human SARM1 that has constitutive NADase activity.

118. A method in accordance with claim 113, wherein the polypeptide is a fragment of human SARM1 that has constitutive NADase activity.

119. A method in accordance with any one of claims 113-117, wherein the at least one NADase cleavage product is ADPR.

120. A method in accordance with any one of claims 113-117, wherein the at least one NADase cleavage product is Nam.

121. A method in accordance with any one of claims 113-117, wherein the quantifying NAD+ comprises performing an HPLC analysis.

122. A method in accordance with any one of claims 113-117, wherein the quantifying the at least one NADase cleavage product comprises performing an HPLC analysis.

123. A method in accordance with any one of claims 113-117, wherein the quantifying NAD+ in the lysate comprises performing a chemiluminescence assay.

124. A method in accordance with any one of claims 113-117, wherein the mixture comprises a cell lysate comprising the polypeptide.

125. A method in accordance with any one of claims 113-117, wherein cell lysate is a lysate of NRK1-HEK293T cells comprising the polypeptide.

126. A method in accordance with claim 113, wherein the polypeptide is a SARM-TIR fragment.

127. A method in accordance with claim 113, wherein the polypeptide consists of human SARM1 residues 410 to 721.

128. A method in accordance with claim 113, wherein the polypeptide consists of human SARM1 residues 560-724.

129. A method in accordance with claim 113, wherein the polypeptide is a SARM1 polypeptide deleted for an N-terminal auto-inhibitory domain.

130. A method in accordance with claim 113, wherein the candidate inhibitor compound is identified as an NADase inhibitor if the molar ratio of NAD+ to the at least one cleavage product is greater than 4:1.

131. A method of inhibiting SARM1 NADase activity comprising contacting a SARM1 polypeptide with a proton pump inhibitor.

132. A method of inhibiting SARM1 NADase activity comprising contacting a SARM1 polypeptide with a compound of formula I^{C}: 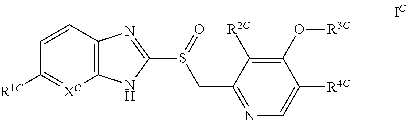
or a pharmaceutically acceptable salt thereof,
wherein:
X^{C} is N or C;
R^{1C} is H, C₁-C₅ alkyl, C₁-C₅ alkoxy, or C₁-C₅ haloalkoxy;
R^{2C} is C₁-C₅ alkyl or C₁-C₅ alkoxy;
R^{3C} is C₁-C₁₀ alkyl, C₁-C₁₀ haloalkyl or an ether; and
R^{4C} is H, C₁-C₅ alkyl or C₁-C₅ alkoxy.

133. A method of inhibiting SARM1 NADase activity in accordance with claim 108, wherein the compound or salt thereof is selected from the group consisting of tenatoprazole, pantoprazole sodium, dexlansoprazole, esomeprazole magnesium hydrate and rabeprazole sodium.

134. A compound of formula I^{C}, or a pharmaceutically acceptable salt thereof, for use in the treatment of an axonopathy: 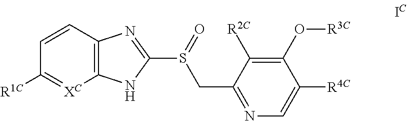
wherein:
X^{C} is N or C;
R^{1C} is H, C₁-C₅ alkyl, C₁-C₅ alkoxy, or C₁-C₅ haloalkoxy;
R^{2C} is C₁-C₅ alkyl or C₁-C₅ alkoxy;
R^{3C} is C₁-C₁₀ alkyl, C₁-C₁₀ haloalkyl or an ether; and
R^{4C} is H, C₁-C₅ alkyl or C₁-C₅ alkoxy.

135. A compound in accordance with claim 110, wherein the compound or salt thereof is selected from the group consisting of tenatoprazole, pantoprazole sodium, dexlansoprazole, esomeprazole magnesium hydrate, lansoprazole, omeprazole or rabeprazole sodium.

136. A method of identifying a SARM1 NADase inhibitor, comprising:
a. providing a mixture comprising i) a full-length SARM1, ii) NAD+ and iii) a candidate inhibitor, wherein the full-length SARM1 has constitutive NADase activity;
b. incubating the mixture;
c. quantifying NAD+ and ADPR in the mixture after the incubating;
d. determining the molar ratio of NAD+:ADPR; and
e. identifying the candidate inhibitor compound as an NADase inhibitor if the molar ratio is greater than that of a control mixture that does not contain the candidate inhibitor.

137. The method of claim 136, wherein the quantifying NAD+ and ADPR in the mixture comprises performing an HPLC analysis.

138. The method of claim 136, wherein the mixture comprises a cell lysate comprising the full-length SARM1.

139. A method of identifying a SARM1 NADase inhibitor, comprising:
a. providing a mixture comprising a solid support to which is bound i) a full-length SARM1 and at least one tag, ii) NAD+, and iii) a candidate inhibitor;
b. incubating the mixture;
c. quantifying the NAD+ after the incubating; and
d. identifying the candidate inhibitor compound as an NADase inhibitor if the concentration of NAD+ is greater than that of a control.

140. A method of identifying a SARM1 NADase inhibitor, comprising:
a. providing a mixture comprising i) a full-length SARM1, ii) NAD+ and iii) a candidate inhibitor, wherein the full-length SARM1 has constitutive NADase activity;
b. incubating the mixture;
c. quantifying NAD+ in the mixture after the incubating; and
d. identifying the candidate inhibitor compound as an NADase inhibitor if the amount of NAD+ is greater than that of a control mixture that does not contain the candidate inhibitor.

141. A method of identifying a SARM1 NADase inhibitor, comprising:
a) providing a mixture comprising i) a full-length SARM1 that has constitutive NADase activity, ii) NAD+ and iii) a candidate inhibitor, wherein the full-length SARM1 has constitutive NADase activity;
b) incubating the mixture;
c) quantifying NAD+ and at least one NADase cleavage product in the mixture after the incubating; and
d) identifying the candidate inhibitor compound as an NADase inhibitor if the molar ratio of NAD+ to the at least one NADase cleavage product is greater than that of a control mixture that does not contain the candidate inhibitor.

142. The method of claim 141, wherein the at least one NADase cleavage product is ADPR.

143. The method of claim 141, wherein the at least one NADase cleavage product is Nam.

144. The method of claim 141, wherein the quantifying NAD+ comprises performing an HPLC analysis.

145. A SARM1 polypeptide comprising at least a functional fragment of a SARM1 N-terminal auto-inhibitory domain, at least a functional fragment of one or more SAM domains, and at least a functional fragment of a SARM1 TIR domain, wherein the SARM1 polypeptide lacks a mitochondrial targeting sequence.


SEQUENCE LISTING (16 sequences)

SEQ ID NO: 1 - protein, 316 aa, Homo sapiens
VPSWKEAEVQTWLQQIGFSKYCESFREQQVDGDLLLRLTEEELQTDLGMKSGITRKRFFRELTELKTFANYSTCDRSNLADWLGSLDPRFRQYTYGLVSCGLDRSLLHRVSEQQLLEDCGIHLGVHRARILTAAREMLHSPLPCTGGKPSGDTPDVFISYRRNSGSQLASLLKVHLQLHGFSVFIDVEKLEAGKFEDKLIQSVMGARNFVLVLSPGALDKCMQDHDCKDWVHKEIVTALSCGKNIVPIIDGFEWPEPQVLPEDMQAVLTFNGIKWSHEYQEATIEKIIRFLQGRSSRDSSAGSDTSLEGAAPMGPT

SEQ ID NO: 2 - protein, 164 aa, Homo sapiens
TPDVFISYRRNSGSQLASLLKVHLQLHGFSVFIDVEKLEAGKFEDKLIQSVMGARNFVLVLSPGALDKCMQDHDCKDWVHKEIVTALSCGKNIVPIIDGFEWPEPQVLPEDMQAVLTFNGIKWSHEYQEATIEKIIRFLQGRSSRDSSAGSDTSLEGAAPMGPT

SEQ ID NO: 3 - protein, 164 aa, Mus sp.
TPDVFISYRRNSGSQLASLLKVHLQLHGFSVFIDVEKLEAGKFEDKLIQSVIAARNFVLVLSAGALDKCMQDHDCKDWVHKEIVTALSCGKNIVPIIDGFEWPEPQALPEDMQAVLTFNGIKWSHEYQEATIEKIIRFLQGRPSQDSSAGSDTSLEGATPMGLP

SEQ ID NO: 4 - protein, 160 aa, Danio rerio
PDVFISYRRTTGSQLASLLKVHLQLRGFSVFIDVEKLEAGRFEEKLITSVQRARNFILVLSANALDKCMGDVAMKDWVHKEIVTALNGKKNIVPVTDNFVWPDPTSLPEDMSTILKFNGIKWSHEYQEATIEKILRFLEGCPSQEKPDGAKTDKKEPQKK

SEQ ID NO: 5 - protein, 8 aa, synthetic construct
  REGION 1..8: note = source = /note="Description of Artificial Sequence: Synthetic peptide"
WSHPQFEK

SEQ ID NO: 6 - protein, 195 aa, synthetic construct
  REGION 1..195: note = source = /note="Description of Artificial Sequence: Synthetic polypeptide"
MKRFVIGIGGVTNGGKTTLAKSLQKHLPNCSVISQDDFFKPESEIDIDENGFLQYDVLEALNMEKMMSAVSCWMENPGSSAGPAALESAQGVPILIIEGFLLFNYKPLDTIWNRSYFLTVPYEECKRRRSTRVYEPPDPPGYFDGHVWPMYLKHRQEMSSITWDIVYLDGTRSEEDLFSQVYEDVKQELEKQNGL

SEQ ID NO: 7 - DNA, 10809 bp, synthetic construct
  misc_feature 1..10809: note = source = /note="Description of Artificial Sequence: Synthetic polynucleotide"
  source 1..10809: mol_type = other DNA
gtcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagcgcgttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatcggcactgcgtgcgccaattctgcagacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggttaattaagggtgcagcggcctccgcgccgggttttggcgcctcccgcgggcgcccccctcctcacggcgagcgctgccacgtcagacgaagggcgcaggagcgttcctgatccttccgcccggacgctcaggacagcggcccgctgctcataagactcggccttagaaccccagtatcagcagaaggacattttaggacgggacttgggtgactctagggcactggttttctttccagagagcggaacaggcgaggaaaagtagtcccttctcggcgattctgcggagggatctccgtggggcggtgaacgccgatgattatataaggacgcgccgggtgtggcacagctagttccgtcgcagccgggatttgggtcgcggttcttgtttgtggatcgctgtgatcgtcacttggtgagttgcgggctgctgggctggccggggctttcgtggccgccgggccgctcggtgggacggaagcgtgtggagagaccgccaagggctgtagtctgggtccgcgagcaaggttgccctgaactgggggttggggggagcgcacaaaatggcggctgttcccgagtcttgaatggaagacgcttgtaaggcgggctgtgaggtcgttgaaacaaggtggggggcatggtgggcggcaagaacccaaggtcttgaggccttcgctaatgcgggaaagctcttattcgggtgagatgggctggggcaccatctggggaccctgacgtgaagtttgtcactgactggagaactcgggtttgtcgtctggttgcgggggcggcagttatgcggtgccgttgggcagtgcacccgtacctttgggagcgcgcgcctcgtcgtgtcgtgacgtcacccgttctgttggcttataatgcagggtggggccacctgccggtaggtgtgcggtaggcttttctccgtcgcaggacgcagggttcgggcctagggtaggctctcctgaatcgacaggcgccggacctctggtgaggggagggataagtgaggcgtcagtttctttggtcggttttatgtacctatcttcttaagtagctgaagctccggttttgaactatgcgctcggggttggcgagtgtgttttgtgaagttttttaggcaccttttgaaatgtaatcatttgggtcaatatgtaattttcagtgttagactagtaaagcttctgcaggtcgactctagaaaattgtccgctaaattctggccgtttttggcttttttgttagacgaagcttgggctgcaggtcgactctagaggatcatgaagagatttgtcattggaattggtggtgtgacaaacggagggaagacgacactggctaagagcttgcagaagcaccttcccaactgcagcgtcatatctcaggatgacttcttcaagccagagtctgagatagacatagatgaaaatggttttttgcagtatgatgtgcttgaagcgctaaatatggaaaaaatgatgtcagcagtttcctgttggatggaaaacccaggaagctctgcgggaccagcagccttggaaagtgctcaaggggttcccattttaattattgaaggtttccttctctttaattataagcctctggacaccatatggaacagaagttacttcctgaccgttccatatgaagaatgtaagaggagaaggagtaccagagtatatgagcctccagaccctccagggtacttcgatggccacgtgtggcccatgtacctaaagcacagacaggaaatgagctccatcacctgggacattgtttacctggatggaacaaggtctgaagaggacctcttctctcaggtgtatgaagatgtcaagcaggaactagagaagcaaaatggtttggactataaagatgatgatgataagtaagctagctaccggtgatccgcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataatatggccacaaccatggatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgagaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgacctcgagacctagaaaaacatggagcaatcacaagtagcaatacagcagctaccaatgctgattgtgcctggctagaagcacaagaggaggaggaggtgggttttccagtcacacctcaggtacctttaagaccaatgacttacaaggcagctgtagatcttagccactttttaaaagaaaaggggggactggaagggctaattcactcccaacgaagacaagatatccttgatctgtggatctaccacacacaaggctacttccctgattggcagaactacacaccagggccagggatcagatatccactgacctttggatggtgctacaagctagtaccagttgagcaagagaaggtagaagaagccaatgaaggagagaacacccgcttgttacaccctgtgagcctgcatgggatggatgacccggagagagaagtattagagtggaggtttgacagccgcctagcatttcatcacatggcccgagagctgcatccggactgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgttgacaattaatcatcggcatagtatatcggcatagtataatacgacaaggtgaggaactaaaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgacacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgac

SEQ ID NO: 8 - protein, 365 aa, synthetic construct
  REGION 1..365: note = source = /note="Description of Artificial Sequence: Synthetic polypeptide"
MSAWSHPQFEKGGGSGGGSGGSAWSHPQFEKGGGSSGGGGGGSSGGGASVPSWKEAEVQTWLQQIGFSKYCESFREQQVDGDLLLRLTEEELQTDLGMKSGITRKRFFRELTELKTFANYSTCDRSNLADWLGSLDPRFRQYTYGLVSCGLDRSLLHRVSEQQLLEDCGIHLGVHRARILTAAREMLHSPLPCTGGKPSGDTPDVFISYRRNSGSQLASLLKVHLQLHGFSVFIDVEKLEAGKFEDKLIQSVMGARNFVLVLSPGALDKCMQDHDCKDWVHKEIVTALSCGKNIVPIIDGFEWPEPQVLPEDMQAVLTFNGIKWSHEYQEATIEKIIRFLQGRSSRDSSAGSDTSLEGAAPMGPT

SEQ ID NO: 9 - DNA, 10329 bp, synthetic construct
  misc_feature 1..10329: note = source = /note="Description of Artificial Sequence: Synthetic polynucleotide"
  source 1..10329: mol_type = other DNA
gtcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagcgcgttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatcggcactgcgtgcgccaattctgcagacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggttaattaagggtgcagcggcctccgcgccgggttttggcgcctcccgcgggcgcccccctcctcacggcgagcgctgccacgtcagacgaagggcgcaggagcgttcctgatccttccgcccggacgctcaggacagcggcccgctgctcataagactcggccttagaaccccagtatcagcagaaggacattttaggacgggacttgggtgactctagggcactggttttctttccagagagcggaacaggcgaggaaaagtagtcccttctcggcgattctgcggagggatctccgtggggcggtgaacgccgatgattatataaggacgcgccgggtgtggcacagctagttccgtcgcagccgggatttgggtcgcggttcttgtttgtggatcgctgtgatcgtcacttggtgagttgcgggctgctgggctggccggggctttcgtggccgccgggccgctcggtgggacggaagcgtgtggagagaccgccaagggctgtagtctgggtccgcgagcaaggttgccctgaactgggggttggggggagcgcacaaaatggcggctgttcccgagtcttgaatggaagacgcttgtaaggcgggctgtgaggtcgttgaaacaaggtggggggcatggtgggcggcaagaacccaaggtcttgaggccttcgctaatgcgggaaagctcttattcgggtgagatgggctggggcaccatctggggaccctgacgtgaagtttgtcactgactggagaactcgggtttgtcgtctggttgcgggggcggcagttatgcggtgccgttgggcagtgcacccgtacctttgggagcgcgcgcctcgtcgtgtcgtgacgtcacccgttctgttggcttataatgcagggtggggccacctgccggtaggtgtgcggtaggcttttctccgtcgcaggacgcagggttcgggcctagggtaggctctcctgaatcgacaggcgccggacctctggtgaggggagggataagtgaggcgtcagtttctttggtcggttttatgtacctatcttcttaagtagctgaagctccggttttgaactatgcgctcggggttggcgagtgtgttttgtgaagttttttaggcaccttttgaaatgtaatcatttgggtcaatatgtaattttcagtgttagactagtaaagcttctgcaggtcgactctagaaaattgtccgctaaattctggccgtttttggcttttttgttagacgaagcttgggctgcaggtcgactctagaggatccggatccgccaccatgtcagcttggagccacccacaattcgaaaaaggcggtggctcaggcggtggctcaggtggctcagcttggagccacccacaattcgaaaaaggcggtggctcatctggcggaggtggcggtggctcatctggcggaggtgctagcgtgcccagctggaaggaggccgaggttcagacgtggctgcagcagatcggtttctccaagtactgcgagagcttccgggagcagcaggtggatggcgacctgcttctgcggctcacggaggaggaactccagaccgacctgggcatgaaatcgggcatcacccgcaagaggttctttagggagctcacggagctcaagaccttcgccaactattctacgtgcgaccgcagcaacctggcggactggctgggcagcctggacccgcgcttccgccagtacacctacggcctggtcagctgcggcctggaccgctccctgctgcaccgcgtgtctgagcagcagctgctggaagactgcggcatccacctgggcgtgcaccgcgcccgcatcctcacggcggccagagaaatgctacactccccgctgccctgtactggtggcaaacccagtggggacactccagatgtcttcatcagctaccgccggaactcaggttcccagctggccagtctcctgaaggtgcacctgcagctgcatggcttcagtgtcttcattgatgtggagaagctggaagcaggcaagttcgaggacaaactcatccagagtgtcatgggtgcccgcaactttgtgttggtgctatcacctggagcactggacaagtgcatgcaagaccatgactgcaaggattgggtgcataaggagattgtgactgctttaagctgcggcaagaacattgtgcccatcattgatggcttcgagtggcctgagccccaggtcctgcctgaggacatgcaggctgtgcttactttcaacggtatcaagtggtcccacgaataccaggaggccaccattgagaagatcatccgcttcctgcagggccgctcctcccgggactcatctgcaggctctgacaccagtttggagggtgctgcacccatgggtccaacctaaactctagaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgacctcgagacctagaaaaacatggagcaatcacaagtagcaatacagcagctaccaatgctgattgtgcctggctagaagcacaagaggaggaggaggtgggttttccagtcacacctcaggtacctttaagaccaatgacttacaaggcagctgtagatcttagccactttttaaaagaaaaggggggactggaagggctaattcactcccaacgaagacaagatatccttgatctgtggatctaccacacacaaggctacttccctgattggcagaactacacaccagggccagggatcagatatccactgacctttggatggtgctacaagctagtaccagttgagcaagagaaggtagaagaagccaatgaaggagagaacacccgcttgttacaccctgtgagcctgcatgggatggatgacccggagagagaagtattagagtggaggtttgacagccgcctagcatttcatcacatggcccgagagctgcatccggactgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgttgacaattaatcatcggcatagtatatcggcatagtataatacgacaaggtgaggaactaaaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgacacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgac

SEQ ID NO: 10 - protein, 211 aa, synthetic construct
  REGION 1..211: note = source = /note="Description of Artificial Sequence: Synthetic polypeptide"
MSAWSHPQFEKGGGSGGGSGGSAWSHPQFEKGGGSSGGGASTPDVFISYRRNSGSQLASLLKVHLQLHGFSVFIDVEKLEAGKFEDKLIQSVMGARNFVLVLSPGALDKCMQDHDCKDWVHKEIVTALSCGKNIVPIIDGFEWPEPQVLPEDMQAVLTFNGIKWSHEYQEATIEKIIRFLQGRSSRDSSAGSDTSLEGAAPMGPTHHHHHH

SEQ ID NO: 11 - protein, 211 aa, synthetic construct
  REGION 1..211: note = source = /note="Description of Artificial Sequence: Synthetic polypeptide"
MSAWSHPQFEKGGGSGGGSGGSAWSHPQFEKGGGSSGGGASTPDVFISYRRNSGSQLASLLKVHLQLHGFSVFIDVEKLEAGKFEDKLIQSVIAARNFVLVLSAGALDKCMQDHDCKDWVHKEIVTALSCGKNIVPIIDGFEWPEPQALPEDMQAVLTFNGIKWSHEYQEATIEKIIRFLQGRPSQDSSAGSDTSLEGATPMGLPHHHHHH

SEQ ID NO: 12 - protein, 207 aa, synthetic construct
  REGION 1..207: note = source = /note="Description of Artificial Sequence: Synthetic polypeptide"
MSAWSHPQFEKGGGSGGGSGGSAWSHPQFEKGGGSSGGGASPDVFISYRRTTGSQLASLLKVHLQLRGFSVFIDVEKLEAGRFEEKLITSVQRARNFILVLSANALDKCMGDVAMKDWVHKEIVTALNGKKNIVPVTDNFVWPDPTSLPEDMSTILKFNGIKWSHEYQEATIEKILRFLEGCPSQEKPDGAKTDKKEPQKKHHHHHH

SEQ ID NO: 13 - protein, 724 aa, Homo sapiens
MVLTLLLSAYKLCRFFAMSGPRPGAERLAVPGPDGGGGTGPWWAAGGRGPREVSPGAGTEVQDALERALPELQQALSALKQAGGARAVGAGLAEVFQLVEEAWLLPAVGREVAQGLCDAIRLDGGLDLLLRLLQAPELETRVQAARLLEQILVAENRDRVARIGLGVILNLAKEREPVELARSVAGILEHMFKHSEETCQRLVAAGGLDAVLYWCRRTDPALLRHCALALGNCALHGGQAVQRRMVEKRAAEWLFPLAFSKEDELLRLHACLAVAVLATNKEVEREVERSGTLALVEPLVASLDPGRFARCLVDASDTSQGRGPDDLQRLVPLLDSNRLEAQCIGAFYLCAEAAIKSLQGKTKVFSDIGAIQSLKRLVSYSTNGTKSALAKRALRLLGEEVPRPILPSVPSWKEAEVQTWLQQIGFSKYCESFREQQVDGDLLLRLTEEELQTDLGMKSGITRKRFFRELTELKTFANYSTCDRSNLADWLGSLDPRFRQYTYGLVSCGLDRSLLHRVSEQQLLEDCGIHLGVHRARILTAAREMLHSPLPCTGGKPSGDTPDVFISYRRNSGSQLASLLKVHLQLHGFSVFIDVEKLEAGKFEDKLIQSVMGARNFVLVLSPGALDKCMQDHDCKDWVHKEIVTALSCGKNIVPIIDGFEWPEPQVLPEDMQAVLTFNGIKWSHEYQEATIEKIIRFLQGRSSRDSSAGSDTSLEGAAPMGPT

SEQ ID NO: 14 - DNA, 2193 bp, Homo sapiens
  source 1..2193: mol_type = other DNA
atggtcctgacgctgcttctctccgcctacaagctgtgtcgcttcttcgccatgtcgggcccacggccgggcgccgagcggctggcggtgcctgggccagatgggggcggtggcacgggcccatggtgggctgcgggtggccgcgggccccgcgaagtgtcgccgggggcaggcaccgaggtgcaggacgccctggagcgcgcgctgccggagctgcagcaggccttgtccgcgctgaagcaggcgggcggcgcgcgggccgtgggcgccggcctggccgaggtcttccaactggtggaggaggcctggctgctgccggccgtgggccgcgaggtagcccagggtctgtgcgacgccatccgcctcgatggcggcctcgacctgctgttgcggctgctgcaggcgccggagttggagacgcgtgtgcaggccgcgcgcctgctggagcagatcctggtggctgagaaccgagaccgcgtggcgcgcattgggctgggcgtgatcctgaacctggcgaaggaacgcgaacccgtagagctggcgcggagcgtggcaggcatcttggagcacatgttcaagcattcggaggagacatgccagaggctggtggcggccggcggcctggacgcggtgctgtattggtgccgccgcacggaccccgcgctgctgcgccactgcgcgctggcgctgggcaactgcgcgctgcacgggggccaggcggtgcagcgacgcatggtagagaagcgcgcagccgagtggctcttcccgctcgccttctccaaggaggacgagctgcttcggctgcacgcctgcctcgcagtagcggtgttggcgactaacaaggaggtggagcgcgaggtggagcgctcgggcacgctggcgctcgtggagccgcttgtggcctcgctggaccctggccgcttcgcccgctgtctggtggacgccagcgacacaagccagggccgcgggcccgacgacctgcagcgcctcgtgccgttgctcgactctaaccgcttggaggcgcagtgcatcggggctttctacctctgcgccgaggctgccatcaagagcctgcaaggcaagaccaaggtgttcagcgacatcggcgccatccagagcctgaaacgcctggtttcctactctaccaatggcactaagtcggcgctggccaagcgcgcgctgcgcctgctgggcgaggaggtgccacggcccatcctgccctccgtgcccagctggaaggaggccgaggttcagacgtggctgcagcagatcggtttctccaagtactgcgagagcttccgggagcagcaggtggatggcgacctgcttctgcggctcacggaggaggaactccagaccgacctgggcatgaaatcgggcatcacccgcaagaggttctttagggagctcacggagctcaagaccttcgccaactattctacgtgcgaccgcagcaacctggcggactggctgggcagcctggacccgcgcttccgccagtacacctacggcctggtcagctgcggcctggaccgctccctgctgcaccgcgtgtctgagcagcagctgctggaagactgcggcatccacctgggcgtgcaccgcgcccgcatcctcacggcggccagagaaatgctacactccccgctgccctgtactggtggcaaacccagtggggacactccagatgtcttcatcagctaccgccggaactcaggttcccagctggccagtctcctgaaggtgcacctgcagctgcatggcttcagtgtcttcattgatgtggagaagctggaagcaggcaagttcgaggacaaactcatccagagtgtcatgggtgcccgcaactttgtgttggtgctatcacctggagcactggacaagtgcatgcaagaccatgactgcaaggattgggtgcataaggagattgtgactgctttaagctgcggcaagaacattgtgcccatcattgatggcttcgagtggcctgagccccaggtcctgcctgaggacatgcaggctgtgcttactttcaacggtatcaagtggtcccacgaataccaggaggccaccattgagaagatcatccgcttcctgcagggccgctcctcccgggactcatctgcaggctctgacaccagtttggagggtgctgcacccatgggtccaacctaaccagtccccagttcccca

SEQ ID NO: 15 - protein, 166 aa, synthetic construct
  REGION 1..166: note = source = /note="Description of Artificial Sequence: Synthetic polypeptide"
GDTPDVFISYRRNSGSQLASLLKVHLQLHGFSVFIDVEKLEAGKFEDKLIQSVMGARNFVLVLSPGALDKCMQDHDCKDWVHKEIVTALSCGKNIVPIIDGFEWPEPQVLPEDMQAVLTFNGIKWSHEYQEATIEKIIRFLQGRSSRDSSAGSDTSLEGAAPMGPT

SEQ ID NO: 16 - protein, 154 aa, synthetic construct
  REGION 1..154: note = source = /note="Description of Artificial Sequence: Synthetic polypeptide"
VGSVFLGGPFRQLVDPRTGVMSSGDQNVFSRLIEHFESRGTTVYNAHRREAWGAEFLSPAEATRLDHDEIKAADVFVAFPGVPASPGTHVEIGWASGMGKPMVLLLERDEDYAFLVTGLESQANVEILRFSGTEEIVERLDGAVARVLGRAGEP